(19) United States
(12) Patent Application Publication
(10) Pub. No.: US 2025/0197413 A1
(43) Pub. Date: Jun. 19, 2025

(54) RXFP1 RECEPTOR AGONISTS

(71) Applicant: ELI LILLY AND COMPANY, Indianapolis, IN (US)

(72) Inventors: Erin ANDERSON, Boulder, CO (US); Todd FIELDS, New Palestine, IN (US); Autumn Rose FLYNN, Erie, CO (US); Megan MCKENNEY HALL, Boulder, CO (US); Richard Duane JOHNSTON, Greenfield, IN (US); Jason Eric LAMAR, Indianapolis, IN (US); Christopher MAYNE, Boulder, CO (US); Elizabeth MCFADDIN, Nederland, CO (US); Andrew METCALF, Erie, CO (US); Tarek SAMMAKIA, Boulder, CO (US); Zhicheng SUN, Thornton, CO (US)

(21) Appl. No.: 18/979,982

(22) Filed: Dec. 13, 2024

Related U.S. Application Data
(60) Provisional application No. 63/662,768, filed on Jun. 21, 2024.
(60) Provisional application No. 63/610,493, filed on Dec. 15, 2023.

(51) Int. Cl. C07D 491/08; A61K 31/428; A61K 31/4355; A61K 31/438; A61K 31/439; A61K 31/4439; A61K 31/444; A61K 31/501; A61K 31/5386; A61K 31/553; C07D 277/68; C07D 417/04; C07D 491/048; C07D 491/052; C07D 491/107; C07D 495/08; C07D 495/10; C07D 498/08; C07D 519/00

(52) U.S. Cl. CPC C07D 491/08; A61K 31/428; A61K 31/4355; A61K 31/438; A61K 31/439; A61K 31/4439; A61K 31/444; A61K 31/501; A61K 31/5386; A61K 31/553; C07D 277/68; C07D 417/04; C07D 491/048; C07D 491/052; C07D 491/107; C07D 495/08; C07D 495/10; C07D 498/08; C07D 519/00

(57) ABSTRACT

The present invention provides compounds of the formula:

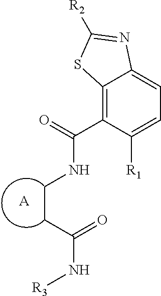

wherein Ring A, R₁, R₂, and R₃ are as described herein, pharmaceutically acceptable salts thereof, and methods of using these compounds and pharmaceutically acceptable salts thereof for treating patients for cardiovascular, pulmonary and/or renal conditions, diseases and/or disorders.

### FIELD OF THE INVENTION

The present invention relates to small molecule RXFP1 agonists and uses thereof.

### BACKGROUND OF THE INVENTION

Human relaxin-2 (relaxin) is an insulin-like peptide known to regulate cardiovascular, renal, and pulmonary adaptations during pregnancy. Preclinical studies and recent clinical trials with a short-acting recombinant relaxin, serelaxin, have shown the promise of recombinant relaxin as a therapeutic agent in the treatment of cardiovascular and fibrotic diseases.

However, relaxin has several limitations such as poor oral bioavailability, a short half-life, high costs of production and storage. Similarly, peptide agonists of relaxin family peptide receptor-1 (RXFP1) require subcutaneous/intravenous administration.

Non-peptidic, small molecule modulators of RXFP1 also have been sought. For example, McBride A et al. (2017) Scientific Reports 7:10806 discusses small-molecule positive allosteric modulators of RXFP1. WO2023/076626, WO2023/077040, and WO2023/077070 each disclose small molecule analogs useful as RXFP1 receptor agonists.

There remains a need to provide alternative, small molecule RXFP1 agonists. In particular, there is a need to provide additional orally deliverable RXFP1 agonists that are useful for treating cardiovascular, pulmonary and/or renal conditions, diseases and/or disorders. Further, there is a desire to provide RXFP1 receptor agonists that exhibit better pharmacokinetic/pharmacodynamic properties. Also, there is a need to provide RXFP1 receptor agonists that exhibit efficacy with reduced or minimized untoward or undesired effects. More particularly, there is a need to provide small molecule RXFP1 receptor agonists which are selective for RXFP1 over RXFP2. The present invention addresses one or more of these needs by providing novel small molecule RXFP1 agonists.

### SUMMARY OF THE INVENTION

Disclosed herein are the compounds of Formula I

A compound of the formula:

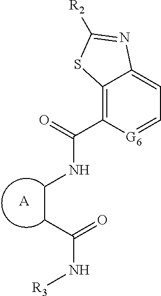

wherein:
Ring A is a group of the formula

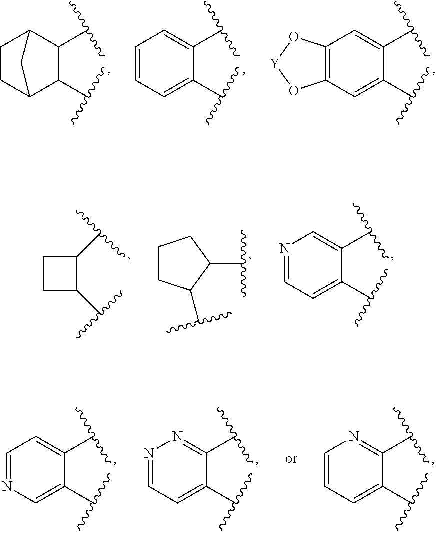

wherein each of

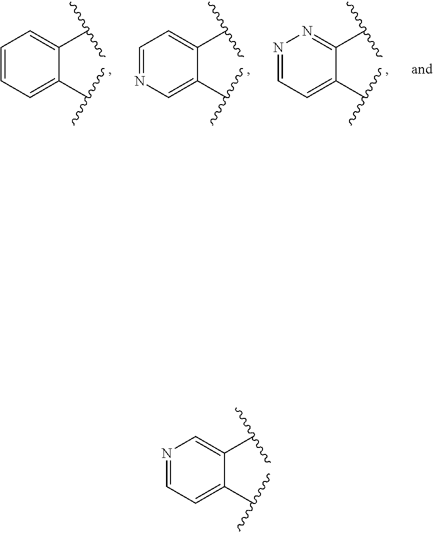 [SMILES: *c1ccccc1* ; *c1ccncc1* ; *c1ccnnc1* ; *c1ccncc1*]

is optionally substituted by one or more substituents selected from halogens, —CN, C₁₋₄ alkyl, —S—C₁₋₃ alkyl, and C₁₋₃ alkoxy, wherein each C₁₋₄ alkyl and C₁₋₃ alkoxy is independently optionally substituted by one or more substituents selected from halogens and —CN;
—Y— is a C₁₋₃ alkylene;
—G₆— is —C(R₁)— or —N—;
R₁ is C₁₋₃ alkoxy;
R₂ is —OCH₂COOH or an N-linked 4-7 membered heterocycle; wherein the N-linked 4-7 membered heterocycle is selected from an azetidine, pyrrolidine, morpholine, piperazine, piperidine, and oxazepane; wherein the heterocycle is optionally bridged by, fused with, or spiro-linked with a C₁₋₄ alkylenyl, or a 1-5 membered heteroalkyl; wherein the heterocycle, the C₁₋₄ alkylenyl, and the 1-5 membered heteroalkyl are each optionally substituted with 1-3 substituents each independently selected from halogen, oxo, C₁₋₄ alkyl, C₁₋₄ haloalkyl, —OH, and —COOH;
R₃ is selected from the group consisting of

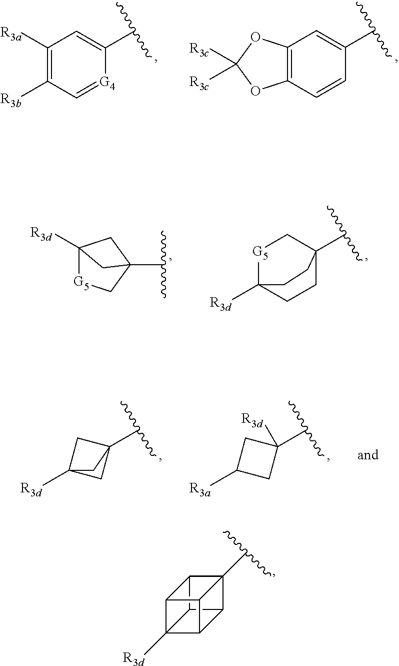

wherein

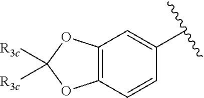

is optionally substituted with one to three halogens;
—G₄— is —CH— or —N—;
—G₅— is —CH₂— or —O—;
R₃ₐ, at each occurrence, is independently —H, one or two halogens, —SF₅, —SO₂CF₃, —SCF₃, —CN, C₁₋₄ alkyl, C₃₋₆ cycloalkyl, or C₁₋₃ alkoxy, wherein the C₁₋₄ alkyl, or C₁₋₃ alkoxy are optionally substituted with one, two, or three halogens;
R_{3b}, at each occurrence, is independently —H, halogen, difluoromethyl, trifluoromethyl, methoxy, trifluoromethoxy, —CN, or C₁₋₄ alkyl;
R_{3c}, at each occurrence, is independently —H or halogen;
R_{3d} is —H, halogen, —CN, C₁₋₄ alkyl, C₃₋₆ cycloalkyl, or C₁₋₃ alkoxy, wherein the C₁₋₄ alkyl or C₁₋₃ alkoxy are optionally substituted with one, two, or three halogens; or a pharmaceutically acceptable salt thereof.

The following are further numbered aspects of the invention:

1. A compound of the formula:

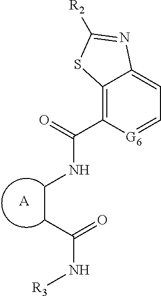

wherein:
Ring A is a group of the formula

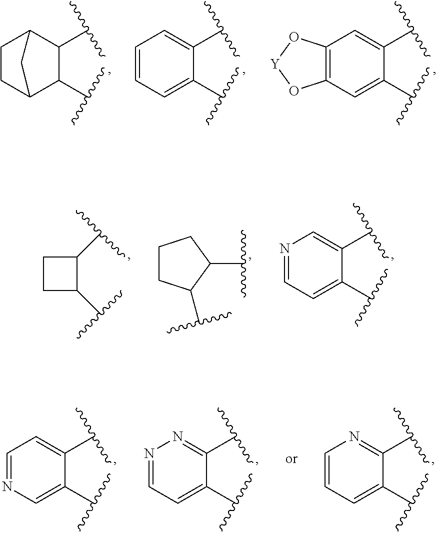

wherein each of

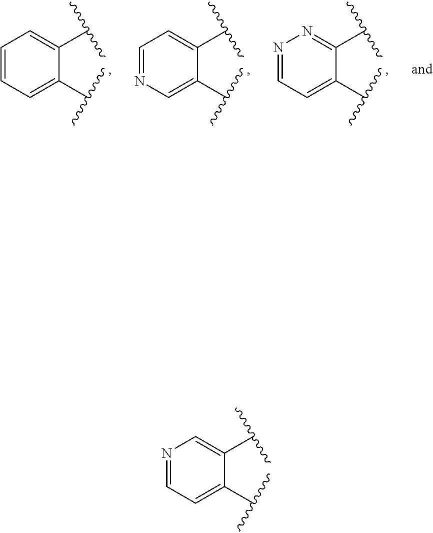 [SMILES: *c1ccccc1* ; *c1ccncc1* ; *c1ccnnc1* ; *c1ccncc1*]

is optionally substituted by one or more substituents selected from halogens, —CN, C₁₋₄ alkyl, and C₁₋₃ alkoxy, wherein each C₁₋₄ alkyl and C₁₋₃ alkoxy is independently optionally substituted by one or more substituents selected from halogens and —CN;
—Y— is a C₁₋₃ alkylene;
—G₆— is —C(R₁)— or —N—;
R₁ is C₁₋₃ alkoxy;
R₂ is —OCH₂COOH or an N-linked 4-7 membered heterocycle; wherein the N-linked 4-7 membered heterocycle is selected from an azetidine, pyrrolidine, morpholine, piperazine, piperidine, and oxazepane; wherein the heterocycle is optionally bridged by, fused with, or spiro-linked with a C₁₋₄ alkylenyl, or a 1-5 membered heteroalkyl; wherein the heterocycle, the C₁₋₄ alkylenyl, and the 1-5 membered heteroalkyl are each optionally substituted with 1-3 substituents each independently selected from halogen, oxo, C₁₋₄ alkyl, C₁₋₄ haloalkyl, —OH, and —COOH;
R₃ is selected from the group consisting of

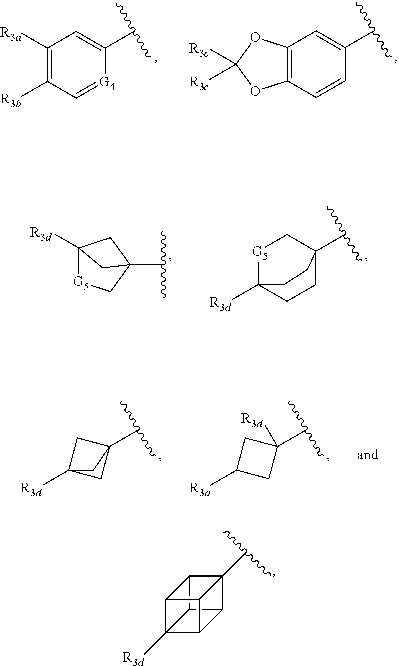

wherein

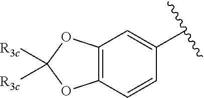

is optionally substituted with one to three halogens;
—G₄— is —CH— or —N—;
—G₅— is —CH₂— or —O—;
R₃ₐ, at each occurrence, is independently —H, halogen, —SF₅, —SO₂CF₃, —SCF₃, —CN, C₁₋₄ alkyl, C₃₋₆ cycloalkyl, or C₁₋₃ alkoxy, wherein the C₁₋₄ alkyl and C₁₋₃ alkoxy are optionally substituted with one, two or three halogens;
R_{3b} is independently —H, halogen, difluoromethyl, trifluoromethyl, methoxy, trifluoromethoxy, —CN, or C₁₋₄ alkyl;
R_{3c}, at each occurrence, is independently —H or halogen;
R_{3d} is —H, halogen, —CN, C₁₋₄ alkyl, C₃₋₆ cycloalkyl, or C₁₋₃ alkoxy, wherein the C₁₋₄ alkyl and C₁₋₃ alkoxy are optionally substituted with one, two or three halogens; or a pharmaceutically acceptable salt thereof.

Disclosed herein are the compounds of Formula I or pharmaceutically acceptable salts thereof, and methods of using the compounds of Formula I or pharmaceutically acceptable salts thereof, for treating patients for cardiovascular, pulmonary and/or renal conditions, diseases and/or disorders.

Also provided herein are methods of using the compounds of Formula I, pharmaceutically acceptable salts thereof, and pharmaceutical compositions thereof, to treat cardiovascular, pulmonary and/or renal conditions, diseases and/or disorders. The methods include administering a therapeutically effective amount of a compound of Formula I, or a pharmaceutically acceptable salt thereof, to a patient in need thereof.

Further provided herein, are compounds of Formula I, and pharmaceutically acceptable salts thereof, for use in therapy. Additionally provided herein, are the compounds of Formula I, and pharmaceutically acceptable salts thereof, for use in the treatment of cardiovascular, pulmonary and/or renal conditions, diseases and/or disorders. Also additionally provided herein is the use of compounds of Formula I, or pharmaceutically acceptable salts thereof, in the manufacture of a medicament for treating cardiovascular, pulmonary and/or renal conditions, diseases and/or disorders.

### DETAILED DESCRIPTION OF THE INVENTION

Novel RXFP1 receptor agonists are described herein. These new compounds could address some or all of the needs noted above for novel RXFP1 receptor agonists in the treatment of cardiovascular, pulmonary and/or renal conditions, diseases and/or disorders.

The following are further numbered aspects of the invention:

1. A compound of the formula:

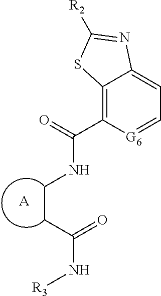

wherein:
Ring A is a group of the formula

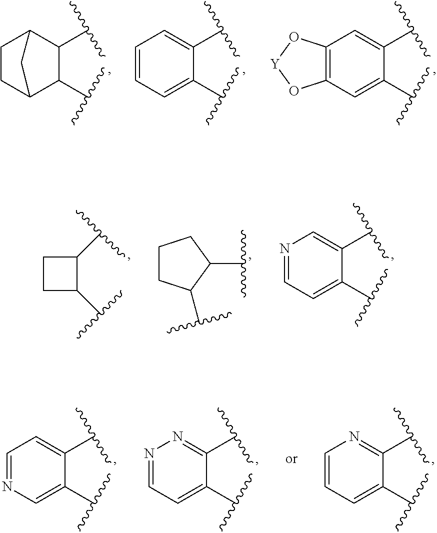

wherein each of

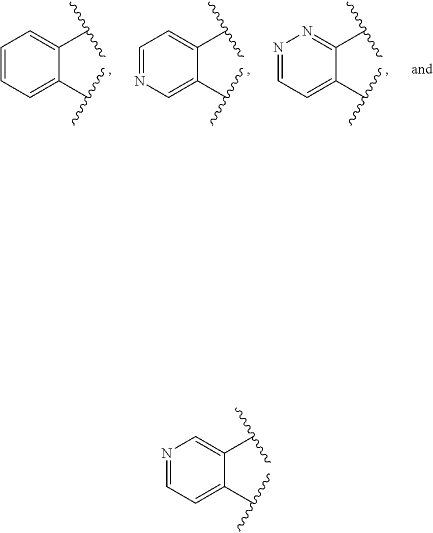 [SMILES: *c1ccccc1* ; *c1ccncc1* ; *c1ccnnc1* ; *c1ccncc1*]

is optionally substituted by one or more substituents selected from halogens, —CN, C₁₋₄ alkyl, and C₁₋₃ alkoxy, wherein each C₁₋₄ alkyl and C₁₋₃ alkoxy is independently optionally substituted by one or more substituents selected from halogens and —CN;
—Y— is a C₁₋₃ alkylene;
—G₆— is —C(R₁)— or —N—;
R₁ is C₁₋₃ alkoxy;
R₂ is —OCH₂COOH or an N-linked 4-7 membered heterocycle; wherein the N-linked 4-7 membered heterocycle is selected from an azetidine, pyrrolidine, morpholine, piperazine, piperidine, and oxazepane; wherein the heterocycle is optionally bridged by, fused with, or spiro-linked with a C₁₋₄ alkylenyl, or a 1-5 membered heteroalkyl; wherein the heterocycle, the C₁₋₄ alkylenyl, and the 1-5 membered heteroalkyl are each optionally substituted with 1-3 substituents each independently selected from halogen, oxo, C₁₋₄ alkyl, C₁₋₄ haloalkyl, —OH, and —COOH;
R₃ is selected from the group consisting of

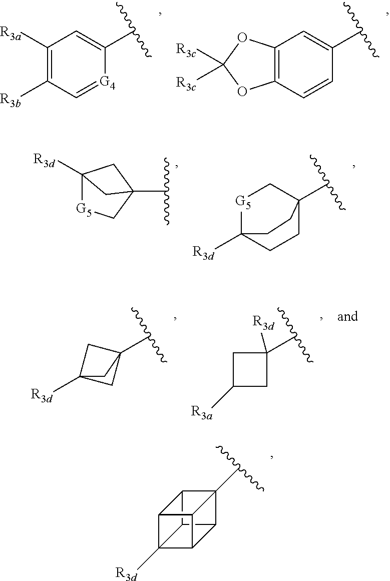

wherein

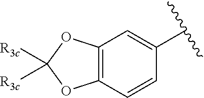

is optionally substituted with one to three halogens;
—G₄— is —CH— or —N—;
—G₅— is —CH₂— or —O—;
R₃ₐ, at each occurrence, is independently —H, halogen, —SF₅, —SO₂CF₃, —SCF₃, —CN, C₁₋₄ alkyl, C₃₋₆ cycloalkyl, or C₁₋₃ alkoxy, wherein the C₁₋₄ alkyl and C₁₋₃ alkoxy are optionally substituted with one, two or three halogens;
R_{3b} is independently —H, halogen, difluoromethyl, trifluoromethyl, methoxy, trifluoromethoxy, —CN, or C₁₋₄ alkyl;
R_{3c}, at each occurrence, is independently —H or halogen;
R_{3d} is —H, halogen, —CN, C₁₋₄ alkyl, C₃₋₆ cycloalkyl, or C₁₋₃ alkoxy, wherein the C₁₋₄ alkyl and C₁₋₃ alkoxy are optionally substituted with one, two, or three halogens; or a pharmaceutically acceptable salt thereof.

2. The compound according to aspect 1 wherein:
Ring A is a group of the formula

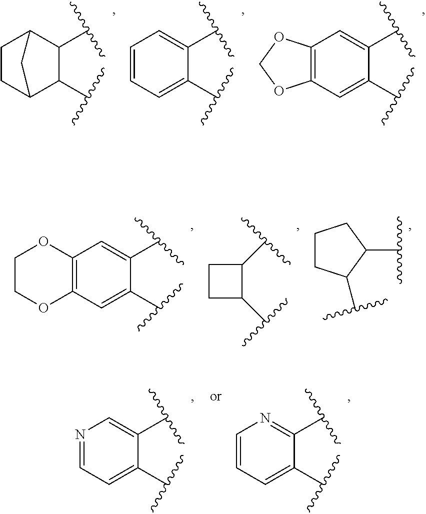

wherein each of

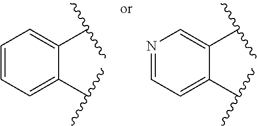 [SMILES: *c1ccccc1* ; *c1ccncc1*]

is optionally substituted by one or two halogens, C₁₋₄ alkyl, or C₁₋₃ alkoxy, wherein each C₁₋₄ alkyl or C₁₋₃ alkoxy is independently optionally substituted by one or more halogens or —CN;
R₂ is a group of the formula

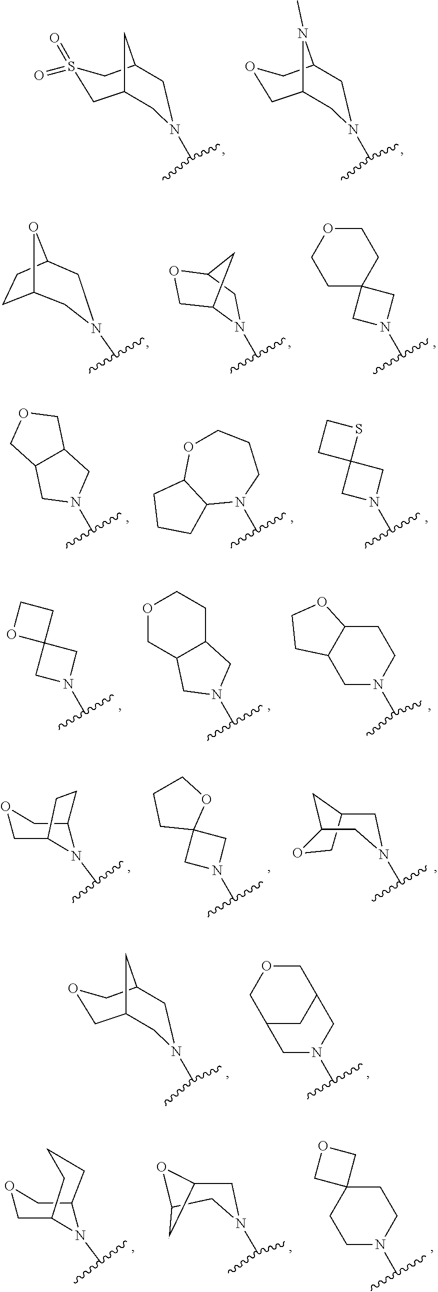

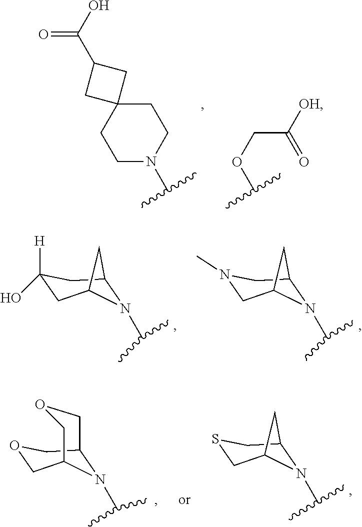

wherein

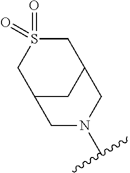 [SMILES: *N1CC2CC(C1)CS(=O)(=O)C2]

is optionally substituted with one or two halogens, wherein

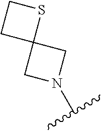 [SMILES: *N1CC2(CCS2)C1]

is optionally substituted with one or two oxo;
wherein R₃ is selected from the group consisting of

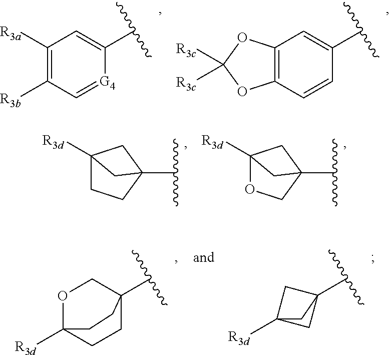

R₃ₐ, at each occurrence, is independently —H, halogen, —SF₅, —SO₂CF₃, —SCF₃, C₁₋₄ alkyl, or C₁₋₃ alkoxy, wherein the C₁₋₄ alkyl and C₁₋₃ alkoxy are optionally substituted with one or two halogens;
R_{3d} is —H, halogen, —CN, C₁₋₄ alkyl, or C₁₋₃ alkoxy, wherein the C₁₋₄ alkyl or C₁₋₃ alkoxy are optionally substituted with one or two halogens; and
—G₆— is —C(R₁)—; or a pharmaceutically acceptable salt thereof.

3. The compound according to aspect 1 wherein:
Ring A is a group of the formula

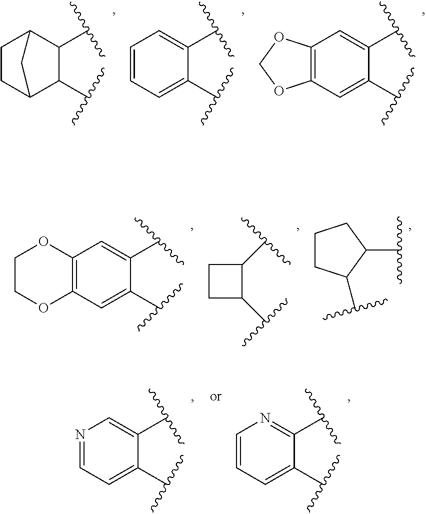

wherein each of

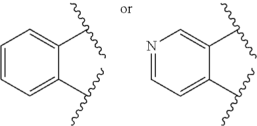 [SMILES: *c1ccccc1* ; *c1ccncc1*]

are optionally substituted by one or two halogens, C₁₋₄ alkyl, or C₁₋₃ alkoxy, wherein each C₁₋₄ alkyl or C₁₋₃ alkoxy is independently optionally substituted by one or more of halogens or —CN;
R₂ is a group of the formula

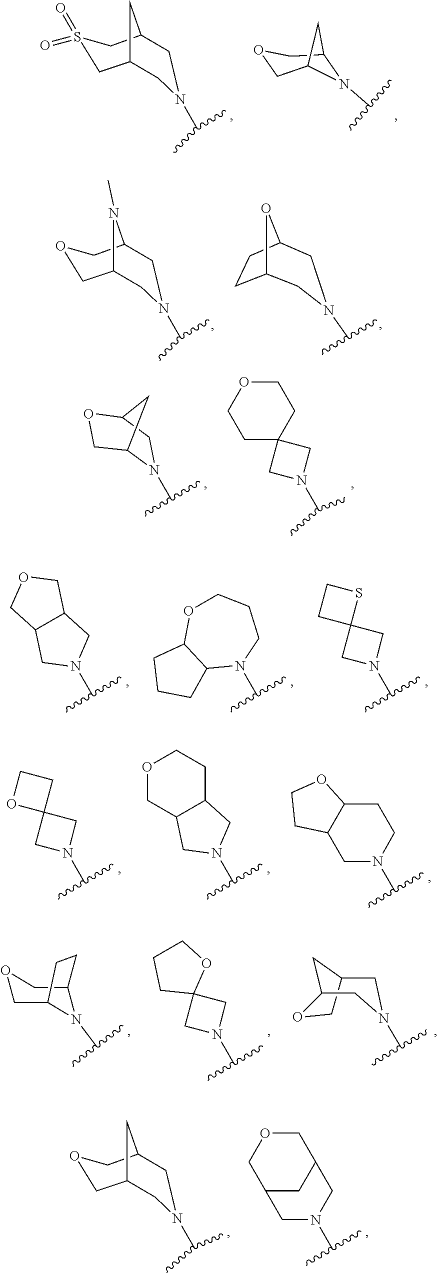

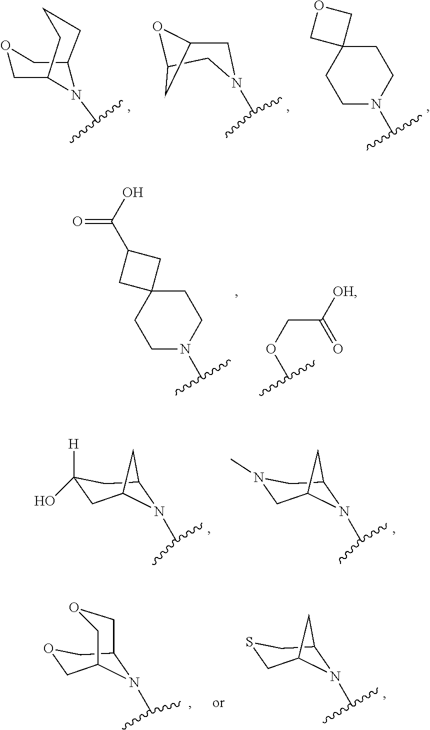

wherein

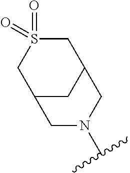 [SMILES: *N1CC2CC(C1)CS(=O)(=O)C2]

is optionally substituted with one or two halogens, wherein

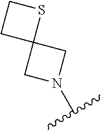 [SMILES: *N1CC2(CCS2)C1]

is optionally substituted with one or two oxo;
wherein R₃ is selected from the group consisting of

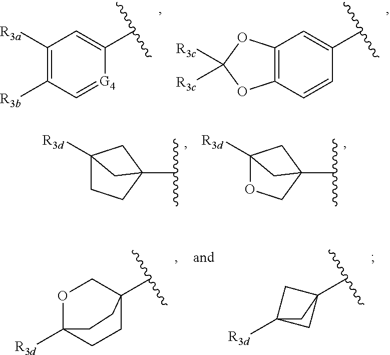

R₃ₐ, at each occurrence, is independently —H, halogen, —SF₅, —SO₂CF₃, —SCF₃, C₁₋₄ alkyl, or C₁₋₃ alkoxy, wherein the C₁₋₄ alkyl or C₁₋₃ alkoxy are optionally substituted with one or two halogens;
R_{3d} is —H, halogen, —CN, C₁₋₄ alkyl, or C₁₋₃ alkoxy, wherein the C₁₋₄ alkyl or C₁₋₃ alkoxy are optionally substituted with one or two halogens; and
—G₆— is —C(R₁)—; or a pharmaceutically acceptable salt thereof.

4. The compound according to aspect 1 wherein Ring A is a group of the formula

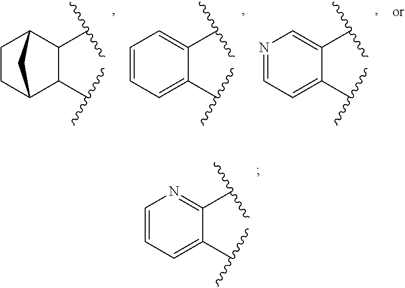

R₂ is —OCH₂COOH or an N-linked 4-7 membered heterocycle; wherein the N-linked 4-7 membered heterocycle is selected from an azetidine, pyrrolidine, morpholine, piperazine, piperidine, or oxazepane; wherein the heterocycle is optionally bridged by, fused with, or spiro-linked with a C₁₋₄ alkylenyl, or 1-5 membered heteroalkyl; wherein the heterocycle, the C₁₋₄ alkylenyl, and 1-5 membered heteroalkyl are each optionally substituted with 1-3 substituents each independently selected from halogen, C₁₋₄ alkyl, C₁₋₄ haloalkyl, and —COOH;
R₃ is a group of the formula:

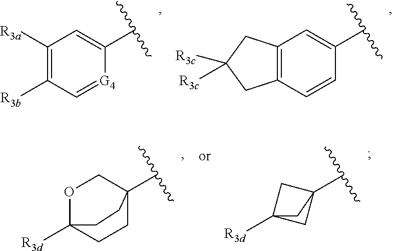

R₃ₐ is —H, halogen, —SF₅, —SO₂CF₃, —SCF₃, C₁₋₄ alkyl, or C₁₋₃ alkoxy, wherein the C₁₋₄ alkyl or C₁₋₃ alkoxy are optionally substituted with one or more halogens;
each R_{3c} is independently —H, or —F;
R_{3d} is —H, halogen, C₁₋₄ alkyl, or C₁₋₃ alkoxy, wherein the C₁₋₄ alkyl or C₁₋₃ alkoxy are optionally substituted with one or more halogens; and
—G₆— is —C(R₁)—; or a pharmaceutically acceptable salt thereof.

5. The compound according to any one of aspects 1 to 3 wherein Ring A is a group of the formula

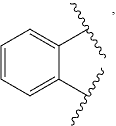 [SMILES: *c1ccccc1*]

wherein

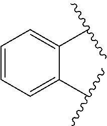 [SMILES: *c1ccccc1*]

is optionally substituted by one or two halogens, C₁₋₄ alkyl, or C₁₋₃ alkoxy, wherein each C₁₋₄ alkyl and C₁₋₃ alkoxy is independently optionally substituted by one or more selected from halogens and —CN;
or a pharmaceutically acceptable salt thereof.

6. The compound according to any one of aspects 1 to 3 wherein Ring A is a group of the formula

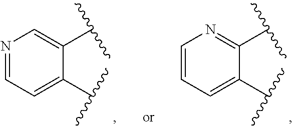 [SMILES: *c1ccncc1* ; *c1cccnc1*]

wherein

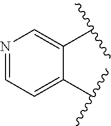 [SMILES: *c1ccncc1*]

is optionally substituted by one or two halogens, C₁₋₄ alkyl, or C₁₋₃ alkoxy, wherein each C₁₋₄ alkyl and C₁₋₃ alkoxy is independently optionally substituted by one or more substituents selected from halogens and —CN;
or a pharmaceutically acceptable salt thereof.

7. The compound according to aspect 1 wherein Ring A is a group of the formula

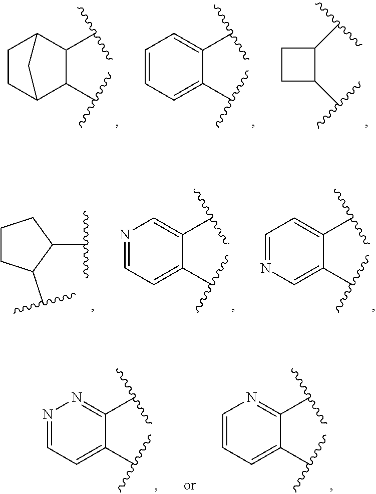

wherein each of

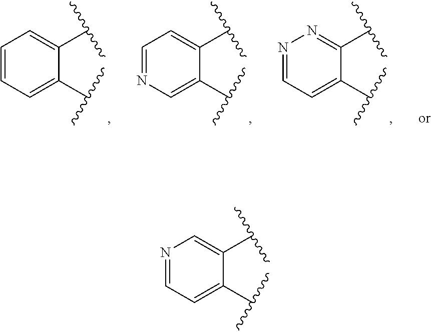 [SMILES: *c1ccccc1* ; *c1ccncc1* ; *c1ccnnc1* ; *c1ccncc1*]

are optionally substituted by one or more substituents selected from halogens, —CN, C₁₋₄ alkyl, —S—C₁₋₃ alkyl, and C₁₋₃ alkoxy, wherein each C₁₋₄ alkyl and C₁₋₃ alkoxy is independently optionally substituted by one or more substituents selected from halogens and —CN; or a pharmaceutically acceptable salt thereof.

8. The compound according to any one of aspects 1 to 3 wherein Ring A is a group of the

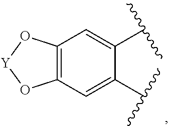

or a pharmaceutically acceptable salt thereof.

9. The compound according to any one of aspects 1 to 4 wherein Ring A is a group of the formula

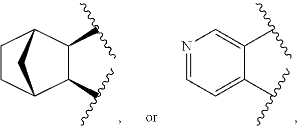 [SMILES: *[C@@H]1[C@H]2CC[C@H](C2)[C@@H]1* ; *c1ccncc1*]

or a pharmaceutically acceptable salt thereof.

10. The compound according to aspect 9 of the formula

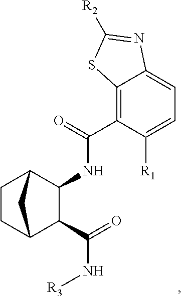

or a pharmaceutically acceptable salt thereof.

11. The compound according to aspect 9 of the formula

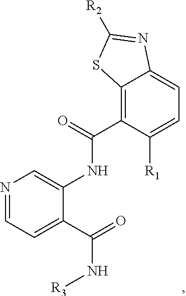

or a pharmaceutically acceptable salt thereof.

12. The compound according to any one of aspects 1-11, wherein —G₆— is —C(R₁)—, or a pharmaceutically acceptable salt thereof.

13. The compound according to aspect 12, wherein R₁ is methoxy, ethoxy, or isopropoxy or a pharmaceutically acceptable salt thereof.

14. The compound according to aspect 13, wherein R₁ is methoxy, or a pharmaceutically acceptable salt thereof.

15. The compound according to any one of aspects 1-11, wherein R₃ is a group of the formula

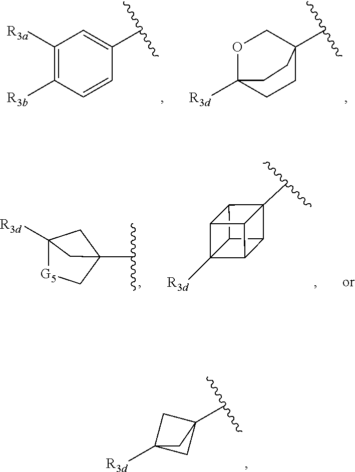

or a pharmaceutically acceptable salt thereof.

16. The compound according to aspect 15, wherein R_{3d} is trifluoromethyl; or a pharmaceutically acceptable salt thereof.

17. The compound according to aspect 15, wherein R₃ is a group of the formula

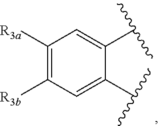

or a pharmaceutically acceptable salt thereof.

18. The compound according to aspect 16, wherein R₃ₐ is —F, —Cl, —Br, difluoromethyl, difluorochloromethyl, trifluoromethyl, trifluoromethoxy, or difluoromethoxy, and R_{3b} is —H, —F, —Cl, —Br, trifluoromethyl, —CN, or methyl, or a pharmaceutically acceptable salt thereof.

19. The compound according to aspect 18 wherein R₃ is a group of the formula

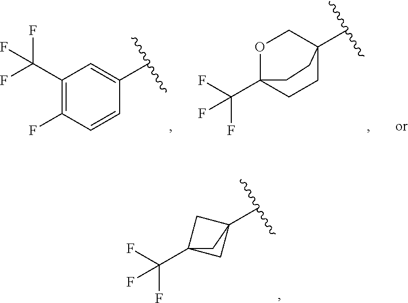 [SMILES: *c1ccc(F)c(C(F)(F)F)c1 ; *C12CCC(C(F)(F)F)(CC1)OC2 ; *C12CC(C(F)(F)F)(C1)C2]

or a pharmaceutically acceptable salt thereof.

20. The compound according to aspect 19, wherein R₃ is a group of the formula

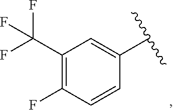 [SMILES: *c1ccc(F)c(C(F)(F)F)c1]

or a pharmaceutically acceptable salt thereof.

21. The compound according to aspect 20 of the formula

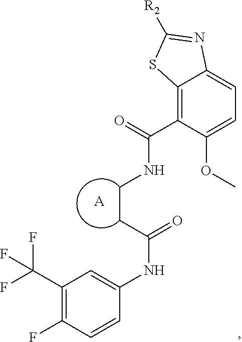

or a pharmaceutically acceptable salt thereof.

22. The compound according to any one of aspects 1, 3, 5-21, wherein R₂ is a group of the formula

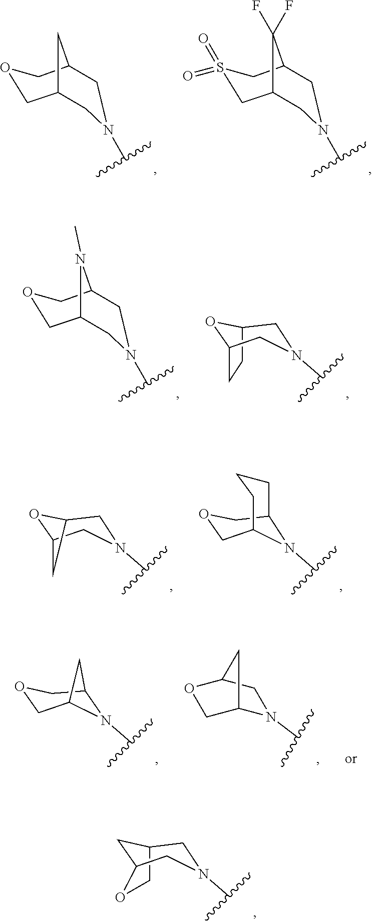

or a pharmaceutically acceptable salt thereof.

23. The compound according to any one of aspects 1-21, wherein R₂ is a group of the formula

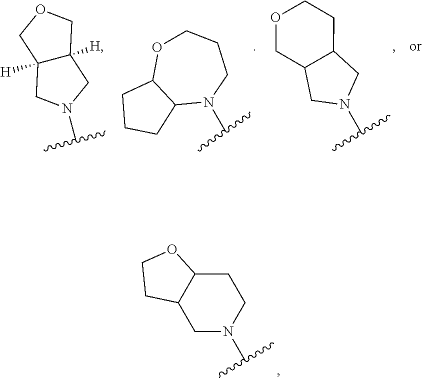

or a pharmaceutically acceptable salt thereof.

24. The compound according to any one of aspects 1-3, 5-21, wherein R₂ is a group of the formula

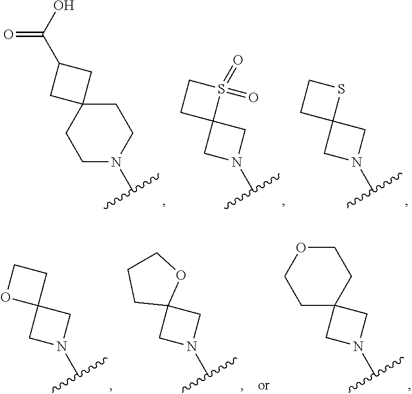 [SMILES: *N1CCC2(CC1)CC(C(=O)O)C2 ; *N1CC2(CCS2(=O)=O)C1 ; *N1CC2(CCS2)C1 ; *N1CC2(CCO2)C1 ; *N1CC2(CCCO2)C1 ; *N1CC2(CCOCC2)C1]

or a pharmaceutically acceptable salt thereof.

25. The compound according to any one of aspects 1-21, wherein R₂ is a group of the formula

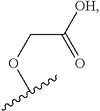 [SMILES: *OCC(=O)O]

or a pharmaceutically acceptable salt thereof.

26. The compound according to aspect 1 selected from the group consisting of

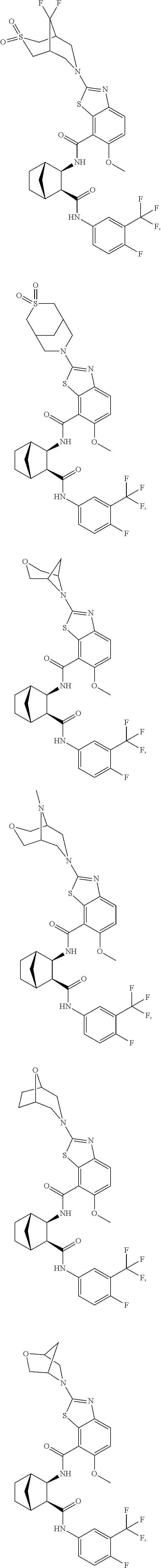

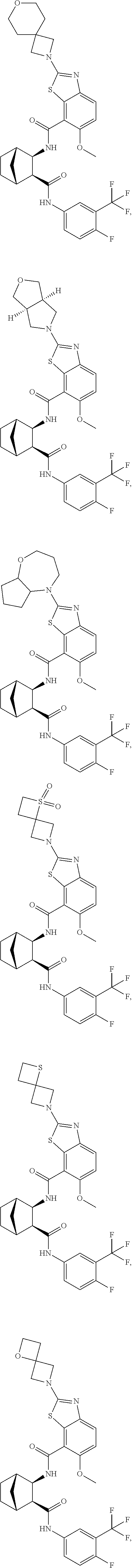

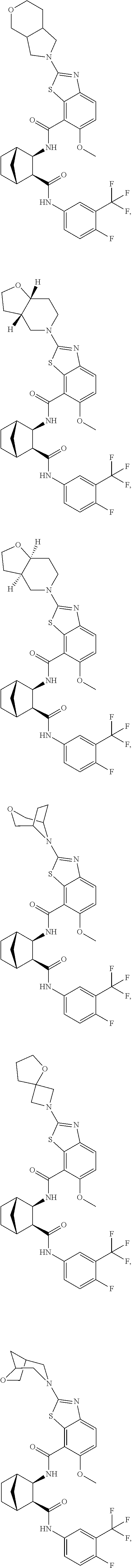

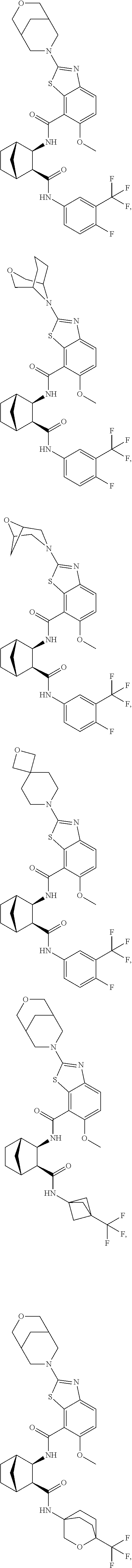

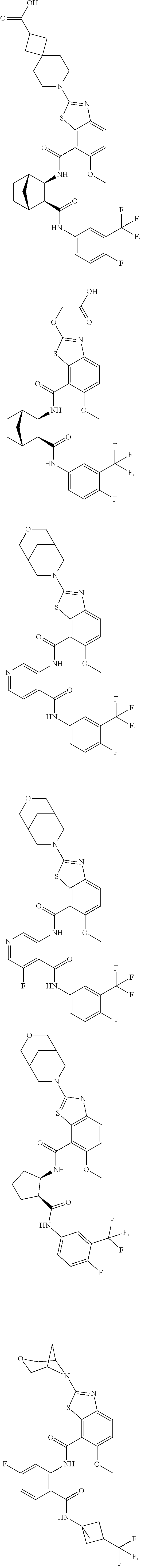

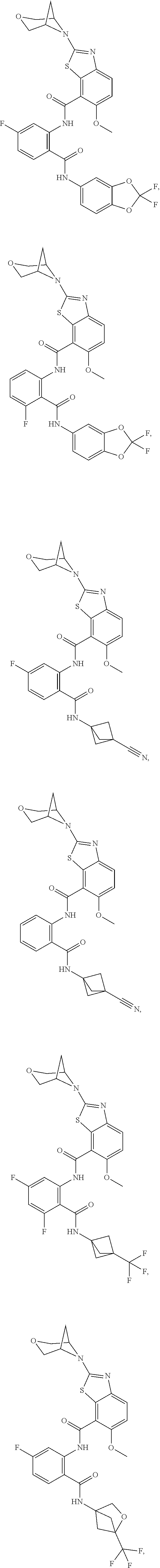 [SMILES: COc1ccc2nc(N3C4COCC3C4)sc2c1C(=O)Nc1cc(F)ccc1C(=O)Nc1ccc2c(c1)OC(F)(F)O2 ; COc1ccc2nc(N3C4COCC3C4)sc2c1C(=O)Nc1cccc(F)c1C(=O)Nc1ccc2c(c1)OC(F)(F)O2 ; COc1ccc2nc(N3C4COCC3C4)sc2c1C(=O)Nc1cc(F)ccc1C(=O)NC12CC(C#N)(C1)C2 ; COc1ccc2nc(N3C4COCC3C4)sc2c1C(=O)Nc1ccccc1C(=O)NC12CC(C#N)(C1)C2 ; COc1ccc2nc(N3C4COCC3C4)sc2c1C(=O)Nc1cc(F)cc(F)c1C(=O)NC12CC(C(F)(F)F)(C1)C2 ; COc1ccc2nc(N3C4COCC3C4)sc2c1C(=O)Nc1cc(F)ccc1C(=O)NC12COC(C(F)(F)F)(C1)C2]

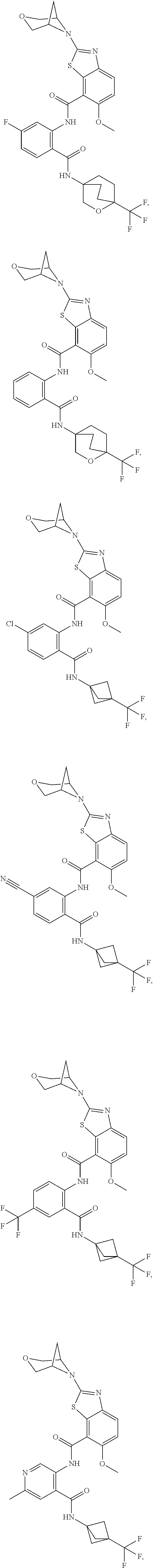 [SMILES: COc1ccc2nc(N3C4COCC3C4)sc2c1C(=O)Nc1cc(F)ccc1C(=O)NC12CCC(C(F)(F)F)(CC1)OC2 ; COc1ccc2nc(N3C4COCC3C4)sc2c1C(=O)Nc1ccccc1C(=O)NC12CCC(C(F)(F)F)(CC1)OC2 ; COc1ccc2nc(N3C4COCC3C4)sc2c1C(=O)Nc1cc(Cl)ccc1C(=O)NC12CC(C(F)(F)F)(C1)C2 ; COc1ccc2nc(N3C4COCC3C4)sc2c1C(=O)Nc1cc(C#N)ccc1C(=O)NC12CC(C(F)(F)F)(C1)C2 ; COc1ccc2nc(N3C4COCC3C4)sc2c1C(=O)Nc1ccc(C(F)(F)F)cc1C(=O)NC12CC(C(F)(F)F)(C1)C2 ; COc1ccc2nc(N3C4COCC3C4)sc2c1C(=O)Nc1cnc(C)cc1C(=O)NC12CC(C(F)(F)F)(C1)C2]

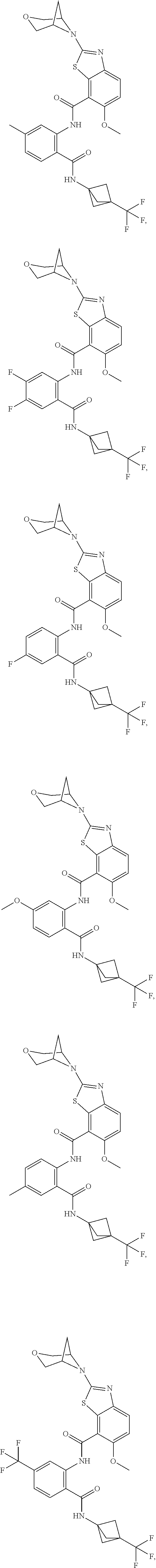 [SMILES: COc1ccc2nc(N3C4COCC3C4)sc2c1C(=O)Nc1cc(C)ccc1C(=O)NC12CC(C(F)(F)F)(C1)C2 ; COc1ccc2nc(N3C4COCC3C4)sc2c1C(=O)Nc1cc(F)c(F)cc1C(=O)NC12CC(C(F)(F)F)(C1)C2 ; COc1ccc2nc(N3C4COCC3C4)sc2c1C(=O)Nc1ccc(F)cc1C(=O)NC12CC(C(F)(F)F)(C1)C2 ; COc1ccc(C(=O)NC23CC(C(F)(F)F)(C2)C3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CC(C(F)(F)F)(C1)C2 ; COc1ccc2nc(N3C4COCC3C4)sc2c1C(=O)Nc1cc(C(F)(F)F)ccc1C(=O)NC12CC(C(F)(F)F)(C1)C2]

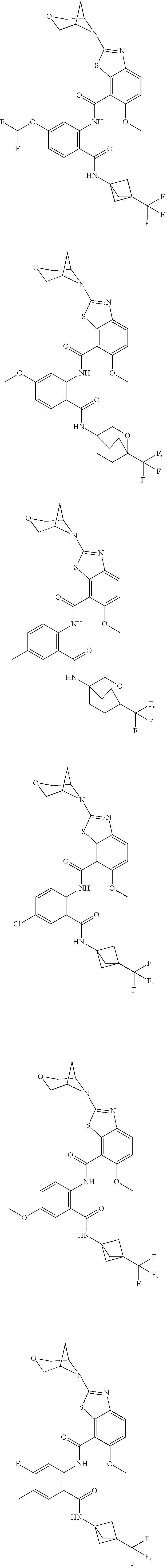 [SMILES: COc1ccc2nc(N3C4COCC3C4)sc2c1C(=O)Nc1cc(OC(F)F)ccc1C(=O)NC12CC(C(F)(F)F)(C1)C2 ; COc1ccc(C(=O)NC23CCC(C(F)(F)F)(CC2)OC3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CCC(C(F)(F)F)(CC1)OC2 ; COc1ccc2nc(N3C4COCC3C4)sc2c1C(=O)Nc1ccc(Cl)cc1C(=O)NC12CC(C(F)(F)F)(C1)C2 ; COc1ccc(NC(=O)c2c(OC)ccc3nc(N4C5COCC4C5)sc23)c(C(=O)NC23CC(C(F)(F)F)(C2)C3)c1 ; COc1ccc2nc(N3C4COCC3C4)sc2c1C(=O)Nc1cc(F)c(C)cc1C(=O)NC12CC(C(F)(F)F)(C1)C2]

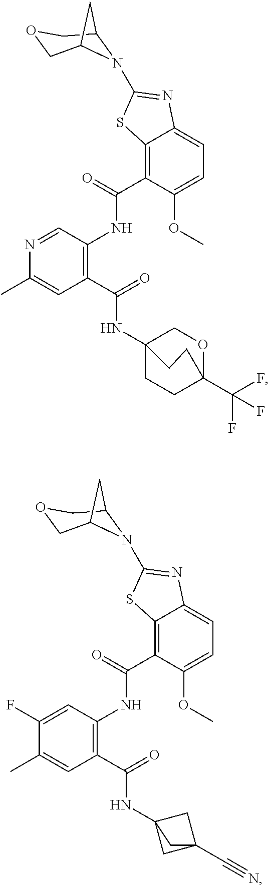 [SMILES: COc1ccc2nc(N3C4COCC3C4)sc2c1C(=O)Nc1cnc(C)cc1C(=O)NC12CCC(C(F)(F)F)(CC1)OC2 ; COc1ccc2nc(N3C4COCC3C4)sc2c1C(=O)Nc1cc(F)c(C)cc1C(=O)NC12CC(C#N)(C1)C2]

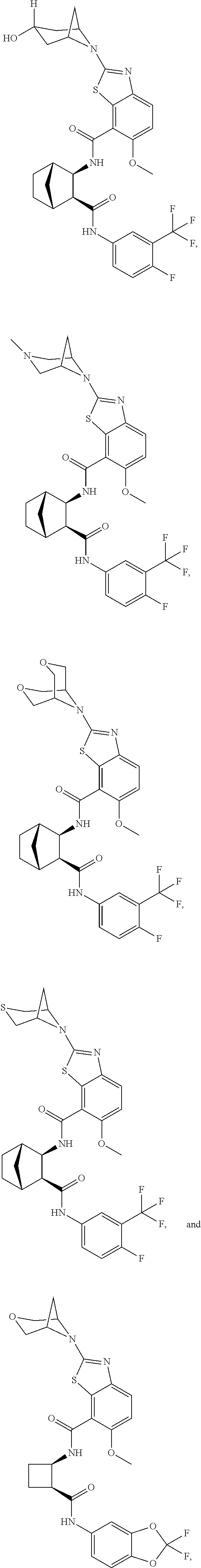 [SMILES: COc1ccc2nc(N3C4CC(O)CC3C4)sc2c1C(=O)N[C@@H]1[C@H]2CC[C@H](C2)[C@@H]1C(=O)Nc1ccc(F)c(C(F)(F)F)c1 ; COc1ccc2nc(N3C4CC3CN(C)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3C4CSCC3C4)sc2c1C(=O)N[C@@H]1[C@H]2CC[C@H](C2)[C@@H]1C(=O)Nc1ccc(F)c(C(F)(F)F)c1 ; "and" ; COc1ccc2nc(N3C4COCC3C4)sc2c1C(=O)N[C@@H]1CC[C@@H]1C(=O)Nc1ccc2c(c1)OC(F)(F)O2]

or a pharmaceutically acceptable salt thereof.

27. The compound according to aspect 1 selected from

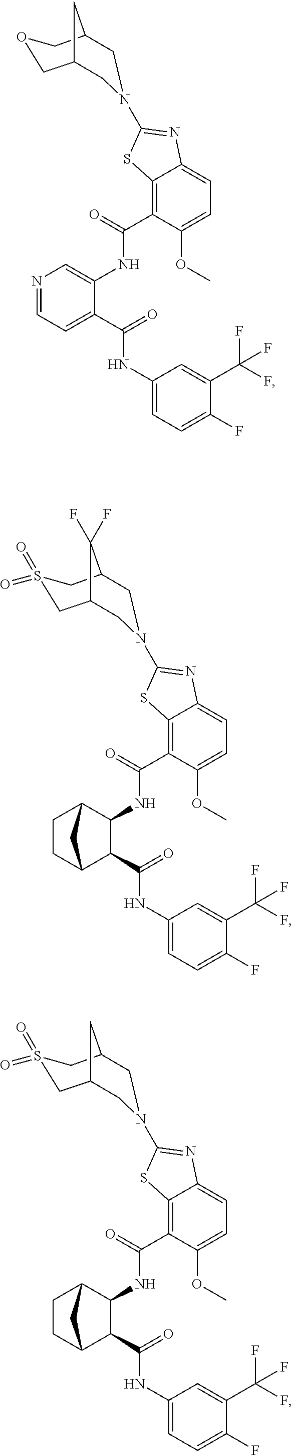 [SMILES: COc1ccc2nc(N3CC4COCC(C4)C3)sc2c1C(=O)Nc1cnccc1C(=O)Nc1ccc(F)c(C(F)(F)F)c1 ; COc1ccc2nc(N3CC4CS(=O)(=O)CC(C3)C4(F)F)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)CS(=O)(=O)C4)sc2c1C(=O)N[C@@H]1[C@H]2CC[C@H](C2)[C@@H]1C(=O)Nc1ccc(F)c(C(F)(F)F)c1]

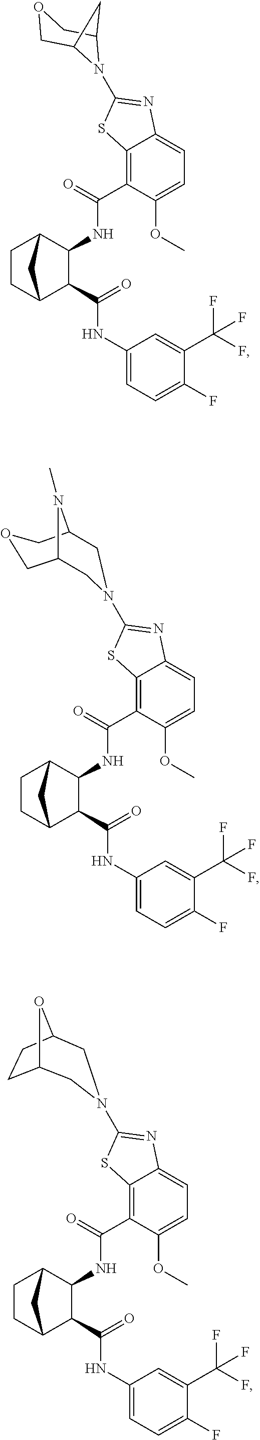 [SMILES: COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C3)N4C)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1]

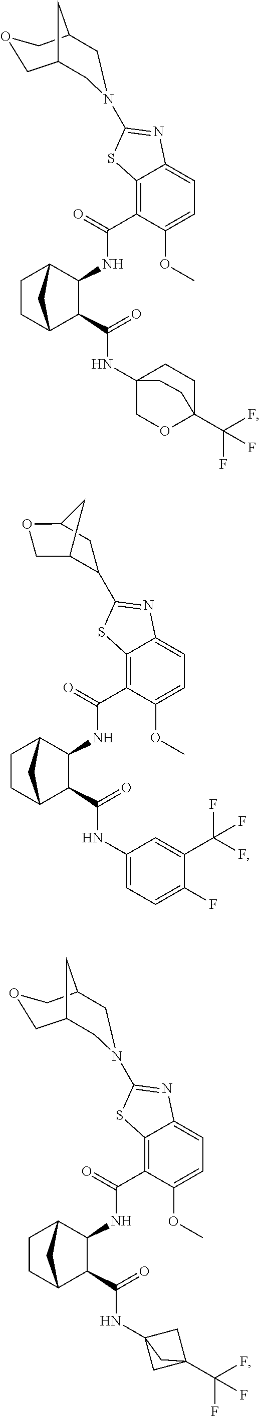 [SMILES: COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2 ; COc1ccc2nc(C3CC4CC3CO4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2]

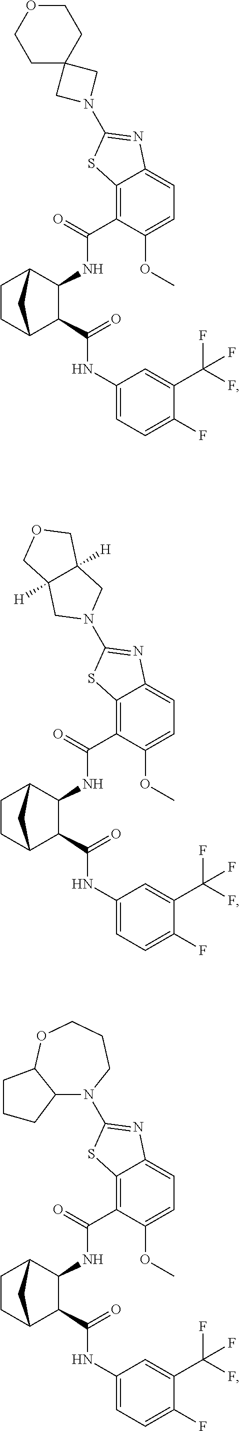 [SMILES: COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCCOC4CCCC43)sc2c1C(=O)N[C@@H]1[C@H]2CC[C@H](C2)[C@@H]1C(=O)Nc1ccc(F)c(C(F)(F)F)c1]

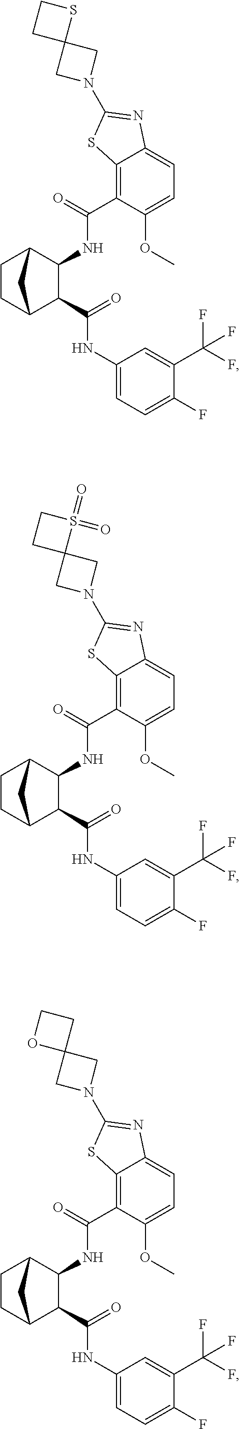 [SMILES: COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1]

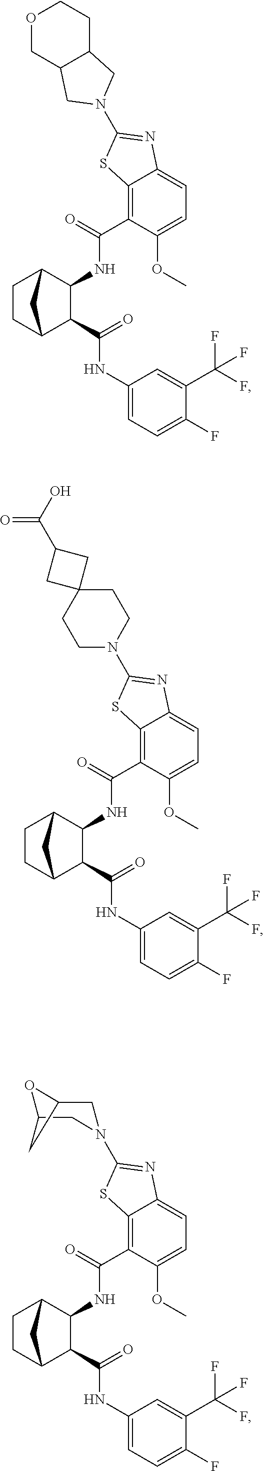 [SMILES: COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCC4(CC3)CC(C(=O)O)C4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1]

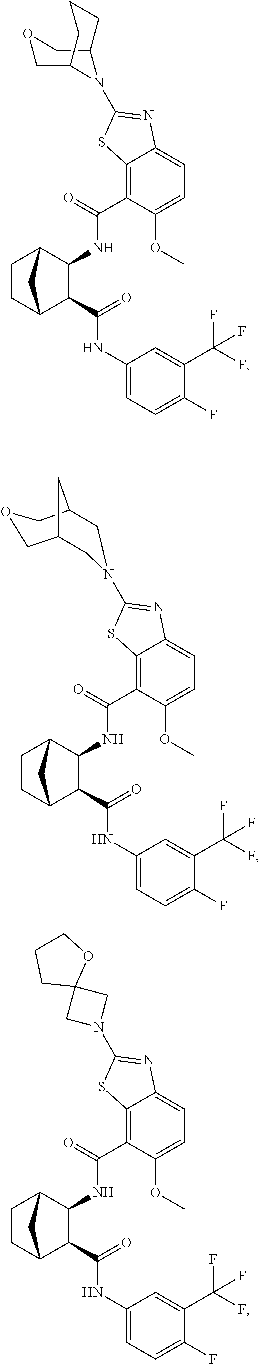 [SMILES: COc1ccc2nc(N3C4CCCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1]

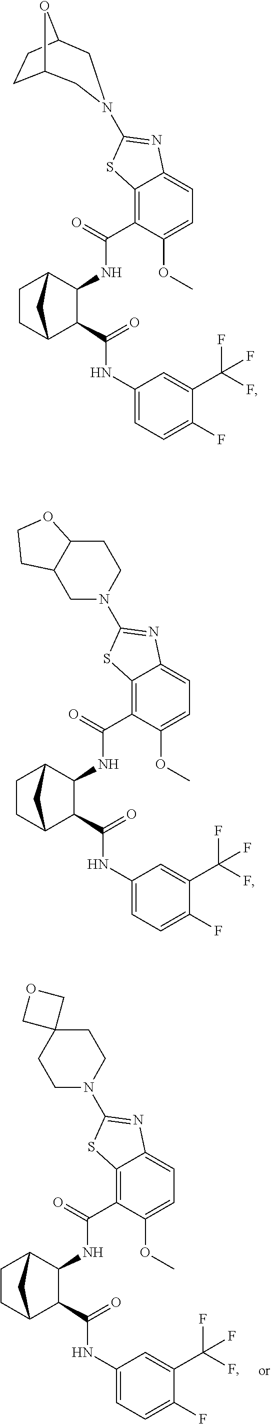 [SMILES: COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CCC4OCCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1]

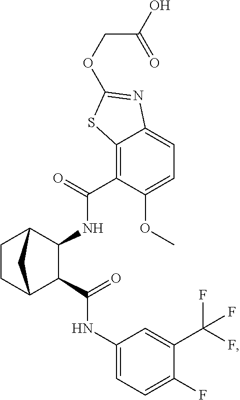 [SMILES: COc1ccc2nc(OCC(=O)O)sc2c1C(=O)N[C@@H]1[C@H]2CC[C@H](C2)[C@@H]1C(=O)Nc1ccc(F)c(C(F)(F)F)c1]

or a pharmaceutically acceptable salt thereof.

28. The compound according to aspect 1 selected from the group consisting of

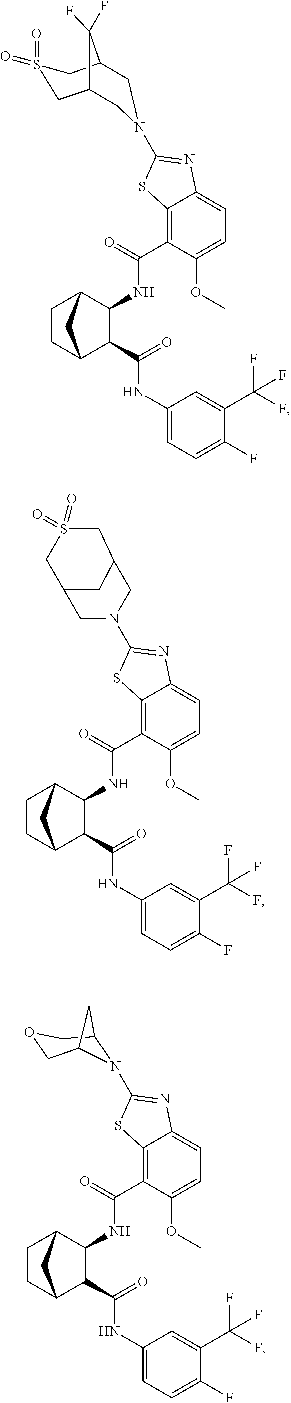

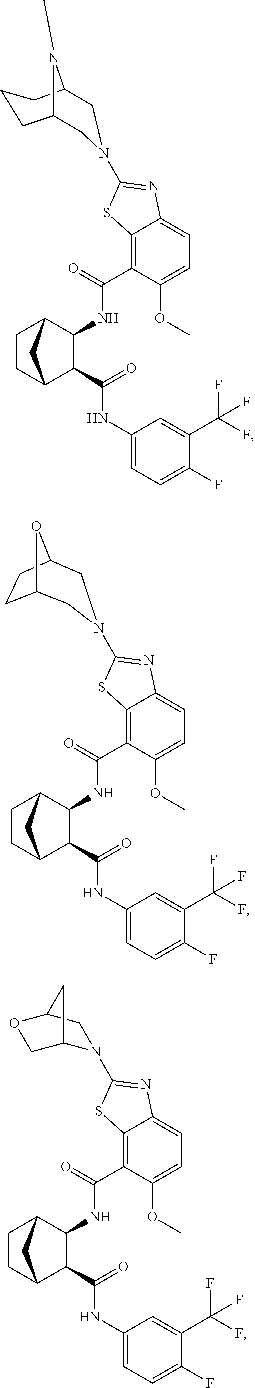 [SMILES: COc1ccc2nc(N3CC4CCCC(C3)N4C)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1]

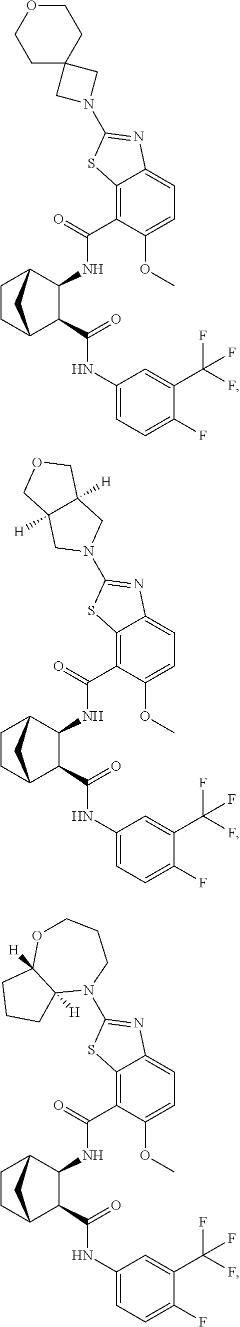 [SMILES: COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCCO[C@H]4CCC[C@@H]43)sc2c1C(=O)N[C@@H]1[C@H]2CC[C@H](C2)[C@@H]1C(=O)Nc1ccc(F)c(C(F)(F)F)c1]

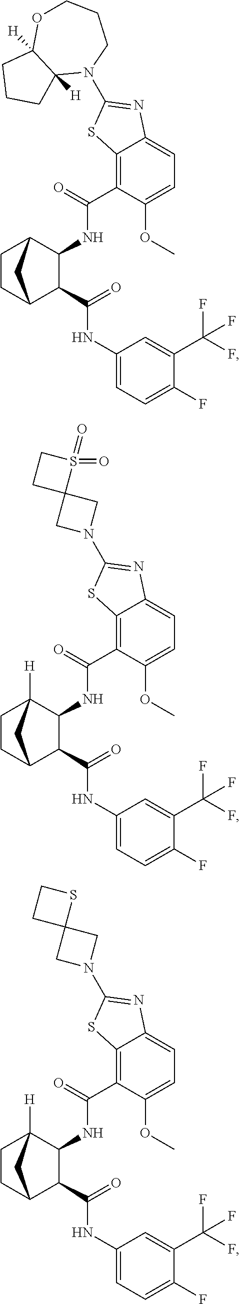 [SMILES: COc1ccc2nc(N3CCCO[C@@H]4CCC[C@H]43)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1]

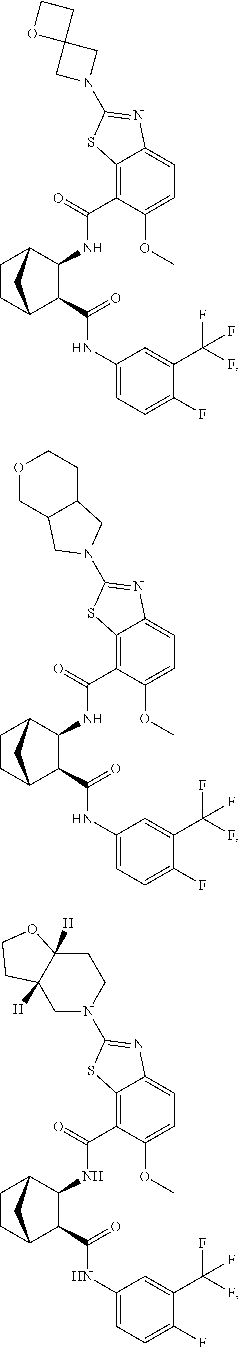 [SMILES: COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@H]4OCC[C@H]4C3)sc2c1C(=O)N[C@@H]1[C@H]2CC[C@H](C2)[C@@H]1C(=O)Nc1ccc(F)c(C(F)(F)F)c1]

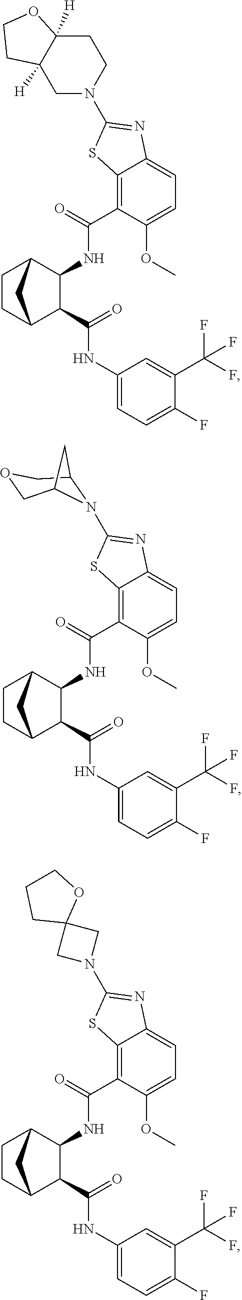 [SMILES: COc1ccc2nc(N3CC[C@@H]4OCC[C@@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1]

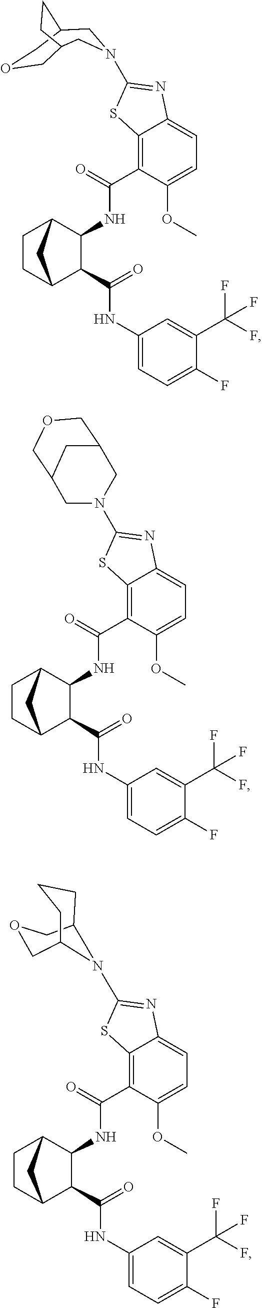 [SMILES: COc1ccc2nc(N3CC4CCC(COC4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCCC(COC4)C3)sc2c1C(=O)N[C@@H]1[C@H]2CC[C@H](C2)[C@@H]1C(=O)Nc1ccc(F)c(C(F)(F)F)c1]

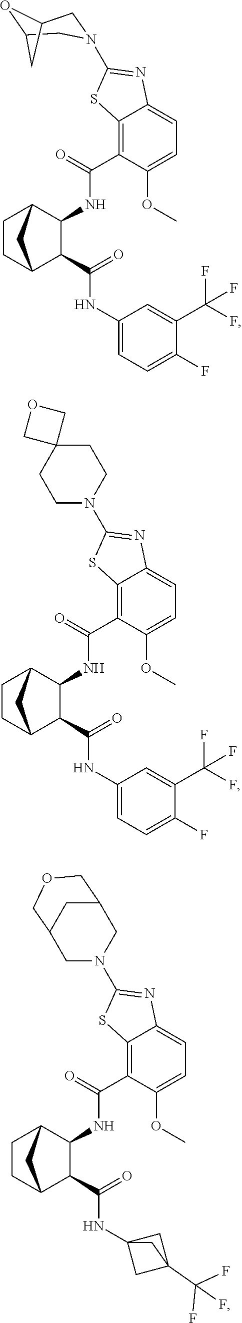 [SMILES: COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2]

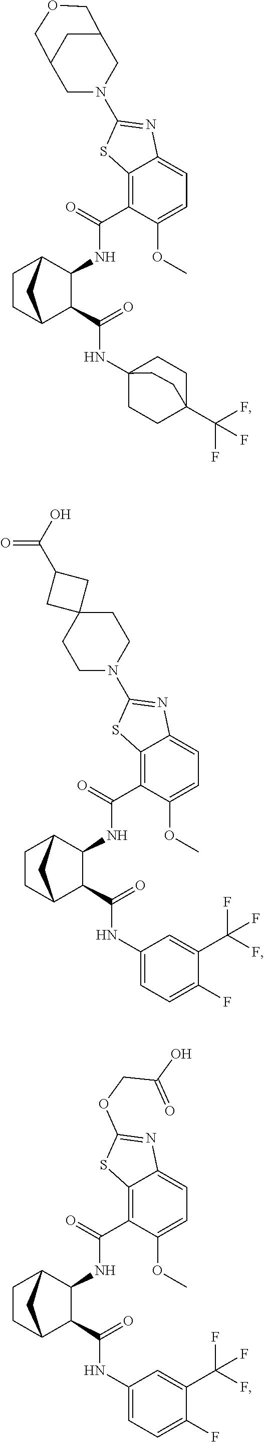 [SMILES: COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)CC2 ; COc1ccc2nc(N3CCC4(CC3)CC(C(=O)O)C4)sc2c1C(=O)N[C@@H]1[C@H]2CC[C@H](C2)[C@@H]1C(=O)Nc1ccc(F)c(C(F)(F)F)c1 ; COc1ccc2nc(OCC(=O)O)sc2c1C(=O)N[C@@H]1[C@H]2CC[C@H](C2)[C@@H]1C(=O)Nc1ccc(F)c(C(F)(F)F)c1]

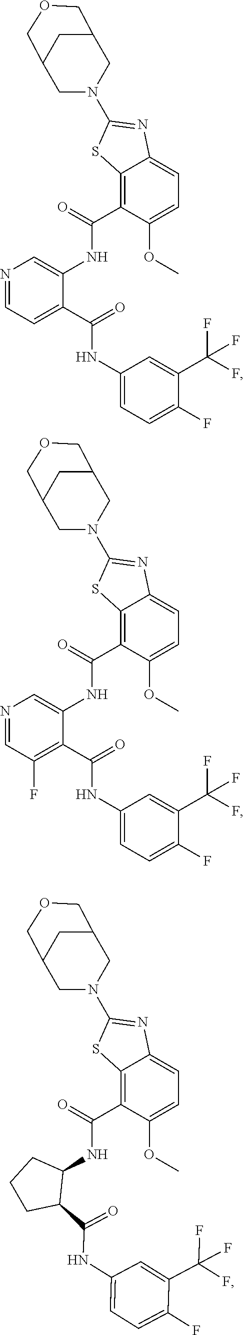 [SMILES: COc1ccc2nc(N3CC4COCC(C4)C3)sc2c1C(=O)Nc1cnccc1C(=O)Nc1ccc(F)c(C(F)(F)F)c1 ; COc1ccc2nc(N3CC4COCC(C4)C3)sc2c1C(=O)Nc1cncc(F)c1C(=O)Nc1ccc(F)c(C(F)(F)F)c1 ; COc1ccc2nc(N3CC4COCC(C4)C3)sc2c1C(=O)N[C@@H]1CCC[C@@H]1C(=O)Nc1ccc(F)c(C(F)(F)F)c1]

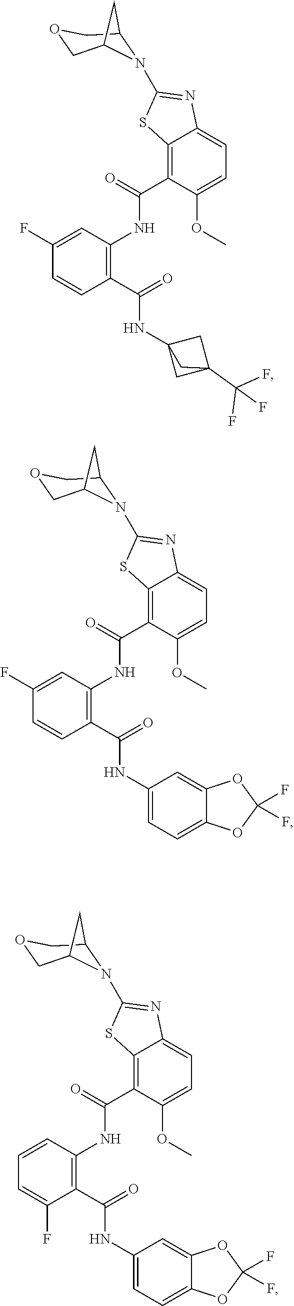 [SMILES: COc1ccc2nc(N3C4COCC3C4)sc2c1C(=O)Nc1cc(F)ccc1C(=O)NC12CC(C(F)(F)F)(C1)C2 ; COc1ccc2nc(N3C4COCC3C4)sc2c1C(=O)Nc1cc(F)ccc1C(=O)Nc1ccc2c(c1)OC(F)(F)O2 ; COc1ccc2nc(N3C4COCC3C4)sc2c1C(=O)Nc1cccc(F)c1C(=O)Nc1ccc2c(c1)OC(F)(F)O2]

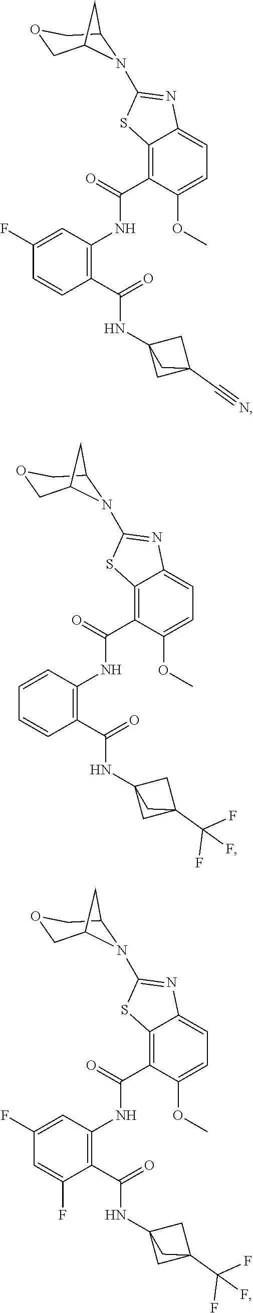 [SMILES: COc1ccc2nc(N3C4COCC3C4)sc2c1C(=O)Nc1cc(F)ccc1C(=O)NC12CC(C#N)(C1)C2 ; COc1ccc2nc(N3C4COCC3C4)sc2c1C(=O)Nc1ccccc1C(=O)NC12CC(C(F)(F)F)(C1)C2 ; COc1ccc2nc(N3C4COCC3C4)sc2c1C(=O)Nc1cc(F)cc(F)c1C(=O)NC12CC(C(F)(F)F)(C1)C2]

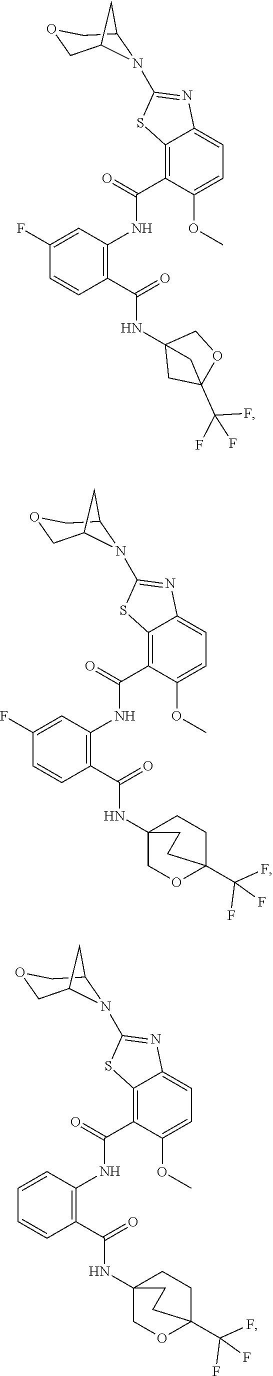 [SMILES: COc1ccc2nc(N3C4COCC3C4)sc2c1C(=O)Nc1cc(F)ccc1C(=O)NC12COC(C(F)(F)F)(C1)C2 ; COc1ccc2nc(N3C4COCC3C4)sc2c1C(=O)Nc1cc(F)ccc1C(=O)NC12CCC(C(F)(F)F)(CC1)OC2 ; COc1ccc2nc(N3C4COCC3C4)sc2c1C(=O)Nc1ccccc1C(=O)NC12CCC(C(F)(F)F)(CC1)OC2]

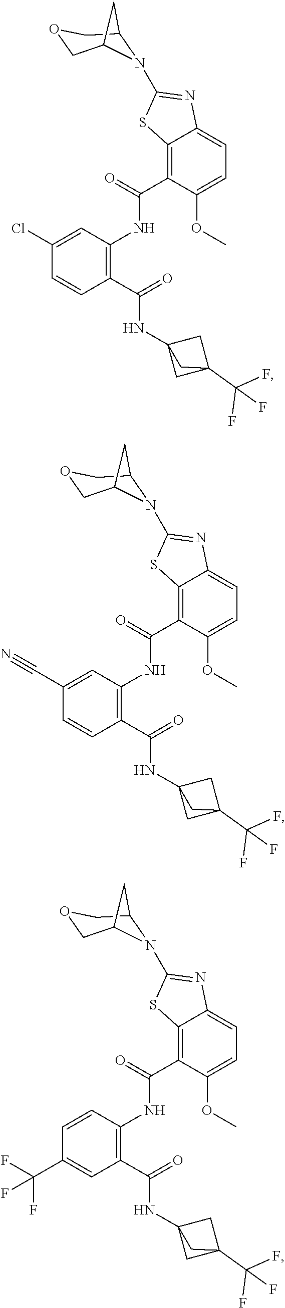 [SMILES: COc1ccc2nc(N3C4COCC3C4)sc2c1C(=O)Nc1cc(Cl)ccc1C(=O)NC12CC(C(F)(F)F)(C1)C2 ; COc1ccc2nc(N3C4COCC3C4)sc2c1C(=O)Nc1cc(C#N)ccc1C(=O)NC12CC(C(F)(F)F)(C1)C2 ; COc1ccc2nc(N3C4COCC3C4)sc2c1C(=O)Nc1ccc(C(F)(F)F)cc1C(=O)NC12CC(C(F)(F)F)(C1)C2]

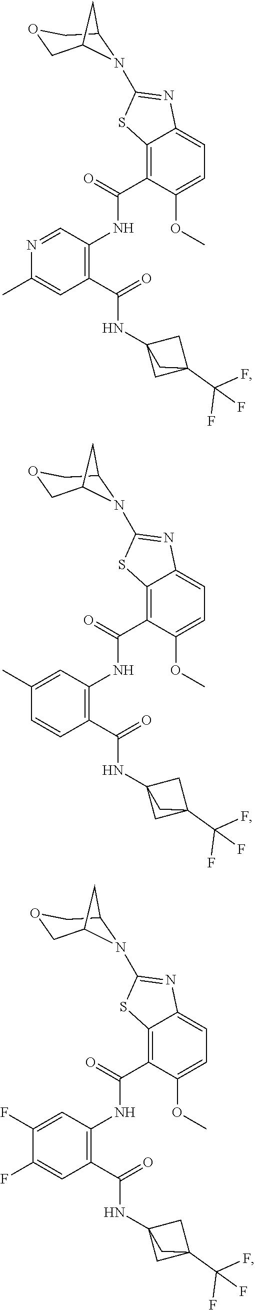 [SMILES: COc1ccc2nc(N3C4COCC3C4)sc2c1C(=O)Nc1cnc(C)cc1C(=O)NC12CC(C(F)(F)F)(C1)C2 ; COc1ccc2nc(N3C4COCC3C4)sc2c1C(=O)Nc1cc(C)ccc1C(=O)NC12CC(C(F)(F)F)(C1)C2 ; COc1ccc2nc(N3C4COCC3C4)sc2c1C(=O)Nc1cc(F)c(F)cc1C(=O)NC12CC(C(F)(F)F)(C1)C2]

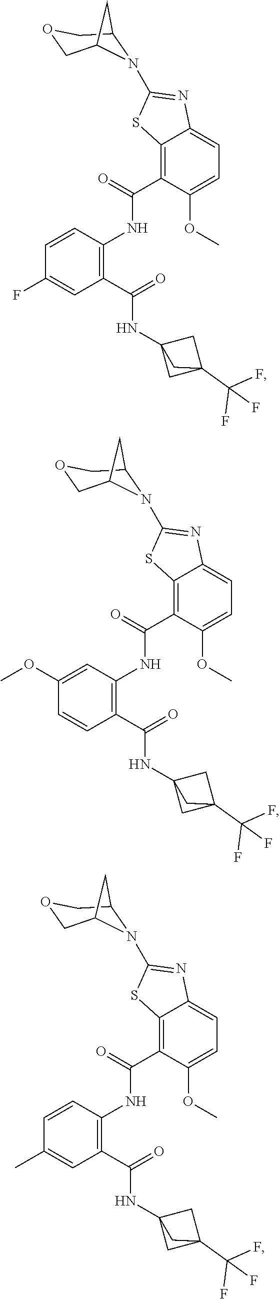 [SMILES: COc1ccc2nc(N3C4COCC3C4)sc2c1C(=O)Nc1ccc(F)cc1C(=O)NC12CC(C(F)(F)F)(C1)C2 ; COc1ccc(C(=O)NC23CC(C(F)(F)F)(C2)C3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CC(C(F)(F)F)(C1)C2]

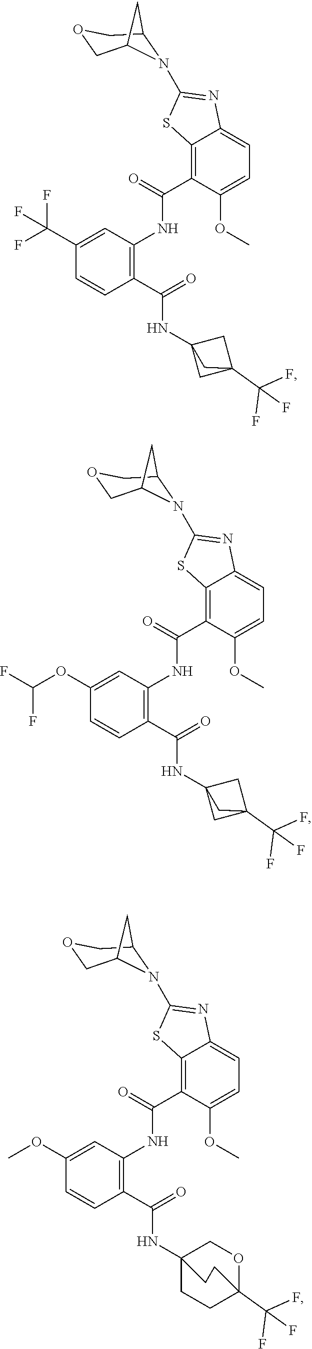 [SMILES: COc1ccc2nc(N3C4COCC3C4)sc2c1C(=O)Nc1cc(C(F)(F)F)ccc1C(=O)NC12CC(C(F)(F)F)(C1)C2 ; COc1ccc2nc(N3C4COCC3C4)sc2c1C(=O)Nc1cc(OC(F)F)ccc1C(=O)NC12CC(C(F)(F)F)(C1)C2 ; COc1ccc(C(=O)NC23CCC(C(F)(F)F)(CC2)OC3)c(NC(=O)c2c(OC)ccc3nc(N4C5COCC4C5)sc23)c1]

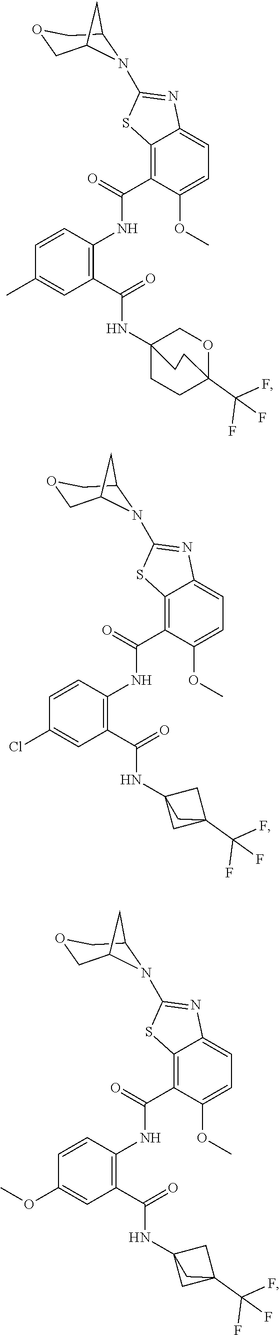 [SMILES: COc1ccc2nc(N3C4COCC3C4)sc2c1C(=O)Nc1ccc(C)cc1C(=O)NC12CCC(C(F)(F)F)(CC1)OC2 ; COc1ccc2nc(N3C4COCC3C4)sc2c1C(=O)Nc1ccc(Cl)cc1C(=O)NC12CC(C(F)(F)F)(C1)C2 ; COc1ccc(NC(=O)c2c(OC)ccc3nc(N4C5COCC4C5)sc23)c(C(=O)NC23CC(C(F)(F)F)(C2)C3)c1]

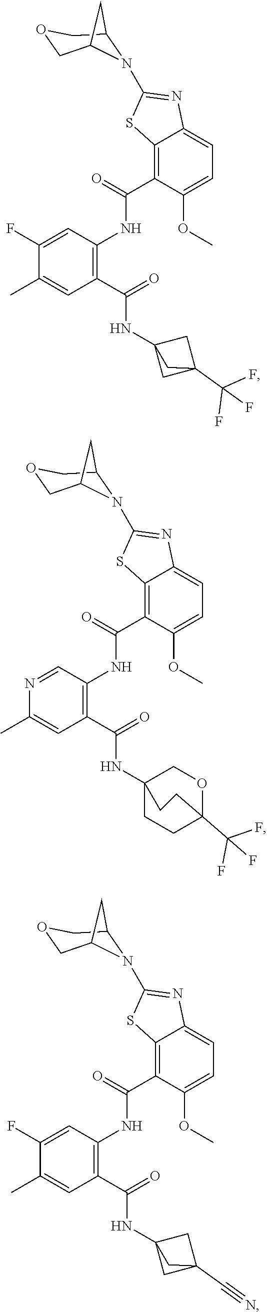 [SMILES: COc1ccc2nc(N3C4COCC3C4)sc2c1C(=O)Nc1cc(F)c(C)cc1C(=O)NC12CC(C(F)(F)F)(C1)C2 ; COc1ccc2nc(N3C4COCC3C4)sc2c1C(=O)Nc1cnc(C)cc1C(=O)NC12CCC(C(F)(F)F)(CC1)OC2 ; COc1ccc2nc(N3C4COCC3C4)sc2c1C(=O)Nc1cc(F)c(C)cc1C(=O)NC12CC(C#N)(C1)C2]

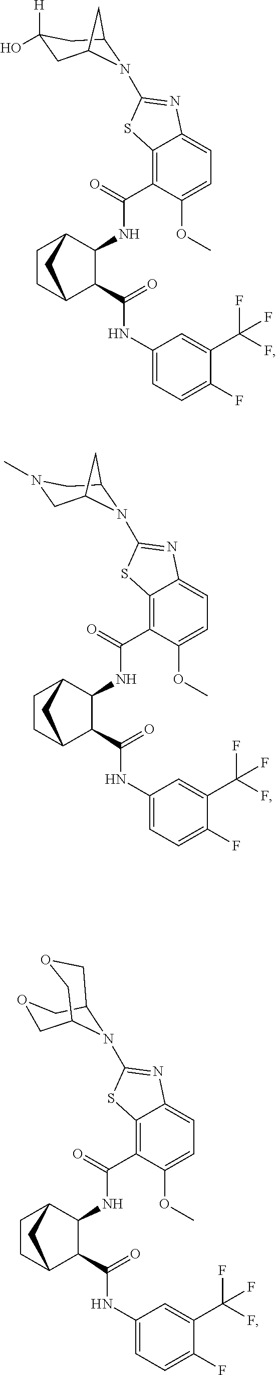 [SMILES: COc1ccc2nc(N3C4CC(O)CC3C4)sc2c1C(=O)N[C@@H]1[C@H]2CC[C@H](C2)[C@@H]1C(=O)Nc1ccc(F)c(C(F)(F)F)c1 ; COc1ccc2nc(N3C4CC3CN(C)C4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(COC4)C3)sc2c1C(=O)N[C@@H]1[C@H]2CC[C@H](C2)[C@@H]1C(=O)Nc1ccc(F)c(C(F)(F)F)c1]

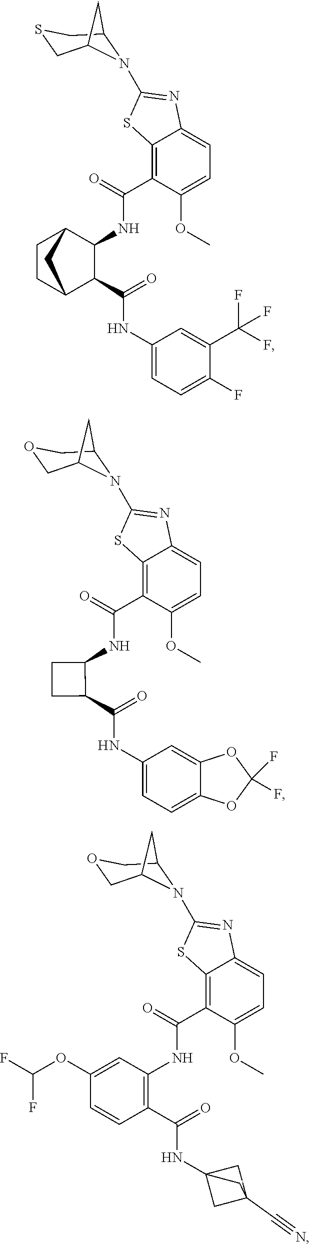

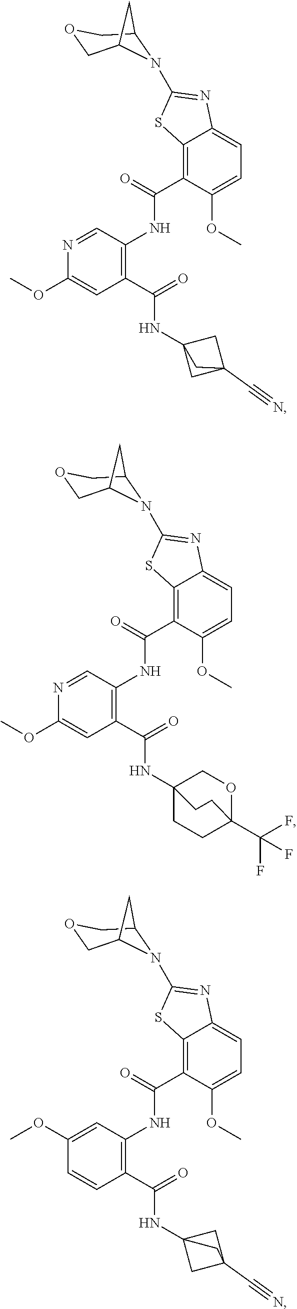 [SMILES: COc1cc(C(=O)NC23CC(C#N)(C2)C3)c(NC(=O)c2c(OC)ccc3nc(N4C5COCC4C5)sc23)cn1 ; COc1cc(C(=O)NC23CCC(C(F)(F)F)(CC2)OC3)c(NC(=O)c2c(OC)ccc3nc(N4C5COCC4C5)sc23)cn1 ; COc1ccc(C(=O)NC23CC(C#N)(C2)C3)c(NC(=O)c2c(OC)ccc3nc(N4C5COCC4C5)sc23)c1]

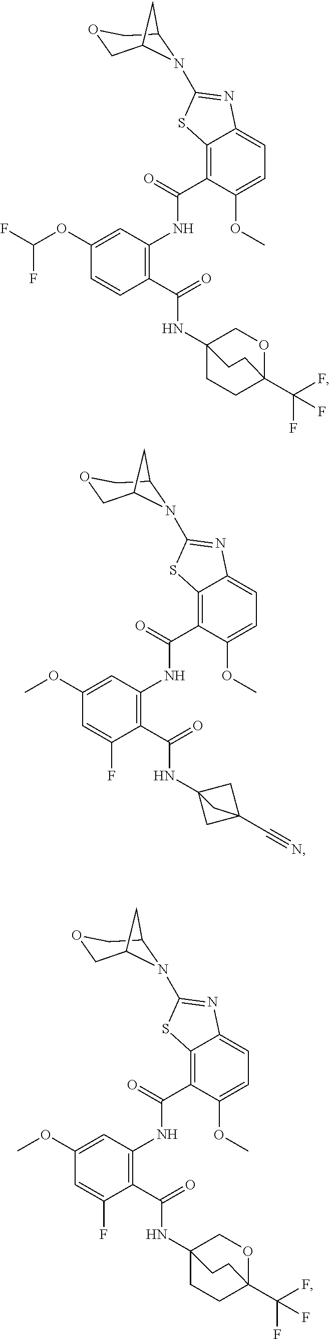 [SMILES: COc1ccc2nc(N3C4COCC3C4)sc2c1C(=O)Nc1cc(OC(F)F)ccc1C(=O)NC12CCC(C(F)(F)F)(CC1)OC2 ; COc1cc(F)c(C(=O)NC23CC(C#N)(C2)C3)c(NC(=O)c2c(OC)ccc3nc(N4C5COCC4C5)sc23)c1 ; COc1cc(F)c(C(=O)NC23CCC(C(F)(F)F)(CC2)OC3)c(NC(=O)c2c(OC)ccc3nc(N4C5COCC4C5)sc23)c1]

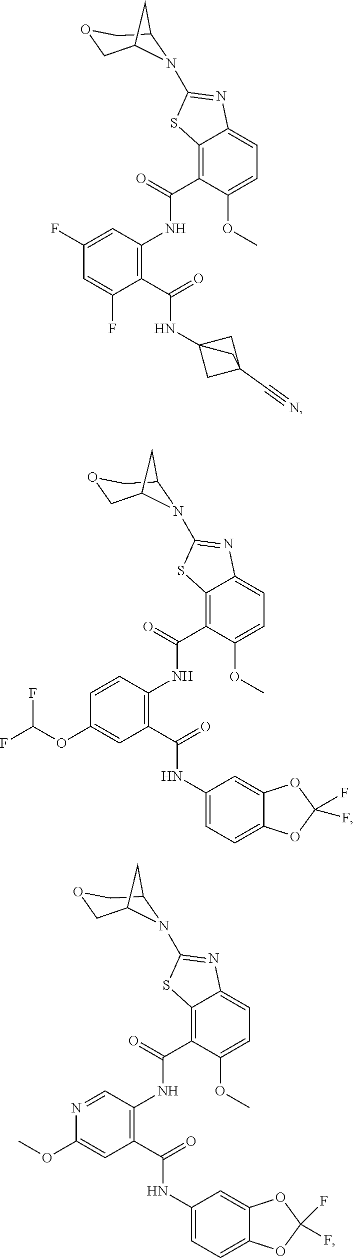 [SMILES: COc1ccc2nc(N3C4COCC3C4)sc2c1C(=O)Nc1cc(F)cc(F)c1C(=O)NC12CC(C#N)(C1)C2 ; COc1ccc2nc(N3C4COCC3C4)sc2c1C(=O)Nc1ccc(OC(F)F)cc1C(=O)Nc1ccc2c(c1)OC(F)(F)O2 ; COc1cc(C(=O)Nc2ccc3c(c2)OC(F)(F)O3)c(NC(=O)c2c(OC)ccc3nc(N4C5COCC4C5)sc23)cn1]

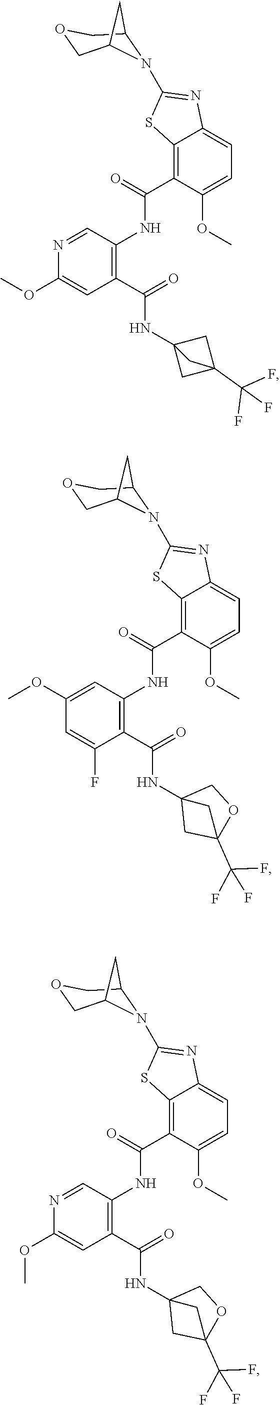 [SMILES: COc1cc(C(=O)NC23CC(C(F)(F)F)(C2)C3)c(NC(=O)c2c(OC)ccc3nc(N4C5COCC4C5)sc23)cn1 ; COc1cc(F)c(C(=O)NC23COC(C(F)(F)F)(C2)C3)c(NC(=O)c2c(OC)ccc3nc(N4C5COCC4C5)sc23)c1 ; COc1cc(C(=O)NC23COC(C(F)(F)F)(C2)C3)c(NC(=O)c2c(OC)ccc3nc(N4C5COCC4C5)sc23)cn1]

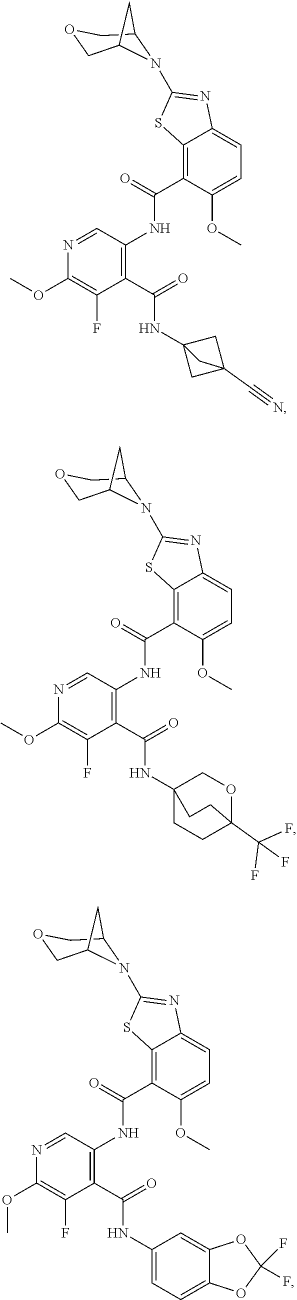 [SMILES: COc1ccc2nc(N3C4COCC3C4)sc2c1C(=O)Nc1cnc(OC)c(F)c1C(=O)NC12CC(C#N)(C1)C2 ; COc1ccc2nc(N3C4COCC3C4)sc2c1C(=O)Nc1cnc(OC)c(F)c1C(=O)NC12CCC(C(F)(F)F)(CC1)OC2 ; COc1ccc2nc(N3C4COCC3C4)sc2c1C(=O)Nc1cnc(OC)c(F)c1C(=O)Nc1ccc2c(c1)OC(F)(F)O2]

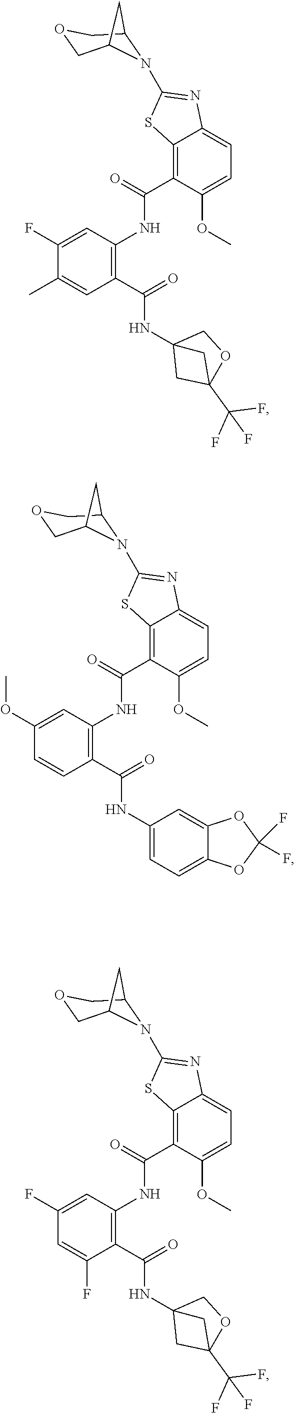 [SMILES: COc1ccc2nc(N3C4COCC3C4)sc2c1C(=O)Nc1cc(F)c(C)cc1C(=O)NC12COC(C(F)(F)F)(C1)C2 ; COc1ccc(C(=O)Nc2ccc3c(c2)OC(F)(F)O3)c(NC(=O)c2c(OC)ccc3nc(N4C5COCC4C5)sc23)c1 ; COc1ccc2nc(N3C4COCC3C4)sc2c1C(=O)Nc1cc(F)cc(F)c1C(=O)NC12COC(C(F)(F)F)(C1)C2]

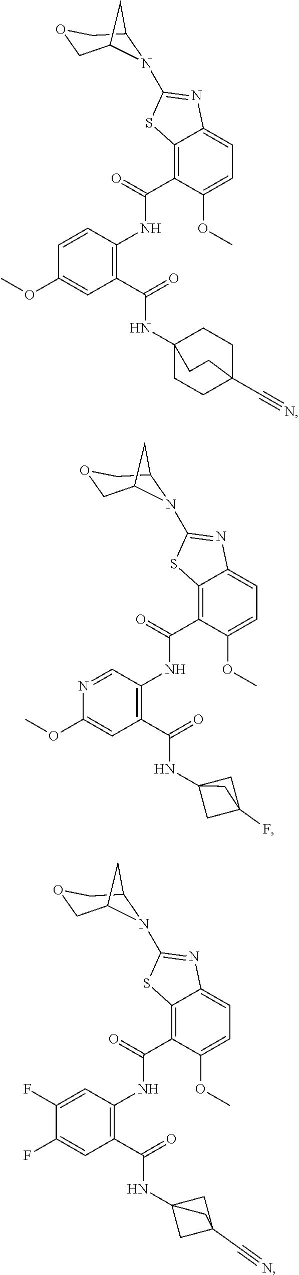 [SMILES: COc1ccc(NC(=O)c2c(OC)ccc3nc(N4C5COCC4C5)sc23)c(C(=O)NC23CCC(C#N)(CC2)CC3)c1 ; COc1cc(C(=O)NC23CC(F)(C2)C3)c(NC(=O)c2c(OC)ccc3nc(N4C5COCC4C5)sc23)cn1 ; COc1ccc2nc(N3C4COCC3C4)sc2c1C(=O)Nc1cc(F)c(F)cc1C(=O)NC12CC(C#N)(C1)C2]

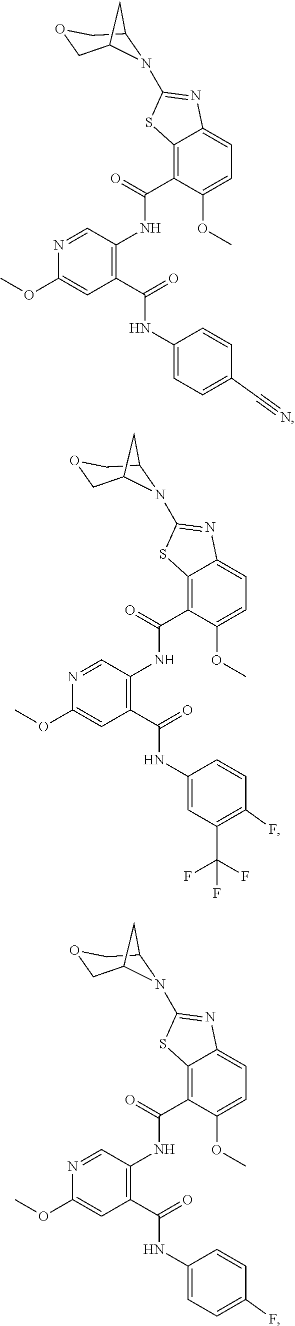 [SMILES: COc1cc(C(=O)Nc2ccc(C#N)cc2)c(NC(=O)c2c(OC)ccc3nc(N4C5COCC4C5)sc23)cn1 ; COc1cc(C(=O)Nc2ccc(F)c(C(F)(F)F)c2)c(NC(=O)c2c(OC)ccc3nc(N4C5COCC4C5)sc23)cn1 ; COc1cc(C(=O)Nc2ccc(F)cc2)c(NC(=O)c2c(OC)ccc3nc(N4C5COCC4C5)sc23)cn1]

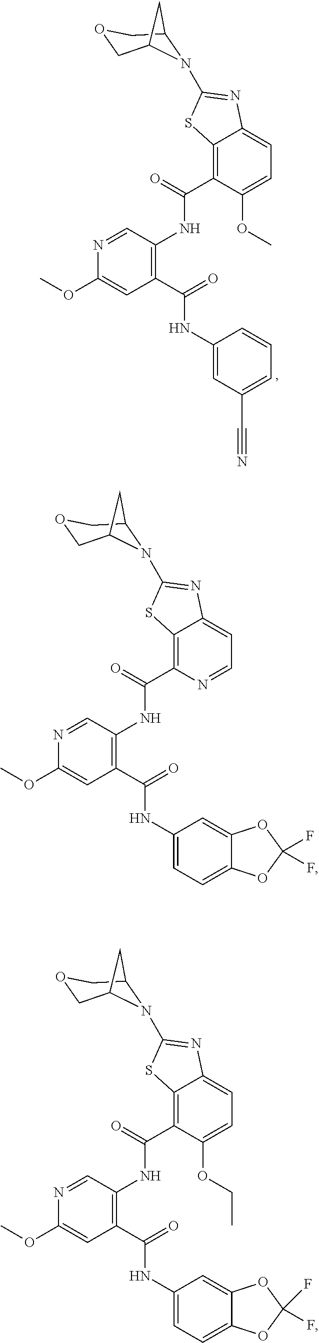

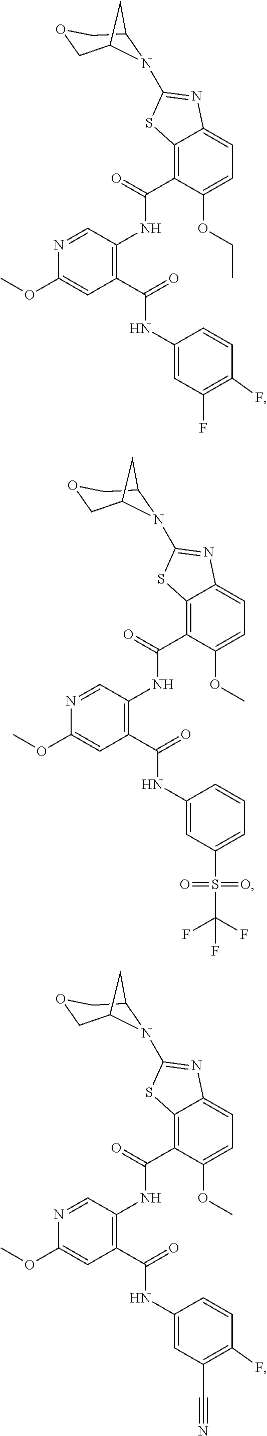 [SMILES: CCOc1ccc2nc(N3C4COCC3C4)sc2c1C(=O)Nc1cnc(OC)cc1C(=O)Nc1ccc(F)c(F)c1 ; COc1cc(C(=O)Nc2cccc(S(=O)(=O)C(F)(F)F)c2)c(NC(=O)c2c(OC)ccc3nc(N4C5COCC4C5)sc23)cn1 ; COc1cc(C(=O)Nc2ccc(F)c(C#N)c2)c(NC(=O)c2c(OC)ccc3nc(N4C5COCC4C5)sc23)cn1]

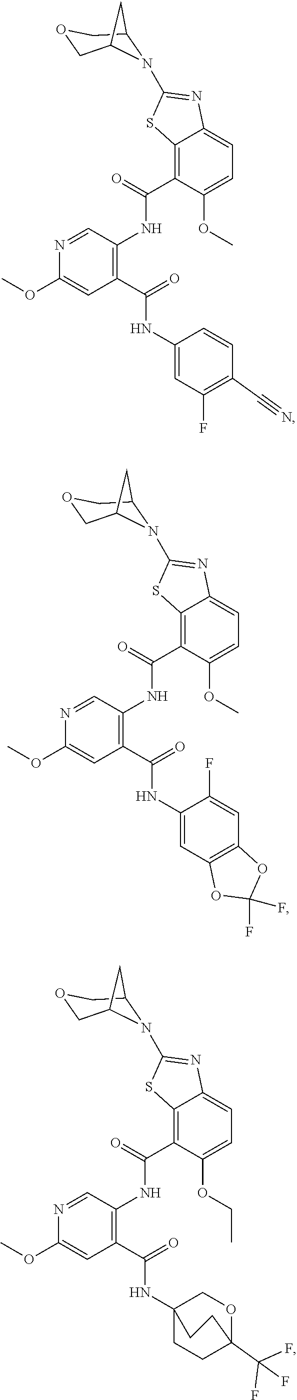 [SMILES: COc1cc(C(=O)Nc2ccc(C#N)c(F)c2)c(NC(=O)c2c(OC)ccc3nc(N4C5COCC4C5)sc23)cn1 ; COc1cc(C(=O)Nc2cc3c(cc2F)OC(F)(F)O3)c(NC(=O)c2c(OC)ccc3nc(N4C5COCC4C5)sc23)cn1 ; CCOc1ccc2nc(N3C4COCC3C4)sc2c1C(=O)Nc1cnc(OC)cc1C(=O)NC12CCC(C(F)(F)F)(CC1)OC2]

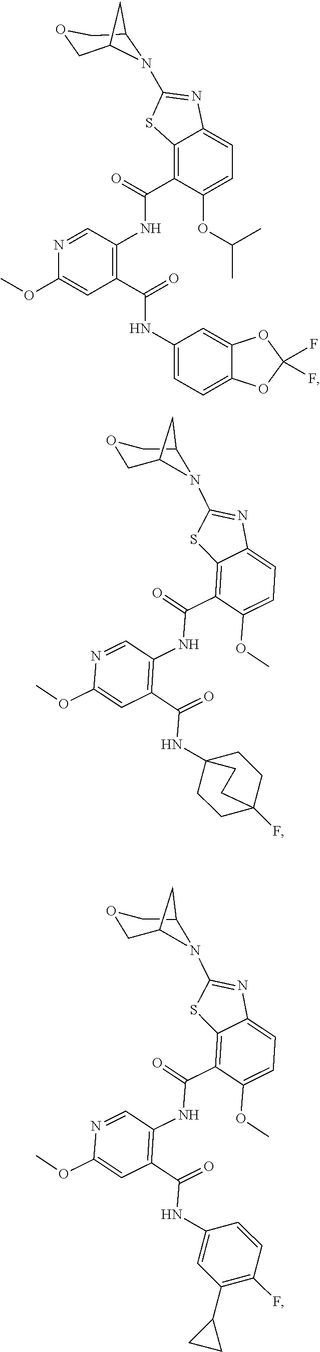

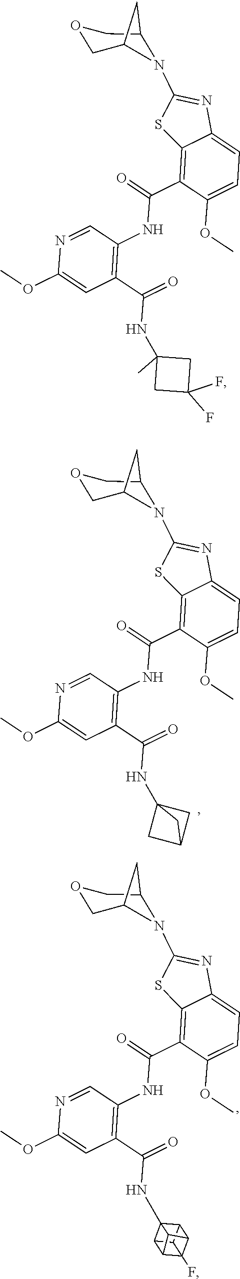

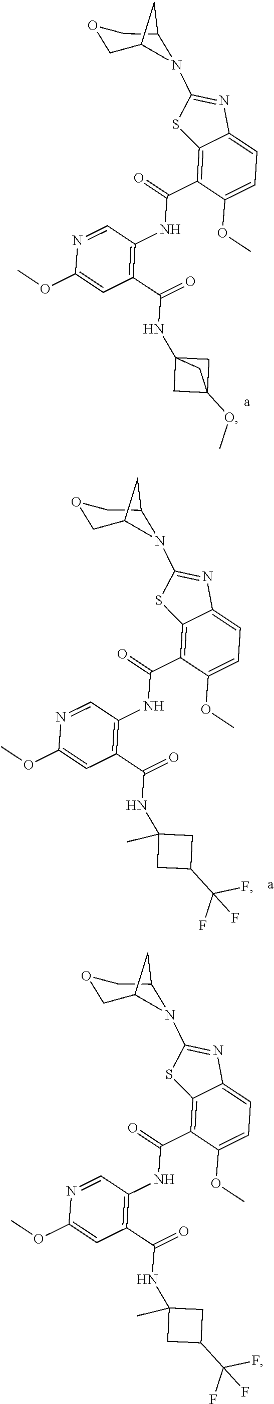 [SMILES: COc1cc(C(=O)NC23CC(OC)(C2)C3)c(NC(=O)c2c(OC)ccc3nc(N4C5COCC4C5)sc23)cn1 ; COc1cc(C(=O)NC2(C)CC(C(F)(F)F)C2)c(NC(=O)c2c(OC)ccc3nc(N4C5COCC4C5)sc23)cn1 ; COc1cc(C(=O)NC2(C)CC(C(F)(F)F)C2)c(NC(=O)c2c(OC)ccc3nc(N4C5COCC4C5)sc23)cn1]

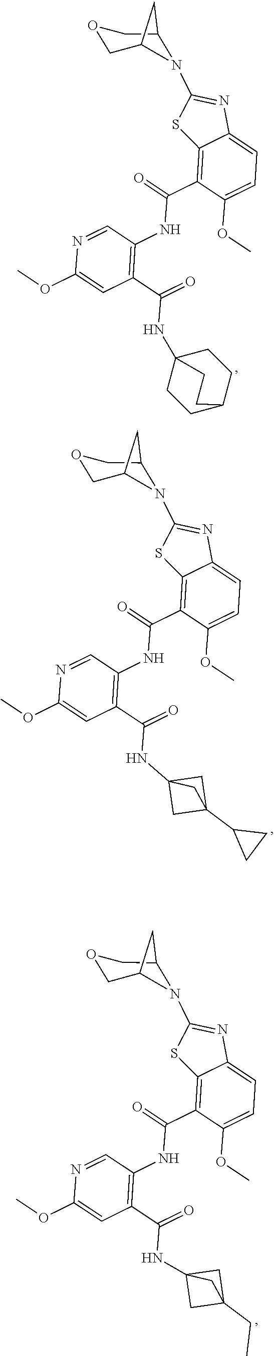 [SMILES: COc1cc(C(=O)NC23CCC(CC2)CC3)c(NC(=O)c2c(OC)ccc3nc(N4C5COCC4C5)sc23)cn1 ; COc1cc(C(=O)NC23CC(C4CC4)(C2)C3)c(NC(=O)c2c(OC)ccc3nc(N4C5COCC4C5)sc23)cn1 ; CCC12CC(NC(=O)c3cc(OC)ncc3NC(=O)c3c(OC)ccc4nc(N5C6COCC5C6)sc34)(C1)C2]

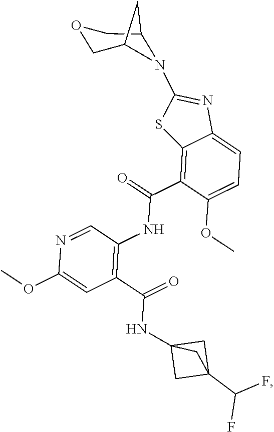 [SMILES: COc1cc(C(=O)NC23CC(C(F)F)(C2)C3)c(NC(=O)c2c(OC)ccc3nc(N4C5COCC4C5)sc23)cn1]

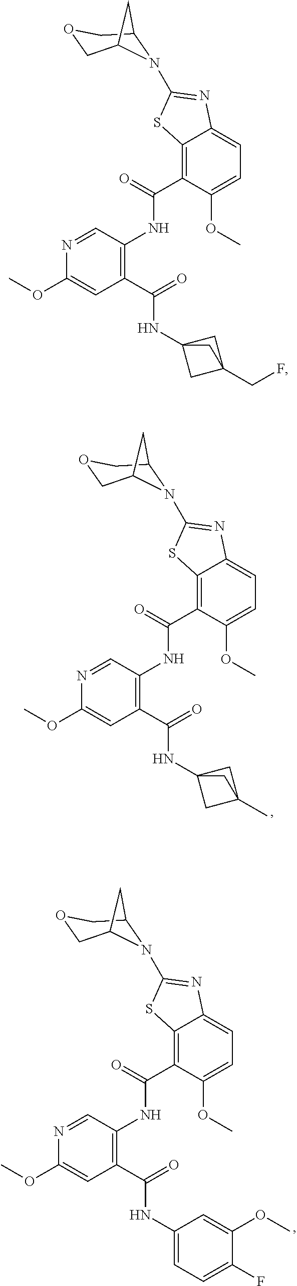

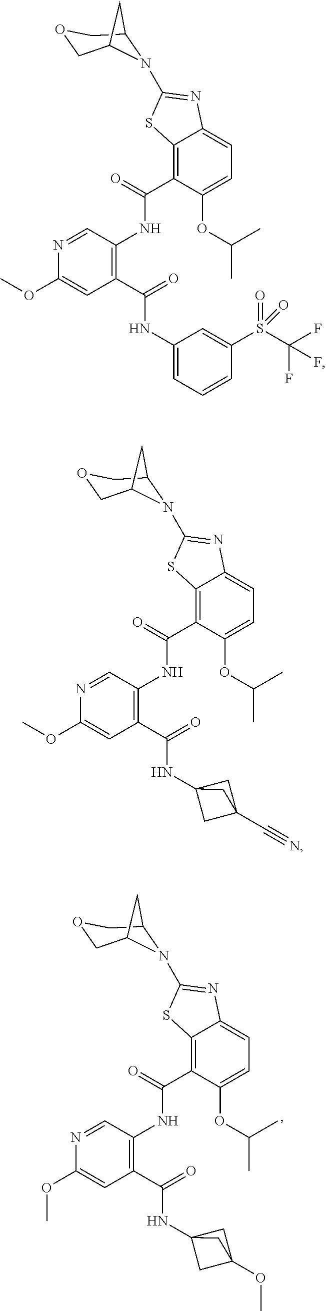 [SMILES: COc1cc(C(=O)Nc2cccc(S(=O)(=O)C(F)(F)F)c2)c(NC(=O)c2c(OC(C)C)ccc3nc(N4C5COCC4C5)sc23)cn1 ; COc1cc(C(=O)NC23CC(C#N)(C2)C3)c(NC(=O)c2c(OC(C)C)ccc3nc(N4C5COCC4C5)sc23)cn1 ; COc1cc(C(=O)NC23CC(OC)(C2)C3)c(NC(=O)c2c(OC(C)C)ccc3nc(N4C5COCC4C5)sc23)cn1]

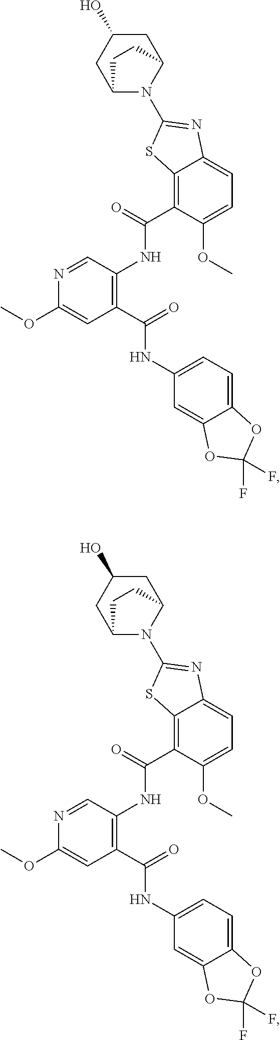 [SMILES: COc1cc(C(=O)Nc2ccc3c(c2)OC(F)(F)O3)c(NC(=O)c2c(OC)ccc3nc(N4[C@@H]5CC[C@H]4C[C@@H](O)C5)sc23)cn1 ; COc1cc(C(=O)Nc2ccc3c(c2)OC(F)(F)O3)c(NC(=O)c2c(OC)ccc3nc(N4[C@@H]5CC[C@H]4C[C@H](O)C5)sc23)cn1]

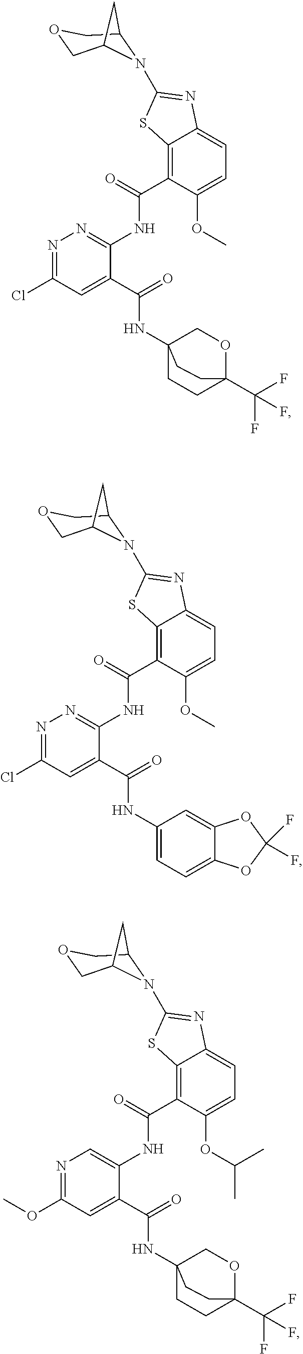 [SMILES: COc1ccc2nc(N3C4COCC3C4)sc2c1C(=O)Nc1nnc(Cl)cc1C(=O)NC12CCC(C(F)(F)F)(CC1)OC2 ; COc1ccc2nc(N3C4COCC3C4)sc2c1C(=O)Nc1nnc(Cl)cc1C(=O)Nc1ccc2c(c1)OC(F)(F)O2 ; COc1cc(C(=O)NC23CCC(C(F)(F)F)(CC2)OC3)c(NC(=O)c2c(OC(C)C)ccc3nc(N4C5COCC4C5)sc23)cn1]

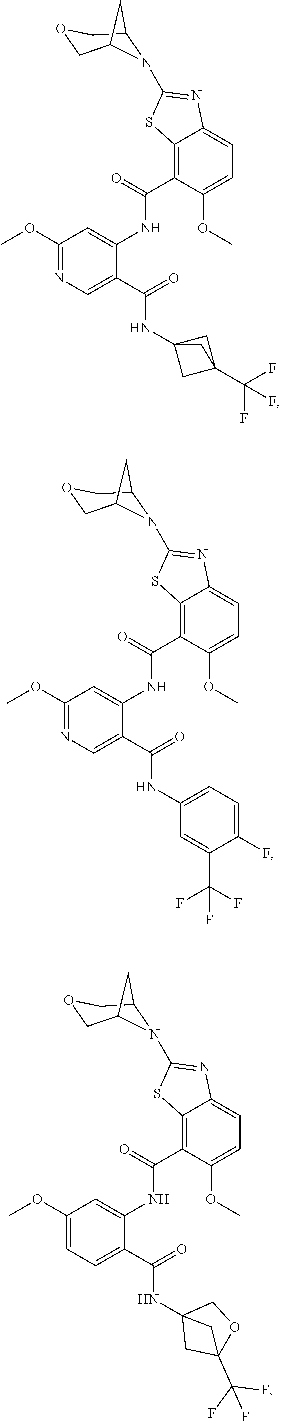 [SMILES: COc1cc(NC(=O)c2c(OC)ccc3nc(N4C5COCC4C5)sc23)c(C(=O)NC23CC(C(F)(F)F)(C2)C3)cn1 ; COc1cc(NC(=O)c2c(OC)ccc3nc(N4C5COCC4C5)sc23)c(C(=O)Nc2ccc(F)c(C(F)(F)F)c2)cn1 ; COc1ccc(C(=O)NC23COC(C(F)(F)F)(C2)C3)c(NC(=O)c2c(OC)ccc3nc(N4C5COCC4C5)sc23)c1]

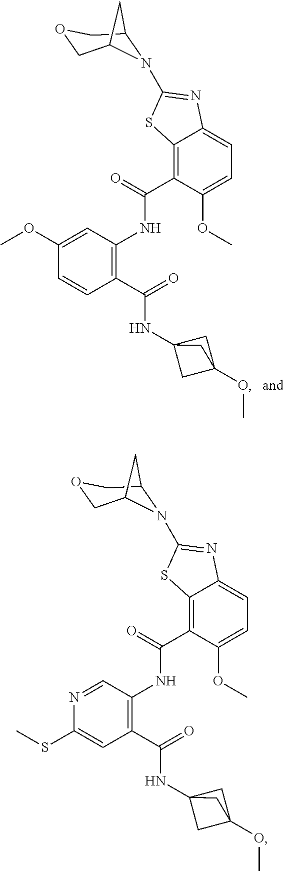

28. The compound according to aspect 1 selected from

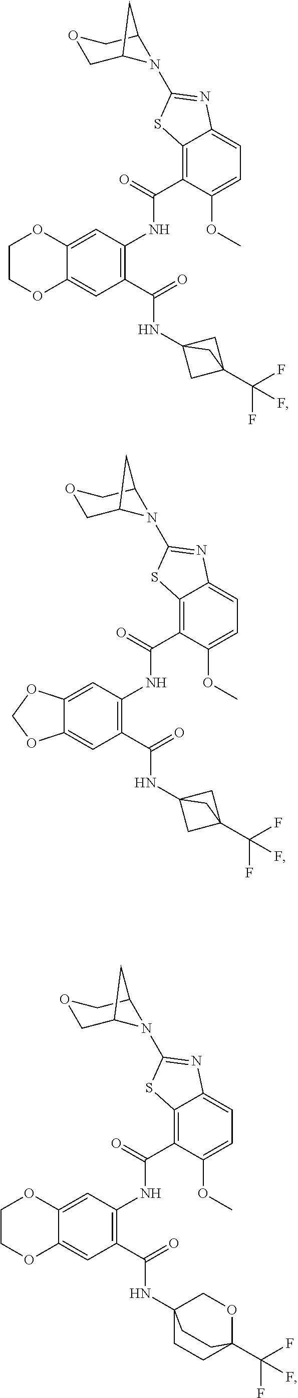 [SMILES: COc1ccc2nc(N3C4COCC3C4)sc2c1C(=O)Nc1cc2c(cc1C(=O)NC13CC(C(F)(F)F)(C1)C3)OCCO2 ; COc1ccc2nc(N3C4COCC3C4)sc2c1C(=O)Nc1cc2c(cc1C(=O)NC13CC(C(F)(F)F)(C1)C3)OCO2 ; COc1ccc2nc(N3C4COCC3C4)sc2c1C(=O)Nc1cc2c(cc1C(=O)NC13CCC(C(F)(F)F)(CC1)OC3)OCCO2]

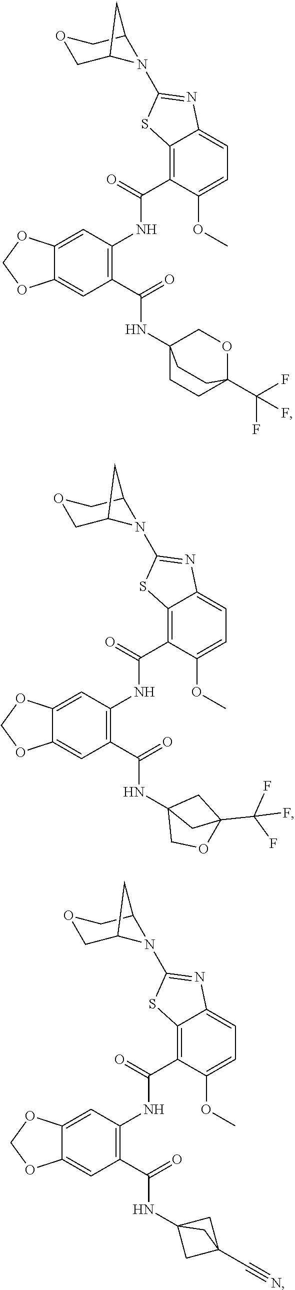 [SMILES: COc1ccc2nc(N3C4COCC3C4)sc2c1C(=O)Nc1cc2c(cc1C(=O)NC13CCC(C(F)(F)F)(CC1)OC3)OCO2 ; COc1ccc2nc(N3C4COCC3C4)sc2c1C(=O)Nc1cc2c(cc1C(=O)NC13COC(C(F)(F)F)(C1)C3)OCO2 ; COc1ccc2nc(N3C4COCC3C4)sc2c1C(=O)Nc1cc2c(cc1C(=O)NC13CC(C#N)(C1)C3)OCO2]

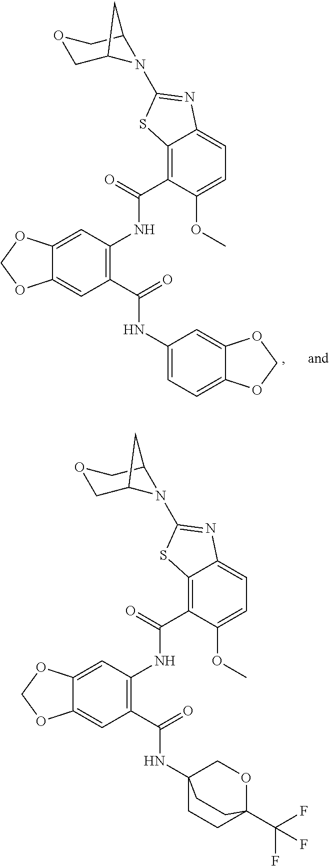 [SMILES: COc1ccc2nc(N3C4COCC3C4)sc2c1C(=O)Nc1cc2c(cc1C(=O)Nc1ccc3c(c1)OCO3)OCO2 ; COc1ccc2nc(N3C4COCC3C4)sc2c1C(=O)Nc1cc2c(cc1C(=O)NC13CCC(C(F)(F)F)(CC1)OC3)OCO2]

and or a pharmaceutically acceptable salt thereof.

29. A pharmaceutical composition comprising a compound as defined in any one of aspects 1-28 or a pharmaceutically acceptable salt thereof, and a pharmaceutically acceptable carrier, diluent or excipient.

30. A method of treating cardiovascular, pulmonary and/or renal conditions, diseases and/or disorders in an individual, the method comprising administering to the individual an effective amount of a compound of any one of aspects 1-28 or a pharmaceutical composition of aspect 29.

31. A compound of any one of aspects 1-28 for use in a therapy.

32. A compound of any one of aspects 1-28 for use in treating cardiovascular, pulmonary and/or renal conditions, diseases and/or disorders.

33. Use of a compound of any one of aspects 1-28 for the manufacture of a medicament for treating cardiovascular, pulmonary and/or renal conditions, diseases and/or disorders.

34. A method according to aspect 30, a compound for use according to aspect 32, or a use according to aspect 33, wherein the disease or disorder to be treated is a cardiovascular condition, disease, or disorder selected from acute heart failure, chronic heart failure, atherosclerosis, coronary artery disease, diabetes, stroke, hypercholesterolemia, hypertension, ischemia, vasoconstriction, or ventricular hypertrophy.

35. A method according to aspect 30, a compound for use according to aspect 32, or a use according to aspect 33, wherein the disease or disorder to be treated is a pulmonary condition, disease, or disorder selected from pulmonary hypertension or chronic obstructive pulmonary disease (COPD).

36. A method according to aspect 30, a compound for use according to aspect 32, or a use according to aspect 33, wherein the disease or disorder to be treated is a renal condition, disease, or disorder selected from acute kidney disease, chronic kidney disease or diabetes nephropathy.

The present invention provides a compound of the formula:

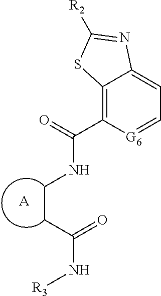

wherein G₆, R₂, Ring A, and R₃, are as defined above, or a pharmaceutically acceptable salt thereof.

In an embodiment, Ring A is a group of the formula

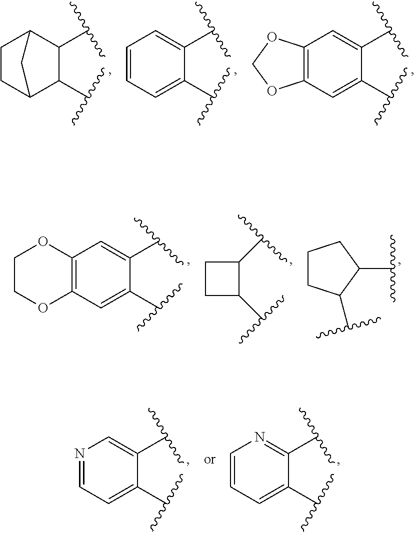

wherein each of

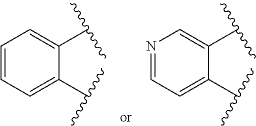 [SMILES: *c1ccccc1* ; *c1ccncc1*]

are optionally substituted by one or two halogens, C₁₋₄ alkyl, or C₁₋₃ alkoxy, wherein each C₁₋₄ alkyl or C₁₋₃ alkoxy is independently optionally substituted by one or more halogens or —CN;
R₂ is a group of the formula

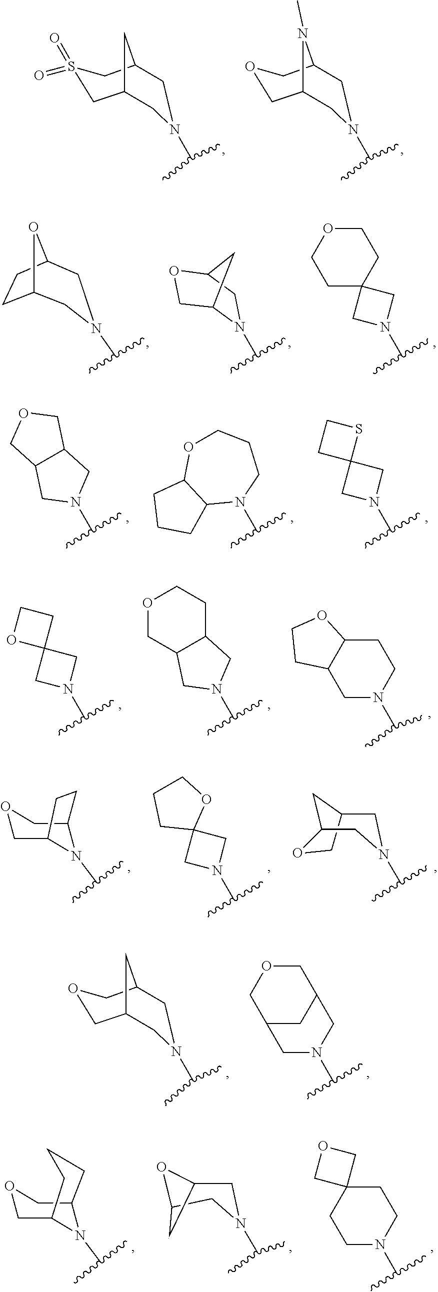

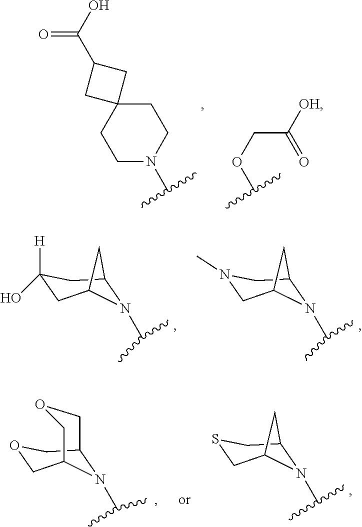

wherein

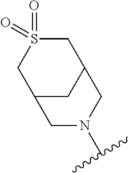 [SMILES: *N1CC2CC(C1)CS(=O)(=O)C2]

is optionally substituted with one or two halogens, wherein

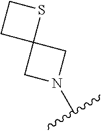 [SMILES: *N1CC2(CCS2)C1]

is optionally substituted with one or two oxo;
wherein R₃ is selected from the group consisting of

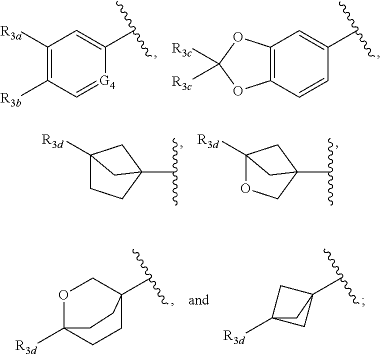

R₃ₐ, at each occurrence, is independently —H, halogen, —SF₅, —SO₂CF₃, —SCF₃, C₁₋₄ alkyl, or C₁₋₃ alkoxy, wherein the C₁₋₄ alkyl, or C₁₋₃ alkoxy are optionally substituted with one or two halogens;
R_{3d} is —H, halogen, —CN, C₁₋₄ alkyl, or C₁₋₃ alkoxy, wherein the C₁₋₄ alkyl or C₁₋₃ alkoxy are optionally substituted with one or two halogens; and
—G₆— is —C(R₁)—; or a pharmaceutically acceptable salt thereof.

In another embodiment, Ring A is a group of the formula

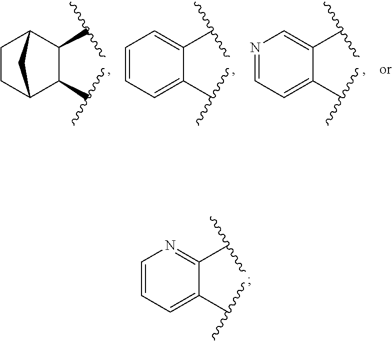

R₂ is —OCH₂COOH or an N-linked 4-7 membered heterocycle; wherein the N-linked 4-7 membered heterocycle is selected from an azetidine, pyrrolidine, morpholine, piperazine, piperidine, or oxazepane; wherein the heterocycle is optionally bridged by, fused with, or spiro-linked with a C₁₋₄ alkylenyl, or 1-5 membered heteroalkyl; wherein the heterocycle, the C₁₋₄ alkylenyl, and 1-5 membered heteroalkyl are each optionally substituted with 1-3 substituents each independently selected from halogen, C₁₋₄ alkyl, C₁₋₄ haloalkyl, and —COOH;
R₃ is a group of the formula:

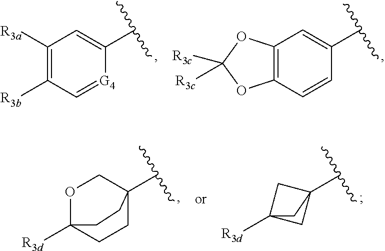

R₃ₐ is —H, halogen, —SF₅, —SO₂CF₃, —SCF₃, C₁₋₄ alkyl, or C₁₋₃ alkoxy, wherein the C₁₋₄ alkyl, or C₁₋₃ alkoxy are optionally substituted with one or more halogens;
each R_{3c} is independently —H, or —F; and
R_{3d} is —H, halogen, C₁₋₄ alkyl, or C₁₋₃ alkoxy, wherein the C₁₋₄ alkyl, or C₁₋₃ alkoxy are optionally substituted with one or more halogens; and
—G₆— is —C(R₁)—; or a pharmaceutically acceptable salt thereof.

In another embodiment, Ring A is a group of the formula

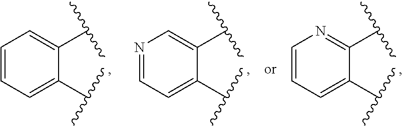 [SMILES: *c1ccccc1* ; *c1ccncc1* ; *c1cccnc1*]

wherein each of

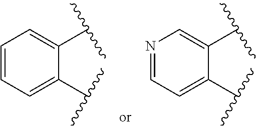 [SMILES: *c1ccccc1* ; *c1ccncc1*]

are optionally substituted by one or more selected from halogens, C₁₋₄ alkyl, and C₁₋₃ alkoxy, wherein each C₁₋₄ alkyl or C₁₋₃ alkoxy is independently optionally substituted by one or more selected from halogens and —CN;
or a pharmaceutically acceptable salt thereof.

In another embodiment, Ring A is a group of the formula

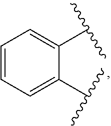 [SMILES: *c1ccccc1*]

wherein

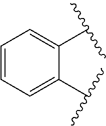 [SMILES: *c1ccccc1*]

is optionally substituted by one or more selected from halogens, C₁₋₄ alkyl, and C₁₋₃ alkoxy, wherein each C₁₋₄ alkyl or C₁₋₃ alkoxy is independently optionally substituted by one or more selected from halogens and —CN;
or a pharmaceutically acceptable salt thereof.

In another embodiment, Ring A is a group of the formula

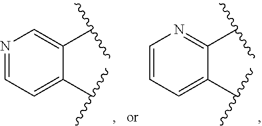 [SMILES: *c1ccncc1* ; *c1cccnc1*]

wherein

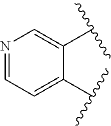 [SMILES: *c1ccncc1*]

is optionally substituted by one or more selected from halogens, C₁₋₄ alkyl, and C₁₋₃ alkoxy, wherein each C₁₋₄ alkyl or C₁₋₃ alkoxy is independently optionally substituted by one or more selected from halogens and —CN;
or a pharmaceutically acceptable salt thereof.

In another embodiment, Ring A is a group of the formula

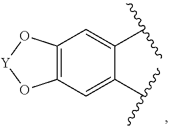

or a pharmaceutically acceptable salt thereof.

In an embodiment, Ring A is selected from the group consisting of

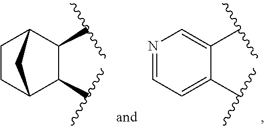 [SMILES: *[C@@H]1[C@H]2CC[C@H](C2)[C@@H]1* ; *c1ccncc1*]

or a pharmaceutically acceptable salt thereof.

In another embodiment, —G₆— is —C(R₁)—, or a pharmaceutically acceptable salt thereof.

In another embodiment, the compound is of the formula

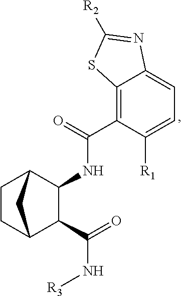

or a pharmaceutically acceptable salt thereof.

In another embodiment, the compound is of the formula

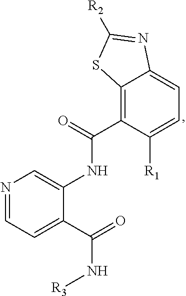

or a pharmaceutically acceptable salt thereof.

In another embodiment, R₁ is methoxy, ethoxy, or isopropoxy or a pharmaceutically acceptable salt thereof.

In another embodiment, R₁ is methoxy, or a pharmaceutically acceptable salt thereof.

In another embodiment, R₃ is selected from the group consisting of

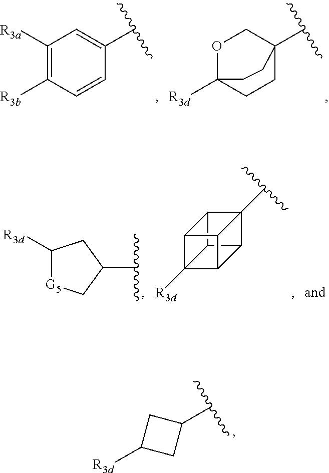

or a pharmaceutically acceptable salt thereof.

In another embodiment, R_{3d} is trifluoromethyl, or a pharmaceutically acceptable salt thereof.

In another embodiment, R₃ is

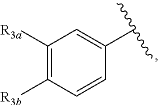

or a pharmaceutically acceptable salt thereof.

In another embodiment, R₃ₐ is —F, —Cl, —Br, difluoromethyl, difluorochloromethyl, trifluoromethyl, trifluoromethoxy, or difluoromethoxy, and R_{3b} is —H, —F, —Cl, —Br, trifluoromethyl, —CN, or methyl, or a pharmaceutically acceptable salt thereof.

In another embodiment, R₃ is selected from the group consisting of

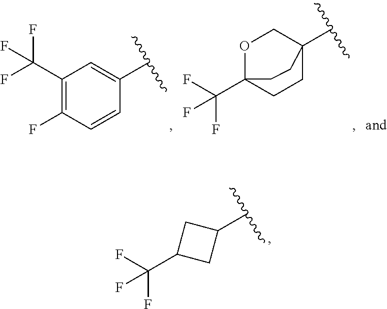

or a pharmaceutically acceptable salt thereof.

In another embodiment, R₃ is

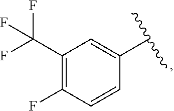 [SMILES: *c1ccc(F)c(C(F)(F)F)c1]

or a pharmaceutically acceptable salt thereof.

In another embodiment, the compound is of the formula

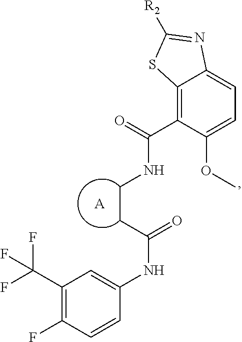

or a pharmaceutically acceptable salt thereof.

In another embodiment, R₂ is selected from the group consisting of

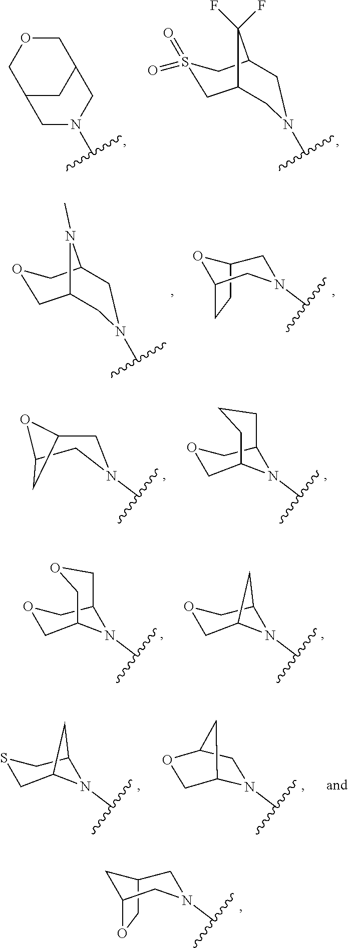

or a pharmaceutically acceptable salt thereof.

In another embodiment, R₂ is a group of the formula

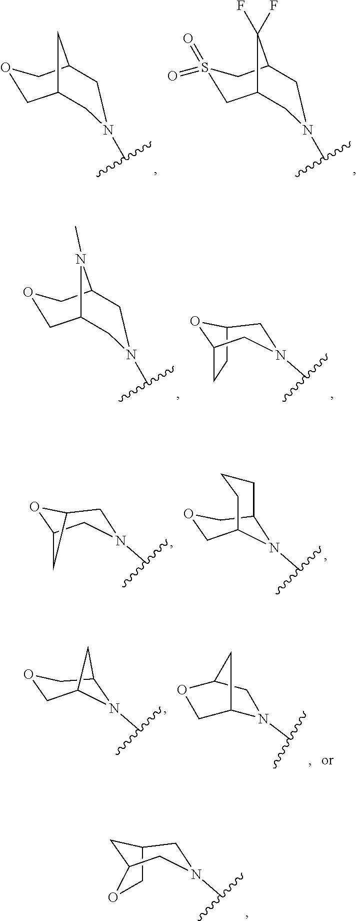

or a pharmaceutically acceptable salt thereof.

In another embodiment, R₂ is a group of the formula

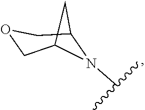 [SMILES: *N1C2COCC1C2]

or a pharmaceutically acceptable salt thereof.

In another embodiment, R₂ is selected from the group consisting of

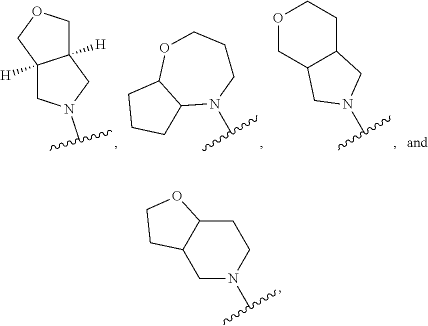

or a pharmaceutically acceptable salt thereof.

In another embodiment, R₂ is selected from the group consisting of

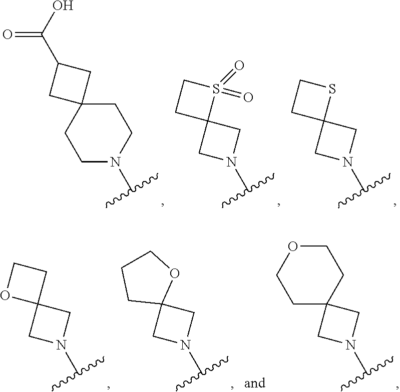

or a pharmaceutically acceptable salt thereof.

In another embodiment, R₂ is

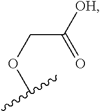 [SMILES: *OCC(=O)O]

or a pharmaceutically acceptable salt thereof.

In another embodiment, the compound is selected from the group consisting of

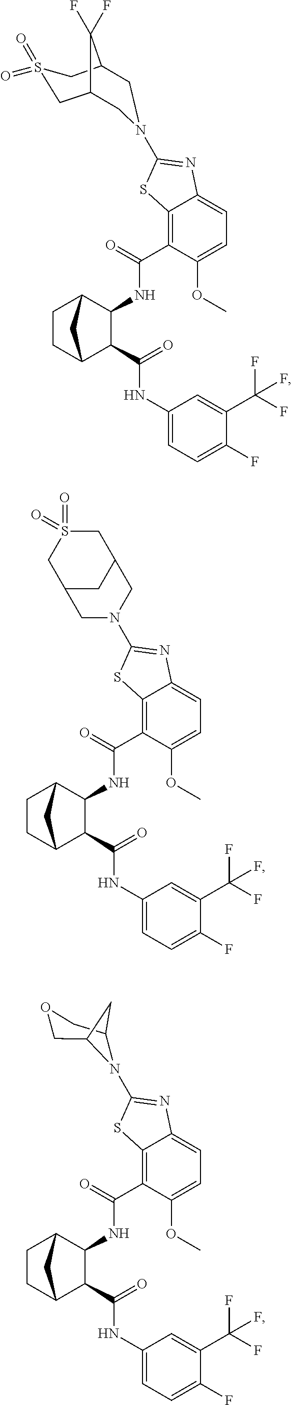 [SMILES: COc1ccc2nc(N3CC4CS(=O)(=O)CC(C3)C4(F)F)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)CS(=O)(=O)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1]

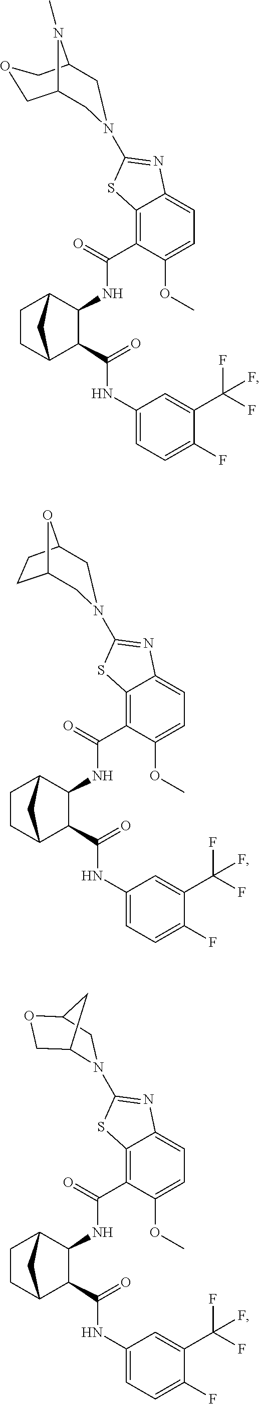 [SMILES: COc1ccc2nc(N3CC4COCC(C3)N4C)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1]

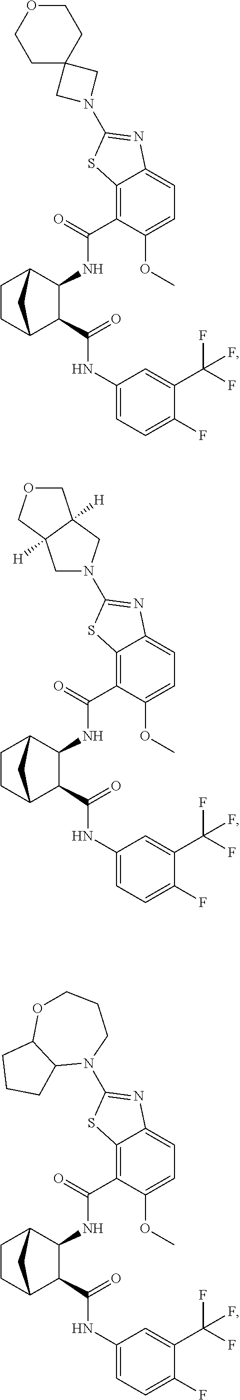 [SMILES: COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCCOC4CCCC43)sc2c1C(=O)N[C@@H]1[C@H]2CC[C@H](C2)[C@@H]1C(=O)Nc1ccc(F)c(C(F)(F)F)c1]

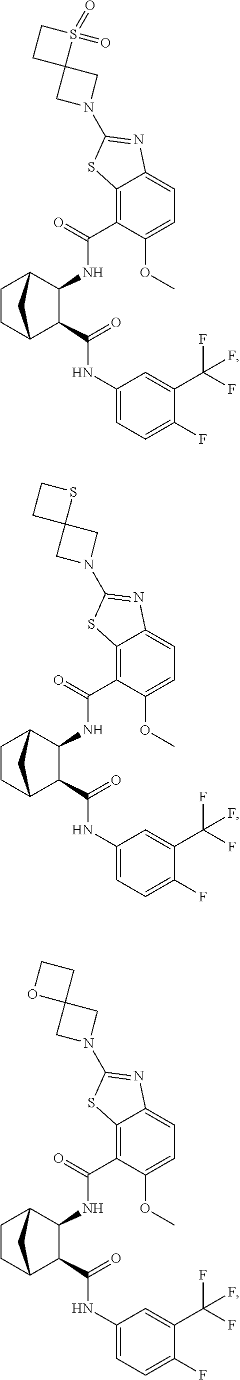 [SMILES: COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1]

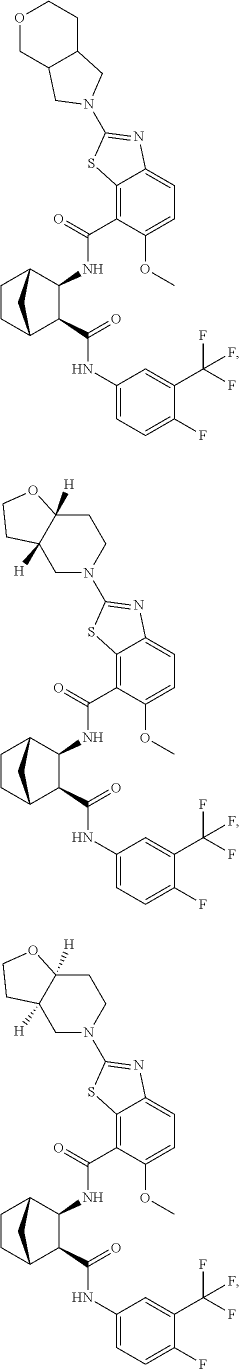 [SMILES: COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@H]4OCC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@@H]4OCC[C@@H]4C3)sc2c1C(=O)N[C@@H]1[C@H]2CC[C@H](C2)[C@@H]1C(=O)Nc1ccc(F)c(C(F)(F)F)c1]

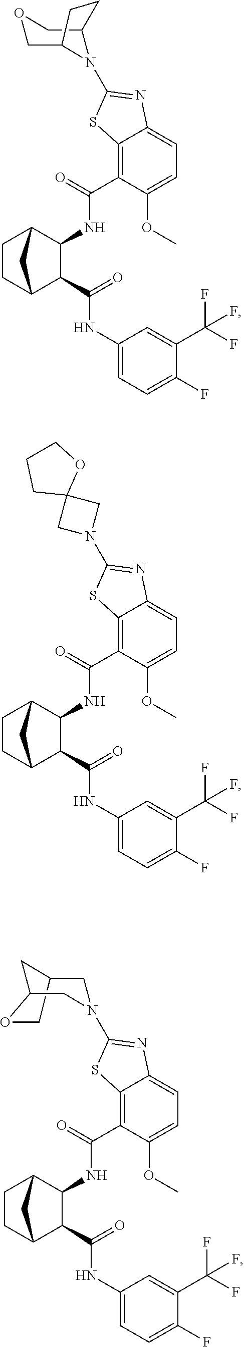 [SMILES: COc1ccc2nc(N3C4CCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4COC(C4)C3)sc2c1C(=O)N[C@@H]1[C@H]2CC[C@H](C2)[C@@H]1C(=O)Nc1ccc(F)c(C(F)(F)F)c1]

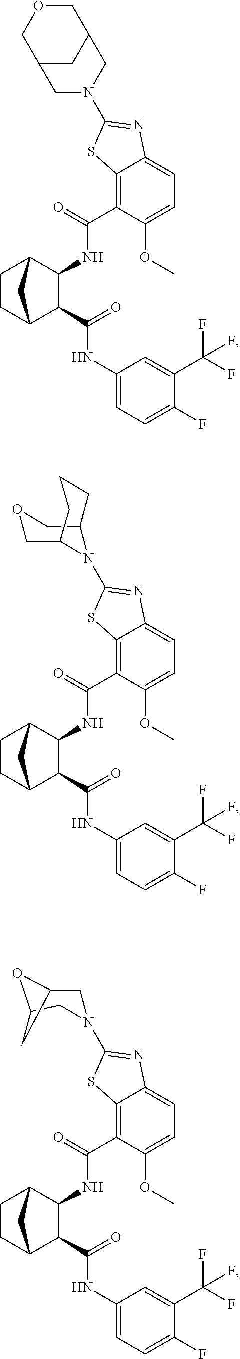 [SMILES: COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C4CCCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1]

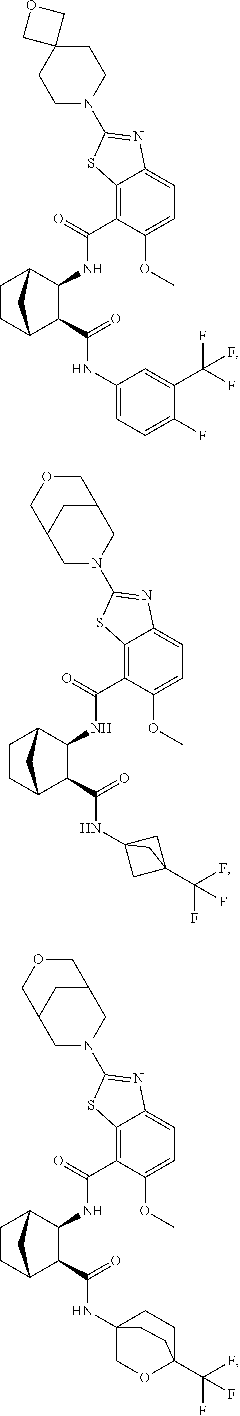 [SMILES: COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2 ; COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2]

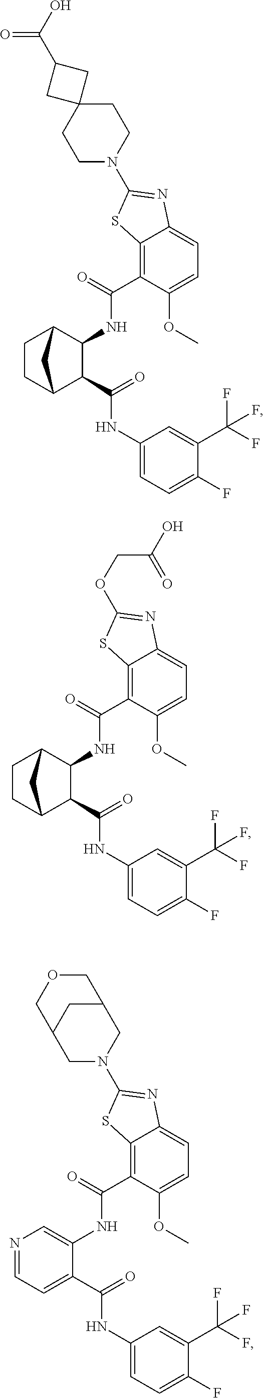

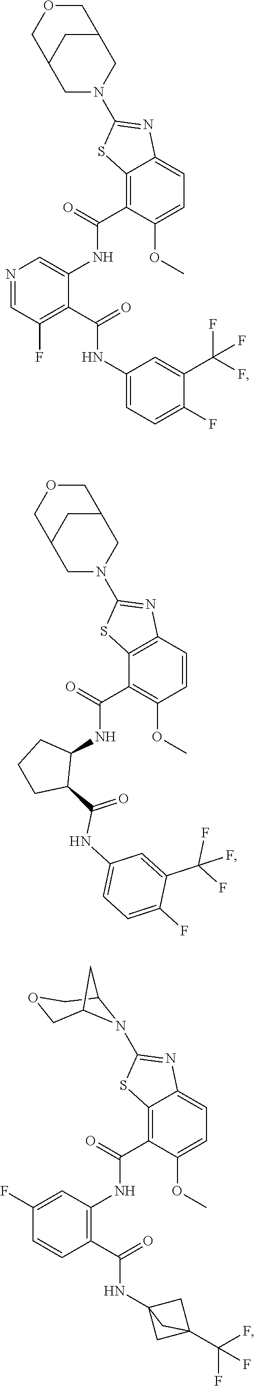 [SMILES: COc1ccc2nc(N3CC4COCC(C4)C3)sc2c1C(=O)Nc1cncc(F)c1C(=O)Nc1ccc(F)c(C(F)(F)F)c1 ; COc1ccc2nc(N3CC4COCC(C4)C3)sc2c1C(=O)N[C@@H]1CCC[C@@H]1C(=O)Nc1ccc(F)c(C(F)(F)F)c1 ; COc1ccc2nc(N3C4COCC3C4)sc2c1C(=O)Nc1cc(F)ccc1C(=O)NC12CC(C(F)(F)F)(C1)C2]

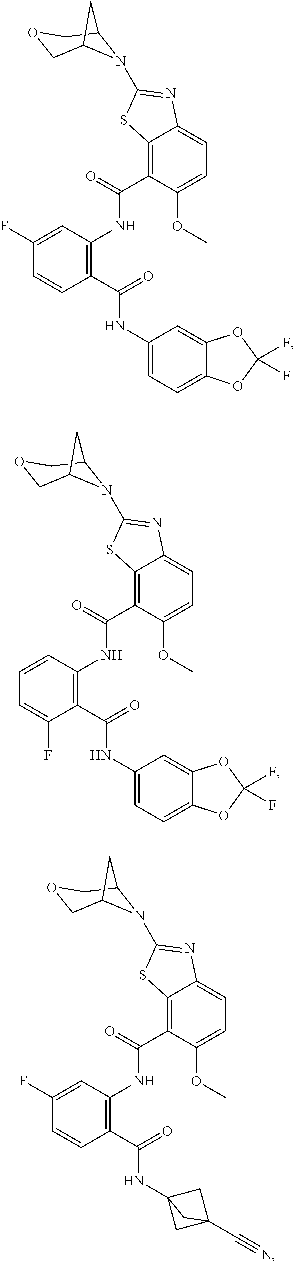 [SMILES: COc1ccc2nc(N3C4COCC3C4)sc2c1C(=O)Nc1cc(F)ccc1C(=O)Nc1ccc2c(c1)OC(F)(F)O2 ; COc1ccc2nc(N3C4COCC3C4)sc2c1C(=O)Nc1cccc(F)c1C(=O)Nc1ccc2c(c1)OC(F)(F)O2 ; COc1ccc2nc(N3C4COCC3C4)sc2c1C(=O)Nc1cc(F)ccc1C(=O)NC12CC(C#N)(C1)C2]

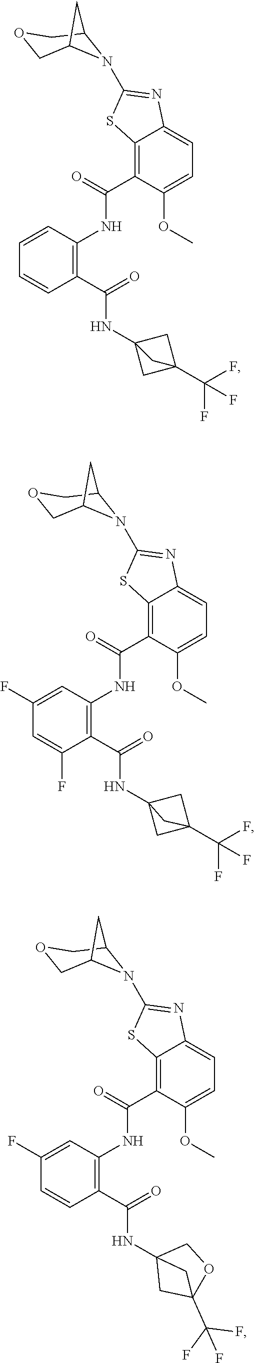 [SMILES: COc1ccc2nc(N3C4COCC3C4)sc2c1C(=O)Nc1ccccc1C(=O)NC12CC(C(F)(F)F)(C1)C2 ; COc1ccc2nc(N3C4COCC3C4)sc2c1C(=O)Nc1cc(F)cc(F)c1C(=O)NC12CC(C(F)(F)F)(C1)C2 ; COc1ccc2nc(N3C4COCC3C4)sc2c1C(=O)Nc1cc(F)ccc1C(=O)NC12COC(C(F)(F)F)(C1)C2]

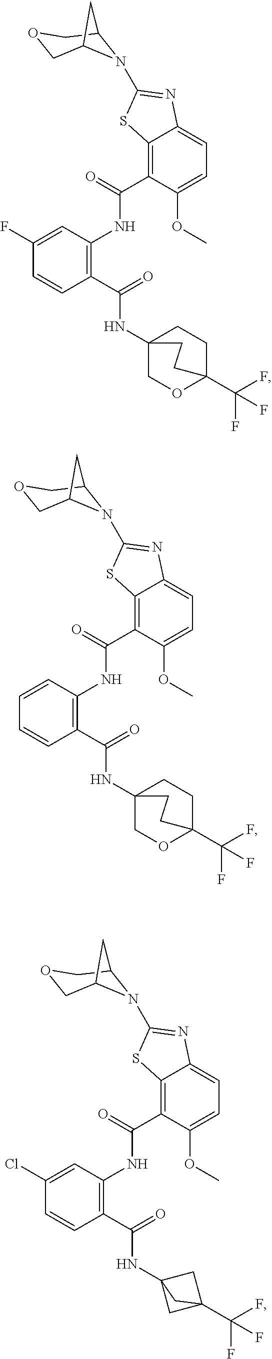 [SMILES: COc1ccc2nc(N3C4COCC3C4)sc2c1C(=O)Nc1cc(F)ccc1C(=O)NC12CCC(C(F)(F)F)(CC1)OC2 ; COc1ccc2nc(N3C4COCC3C4)sc2c1C(=O)Nc1ccccc1C(=O)NC12CCC(C(F)(F)F)(CC1)OC2 ; COc1ccc2nc(N3C4COCC3C4)sc2c1C(=O)Nc1cc(Cl)ccc1C(=O)NC12CC(C(F)(F)F)(C1)C2]

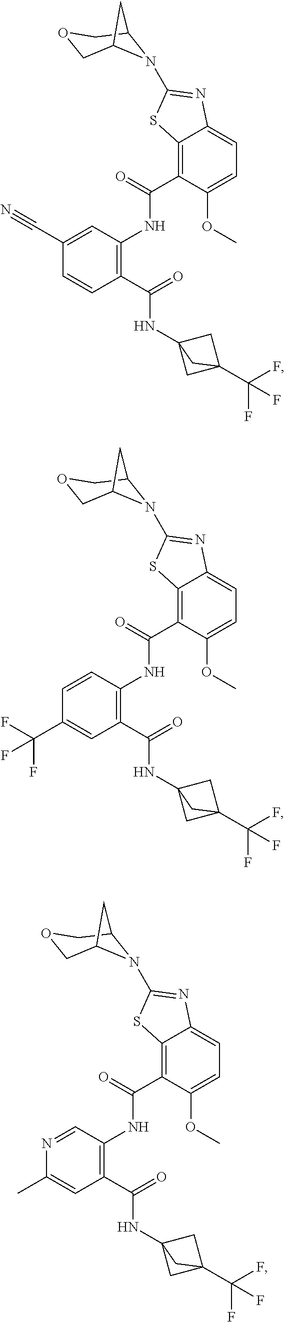 [SMILES: COc1ccc2nc(N3C4COCC3C4)sc2c1C(=O)Nc1cc(C#N)ccc1C(=O)NC12CC(C(F)(F)F)(C1)C2 ; COc1ccc2nc(N3C4COCC3C4)sc2c1C(=O)Nc1ccc(C(F)(F)F)cc1C(=O)NC12CC(C(F)(F)F)(C1)C2 ; COc1ccc2nc(N3C4COCC3C4)sc2c1C(=O)Nc1cnc(C)cc1C(=O)NC12CC(C(F)(F)F)(C1)C2]

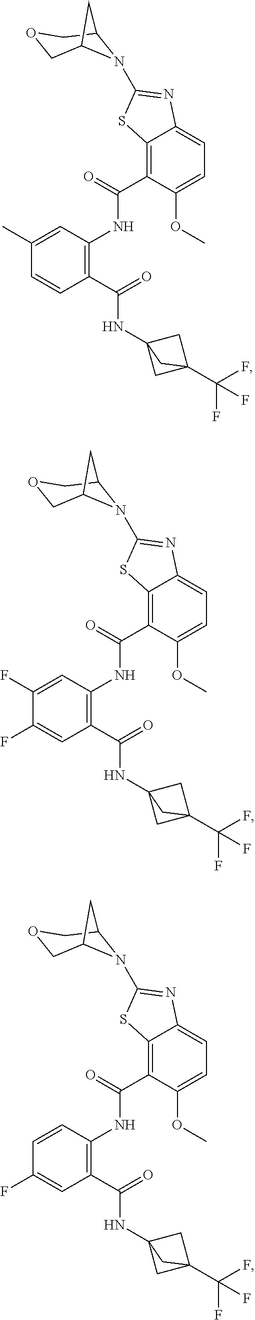 [SMILES: COc1ccc2nc(N3C4COCC3C4)sc2c1C(=O)Nc1cc(C)ccc1C(=O)NC12CC(C(F)(F)F)(C1)C2 ; COc1ccc2nc(N3C4COCC3C4)sc2c1C(=O)Nc1cc(F)c(F)cc1C(=O)NC12CC(C(F)(F)F)(C1)C2 ; COc1ccc2nc(N3C4COCC3C4)sc2c1C(=O)Nc1ccc(F)cc1C(=O)NC12CC(C(F)(F)F)(C1)C2]

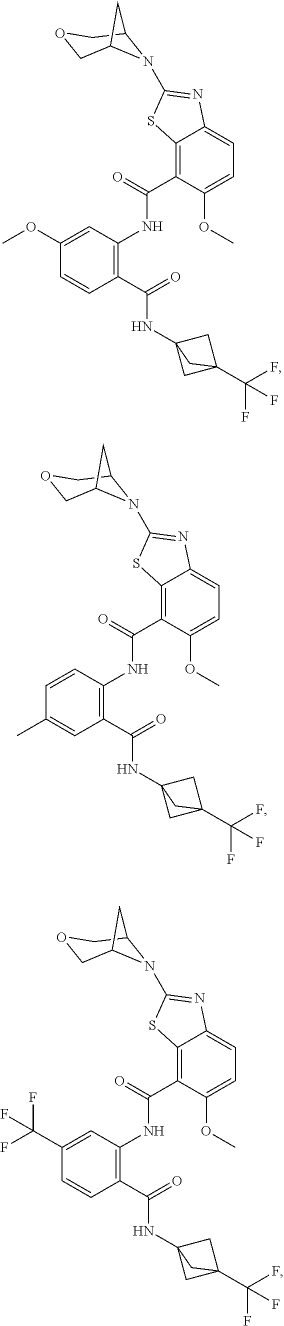 [SMILES: COc1ccc(C(=O)NC23CC(C(F)(F)F)(C2)C3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CC(C(F)(F)F)(C1)C2 ; COc1ccc2nc(N3C4COCC3C4)sc2c1C(=O)Nc1cc(C(F)(F)F)ccc1C(=O)NC12CC(C(F)(F)F)(C1)C2]

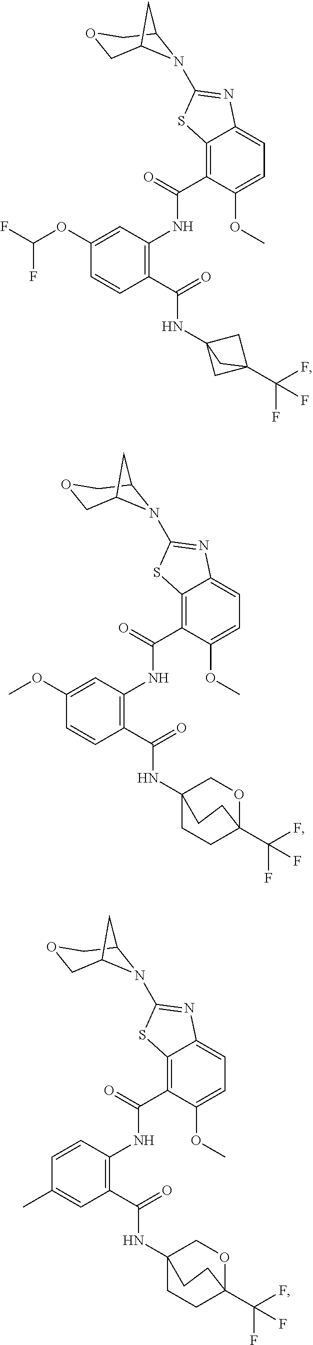 [SMILES: COc1ccc2nc(N3C4COCC3C4)sc2c1C(=O)Nc1cc(OC(F)F)ccc1C(=O)NC12CC(C(F)(F)F)(C1)C2 ; COc1ccc(C(=O)NC23CCC(C(F)(F)F)(CC2)OC3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CCC(C(F)(F)F)(CC1)OC2]

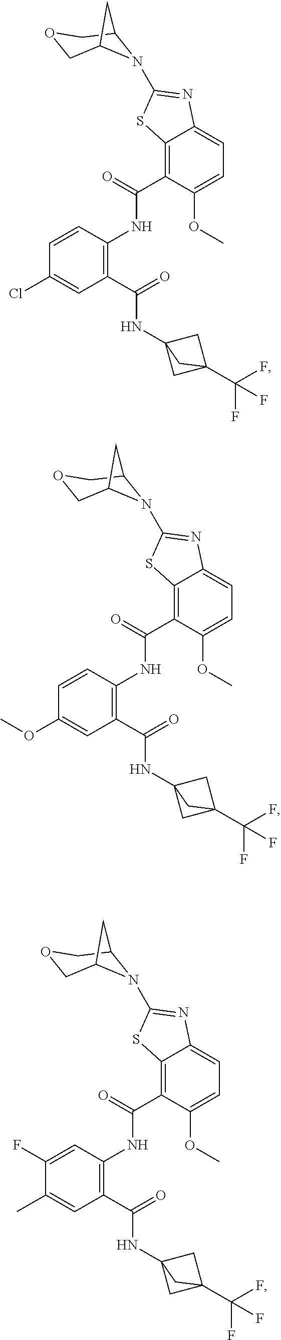 [SMILES: COc1ccc2nc(N3C4COCC3C4)sc2c1C(=O)Nc1ccc(Cl)cc1C(=O)NC12CC(C(F)(F)F)(C1)C2 ; COc1ccc(NC(=O)c2c(OC)ccc3nc(N4C5COCC4C5)sc23)c(C(=O)NC23CC(C(F)(F)F)(C2)C3)c1 ; COc1ccc2nc(N3C4COCC3C4)sc2c1C(=O)Nc1cc(F)c(C)cc1C(=O)NC12CC(C(F)(F)F)(C1)C2]

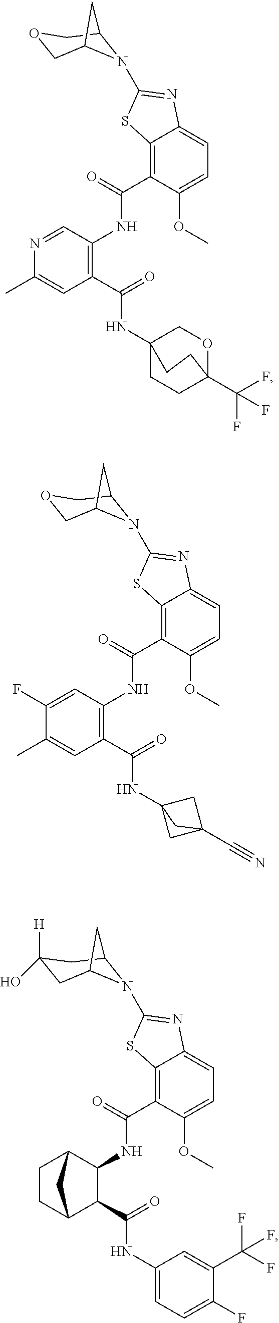 [SMILES: COc1ccc2nc(N3C4COCC3C4)sc2c1C(=O)Nc1cnc(C)cc1C(=O)NC12CCC(C(F)(F)F)(CC1)OC2 ; COc1ccc2nc(N3C4COCC3C4)sc2c1C(=O)Nc1cc(F)c(C)cc1C(=O)NC12CC(C#N)(C1)C2 ; COc1ccc2nc(N3C4CC(O)CC3C4)sc2c1C(=O)N[C@@H]1[C@H]2CC[C@H](C2)[C@@H]1C(=O)Nc1ccc(F)c(C(F)(F)F)c1]

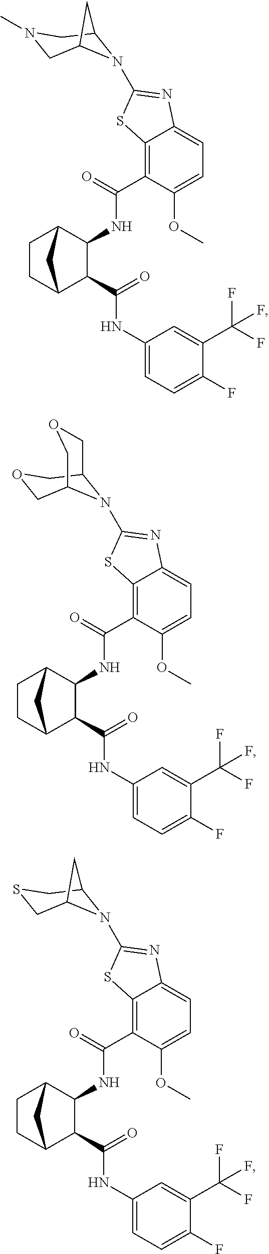 [SMILES: COc1ccc2nc(N3C4CC3CN(C)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3C4CSCC3C4)sc2c1C(=O)N[C@@H]1[C@H]2CC[C@H](C2)[C@@H]1C(=O)Nc1ccc(F)c(C(F)(F)F)c1]

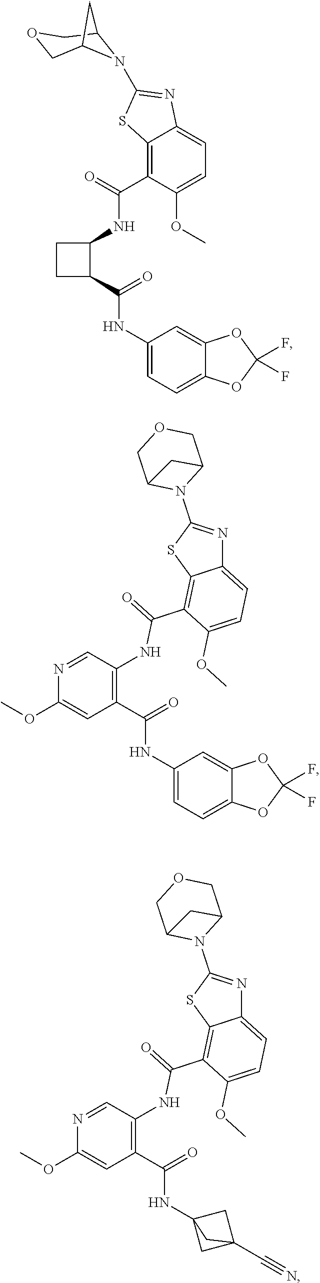

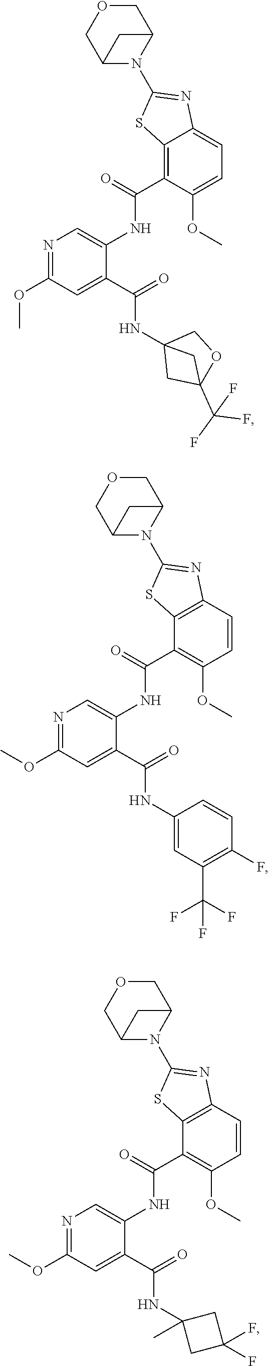 [SMILES: COc1cc(C(=O)NC23COC(C(F)(F)F)(C2)C3)c(NC(=O)c2c(OC)ccc3nc(N4C5COCC4C5)sc23)cn1 ; COc1cc(C(=O)Nc2ccc(F)c(C(F)(F)F)c2)c(NC(=O)c2c(OC)ccc3nc(N4C5COCC4C5)sc23)cn1 ; COc1cc(C(=O)NC2(C)CC(F)(F)C2)c(NC(=O)c2c(OC)ccc3nc(N4C5COCC4C5)sc23)cn1]

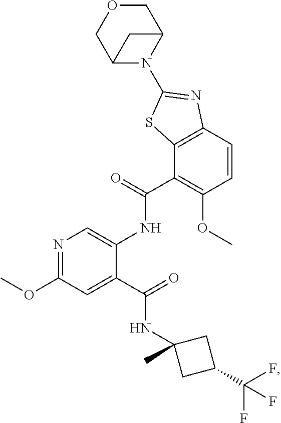 [SMILES: COc1cc(C(=O)N[C@]2(C)C[C@H](C(F)(F)F)C2)c(NC(=O)c2c(OC)ccc3nc(N4C5COCC4C5)sc23)cn1]

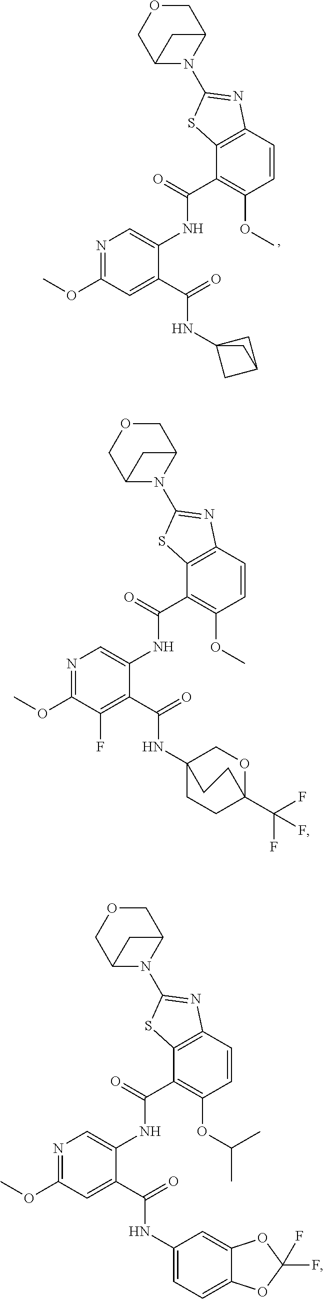

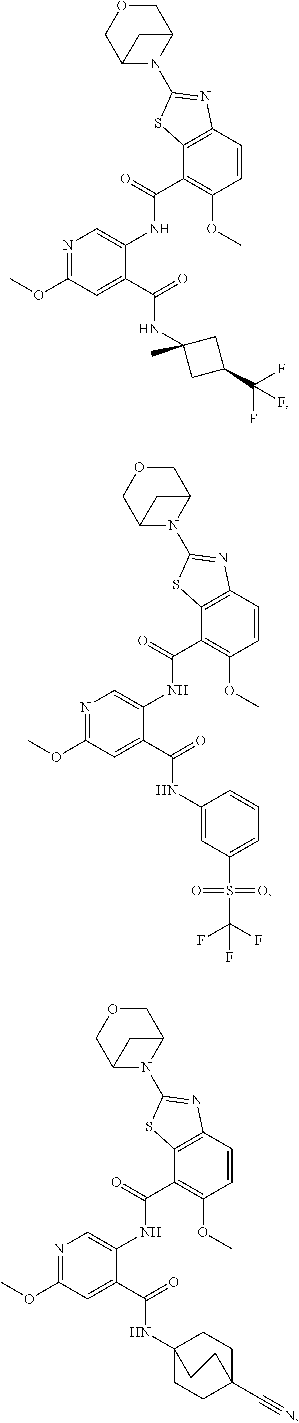 [SMILES: COc1cc(C(=O)N[C@]2(C)C[C@@H](C(F)(F)F)C2)c(NC(=O)c2c(OC)ccc3nc(N4C5COCC4C5)sc23)cn1 ; COc1cc(C(=O)Nc2cccc(S(=O)(=O)C(F)(F)F)c2)c(NC(=O)c2c(OC)ccc3nc(N4C5COCC4C5)sc23)cn1 ; COc1cc(C(=O)NC23CCC(C#N)(CC2)CC3)c(NC(=O)c2c(OC)ccc3nc(N4C5COCC4C5)sc23)cn1]

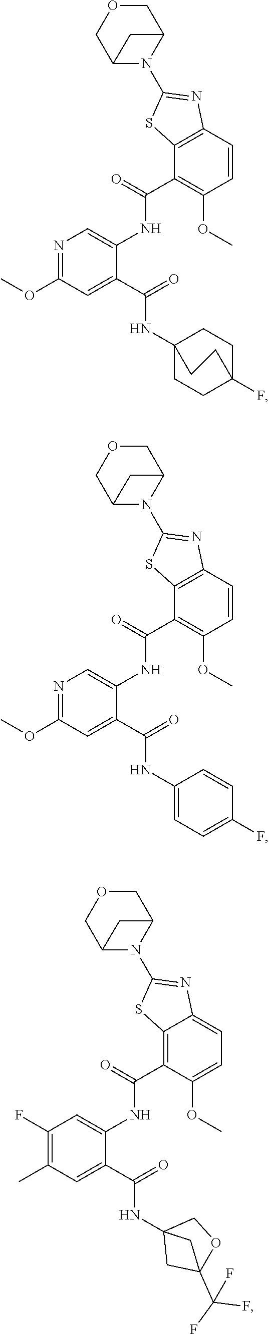 [SMILES: COc1cc(C(=O)NC23CCC(F)(CC2)CC3)c(NC(=O)c2c(OC)ccc3nc(N4C5COCC4C5)sc23)cn1 ; COc1cc(C(=O)Nc2ccc(F)cc2)c(NC(=O)c2c(OC)ccc3nc(N4C5COCC4C5)sc23)cn1 ; COc1ccc2nc(N3C4COCC3C4)sc2c1C(=O)Nc1cc(F)c(C)cc1C(=O)NC12COC(C(F)(F)F)(C1)C2]

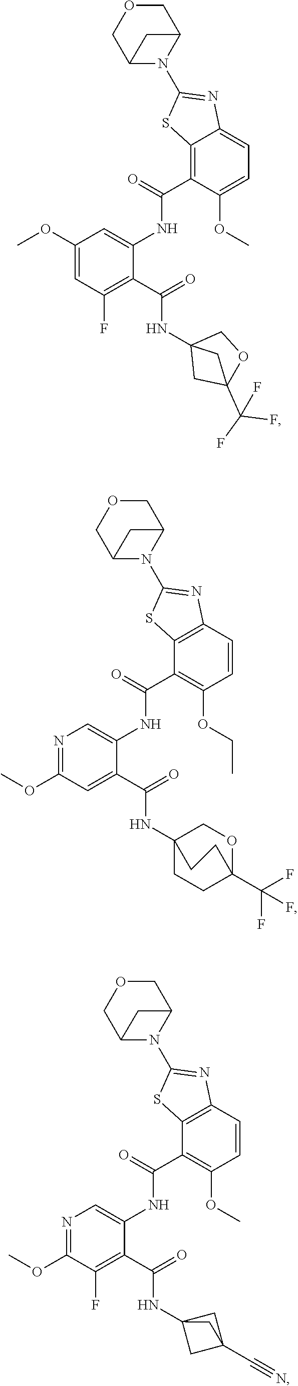 [SMILES: COc1cc(F)c(C(=O)NC23COC(C(F)(F)F)(C2)C3)c(NC(=O)c2c(OC)ccc3nc(N4C5COCC4C5)sc23)c1 ; CCOc1ccc2nc(N3C4COCC3C4)sc2c1C(=O)Nc1cnc(OC)cc1C(=O)NC12CCC(C(F)(F)F)(CC1)OC2 ; COc1ccc2nc(N3C4COCC3C4)sc2c1C(=O)Nc1cnc(OC)c(F)c1C(=O)NC12CC(C#N)(C1)C2]

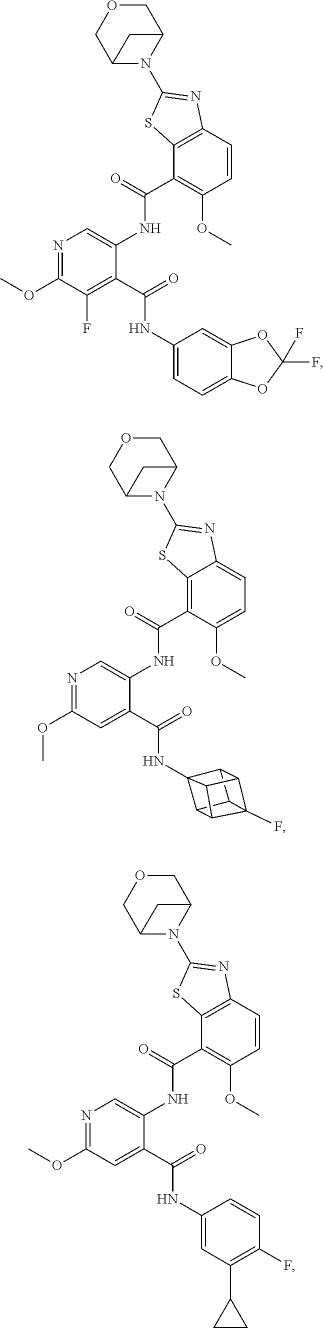 [SMILES: COc1ccc2nc(N3C4COCC3C4)sc2c1C(=O)Nc1cnc(OC)c(F)c1C(=O)Nc1ccc2c(c1)OC(F)(F)O2 ; COc1cc(C(=O)NC23C4C5C2C2C3C4C52F)c(NC(=O)c2c(OC)ccc3nc(N4C5COCC4C5)sc23)cn1 ; COc1cc(C(=O)Nc2ccc(F)c(C3CC3)c2)c(NC(=O)c2c(OC)ccc3nc(N4C5COCC4C5)sc23)cn1]

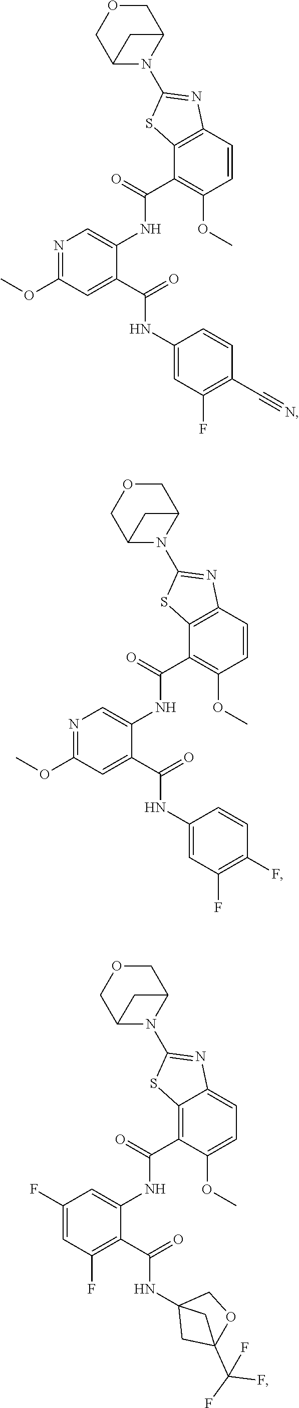 [SMILES: COc1cc(C(=O)Nc2ccc(C#N)c(F)c2)c(NC(=O)c2c(OC)ccc3nc(N4C5COCC4C5)sc23)cn1 ; COc1cc(C(=O)Nc2ccc(F)c(F)c2)c(NC(=O)c2c(OC)ccc3nc(N4C5COCC4C5)sc23)cn1 ; COc1ccc2nc(N3C4COCC3C4)sc2c1C(=O)Nc1cc(F)cc(F)c1C(=O)NC12COC(C(F)(F)F)(C1)C2]

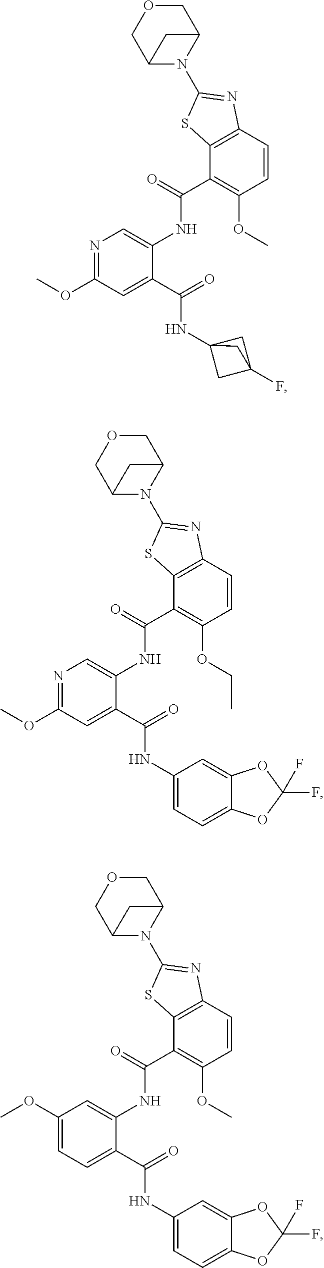 [SMILES: COc1cc(C(=O)NC23CC(F)(C2)C3)c(NC(=O)c2c(OC)ccc3nc(N4C5COCC4C5)sc23)cn1 ; CCOc1ccc2nc(N3C4COCC3C4)sc2c1C(=O)Nc1cnc(OC)cc1C(=O)Nc1ccc2c(c1)OC(F)(F)O2 ; COc1ccc(C(=O)Nc2ccc3c(c2)OC(F)(F)O3)c(NC(=O)c2c(OC)ccc3nc(N4C5COCC4C5)sc23)c1]

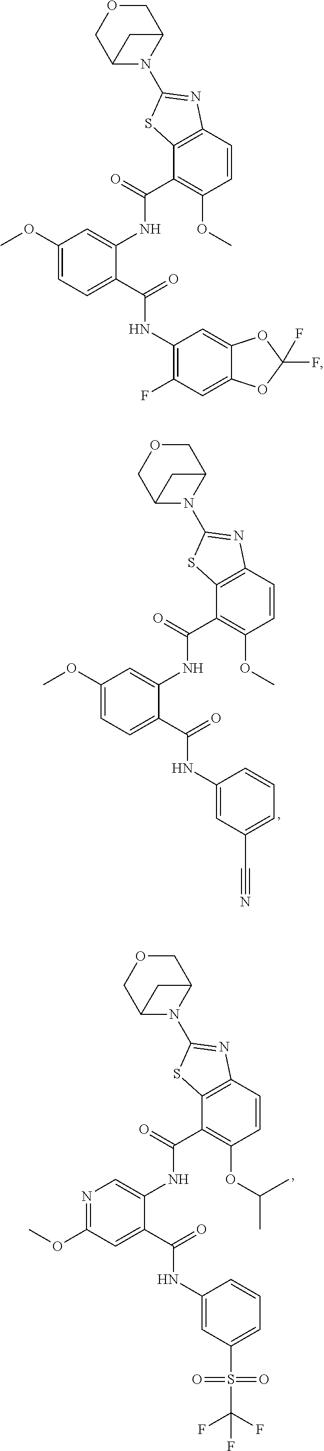 [SMILES: COc1ccc(C(=O)Nc2cc3c(cc2F)OC(F)(F)O3)c(NC(=O)c2c(OC)ccc3nc(N4C5COCC4C5)sc23)c1 ; COc1ccc(C(=O)Nc2cccc(C#N)c2)c(NC(=O)c2c(OC)ccc3nc(N4C5COCC4C5)sc23)c1 ; COc1cc(C(=O)Nc2cccc(S(=O)(=O)C(F)(F)F)c2)c(NC(=O)c2c(OC(C)C)ccc3nc(N4C5COCC4C5)sc23)cn1]

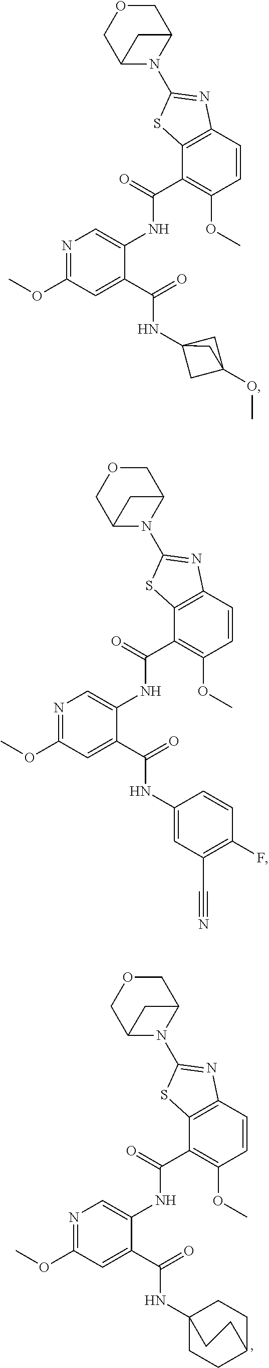

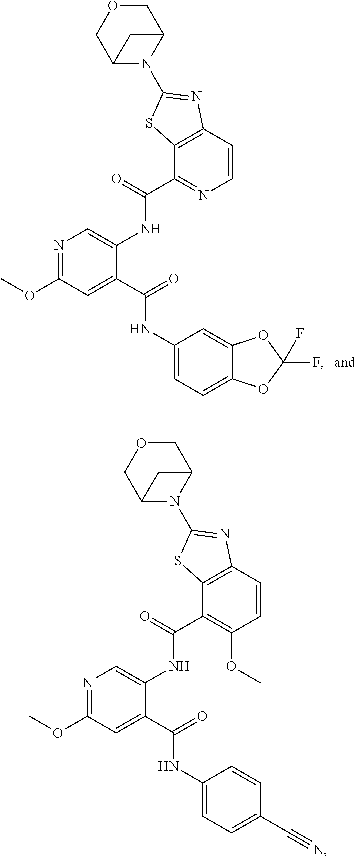 [SMILES: COc1cc(C(=O)Nc2ccc3c(c2)OC(F)(F)O3)c(NC(=O)c2nccc3nc(N4C5COCC4C5)sc23)cn1 ; "and" ; COc1cc(C(=O)Nc2ccc(C#N)cc2)c(NC(=O)c2c(OC)ccc3nc(N4C5COCC4C5)sc23)cn1]

or a pharmaceutically acceptable salt thereof.

In another embodiment, the compound is selected from the group consisting of

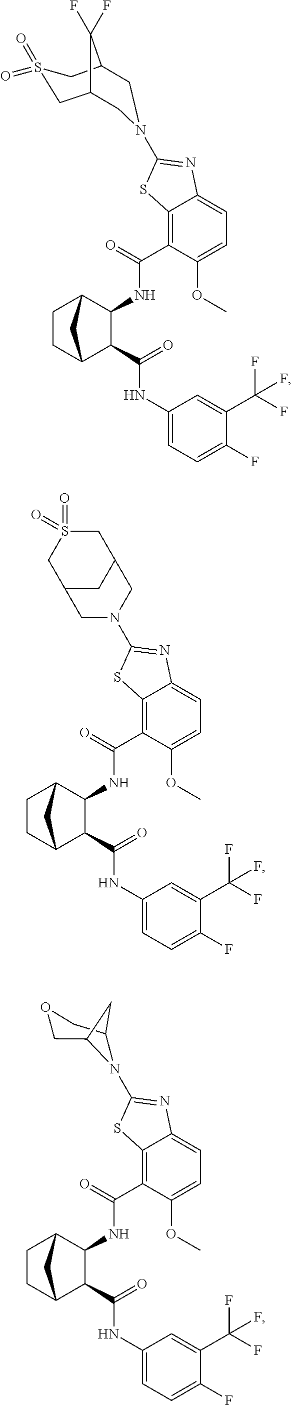 [SMILES: COc1ccc2nc(N3CC4CS(=O)(=O)CC(C3)C4(F)F)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)CS(=O)(=O)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1]

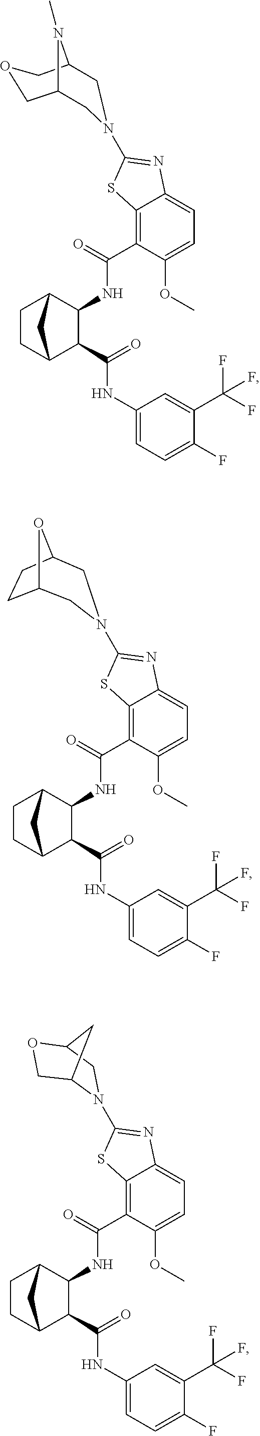 [SMILES: COc1ccc2nc(N3CC4COCC(C3)N4C)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1]

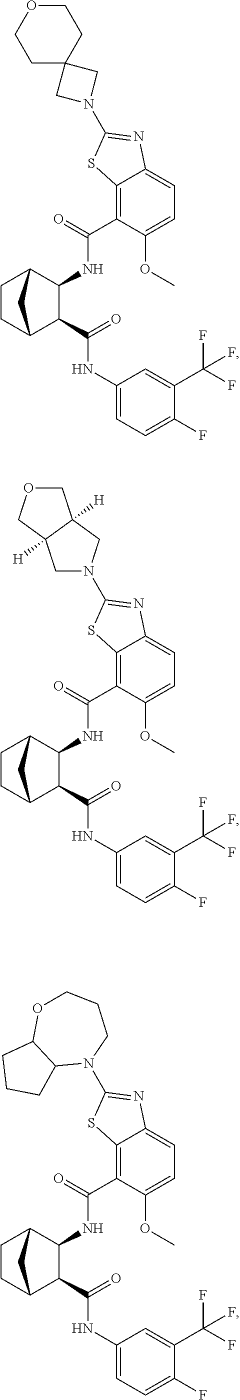 [SMILES: COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCCOC4CCCC43)sc2c1C(=O)N[C@@H]1[C@H]2CC[C@H](C2)[C@@H]1C(=O)Nc1ccc(F)c(C(F)(F)F)c1]

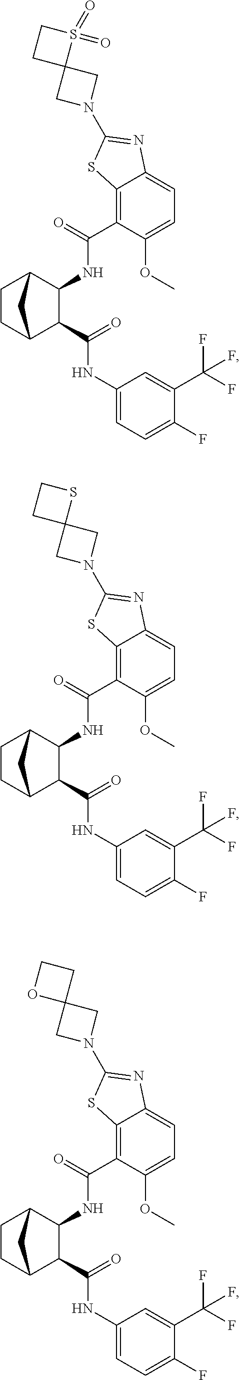 [SMILES: COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1]

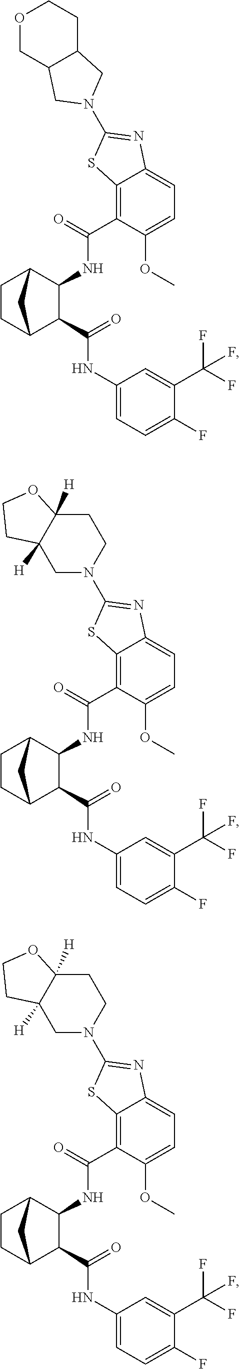 [SMILES: COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@H]4OCC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@@H]4OCC[C@@H]4C3)sc2c1C(=O)N[C@@H]1[C@H]2CC[C@H](C2)[C@@H]1C(=O)Nc1ccc(F)c(C(F)(F)F)c1]

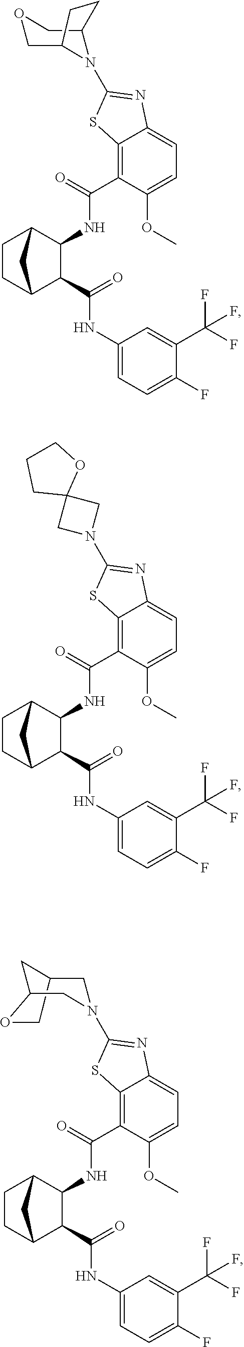 [SMILES: COc1ccc2nc(N3C4CCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4COC(C4)C3)sc2c1C(=O)N[C@@H]1[C@H]2CC[C@H](C2)[C@@H]1C(=O)Nc1ccc(F)c(C(F)(F)F)c1]

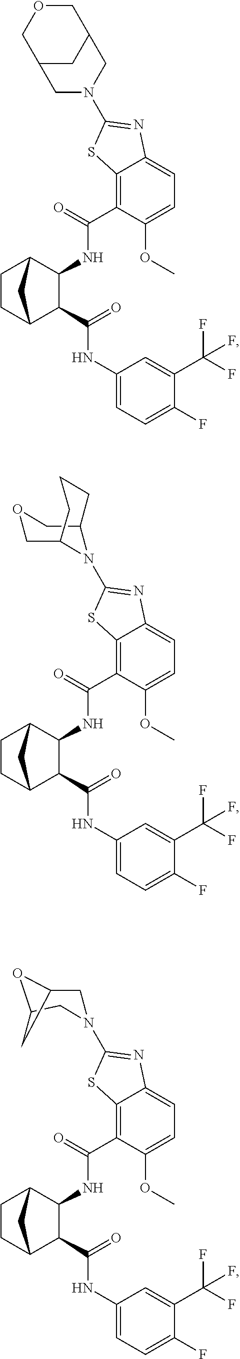 [SMILES: COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C4CCCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1]

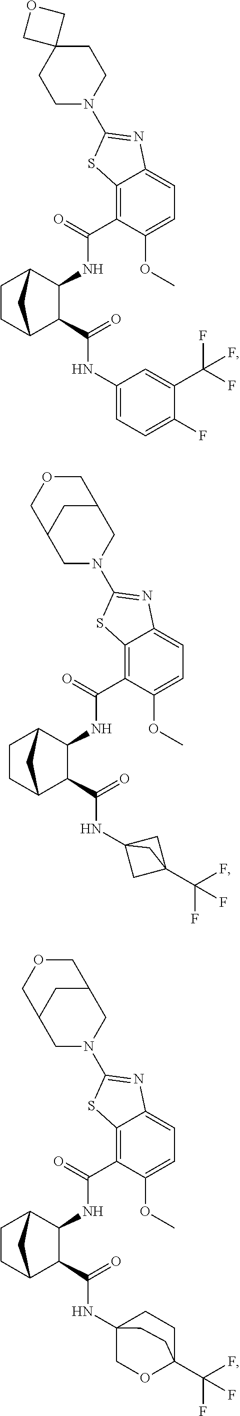 [SMILES: COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2 ; COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2]

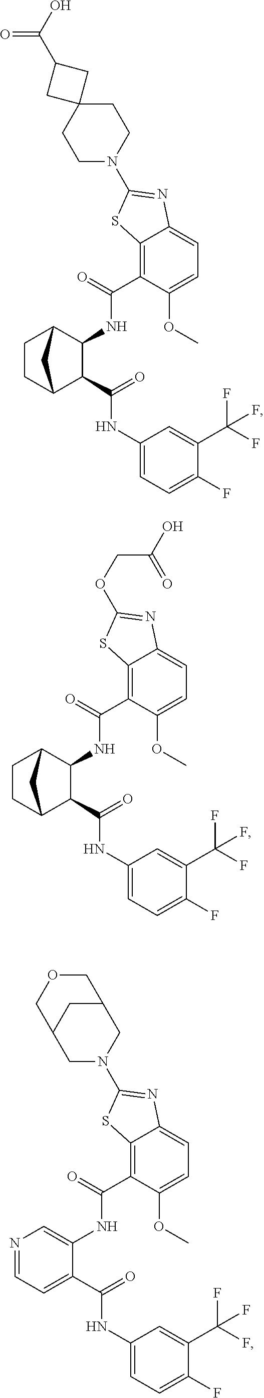

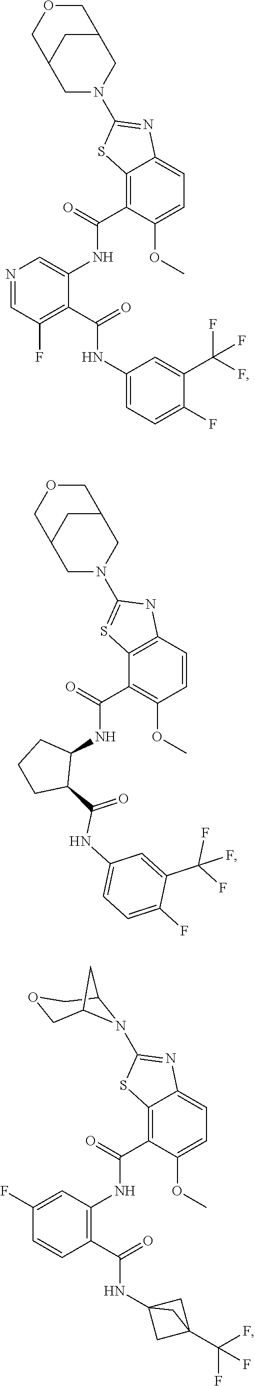

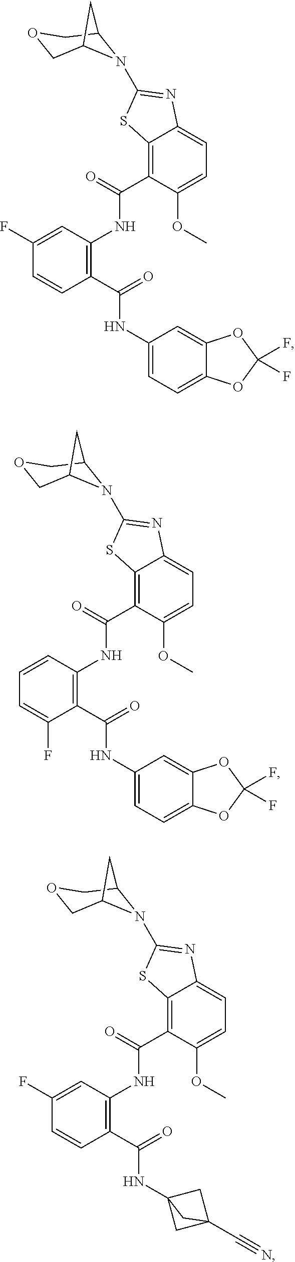 [SMILES: COc1ccc2nc(N3C4COCC3C4)sc2c1C(=O)Nc1cc(F)ccc1C(=O)Nc1ccc2c(c1)OC(F)(F)O2 ; COc1ccc2nc(N3C4COCC3C4)sc2c1C(=O)Nc1cccc(F)c1C(=O)Nc1ccc2c(c1)OC(F)(F)O2 ; COc1ccc2nc(N3C4COCC3C4)sc2c1C(=O)Nc1cc(F)ccc1C(=O)NC12CC(C#N)(C1)C2]

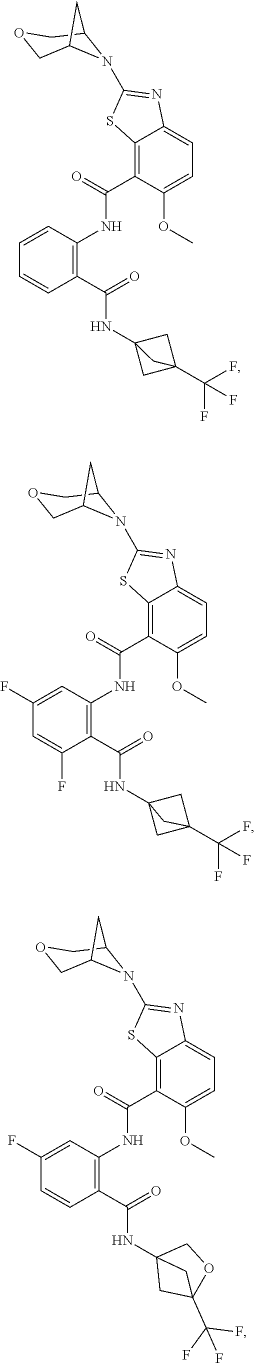 [SMILES: COc1ccc2nc(N3C4COCC3C4)sc2c1C(=O)Nc1ccccc1C(=O)NC12CC(C(F)(F)F)(C1)C2 ; COc1ccc2nc(N3C4COCC3C4)sc2c1C(=O)Nc1cc(F)cc(F)c1C(=O)NC12CC(C(F)(F)F)(C1)C2 ; COc1ccc2nc(N3C4COCC3C4)sc2c1C(=O)Nc1cc(F)ccc1C(=O)NC12COC(C(F)(F)F)(C1)C2]

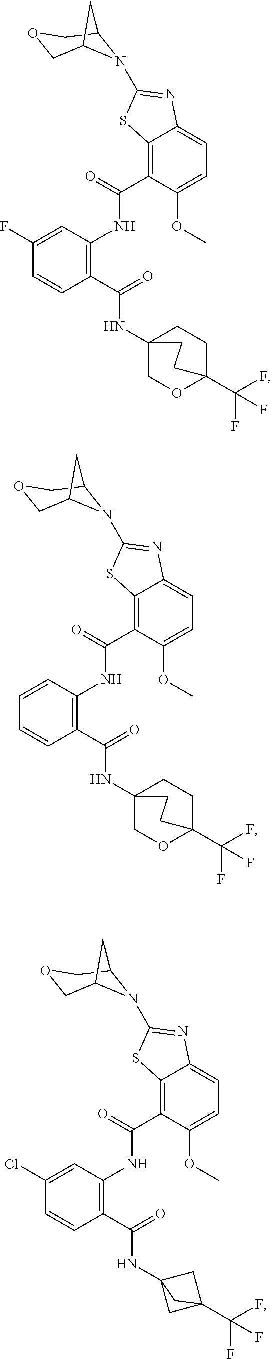 [SMILES: COc1ccc2nc(N3C4COCC3C4)sc2c1C(=O)Nc1cc(F)ccc1C(=O)NC12CCC(C(F)(F)F)(CC1)OC2 ; COc1ccc2nc(N3C4COCC3C4)sc2c1C(=O)Nc1ccccc1C(=O)NC12CCC(C(F)(F)F)(CC1)OC2 ; COc1ccc2nc(N3C4COCC3C4)sc2c1C(=O)Nc1cc(Cl)ccc1C(=O)NC12CC(C(F)(F)F)(C1)C2]

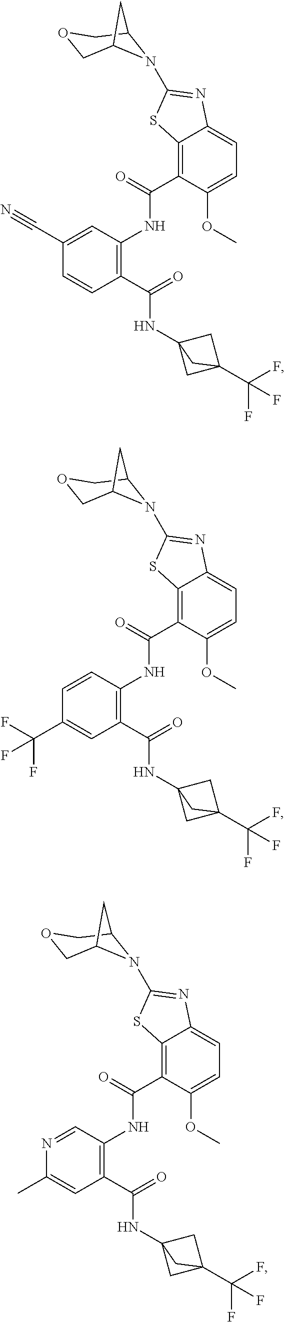 [SMILES: COc1ccc2nc(N3C4COCC3C4)sc2c1C(=O)Nc1cc(C#N)ccc1C(=O)NC12CC(C(F)(F)F)(C1)C2 ; COc1ccc2nc(N3C4COCC3C4)sc2c1C(=O)Nc1ccc(C(F)(F)F)cc1C(=O)NC12CC(C(F)(F)F)(C1)C2 ; COc1ccc2nc(N3C4COCC3C4)sc2c1C(=O)Nc1cnc(C)cc1C(=O)NC12CC(C(F)(F)F)(C1)C2]

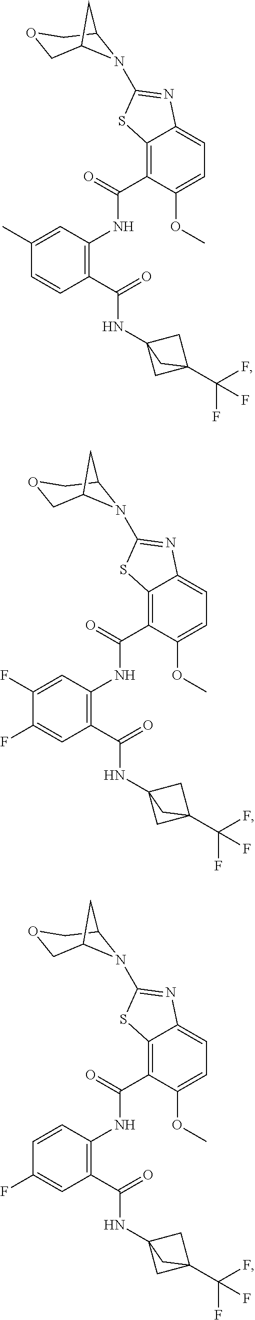 [SMILES: COc1ccc2nc(N3C4COCC3C4)sc2c1C(=O)Nc1cc(C)ccc1C(=O)NC12CC(C(F)(F)F)(C1)C2 ; COc1ccc2nc(N3C4COCC3C4)sc2c1C(=O)Nc1cc(F)c(F)cc1C(=O)NC12CC(C(F)(F)F)(C1)C2 ; COc1ccc2nc(N3C4COCC3C4)sc2c1C(=O)Nc1ccc(F)cc1C(=O)NC12CC(C(F)(F)F)(C1)C2]

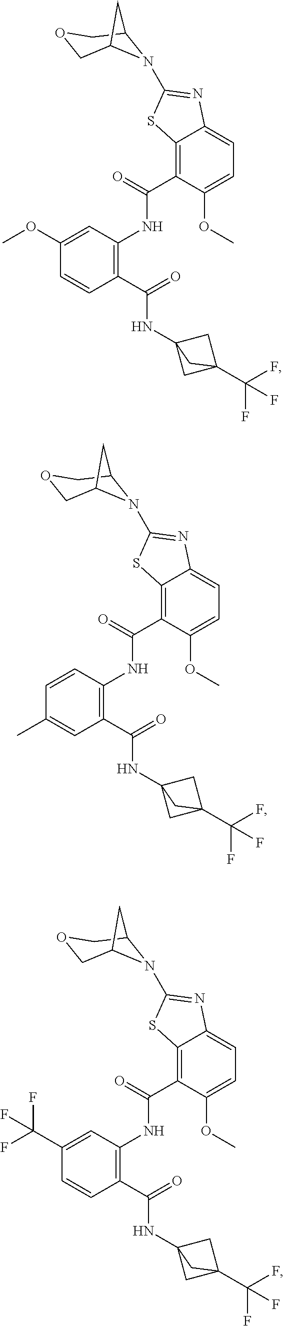 [SMILES: COc1ccc(C(=O)NC23CC(C(F)(F)F)(C2)C3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CC(C(F)(F)F)(C1)C2 ; COc1ccc2nc(N3C4COCC3C4)sc2c1C(=O)Nc1cc(C(F)(F)F)ccc1C(=O)NC12CC(C(F)(F)F)(C1)C2]

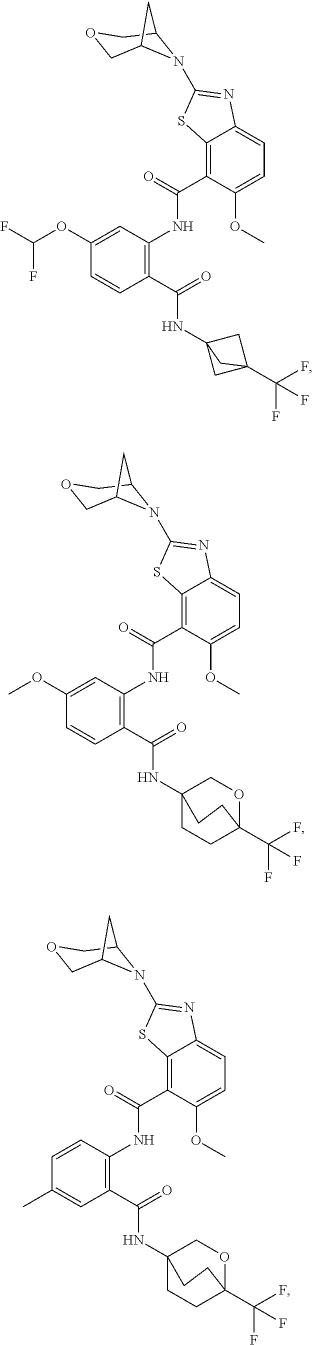 [SMILES: COc1ccc2nc(N3C4COCC3C4)sc2c1C(=O)Nc1cc(OC(F)F)ccc1C(=O)NC12CC(C(F)(F)F)(C1)C2 ; COc1ccc(C(=O)NC23CCC(C(F)(F)F)(CC2)OC3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CCC(C(F)(F)F)(CC1)OC2]

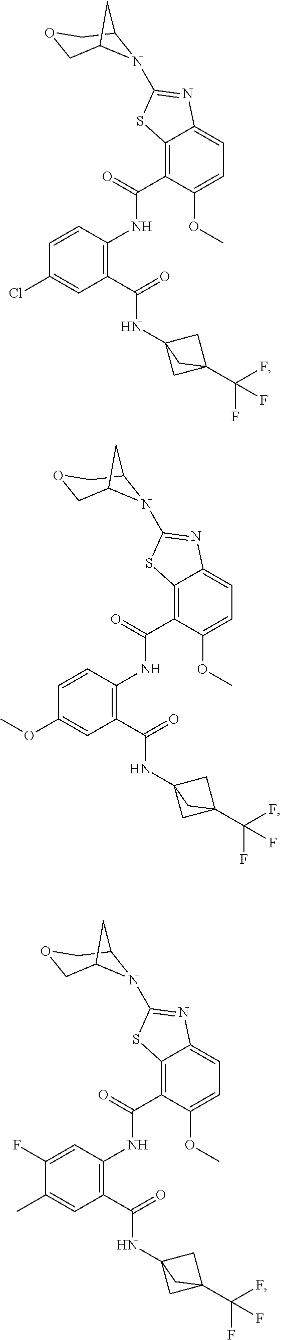 [SMILES: COc1ccc2nc(N3C4COCC3C4)sc2c1C(=O)Nc1ccc(Cl)cc1C(=O)NC12CC(C(F)(F)F)(C1)C2 ; COc1ccc(NC(=O)c2c(OC)ccc3nc(N4C5COCC4C5)sc23)c(C(=O)NC23CC(C(F)(F)F)(C2)C3)c1 ; COc1ccc2nc(N3C4COCC3C4)sc2c1C(=O)Nc1cc(F)c(C)cc1C(=O)NC12CC(C(F)(F)F)(C1)C2]

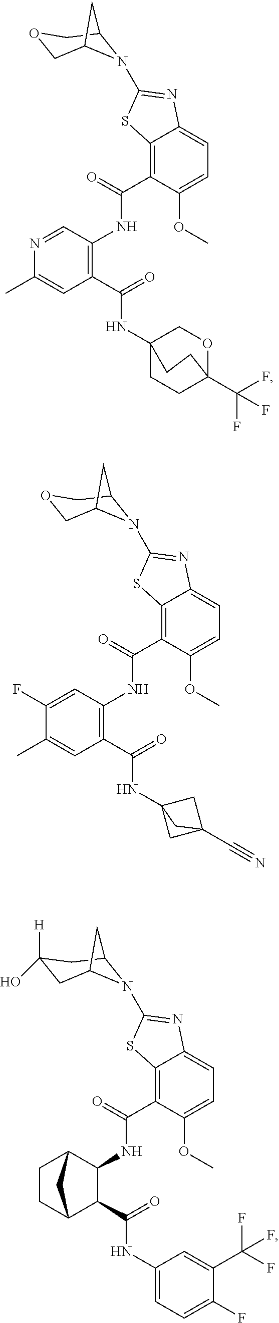 [SMILES: COc1ccc2nc(N3C4COCC3C4)sc2c1C(=O)Nc1cnc(C)cc1C(=O)NC12CCC(C(F)(F)F)(CC1)OC2 ; COc1ccc2nc(N3C4COCC3C4)sc2c1C(=O)Nc1cc(F)c(C)cc1C(=O)NC12CC(C#N)(C1)C2 ; COc1ccc2nc(N3C4CC(O)CC3C4)sc2c1C(=O)N[C@@H]1[C@H]2CC[C@H](C2)[C@@H]1C(=O)Nc1ccc(F)c(C(F)(F)F)c1]

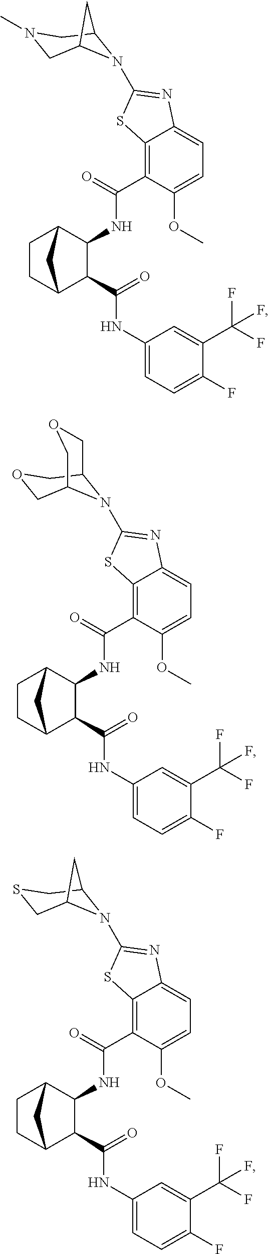 [SMILES: COc1ccc2nc(N3C4CC3CN(C)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3C4CSCC3C4)sc2c1C(=O)N[C@@H]1[C@H]2CC[C@H](C2)[C@@H]1C(=O)Nc1ccc(F)c(C(F)(F)F)c1]

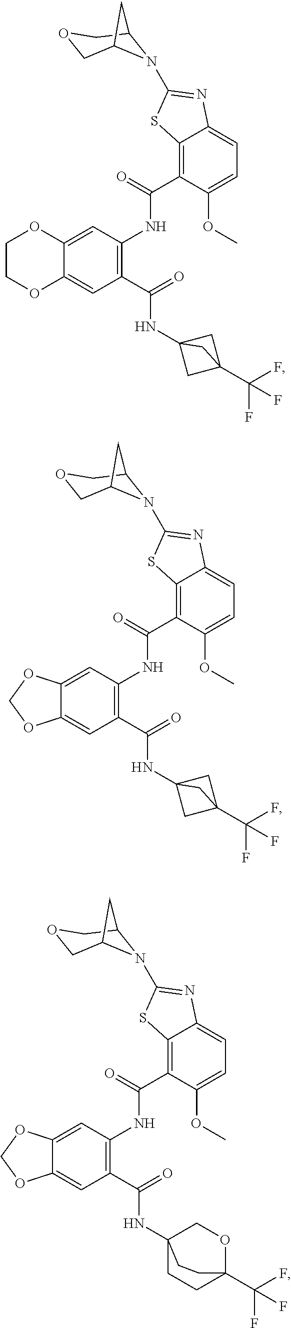 [SMILES: COc1ccc2nc(N3C4COCC3C4)sc2c1C(=O)Nc1cc2c(cc1C(=O)NC13CC(C(F)(F)F)(C1)C3)OCCO2 ; COc1ccc2nc(N3C4COCC3C4)sc2c1C(=O)Nc1cc2c(cc1C(=O)NC13CC(C(F)(F)F)(C1)C3)OCO2 ; COc1ccc2nc(N3C4COCC3C4)sc2c1C(=O)Nc1cc2c(cc1C(=O)NC13CCC(C(F)(F)F)(CC1)OC3)OCO2]

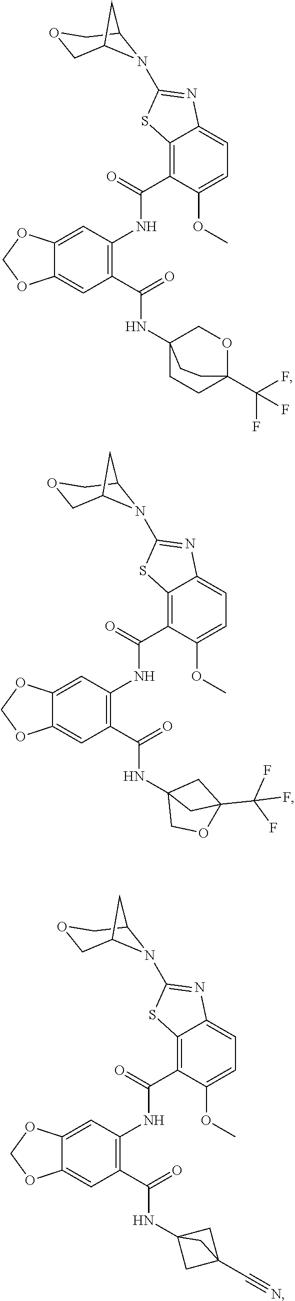 [SMILES: COc1ccc2nc(N3C4COCC3C4)sc2c1C(=O)Nc1cc2c(cc1C(=O)NC13CCC(C(F)(F)F)(CC1)OC3)OCO2 ; COc1ccc2nc(N3C4COCC3C4)sc2c1C(=O)Nc1cc2c(cc1C(=O)NC13COC(C(F)(F)F)(C1)C3)OCO2 ; COc1ccc2nc(N3C4COCC3C4)sc2c1C(=O)Nc1cc2c(cc1C(=O)NC13CC(C#N)(C1)C3)OCO2]

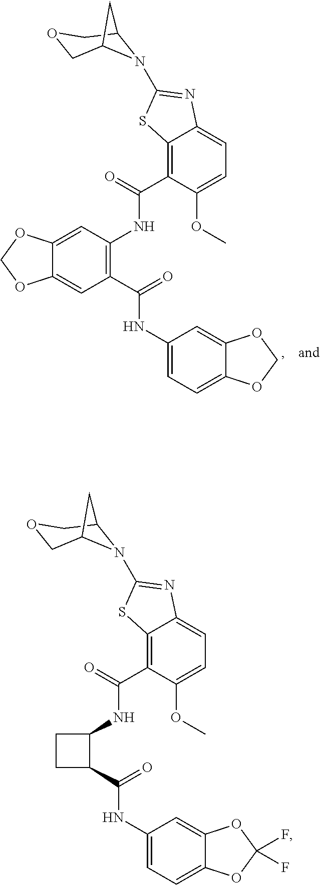 [SMILES: COc1ccc2nc(N3C4COCC3C4)sc2c1C(=O)Nc1cc2c(cc1C(=O)Nc1ccc3c(c1)OCO3)OCO2 ; COc1ccc2nc(N3C4COCC3C4)sc2c1C(=O)N[C@@H]1CC[C@@H]1C(=O)Nc1ccc2c(c1)OC(F)(F)O2]

or a pharmaceutically acceptable salt thereof.

In another embodiment, the compound is selected from

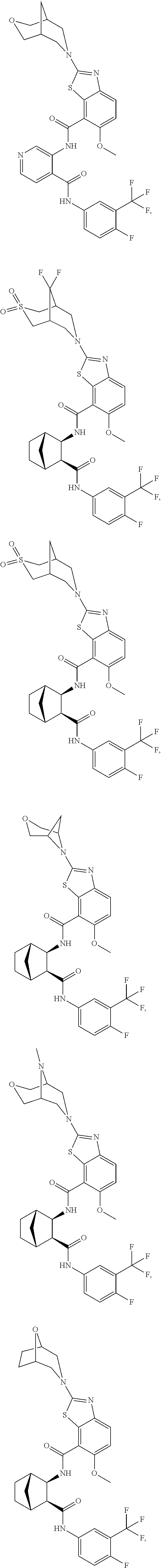

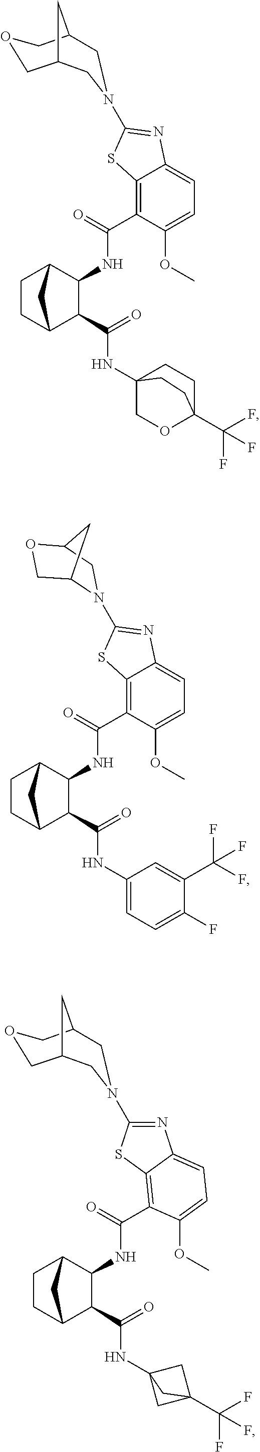 [SMILES: COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2 ; COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2]

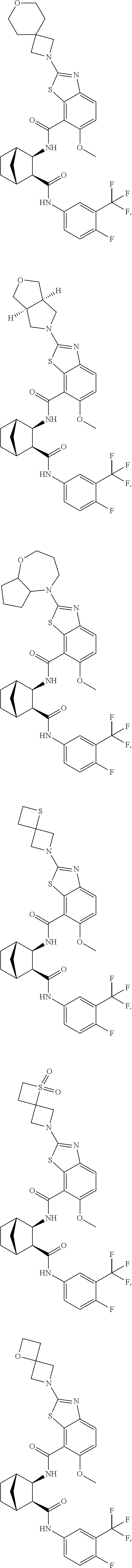

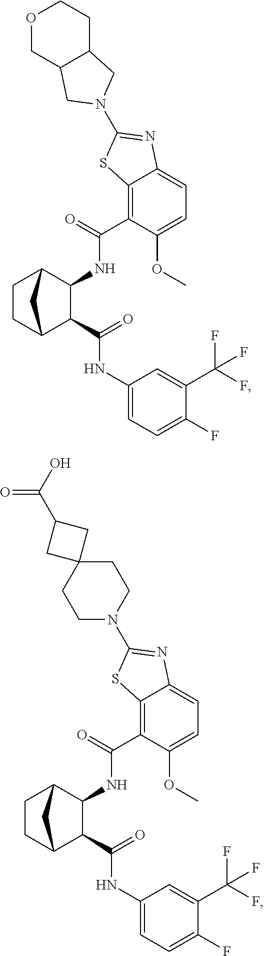 [SMILES: COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCC4(CC3)CC(C(=O)O)C4)sc2c1C(=O)N[C@@H]1[C@H]2CC[C@H](C2)[C@@H]1C(=O)Nc1ccc(F)c(C(F)(F)F)c1]

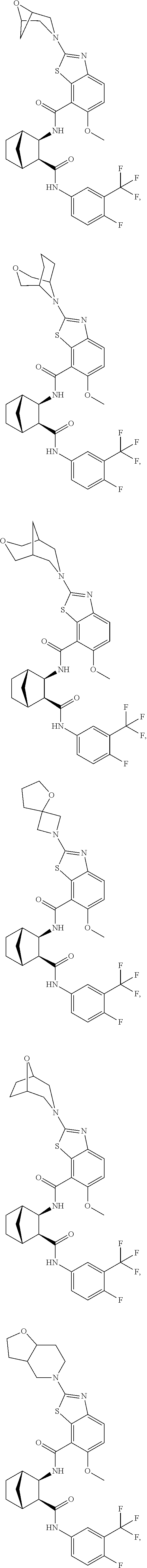

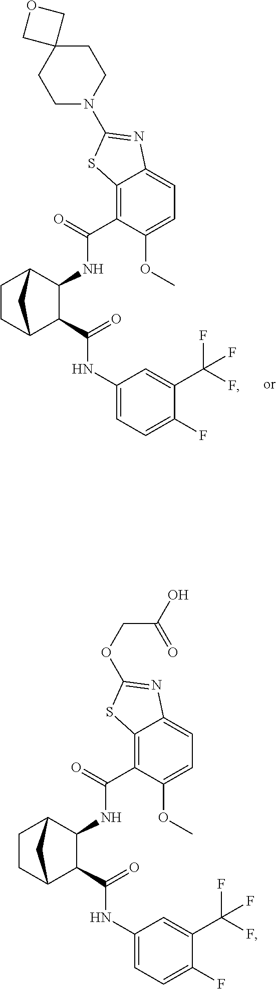 [SMILES: COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1 ; "or" ; COc1ccc2nc(OCC(=O)O)sc2c1C(=O)N[C@@H]1[C@H]2CC[C@H](C2)[C@@H]1C(=O)Nc1ccc(F)c(C(F)(F)F)c1]

or a pharmaceutically acceptable salt thereof.

In another embodiment, a pharmaceutical composition comprises a compound of Formula I or a pharmaceutically acceptable salt thereof, and a pharmaceutically acceptable carrier, diluent, or excipient.

In another embodiment, a method of treating cardiovascular, pulmonary and/or renal conditions, diseases, and/or disorders in an individual, the method comprising administering to the individual an effective amount of a compound of Formula I, or a pharmaceutically acceptable salt thereof, or a pharmaceutical composition of the compound of Formula I, or a pharmaceutically acceptable salt thereof.

In another embodiment, a compound of Formula I, or a pharmaceutically acceptable salt thereof, for use in a therapy.

In another embodiment, a compound of Formula I, or a pharmaceutically acceptable salt thereof, for use in treating cardiovascular, pulmonary and/or renal conditions, diseases, and/or disorders.

In another embodiment, use of a compound of Formula I, or a pharmaceutically acceptable salt thereof, for the manufacture of a medicament for treating cardiovascular, pulmonary and/or renal conditions, diseases, and/or disorders.

In another embodiment, a method of a compound of Formula I, or a pharmaceutically acceptable salt thereof, for use, wherein the disease or disorder to be treated is a cardiovascular condition, disease, or disorder selected from acute heart failure, chronic heart failure, atherosclerosis, coronary artery disease, diabetes, stroke, hypercholesterolemia, hypertension, ischemia, vasoconstriction, or ventricular hypertrophy.

In another embodiment, a method of a compound of Formula I, or a pharmaceutically acceptable salt thereof, for use, wherein the disease or disorder to be treated is a pulmonary condition, disease, or disorder selected from pulmonary hypertension or chronic obstructive pulmonary disease (COPD).

In another embodiment, a method of a compound of Formula I, or a pharmaceutically acceptable salt thereof, for use, wherein the disease or disorder to be treated is a renal condition, disease, or disorder selected from acute kidney disease, chronic kidney disease, or diabetes nephropathy.

In the above embodiments of the compounds of Formula I, or a pharmaceutically acceptable salt thereof, the chemical drawings are shown flat without chiral information. These compounds often have multiple chiral centers and are contemplated to exist in various forms with various combinations of chiral centers. Additionally, these compounds have various enantiomers, diastereomers, and atropisomers that can exist and are included herein.

In an embodiment of a compound of Formula I or a pharmaceutically acceptable salt thereof, the compound is an isotopic derivative of any one of the compounds described herein or a pharmaceutically acceptable salt thereof.

It is understood that the isotopic derivative can be prepared using any of a variety of art-recognized techniques. For example, the isotopic derivatives can generally be prepared by carrying out the procedures disclosed in the schemes and/or in the examples described herein or a pharmaceutically acceptable salt thereof, by substituting an isotopically labeled reagent for a non-isotopically labeled reagent.

In an embodiment of a compound of Formula I, or a pharmaceutically acceptable salt thereof, the compound is a deuterated derivative of any one of the compounds described herein and pharmaceutically acceptable salts thereof.

In the compounds of this invention any atom not specifically designated as a particular isotope is meant to represent any stable isotope of that atom. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

A compound of Formula I, or a pharmaceutically acceptable salt thereof, the compound is selected from

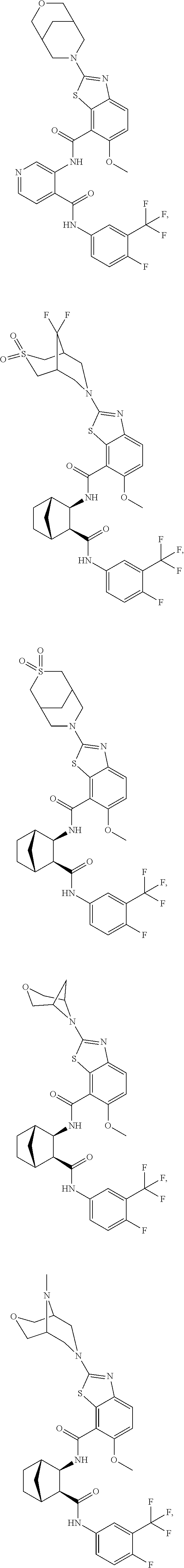 [SMILES: COc1ccc2nc(N3CC4COCC(C4)C3)sc2c1C(=O)Nc1cnccc1C(=O)Nc1ccc(F)c(C(F)(F)F)c1 ; COc1ccc2nc(N3CC4CS(=O)(=O)CC(C3)C4(F)F)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)CS(=O)(=O)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C3)N4C)sc2c1C(=O)N[C@@H]1[C@H]2CC[C@H](C2)[C@@H]1C(=O)Nc1ccc(F)c(C(F)(F)F)c1]

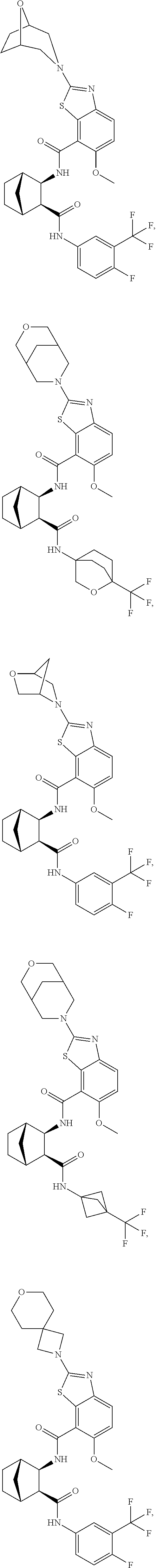 [SMILES: COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2 ; COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2 ; COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1]

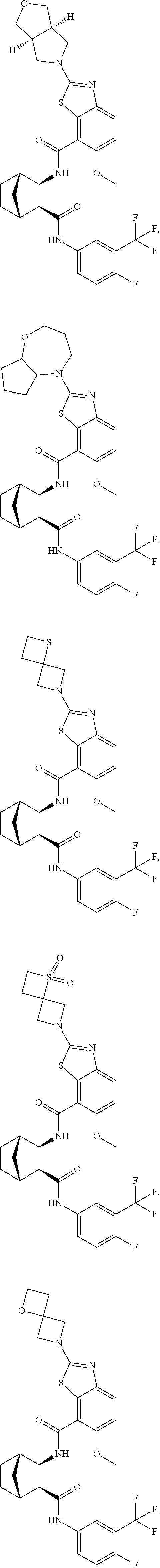 [SMILES: COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCCOC4CCCC43)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1]

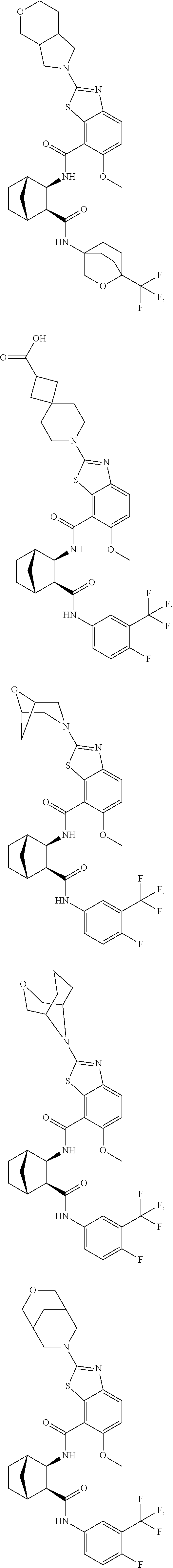 [SMILES: COc1ccc2nc(N3CC4CCOCC4C3)sc2c1C(=O)N[C@@H]1[C@H]2CC[C@H](C2)[C@@H]1C(=O)NC12CCC(C(F)(F)F)(CC1)OC2 ; COc1ccc2nc(N3CCC4(CC3)CC(C(=O)O)C4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3C4CCCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1]

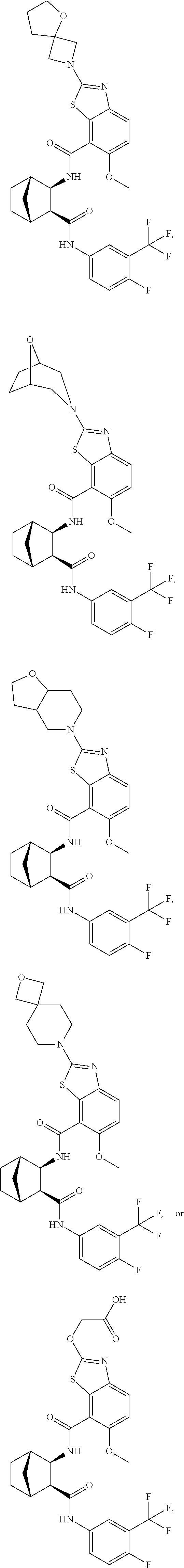 [SMILES: COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CCC4OCCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1 ; COc1ccc2nc(OCC(=O)O)sc2c1C(=O)N[C@@H]1[C@H]2CC[C@H](C2)[C@@H]1C(=O)Nc1ccc(F)c(C(F)(F)F)c1]

or a pharmaceutically acceptable salt thereof.

Also provided herein are pharmaceutical compositions comprising a compound according to Formula I, or a pharmaceutically acceptable salt thereof, examples of which include, but are not limited to, the compounds disclosed herein, and a pharmaceutically acceptable carrier, diluent, or excipient.

Further provided herein are methods of treating cardiovascular, pulmonary and/or renal conditions, diseases and/or disorders, comprising administering to a patient in need thereof, an effective amount of a compound according to Formula I, or a pharmaceutically acceptable salt thereof. Further provided herein are methods of treating cardiovascular conditions, diseases and/or disorders, comprising administering to a patient in need thereof, an effective amount of a compound according to Formula I, or a pharmaceutically acceptable salt thereof. Further provided herein are methods of treating pulmonary conditions, diseases and/or disorders, comprising administering to a patient in need thereof, an effective amount of a compound according to Formula I, or a pharmaceutically acceptable salt thereof. Further provided herein are methods of treating renal conditions, diseases and/or disorders, comprising administering to a patient in need thereof, an effective amount of a compound according to Formula I, or a pharmaceutically acceptable salt thereof.

Further provided herein is a compound according to Formula I or a pharmaceutically acceptable salt thereof for use in therapy. Further provided herein is a compound according to Formula I or a pharmaceutically acceptable salt thereof for use in the treatment of cardiovascular, pulmonary and/or renal conditions, diseases and/or disorders. Further provided herein is a compound according to Formula I or a pharmaceutically acceptable salt thereof for use in therapy. Further provided herein is a compound according to Formula I or a pharmaceutically acceptable salt thereof for use in the treatment of cardiovascular, conditions, diseases and/or disorders. Further provided herein is a compound according to Formula I or a pharmaceutically acceptable salt thereof for use in therapy. Further provided herein is a compound according to Formula I or a pharmaceutically acceptable salt thereof for use in the treatment of pulmonary conditions, diseases and/or disorders. Further provided herein is a compound according to Formula I or a pharmaceutically acceptable salt thereof for use in therapy. Further provided herein is a compound according to Formula I or a pharmaceutically acceptable salt thereof for use in the treatment of renal conditions, diseases and/or disorders.

In the methods or uses herein, the cardiovascular conditions, diseases, and disorders include, but are not limited to, acute heart failure, chronic heart failure, atherosclerosis, coronary artery disease, diabetes, stroke, hypercholesterolemia, hypertension, ischemia, vasoconstriction, and ventricular hypertrophy. In the methods or uses herein, the pulmonary conditions, diseases, and disorders include, but are not limited to, pulmonary hypertension and chronic obstructive pulmonary disease (COPD). In the methods or uses herein, the renal conditions, diseases, and disorders include, but are not limited to, acute kidney disease, chronic kidney disease and diabetes nephropathy.

The methods also can include a step of administering the compound of Formula I, or a pharmaceutically acceptable salt thereof in combination with an effective amount of at least one additional therapeutic agent. Briefly, the standard of care for many of the conditions/diseases/disorders herein includes an anticoagulant, an ACE inhibitor, an ARB, an ARNI, a β-blocker, a diuretic, digitalis, digoxin, hydralazine/isorbide dinitrate, a MRA or other aldosterone antagonist, a SGLT2 inhibitor, a statin and/or an anti-glycemic agent, as well as other therapeutic agents to control comorbidities, including, but not limited to, high cholesterol, high blood pressure, atrial fibrillation, diabetes and obesity. In some instances, the additional therapeutic agent can be administered simultaneously, separately or sequentially with the compound of Formula I, or a pharmaceutically acceptable salt thereof.

Further provided herein is a compound according to Formula I or a pharmaceutically acceptable salt thereof for use in simultaneous, separate, or sequential combination with at least one additional therapeutic agent in the treatment of cardiovascular, pulmonary and/or renal conditions, diseases and/or disorders. Additional therapeutic agents include, but are not limited to, an anticoagulant, an ACE inhibitor, an ARB, an ARNI, a β-blocker, a diuretic, digitalis, digoxin, hydralazine/isorbide dinitrate, a MRA or other aldosterone antagonist, a SGLT2 inhibitor, a statin and/or an anti-glycemic agent, as well as other therapeutic agents to control comorbidities, including, but not limited to, high cholesterol, high blood pressure, atrial fibrillation and diabetes.

The methods or uses herein can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Such methods or uses therefore can include selecting an individual having a cardiovascular condition, disease or disorder or who is predisposed to the same. Alternatively, the methods can include selecting an individual having a pulmonary condition, disease or disorder or who is predisposed to the same. Alternatively, the methods can include selecting an individual having a renal condition, disease or disorder or who is predisposed to the same. In certain instances, the methods can include selecting an individual who is diabetic, hypertensive with kidney function impairment and/or obese.

In some instances, the individual in need is a diabetic, hypertensive with kidney function impairment and/or obese.

The methods or uses also may be combined with diet and exercise and/or may be combined with additional therapeutic agents other than those discussed above.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66 (1), 1-19.

Pharmaceutical compositions containing the compounds of Formula I, or a pharmaceutically acceptable salt thereof, as described herein may be prepared using pharmaceutically acceptable additives. The term “pharmaceutically acceptable additive(s)” as used herein for the pharmaceutical compositions, refers to one or more carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22nd Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

As used herein, “effective amount” means an amount or dose of a compound of formula I, or a pharmaceutically acceptable salt thereof that, upon single or multiple dose administration to an individual in need thereof, provides a desired effect in such an individual under diagnosis or treatment (i.e., may produce a clinically measurable difference in a condition of the individual such as, for example, increased angiogenesis, increased vascular compliance, increased cardiovascular blood flow, increased hepatic blood flow, increased pulmonary blood flow, increased renal blood flow, increased glomerular filtration rate, decreased blood pressure, decreased (or prevented) inflammation and/or reduced (or prevented) fibrosis in the heart, kidney, liver or lung). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the particular compound of Formula I, or a pharmaceutically acceptable salt thereof administered, the mode of administration, the bioavailability characteristics of the preparation administered, the dose regimen selected, the use of concomitant medication, and other relevant circumstances.

As used herein, “treating” or “to treat” means managing and caring for an individual having a condition, disease, disorder or symptom for which compound of formula I, or a pharmaceutically acceptable salt thereof administration is indicated for the purpose of attenuating, restraining, reversing, slowing or stopping progression or severity of the condition, disease, disorder and/or symptom. Treating includes administering a compound of formula I, or a pharmaceutically acceptable salt thereof herein or composition containing a compound of formula I, or a pharmaceutically acceptable salt thereof herein to the individual to prevent the onset of symptoms or complications, alleviating the symptoms or complications, or eliminating the condition, disease, disorder or symptom. Treating includes administering a compound of formula I, or a pharmaceutically acceptable salt thereof or composition containing a compound of formula I, or a pharmaceutically acceptable salt thereof herein to the individual to result in such as, for example, increased angiogenesis, increased vascular compliance, increased cardiovascular blood flow, increased hepatic blood flow, increased pulmonary blood flow, increased renal blood flow, increased glomerular filtration rate, decreased blood pressure, decreased (or prevented) inflammation and/or reduced (or prevented) fibrosis in the heart, kidney, liver or lung). The individual to be treated is a mammal, especially a human.

As used herein, “individual,” “patient” and “subject” are used interchangeably and mean a mammal, especially a human. In certain instances, the individual is further characterized with a condition, disease, disorder and/or symptom that would benefit from administering a compound of formula I, or a pharmaceutically acceptable salt thereof herein.

As used herein, the term halogen means fluoro (F), chloro (Cl), bromo (Br), or iodo (I). As used herein, the term alkyl means saturated linear or branched-chain monovalent hydrocarbon radicals of one to a specified number of carbon atoms, e.g., “C₁₋₄ alkyl” or “C₁₋₃ alkyl.” Examples of alkyls include, but are not limited to, methyl, ethyl, propyl, 1-propyl, isopropyl, butyl, and iso-butyl. As used herein, the terms alkylene or alkylenyl mean saturated linear or branched-chain bivalent hydrocarbon radicals of one to a specified number of carbon atoms, e.g., “C₁₋₃ alkylene.” Examples of alkylenes include, but are not limited to, methylene, ethylene, propylene, 1-propylene, and isopropylene. As used herein, the term alkoxy means saturated linear or branched-chain monovalent hydrocarbon radicals containing a specified number of atoms including both carbon atoms and one or more oxygen atoms, e.g., “C₁₋₃ alkoxy.” For example, C₁₋₃ alkoxy means a saturated linear or branched-chain monovalent hydrocarbon radical containing at least one carbon atom or at least one oxygen, wherein the total number of carbon and oxygen atoms adds up to 1, 2, or 3 atoms. Examples of C₁₋₃ alkoxy groups include, but are not limited to, methoxy, ethoxy, propoxy, 1-propoxy, and isopropoxy.

As used herein, the term heteroalkyl means saturated linear or branched-chain bivalent hydrocarbon radicals containing a specified number of atoms including both carbon atoms and one or more heteroatoms, e.g., “1-5 membered heteroalkyl.” For example, 4 membered heteroalkyl means a saturated linear or branched-chain monovalent hydrocarbon radical containing at least one carbon atom or at least one heteroatom, wherein the total number of carbon and heteroatoms adds up to 4 atoms. Examples of heteroatoms include, but are not limited to, nitrogen, oxygen, and sulfur. Examples of 1-5 membered heteroalkyl include, but are not limited to, —O—CH₂—, —CH₂—O—CH₂—, —O—CH₂—CH₂—, —CH₂—CH₂—O—, —CH₂—SO₂—CH₂—, —O—CH₂—CH₂—CH₂—, —CH₂—CH₂—CH₂—O—, —CH₂—O—CH₂—CH₂—, —CH₂—CH₂—O—CH₂—, —CH₂—CH₂—O—CH₂—CH₂—, —CH₂—CH₂—CH₂—O—CH₂—, —CH₂—O—CH₂—CH₂—CH₂—, —CH₂—CH₂—CH₂—CH₂—O—, —O—CH₂—CH₂—CH₂—CH₂—, —CH₂—SO₂—CH₂—, —CH₂—CH₂—SO₂—, —SO₂—CH₂—CH₂—, —CH₂—S—CH₂—, —CH₂—CH₂—S—, and —S—CH₂—CH₂—.

As used herein, the term “cycloalkyl” means a saturated cyclic hydrocarbon group containing a specified number of carbon atoms. For example, the term “C₃₋₆ cycloalkyl” as used herein refers to a saturated cyclic hydrocarbon group having three, four, five or six carbon atoms. Examples of 3-6 membered cycloalkyl include, cyclopropyl, cyclobutyl, cyclopentyl and cyclohexyl.

As used herein, the term heterocycloalkyl means a saturated cyclic heteroalkyl group containing a specified number of atoms including both carbon atoms and one or more heteroatoms, e.g., “4-7 membered heterocycle”. For example, 7 membered heterocycle means a saturated cyclic hydrocarbon group containing at least one carbon atoms and at least one heteroatoms, wherein the total number of carbon and heteroatoms adds up to 7 atoms. Examples of 4-7 membered heterocycle include, but are not limited to, azetidine, pyrrolidine, morpholine, piperazine, piperidine, and oxazepane.

Certain abbreviations are defined as follows: “ACN” refers to acetonitrile; “DiPEA” refers to N,N-diisopropylethylamine; “DCM” refers to dichloromethane; “DMF” refers to N,N-dimethylformamide; “ES-MS” refers to electrospray mass spectrometry; “Et₂O” refers to diethyl ether; “EtOAc” refers to ethyl acetate; “h” refers to hour(s); “HATU” refers to hexafluorophosphate azabenzotriazole tetramethyl uronium; “HPLC” refers to high-performance liquid chromatography; “MeOH” refers to methanol; “min” refers to minute(s); “NMI” refers to N-methylimidazole; “NMP” refers to N-methyl-2-pyrrolidone; “PG” refers to protecting group; “RT” refers to room temperature; “TBD” refers to triazabicyclodecene; “TCFH” refers to chloro-N,N,N′,N′-tetramethylformamidinium hexafluorophosphate; “TFA” refers to trifluoroacetic acid; “THF” refers to tetrahydrofuran; “T3P” refers to propanephosphonic acid anhydride; “Wt” refers to weight.

Individual isomers, enantiomers, diastereomers, and atropisomers may be separated or resolved at any convenient point in the synthesis of compounds listed below, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994). This description is intended to include all of the isomers, enantiomers, diastereomers, and atropisomers possible for the compounds disclosed herein or that could be made using the compounds disclosed herein. In the molecules described herein, only molecules in which the absolute conformation of a chiral center (or atropisomer conformation) is known have used naming conventions or chemical formula that are drawn to indicate the chirality or atropisomerism. Those of skill in the art will readily understand when other chiral centers are present in the molecules described herein and be able to identify the same.

Compounds of any one of Formula I that are chemically capable of forming salts are readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group. Examples, reactions and conditions for salt formation can be found in Gould, P. L., “Salt selection for basic drugs,” International Journal of Pharmaceutics, 33:201-217 (1986); Bastin, R. J., et al. “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19, (1977).

The compounds of the present invention, or salts thereof, may be prepared by a variety of procedures, some of which are illustrated in the Schemes, Preparations, and Examples below. The specific synthetic steps for each of the routes described may be combined in different ways, or in conjunction with steps from different routes, to prepare compounds or salts of the present invention. The products of each step in the Preparations below can be recovered by conventional methods, including extraction, evaporation, precipitation, chromatography, filtration, trituration, and crystallization.

Additionally, certain intermediates described in the following preparations may contain one or more nitrogen protecting groups. It is understood that protecting groups may be varied as appreciated by one of skill in the art depending on the particular reaction conditions and the particular transformations to be performed. The protection and deprotection conditions are well known to the skilled artisan and are described in the literature (See for example “Greene's Protective Groups in Organic Synthesis”, Fifth Edition, by Peter G. M. Wuts and Theodora W. Greene, John Wiley and Sons, Inc. 2014).

Examples of known procedures and methods include those described in general reference texts such as Comprehensive Organic Transformations, VCH Publishers Inc, 1989; Compendium of Organic Synthetic Methods, Volumes 1-10, 197 4-2002, Wiley Interscience; Advanced Organic Chemistry, Reactions Mechanisms, and Structure, 5th Edition, Michael B. Smith and Jerry March, Wiley Interscience, 2001; Advanced Organic Chemistry, 4th Edition, Part B, Reactions and Synthesis, Francis A Carey and Richard J. Sundberg, Kluwer Academic/Plenum Publishers, 2000, etc., and references cited therein.

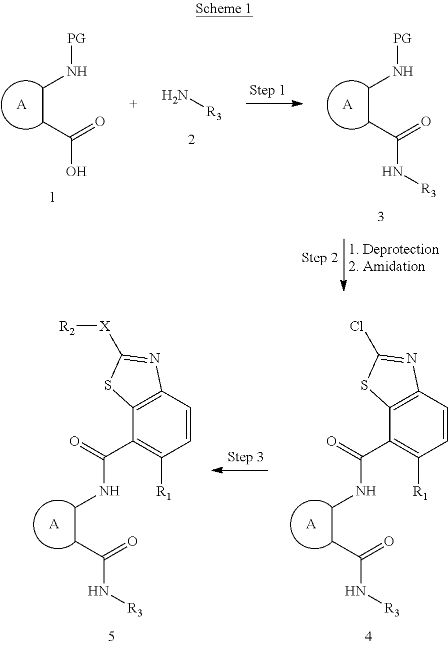

Scheme 1 depicts the preparation of compounds of the present invention beginning with a suitable carboxylic acid 1 and amine 2. The PG moiety on the amine of the carboxylic acid 1 is a standard amine protecting group well known to the skilled artisan, including carbamate and amide protecting groups. The carboxylic acid 1 is reacted with the amine 2 under standard amide coupling conditions to prepare the amide 3 in Step 1. Suitable amide coupling conditions are well known to the skilled person and include reacting a solution of the carboxylic acid 1 and a desired amine 2 in a suitable solvent, such as DCM or DMF, with an appropriate coupling reagent, such as T3P or HATU, in the presence of a suitable organic base, such as DiPEA, with stirring at RT. The removal of the PG moiety on the amine of the amide 3 is achieved under acidic conditions standard to the art. The intermediate amide 4 is obtained by reacting a suitable acid, such as TFA or HCl in Et₂O, to a stirring solution of the amide 3 in an appropriate solvent, such as DCM, at RT for at least 12 h. The intermediate amine is reacted with a suitable carboxylic acid under amide coupling conditions known to a skilled person. A suitable coupling reagent such as TCFH or HATU and an appropriate organic base, such as NMI or DiPEA, are introduced to a stirring solution of a desired carboxylic acid and the intermediate amine at RT in a suitable solvent, such as DMF or ACN.

In Step 3, the intermediate 4 is reacted under nucleophilic aromatic substitution conditions well known to the skilled artisan to give 5. Suitable nucleophilic aromatic substitution conditions include reacting a compound bearing an appropriate halogen on an aromatic moiety, such as chlorine, with a suitable nucleophilic reactant such as an amine or an alcohol. The reaction occurs in the presence of a suitable organic base, such as K₂CO₃ or NaH, in an appropriate organic solvent, such as NMP or DMF, at 80° C. at least. Optionally, CuI can be added to catalyze the nucleophilic aromatic substitution reaction.

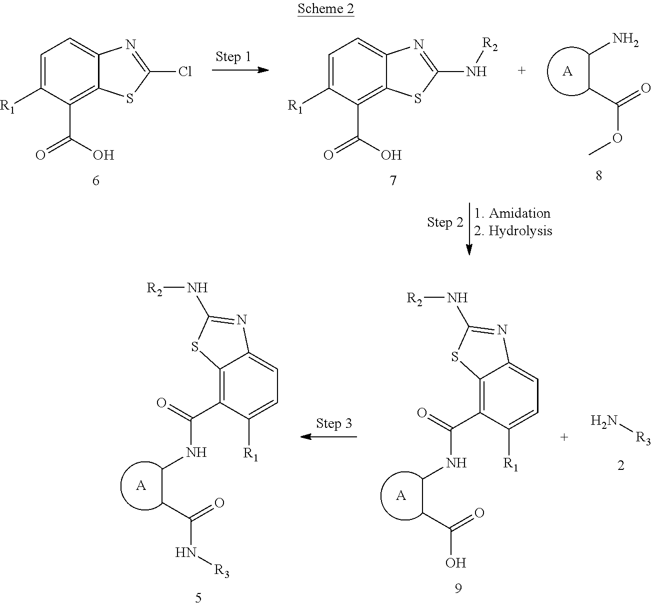

Scheme 2 depicts the preparation of compounds of the present invention beginning with a suitable carboxylic acid 6. In Step 1, the carboxylic acid 6 is reacted with a desired amine under nucleophilic aromatic substitution conditions well known to the skilled artisan to give 7. The reaction occurs in the presence of a suitable organic base, such as K₂CO₃, in an appropriate organic solvent, such as NMP, at 80° C. at least. The resulting carboxylic acid 7 is reacted with a desired amine 8 under standard amide coupling conditions. Suitable amide coupling conditions are well known to the skilled person and include reacting a solution of the carboxylic acid 7 and the amine 8 in a suitable solvent, such as ACN or DCM, with an appropriate coupling reagent, such as TCFH, or oxalyl chloride and DMF, in the presence of a suitable organic base, such as NMI or DiPEA, with stirring at RT for at least 18 h. The ester of intermediate amide is hydrolyzed under standard saponification conditions using an appropriate base, such as LiOH or TBD, in a mixture of suitable organic solvents, such as THF and H₂O, with stirring at RT at least, for at least 48 h.

In Step 3, the resulting carboxylic acid 9 is reacted with an appropriate amine 2 under standard amide coupling conditions and include reacting a solution of the carboxylic acid 9 and the amine 2 in a suitable solvent, such as ACN or NMP, with an appropriate coupling reagent, such as TCFH or T3P, in the presence of a suitable organic base, such as NMI or DiPEA, with stirring at RT for at least 48 h.

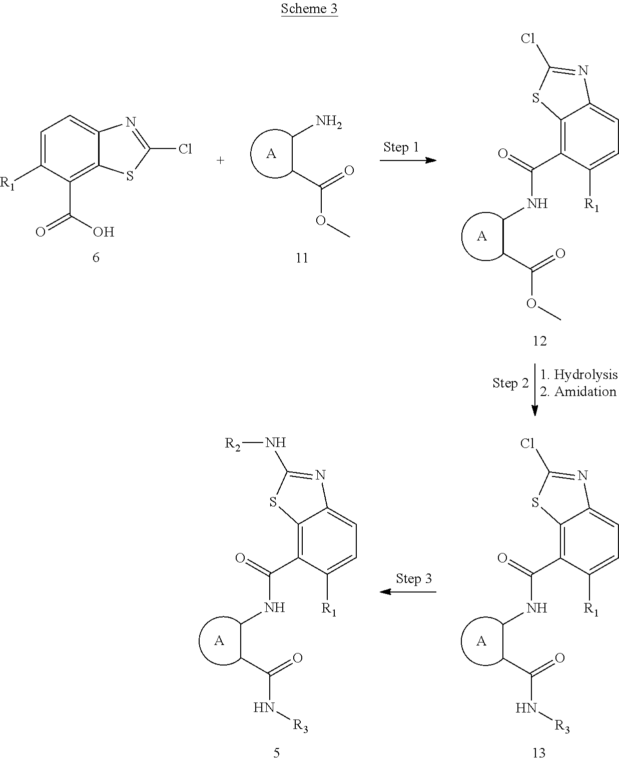

Scheme 3 depicts the preparation of compounds of the present invention beginning with a suitable carboxylic acid 6 and amine 11. The carboxylic acid 6 is reacted with the amine 11 under standard amide coupling conditions to prepare the amide 12 in Step 1. Suitable amide coupling conditions are well known to the skilled person and include reacting a solution of the carboxylic acid 6 and a desired amine 11 in a suitable solvent, such as ACN, with an appropriate coupling reagent, such as TCFH, in the presence of a suitable organic base, such as NMI, with stirring at RT for at least 18 h. The ester of amide 12 is hydrolyzed under standard saponification conditions using an appropriate strong inorganic base, such as LiOH, in a mixture of suitable organic solvents, such as THF and H₂O, with stirring at RT for at least 18 h. The intermediate carboxylic acid is then reacted with a suitable amine under standard amide coupling conditions and include reacting a solution of the carboxylic acid and a desired amine in a suitable solvent, such as ACN, with an appropriate coupling reagent, such as TCFH, in the presence of a suitable organic base, such as NMI, with stirring at RT.

In Step 3, the intermediate amide 13 is reacted under nucleophilic aromatic substitution conditions well known to the skilled artisan to give 5. The reaction occurs in the presence of a suitable organic base, such as K₂CO₃, in an appropriate organic solvent, such as NMP, at 80° C. at least.

In Schemes 1-3 if the R₂-group contains a carboxylic ester, it can be treated under standard saponification conditions using an appropriate strong inorganic base, such as LiOH or TBD, in a mixture of suitable organic solvents, such as THF and H₂O, with stirring at RT for at least 30 min.

### Preparation 1

### 2-(3-Oxa-7-azabicyclo[3.3.1]nonan-7-yl)-6-methoxybenzo[d]thiazole-7-carboxylic Acid

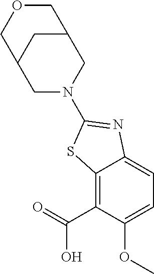 [SMILES: COc1ccc2nc(N3CC4COCC(C4)C3)sc2c1C(=O)O]

A solution of 3-oxa-7-azabicyclo[3.3.1]nonane (1.566 g, 12.31 mmol), 2-chloro-6-methoxybenzo[d]thiazole-7-carboxylic acid (1.0 g, 4.104 mmol) and K₂CO₃ (1.702 g, 12.31 mmol) in NMP (20 mL) was stirred at 80° C. for 48 h. After 48 h, a small portion of the material was purified directly by reverse phase column chromatography (C18, gradient of 0-100% (5% MeOH/ACN) in 10 mM aqueous NH₄HCO₃ pH 10) to provide impure product. The impure fractions and the reaction mixture were combined. Heptane (100 mL) was added and the mixture was filtered. The filter cake was rinsed with heptane (50 mL) and dried under high vacuum to provide the title compound (1.1 g, 3.3 mmol, 80%). ES-MS m/z 335 (M+H).

### Preparation 2

### Methyl (1R,2S,3R,4S)-3-(2-chloro-6-methoxybenzo[d]thiazole-7-carboxamido) bicyclo[2.2.1]heptane-2-carboxylate

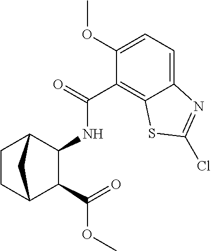 [SMILES: COC(=O)[C@H]1[C@@H]2CC[C@@H](C2)[C@H]1NC(=O)c1c(OC)ccc2nc(Cl)sc12]

To a stirring solution of 2-chloro-6-methoxybenzo[d]thiazole-7-carboxylic acid (1000 mg, 4.104 mmol) and methyl (1R,2S,3R,4S)-3-aminobicyclo[2.2.1]heptane-2-carboxylate (833.4 mg, 4.925 mmol) was added TCFH (2.764 g, 4.925 mmol, 3 mL) and NMI (808.7 mg, 9.850 mmol, 0.8 mL). The reaction mixture was stirred at room temperature for 18 h. After 18 h, H₂O (30 mL) and EtOAc (50 mL) were added to the reaction mixture. The organic layer was separated and the aqueous layer was extracted with EtOAc (100 mL). The combined organic layers were dried over Na₂SO₄, concentrated, and purified by silica gel chromatography (0-80% EtOAc in heptane) to provide the title compound (1025 mg, 2.596 mmol, 63%). ES-MS m/z 395 (M+H).

### Preparation 3

### (1R,2S,3R,4S)-3-(2-Chloro-6-methoxybenzo[d]thiazole-7-carboxamido) bicyclo[2.2.1]heptane-2-carboxylic Acid

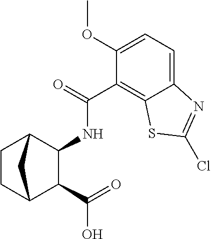 [SMILES: COc1ccc2nc(Cl)sc2c1C(=O)N[C@@H]1[C@H]2CC[C@H](C2)[C@@H]1C(=O)O]

To a stirring solution of methyl (1R,2S,3R,4S)-3-(2-chloro-6-methoxybenzo[d]thiazole-7-carboxamido) bicyclo[2.2.1]heptane-2-carboxylate (975 mg, 2.47 mmol) in THF (20 mL) was added a solution of LiOH (296 mg, 12.3 mmol) in H₂O (5 mL). The reaction mixture was stirred at room temperature for 18 h. The reaction mixture was diluted with EtOAc (50 mL) and acidified with 2 M HCl to ˜pH 4. The organic layer was separated. The aqueous layer was extracted with EtOAc (50 mL). The combined organic layers were dried over Na₂SO₄ and concentrated to provide the title compound (800 mg, 2.10 mmol, 85%). ES-MS m/z 381 (M+H).

### Preparation 4

### tert-Butyl ((1S,2R,3S,4R)-3-((4-fluoro-3-(trifluoromethyl)phenyl)carbamoyl)bicyclo[2.2.1]heptan-2-yl)carbamate

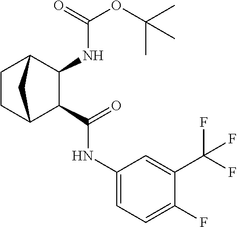 [SMILES: CC(C)(C)OC(=O)N[C@@H]1[C@H]2CC[C@H](C2)[C@@H]1C(=O)Nc1ccc(F)c(C(F)(F)F)c1]

T3P (26 g, 41 mmol, 24 mL) and DIPEA (3.5 g, 27 mmol, 4.8 mL) were added to a stirring solution of (1R,2S,3R,4S)-3-((tert-butoxycarbonyl)amino) bicyclo[2.2.1]heptane-2-carboxylic acid (3.5 g, 14 mmol) and 4-fluoro-3-(trifluoromethyl) aniline (4.9 g, 27 mmol, 5 mL) in DCM (100 mL). The reaction mixture was stirred at room temperature for 48 h. After 48 h, the reaction mixture was concentrated and purified by reverse phase column chromatography (C18, gradient of 0-100% (5% MeOH/ACN) in 10 mM aqueous NH₄HCO₃ pH 10) to provide the title compound (5.7 g, 14 mmol, 100%). ES-MS m/z 416 (M+H).

### Preparation 5

### (1R,2S,3R,4S)-3-Amino-N-(4-fluoro-3-(trifluoromethyl)phenyl) bicyclo[2.2.1]heptane-2-carboxamide

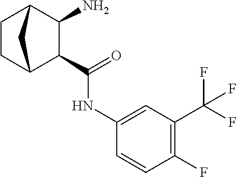 [SMILES: N[C@@H]1[C@H]2CC[C@H](C2)[C@@H]1C(=O)Nc1ccc(F)c(C(F)(F)F)c1]

To a solution of tert-butyl ((1S,2R,3S,4R)-3-((4-fluoro-3-(trifluoromethyl)phenyl)carbamoyl)bicyclo[2.2.1]heptan-2-yl)carbamate (18.5 g, 44.4 mmol) in DCM (317 mL) was added TFA (52.9 mL). The reaction was stirred at 25° C. After 2 hours the reaction was concentrated, then EtOAc (6 mL) was added to dissolve the residue, followed by the addition of pentanes (200 mL). The resulting mixture was stirred vigorously 12 hours to precipitate the product. After 12 hours of stirring, the product was isolated by filtration and rinsing with pentanes, dried under vacuum, to give the title compound (14.0 g, 44.3 mmol, >99%) as a white solid. ES-MS m/z 317 (M+H).

### Preparation 6

### 2-Chloro-N-((1S,2R,3S,4R)-3-((4-fluoro-3-(trifluoromethyl)phenyl)carbamoyl)bicyclo[2.2.1]heptan-2-yl)-6-methoxybenzo[d]thiazole-7-carboxamide

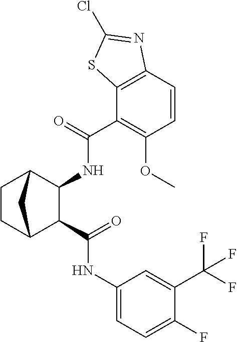 [SMILES: COc1ccc2nc(Cl)sc2c1C(=O)N[C@@H]1[C@H]2CC[C@H](C2)[C@@H]1C(=O)Nc1ccc(F)c(C(F)(F)F)c1]

To a stirring solution of 2-chloro-6-methoxybenzo[d]thiazole-7-carboxylic acid (900 mg, 3.69 mmol) in ACN (30 mL), TCFH (2.49 g, 4.43 mmol, 2 mL) and NMI (728 mg, 8.86 mmol, 0.7 mL) was added, followed by (1R,2S,3R,4S)-3-amino-N-(4-fluoro-3-(trifluoromethyl)phenyl) bicyclo[2.2.1]heptane-2-carboxamide (1.40 g, 4.43 mmol). The reaction mixture was stirred at room temperature for 1 h. After 1 h, H₂O (30 mL) and EtOAc (50 mL) were added to the reaction mixture. The organic layer was separated. The aqueous layer was extracted with EtOAc (100 mL). The combined organic layers were dried over Na₂SO₄, concentrated, and purified by silica gel chromatography (0-80% EtOAc in heptane) to provide the title compound (1 g, 2 mmol, 50%). ES-MS m/z 542 (M+H).

The following were prepared essentially as described in Preparation 6 with the appropriate amine and carboxylic acid:

| Preparation | Chemical name | Structure | ES-MS m/z |
|---|---|---|---|
| 7 | Methyl 3-(2-(3-oxa-7- azabicyclo[3.3.1]nonan-7-yl)-6- methoxybenzo[d]thiazole-7- carboxamido)isonicotinate | 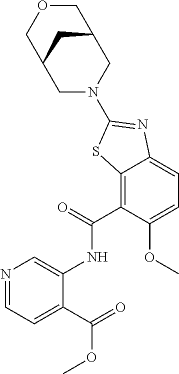 [SMILES: COC(=O)c1ccncc1NC(=O)c1c(OC)ccc2nc(N3C[C@@H]4COC[C@@H](C4)C3)sc12] | 469 (M + H) |
| 8 | 2-Chloro-6-methoxy-N- ((1S,2R,3S,4R)-3-((3- (trifluoromethyl)bicyclo[1.1.1] pentan-1- yl)carbamoyl)bicyclo[2.2.1] heptan-2-yl)benzo[d]thiazole-7- carboxamide^{a} | 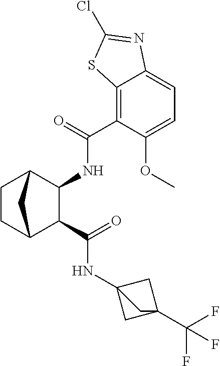 [SMILES: COc1ccc2nc(Cl)sc2c1C(=O)N[C@@H]1[C@H]2CC[C@H](C2)[C@@H]1C(=O)NC12CC(C(F)(F)F)(C1)C2] | 514 (M + H) |
| 9 | 2-Chloro-6-methoxy-N- ((1S,2R,3S,4R)-3-((1- (trifluoromethyl)-2- oxabicyclo[2.2.2]octan-4- yl)carbamoyl)bicyclo[2.2.1] heptan-2-yl)benzo[d]thiazole-7- carboxamide^{b} | 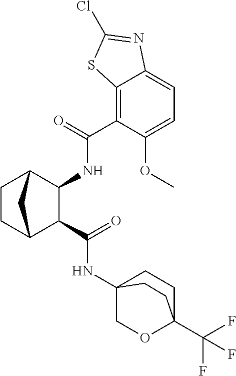 [SMILES: COc1ccc2nc(Cl)sc2c1C(=O)N[C@@H]1[C@H]2CC[C@H](C2)[C@@H]1C(=O)NC12CCC(C(F)(F)F)(CC1)OC2] | 558 (M + H) |

^{a}Starting materials: 3-(trifluoromethyl)bicyclo[1.1.1]pentan-1-amine and (1R,2S,3R,4S)-3-(2-chloro-6-
methoxybenzo[d]thiazole-7-carboxamido)bicyclo[2.2.1]heptane-2-carboxylic acid
^{b}Starting materials: 1-(trifluoromethyl)-2-oxabicyclo[2.2.2]octan-4-amine and (1R,2S,3R,4S)-3-(2-chloro-
6-methoxybenzo[d]thiazole-7-carboxamido)bicyclo[2.2.1]heptane-2-carboxylic acid

### Preparation 10

### 3-(2-(3-Oxa-7-azabicyclo[3.3.1]nonan-7-yl)-6-methoxybenzo[d]thiazole-7-carboxamido)isonicotinic Acid

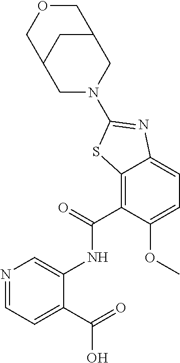 [SMILES: COc1ccc2nc(N3CC4COCC(C4)C3)sc2c1C(=O)Nc1cnccc1C(=O)O]

To a stirring solution of methyl 3-(2-(3-oxa-7-azabicyclo[3.3.1]nonan-7-yl)-6-methoxybenzo[d]thiazole-7-carboxamido) isonicotinate (60 mg, 0.13 mmol) in THF (2 mL) was added a solution of LiOH (50 mg, 2.1 mmol) in H₂O (0.7 mL). The reaction mixture was stirred at room temperature for 48 h. After 48 h, the reaction mixture was purified directly by reverse phase column chromatography (C18, gradient of 0-100% (5% MeOH/ACN) in 10 mM aqueous NH₄HCO₃ pH 10) to provide the title compound (60 mg, 0.12 mmol, 93%). ES-MS m/z 455 (M+H).

### Preparation 11

### Methyl 7-(7-(((1S,2R,3S,4R)-3-((4-fluoro-3-(trifluoromethyl)phenyl)carbamoyl)bicyclo[2.2.1]heptan-2-yl)carbamoyl)-6-methoxybenzo[d]thiazol-2-yl)-7-azaspiro[3.5]nonane-2-carboxylate

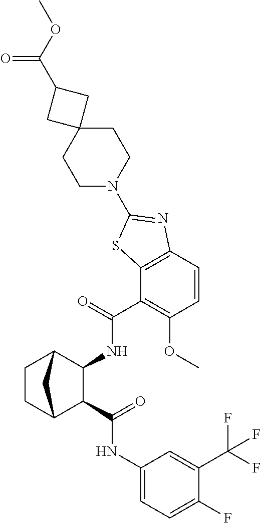 [SMILES: COC(=O)C1CC2(CCN(c3nc4ccc(OC)c(C(=O)N[C@@H]5[C@H]6CC[C@H](C6)[C@@H]5C(=O)Nc5ccc(F)c(C(F)(F)F)c5)c4s3)CC2)C1]

A solution of methyl 7-azaspiro[3.5]nonane-2-carboxylate (51 mg, 0.28 mmol) 2-chloro-N-((1S,2R,3S,4R)-3-((4-fluoro-3-(trifluoromethyl)phenyl)carbamoyl)bicyclo[2.2.1]heptan-2-yl)-6-methoxybenzo[d]thiazole-7-carboxamide (50 mg, 92 μmol) and K₂CO₃ (38 mg, 0.28 mmol) was stirred in DMF (1 mL) at 80° C. for 18 h. The reaction mixture was then purified directly by silica gel chromatography (0-100% EtOAc in heptane) to provide the title product (64 mg, 84 μmol, 91%). ES-MS m/z 689 (M+H).

### Preparation 12

### Methyl 2-((7-(((1S,2R,3S,4R)-3-((4-fluoro-3-(trifluoromethyl)phenyl)carbamoyl)bicyclo[2.2.1]heptan-2-yl)carbamoyl)-6-methoxybenzo[d]thiazol-2-yl)oxy)acetate

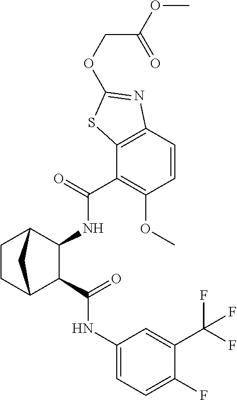 [SMILES: COC(=O)COc1nc2ccc(OC)c(C(=O)N[C@@H]3[C@H]4CC[C@H](C4)[C@@H]3C(=O)Nc3ccc(F)c(C(F)(F)F)c3)c2s1]

To a solution of methyl 2-hydroxyacetate (20 mg, 0.22 mmol, 0.02 mL) and 2-chloro-N-((1S,2R,3S,4R)-3-((4-fluoro-3-(trifluoromethyl)phenyl)carbamoyl)bicyclo[2.2.1]heptan-2-yl)-6-methoxybenzo[d]thiazole-7-carboxamide (40 mg, 74 μmol) in DMF (1 mL) was added NaH (5.3 mg, 0.22 mmol). The reaction mixture was stirred at 50° C. for 6 h. The reaction mixture was then purified directly by silica gel chromatography to provide the title compound (44 mg, 74 μmol, 100%). ES-MS m/z 596 (M+H).

### Preparation 13

### tert-Butyl (5-fluoro-4-((4-fluoro-3-(trifluoromethyl)phenyl)carbamoyl)pyridin-3-yl)carbamate

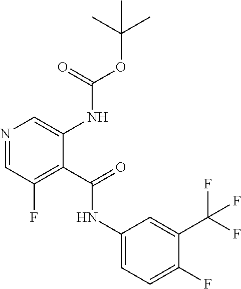 [SMILES: CC(C)(C)OC(=O)Nc1cncc(F)c1C(=O)Nc1ccc(F)c(C(F)(F)F)c1]

A solution of 4-fluoro-3-(trifluoromethyl) aniline (271 mg, 2 mmol), 3-((tert-butoxycarbonyl)amino)-5-fluoroisonicotinic acid (400 mg, 2 mmol), DiPEA (780 μL, 5 mmol) and HATU (1.27 g, 3.27 mmol) in anhydrous DMF (14.5 mL) was stirred at 50° C. for 65 min. The reaction mixture was quenched with H₂O (75 mL), and EtOAc (75 mL) was added. The phases were separated, and the aqueous phase was extracted with EtOAc (3×75 mL). The combined organic layers were washed with saturated aqueous NaCl, dried over Na₂SO₄, filtered, and concentrated under vacuum. The crude was purified by silica gel chromatography (1-50% EtOAc in n-heptane). The desired fractions were combined and concentrated to afford the title compound (350.9 mg, 54%) as an off-white powder. ES-MS m/z 418 (M+H).

### Preparation 14

### 3-Amino-5-fluoro-N-(4-fluoro-3-(trifluoromethyl)phenyl)isonicotinamide; hydrochloride

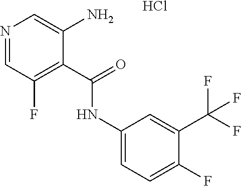 [SMILES: Cl ; Nc1cncc(F)c1C(=O)Nc1ccc(F)c(C(F)(F)F)c1]

A solution of tert-butyl (5-fluoro-4-((4-fluoro-3-(trifluoromethyl)phenyl)carbamoyl)pyridin-3-yl)carbamate (351 mg, 1 mmol) and 2 M HCl in Et₂O (8.20 mL, 16.4 mmol) in DCM (3.0 mL)/MeOH (1.0 mL) was stirred at RT overnight. The reaction mixture was concentrated under reduced pressure then triturated in Et₂O to afford the title compound (294.7 mg, 91%) as a yellow solid. ES-MS m/z 318 (M+H).

### Preparation 15

### (1S,2R)-2-Amino-N-(4-fluoro-3-(trifluoromethyl)phenyl)cyclopentane-1-carboxamide hydrochloride

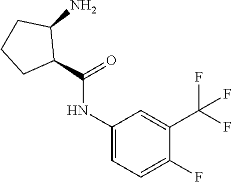 [SMILES: N[C@@H]1CCC[C@@H]1C(=O)Nc1ccc(F)c(C(F)(F)F)c1]

A mixture of (1S,2R)-2-[(tert-butoxycarbonyl)amino]cyclopentane-1-carboxylic acid (500 mg, 2.181 mmol) and 4-fluoro-3-(trifluoromethyl) aniline (470 mg, 2.617 mmol) in ACN (10 mL) was added TCFH (2.45 g, 8.724 mmol) and NMI (0.90 g, 10.905 mmol) at room temperature. The mixture was stirred for 2 h at room temperature. The resulting mixture was diluted with water (20 mL), and was extracted with EtOAc (3×20 mL). The combined organic layers were washed with saturated aqueous NaCl (20 mL), dried over anhydrous Na₂SO₄. After filtration, the filtrate was concentrated under reduced pressure. The residue was purified by silica gel column chromatography using 1:3 EtOAc in petroleum ether. To the product was added HCl (4 M in EtOAc, 5 mL) at 0° C. and the mixture was stirred overnight at RT. The resulting mixture was concentrated under reduced pressure to give the title compound. ES-MS m/z 291 (M+H)

### Preparation 16

### Methyl 2-(2-chloro-6-methoxybenzo[d]thiazole-7-carboxamido)-4-fluorobenzoate

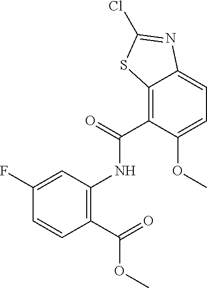 [SMILES: COC(=O)c1ccc(F)cc1NC(=O)c1c(OC)ccc2nc(Cl)sc12]

To a solution of 2-chloro-6-methoxybenzo[d]thiazole-7-carboxylic acid (1.15 g, 4.72 mmol) in DCM (20 mL), oxalyl chloride was added (1.50 g, 5.90 mL, 11.8 mmol), followed by DMF (0.25 mL). After stirring at RT for 2 h, the reaction mixture was concentrated under reduced pressure. The residue was dissolved in DCM (20 mL). To this solution was added methyl 2-amino-4-fluorobenzoate (958 mg, 6 mmol) and DiPEA (2.44 g, 3.25 mL, 18.9 mmol). After stirring for 2 h, the reaction mixture was diluted with DCM (200 mL) and washed with H₂O (3×100 mL) and saturated aqueous NaCl (100 mL). The organics were dried over Na₂SO₄, filtered and concentrated. The residue was dissolved in MeOH (100 mL), and the resulting precipitate was collected and dried under vacuum to give the title compound (1.6 g, 86%) as an off white solid. ES-MS m/z 395 (M+H).

The compounds in the following table were prepared essentially as described in Preparation 16 with the appropriate amine and carboxylic acid.

| Preparation | Chemical name | Structure | ES-MS m/z |
|---|---|---|---|
| 17 | Methyl 2-(2-chloro-6- methoxybenzo[d]thiazole-7- carboxamido)-6- fluorobenzoate | 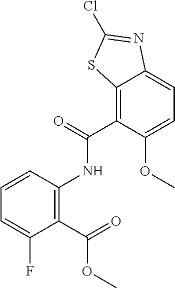 [SMILES: COC(=O)c1c(F)cccc1NC(=O)c1c(OC)ccc2nc(Cl)sc12] | 395 (M + H) |
| 18 | Methyl 2-(2-chloro-6- methoxybenzo[d]thiazole-7- carboxamido)-4,6- difluorobenzoate | 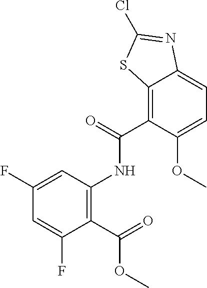 [SMILES: COC(=O)c1c(F)cc(F)cc1NC(=O)c1c(OC)ccc2nc(Cl)sc12] | 413 (M + H) |

The compounds in the following table were prepared essentially as described in Preparation 1 with the appropriate amine and 2-chlorobenzothiazole derivative.

| Preparation | Chemical name | Structure | ES-MS m/z |
|---|---|---|---|
| 19 | Methyl 2-(2-(3-oxa-6- azabicyclo[3.1.1]heptan-6-yl)-6- methoxybenzo[d]thiazole-7- carboxamido)-4-fluorobenzoate | 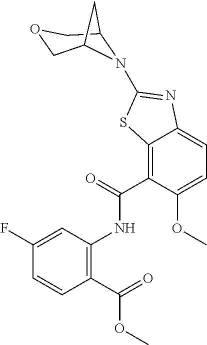 [SMILES: COC(=O)c1ccc(F)cc1NC(=O)c1c(OC)ccc2nc(N3C4COCC3C4)sc12] | 458 (M + H) |
| 20 | Methyl 2-(2-(3-oxa-6- azabicyclo[3.1.1]heptan-6-yl)-6- methoxybenzo[d]thiazole-7- carboxamido)-6-fluorobenzoate | 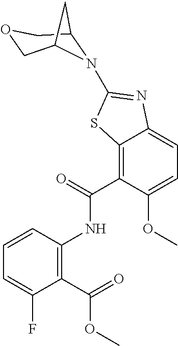 [SMILES: COC(=O)c1c(F)cccc1NC(=O)c1c(OC)ccc2nc(N3C4COCC3C4)sc12] | 458 (M + H) |
| 21 | 2-(3-Oxa-6-azabicyclo[3.1.1]heptan- 6-yl)-6-methoxybenzo[d]thiazole-7- carboxylic acid | 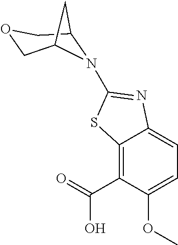 [SMILES: COc1ccc2nc(N3C4COCC3C4)sc2c1C(=O)O] | 307 (M + H) |
| 22 | Methyl 2-(2-(3-oxa-6- azabicyclo[3.1.1]heptan-6-yl)-6- methoxybenzo[d]thiazole-7- carboxamido)-4,6-difluorobenzoate | 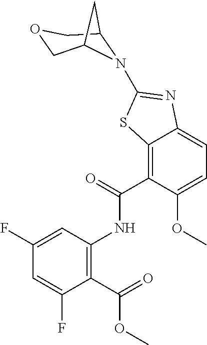 [SMILES: COC(=O)c1c(F)cc(F)cc1NC(=O)c1c(OC)ccc2nc(N3C4COCC3C4)sc12] | 476 (M + H) |

### Preparation 23

### 2-(2-(3-Oxa-6-azabicyclo[3.1.1]heptan-6-yl)-6-methoxybenzo[d]thiazole-7-carboxamido)-4-fluorobenzoic Acid

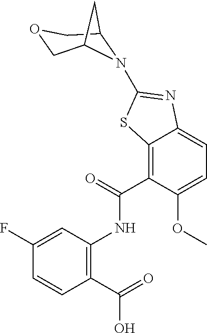 [SMILES: COc1ccc2nc(N3C4COCC3C4)sc2c1C(=O)Nc1cc(F)ccc1C(=O)O]

To a solution of methyl 2-(2-(3-oxa-6-azabicyclo[3.1.1]heptan-6-yl)-6-methoxybenzo[d]thiazole-7-carboxamido)-4-fluorobenzoate (0.60 g, 1.3 mmol) in THF (20 mL) and H₂O (2 mL), was added TBD (0.55 g, 3.9 mmol). The reaction mixture was stirred at 40° C. for 30 min. The reaction mixture was neutralized with aqueous citric acid and a precipitate was formed. The mixture was further diluted with H₂O and the precipitate was collected and dried under vacuum to give the title compound (0.5 g, 90%). ES-MS m/z 444 (M+H).

The compounds in the following table were prepared essentially as described in Preparation 23 using the appropriate ester.

| Preparation | Chemical name | Structure | ES-MS m/z |
|---|---|---|---|
| 24 | 2-(2-(3-Oxa-6- azabicyclo[3.1.1]heptan-6-yl)- 6-methoxybenzo[d]thiazole-7- carboxamido)-6-fluorobenzoic acid | 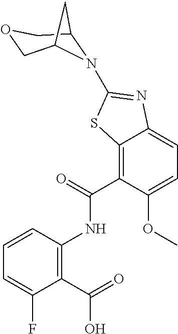 [SMILES: COc1ccc2nc(N3C4COCC3C4)sc2c1C(=O)Nc1cccc(F)c1C(=O)O] | 444 (M + H) |
| 25 | 2-(2-(3-Oxa-6- azabicyclo[3.1.1]heptan-6-yl)- 6-methoxybenzo[d]thiazole-7- carboxamido)-4,6- difluorobenzoic acid | 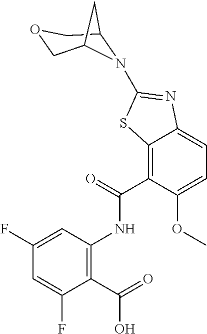 [SMILES: COc1ccc2nc(N3C4COCC3C4)sc2c1C(=O)Nc1cc(F)cc(F)c1C(=O)O] | 462 (M + H) |

The compound in the following table was prepared essentially as described in Preparation 6 using the appropriate amine and carboxylic acid.

| Preparation | Chemical name | Structure | ES-MS m/z |
|---|---|---|---|
| 6859 | Methyl 2-(2-(3-oxa-6- azabicyclo[3.1.1]heptan-6-yl)- 6-methoxybenzo[d]thiazole-7- carboxamido)benzoate | 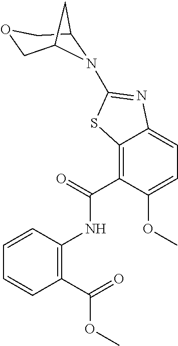 [SMILES: COC(=O)c1ccccc1NC(=O)c1c(OC)ccc2nc(N3C4COCC3C4)sc12] | 440 (M + H) |

The compound in the following table was prepared essentially as described in Preparation 10 using the appropriate ester.

| Preparation | Chemical name | Structure | ES-MS m/z |
|---|---|---|---|
| 27 | 2-(2-(3-Oxa-6- azabicyclo[3.1.1]heptan-6-yl)- 6-methoxybenzo[d]thiazole-7- carboxamido)benzoic acid | 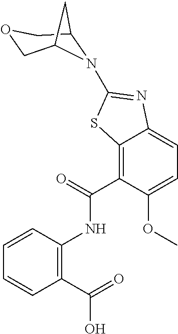 [SMILES: COc1ccc2nc(N3C4COCC3C4)sc2c1C(=O)Nc1ccccc1C(=O)O] | 426 (M + H) |

### Preparation 28

### 2-(2-(3-Oxa-6-azabicyclo[3.1.1]heptan-6-yl)-6-methoxybenzo[d]thiazole-7-carboxamido)-4-chlorobenzoic Acid

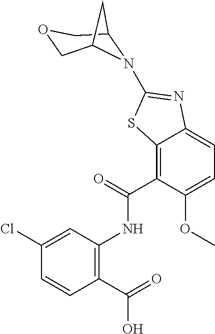 [SMILES: COc1ccc2nc(N3C4COCC3C4)sc2c1C(=O)Nc1cc(Cl)ccc1C(=O)O]

To a solution of 2-(3-oxa-6-azabicyclo[3.1.1]heptan-6-yl)-6-methoxybenzo[d]thiazole-7-carboxylic acid (0.25 g, 0.82 mmol) in NMP (5 mL) was added methyl 2-amino-4-chlorobenzoate (0.23 g, 1.2 mmol), T3P (1.6 g, 1.4 mL, 2.4 mmol), and DiPEA (0.42 g, 0.57 mL, 3.3 mmol). The reaction mixture was stirred at RT overnight. The reaction mixture was diluted with EtOAc (100 mL) and washed with H₂O (3× 50 mL), followed by saturated aqueous NaCl (50 mL). Organics were dried over Na₂SO₄, filtered and concentrated. Then, the residue was dissolved in THF (10 mL) and H₂O (2 mL) and treated with TBD (0.34 g, 2.4 mmol). The reaction mixture was stirred at 45° C. for 1 h then neutralized with a citric acid solution. Aqueous work-up gave the title compound (181 mg, 48%) as an off white solid. ES-MS m/z 460 (M+H).

The compounds in the following table were prepared essentially as described in Preparation 28 using the appropriate amine.

| Preparation | Chemical name | Structure | ES-MS m/z |
|---|---|---|---|
| 29 | 2-(2-(3-Oxa-6- azabicyclo[3.1.1]heptan-6-yl)- 6-methoxybenzo[d]thiazole-7- carboxamido)-4-cyanobenzoic acid | 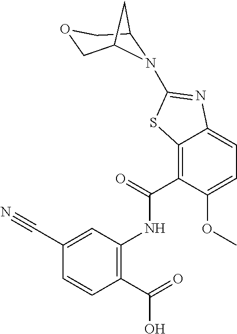 [SMILES: COc1ccc2nc(N3C4COCC3C4)sc2c1C(=O)Nc1cc(C#N)ccc1C(=O)O] | 451 (M + H) |
| 30 | 2-(2-(3-Oxa-6- azabicyclo[3.1.1]heptan-6-yl)- 6-methoxybenzo[d]thiazole-7- carboxamido)-5- (trifluoromethyl)benzoic acid | 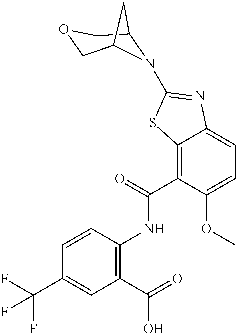 [SMILES: COc1ccc2nc(N3C4COCC3C4)sc2c1C(=O)Nc1ccc(C(F)(F)F)cc1C(=O)O] | 494 (M + H) |
| 31 | 5-(2-(3-Oxa-6- azabicyclo[3.1.1]heptan-6-yl)- 6-methoxybenzo[d]thiazole-7- carboxamido)-2- methylisonicotinic acid | 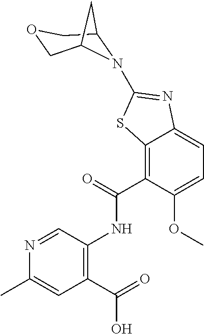 | 441 (M + H) |
| 32 | 2-(2-(3-Oxa-6- azabicyclo[3.1.1]heptan-6-yl)- 6-methoxybenzo[d]thiazole-7- carboxamido)-4- methylbenzoic acid | 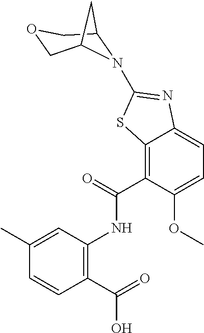 | 440 (M + H) |
| 33 | 2-(2-(3-Oxa-6- azabicyclo[3.1.1]heptan-6-yl)- 6-methoxybenzo[d]thiazole-7- carboxamido)-4,5- difluorobenzoic acid | 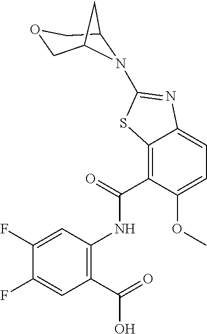 [SMILES: COc1ccc2nc(N3C4COCC3C4)sc2c1C(=O)Nc1cc(F)c(F)cc1C(=O)O] | 462 (M + H) |
| 34 | 2-(2-(3-Oxa-6- azabicyclo[3.1.1]heptan-6-yl)- 6-methoxybenzo[d]thiazole-7- carboxamido)-5-fluorobenzoic acid | 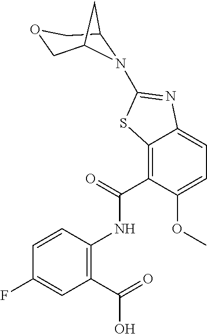 [SMILES: COc1ccc2nc(N3C4COCC3C4)sc2c1C(=O)Nc1ccc(F)cc1C(=O)O] | 444 (M + H) |
| 35 | 2-(2-(3-Oxa-6- azabicyclo[3.1.1]heptan-6-yl)- 6-methoxybenzo[d]thiazole-7- carboxamido)-4- methoxybenzoic acid | 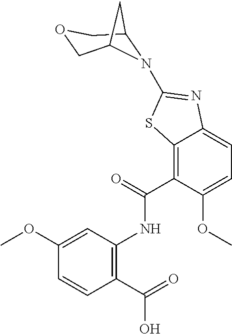 | 456 (M + H) |
| 36 | 2-(2-(3-Oxa-6- azabicyclo[3.1.1]heptan-6-yl)- 6-methoxybenzo[d]thiazole-7- carboxamido)-4- (trifluoromethyl)benzoic acid | 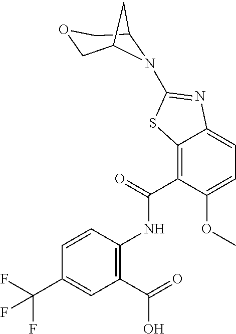 [SMILES: COc1ccc2nc(N3C4COCC3C4)sc2c1C(=O)Nc1ccc(C(F)(F)F)cc1C(=O)O] | 494 (M + H) |
| 37 | 2-(2-(3-Oxa-6- azabicyclo[3.1.1]heptan-6-yl)- 6-methoxybenzo[d]thiazole-7- carboxamido)-4- (difluoromethoxy)benzoic acid | 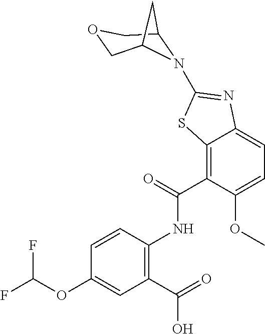 [SMILES: COc1ccc2nc(N3C4COCC3C4)sc2c1C(=O)Nc1ccc(OC(F)F)cc1C(=O)O] | 492 (M + H) |
| 38 | 2-(2-(3-Oxa-6- azabicyclo[3.1.1]heptan-6-yl)- 6-methoxybenzo[d]thiazole-7- carboxamido)-5- methylbenzoic acid | 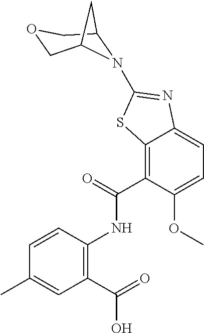 | 440 (M + H) |
| 39 | 2-(2-(3-Oxa-6- azabicyclo[3.1.1]heptan-6-yl)- 6-methoxybenzo[d]thiazole-7- carboxamido)-5- chlorobenzoic acid | 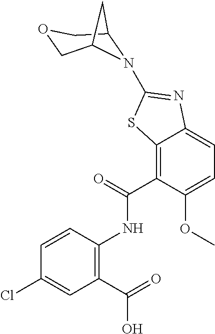 [SMILES: COc1ccc2nc(N3C4COCC3C4)sc2c1C(=O)Nc1ccc(Cl)cc1C(=O)O] | 460 (M + H) |
| 40 | 2-(2-(3-Oxa-6- azabicyclo[3.1.1]heptan-6-yl)- 6-methoxybenzo[d]thiazole-7- carboxamido)-5- methoxybenzoic acid | 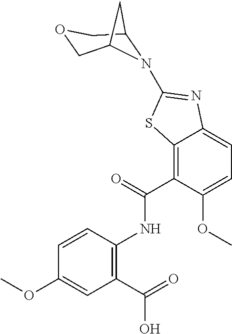 | 460 (M + H) |
| 41 | 2-(2-(3-Oxa-6- azabicyclo[3.1.1]heptan-6-yl)- 6-methoxybenzo[d]thiazole-7- carboxamido)-4-fluoro-5- methylbenzoic acid | 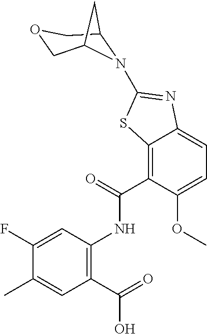 [SMILES: COc1ccc2nc(N3C4COCC3C4)sc2c1C(=O)Nc1cc(F)c(C)cc1C(=O)O] | 458 (M + H) |
| 42 | (1S,2R)-2-(2-Chloro-6- methoxybenzo[d]thiazole-7- carboxamido)cyclobutane-1- carboxylic acid | 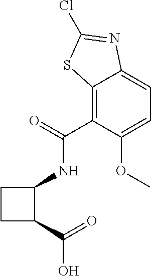 [SMILES: COc1ccc2nc(Cl)sc2c1C(=O)N[C@@H]1CC[C@@H]1C(=O)O] | 341 (M + H) |
| 43 | 7-(2-(3-Oxa-6- azabicyclo[3.1.1]heptan-6-yl)- 6-methoxybenzo[d]thiazole-7- carboxamido)-2,3- dihydrobenzo[b][1,4]dioxine- 6-carboxylic acid | 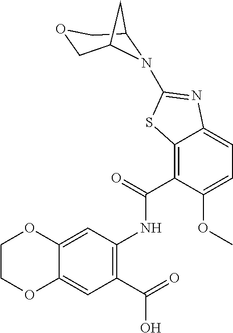 [SMILES: COc1ccc2nc(N3C4COCC3C4)sc2c1C(=O)Nc1cc2c(cc1C(=O)O)OCCO2] | 484 (M + H) |
| 44 | 6-(2-(3-Oxa-6- azabicyclo[3.1.1]heptan-6-yl)- 6-methoxybenzo[d]thiazole-7- carboxamido)benzo[d][1,3] dioxole-5-carboxylic acid | 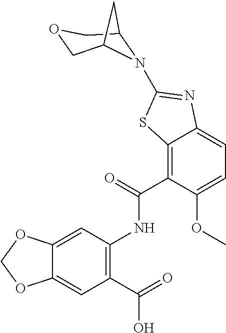 [SMILES: COc1ccc2nc(N3C4COCC3C4)sc2c1C(=O)Nc1cc2c(cc1C(=O)O)OCO2] | 470 (M + H) |

The compound in the following table was prepared essentially as described in Preparation 4 using (1S,2R)-2-(2-chloro-6-methoxybenzo[d]thiazole-7-carboxamido)cyclobutane-1-carboxylic acid and 2,2-difluorobenzo[d][1,3]dioxol-5-amine.

| Preparation | Chemical name | Structure | ES-MS m/z |
|---|---|---|---|
| 45 | 2-Chloro-N-((1R,2S)-2-((2,2- difluorobenzo[d][1,3]dioxol- 5-yl)carbamoyl)cyclobutyl)-6- methoxybenzo[d]thiazole-7- carboxamide | 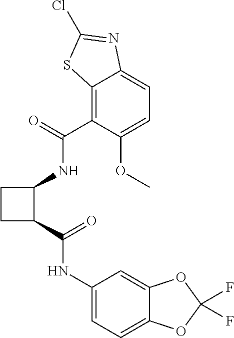 [SMILES: COc1ccc2nc(Cl)sc2c1C(=O)N[C@@H]1CC[C@@H]1C(=O)Nc1ccc2c(c1)OC(F)(F)O2] | 494 (M + H) |

### Example 1

### 2-(9,9-Difluoro-3,3-dioxido-3-thia-7-azabicyclo[3.3.1]nonan-7-yl)-N-((1S,2R,3S,4R)-3-((4-fluoro-3-(trifluoromethyl)phenyl)carbamoyl)bicyclo[2.2.1]heptan-2-yl)-6-methoxybenzo[d]thiazole-7-carboxamide

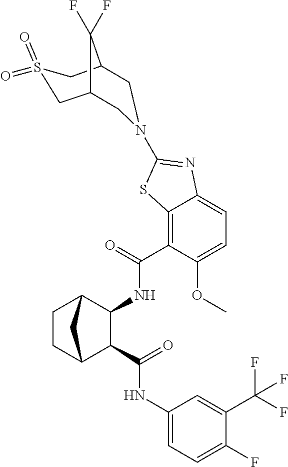 [SMILES: COc1ccc2nc(N3CC4CS(=O)(=O)CC(C3)C4(F)F)sc2c1C(=O)N[C@@H]1[C@H]2CC[C@H](C2)[C@@H]1C(=O)Nc1ccc(F)c(C(F)(F)F)c1]

A solution of 9,9-difluoro-3-thia-7-azabicyclo[3.3.1]nonane 3,3-dioxide (71 mg, 0.36 mmol), 2-chloro-N-((1S,2R,3S,4R)-3-((4-fluoro-3-(trifluoromethyl)phenyl)carbamoyl)bicyclo[2.2.1]heptan-2-yl)-6-methoxybenzo[d]thiazole-7-carboxamide (65 mg, 0.12 mmol) and K₂CO₃ (50 mg, 0.36 mmol) were stirred in NMP (1 mL) at 80° C. for 18 h. Copper (I) iodide (23 mg, 0.12 mmol) was added and the reaction mixture was stirred at 105° C. for 48 h. The reaction mixture was cooled to room temperature and purified directly by reverse phase column chromatography (C18, gradient of 0-100% (5% MeOH/ACN) in 10 mM aqueous NH₄HCO₃ pH 10) to provide the title compound (5 mg, 7 μmol, 6%). ES-MS m/z 717 (M+H).

The following were prepared essentially as described in Example 1 using the appropriate amine:

| Example | Chemical name | Structure | ES-MS m/z |
|---|---|---|---|
| 2 | 2-(3,3-Dioxido-3-thia-7- azabicyclo[3.3.1]nonan-7-yl)-N- ((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 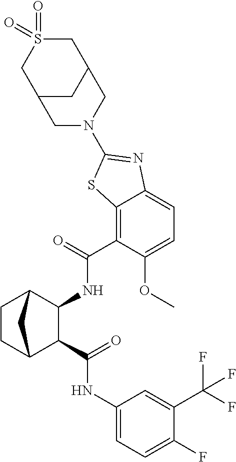 [SMILES: COc1ccc2nc(N3CC4CC(C3)CS(=O)(=O)C4)sc2c1C(=O)N[C@@H]1[C@H]2CC[C@H](C2)[C@@H]1C(=O)Nc1ccc(F)c(C(F)(F)F)c1] | 681 (M + H) |
| 3 | 2-(3-Oxa-6-azabicyclo[3.1.1]heptan- 6-yl)-N-((1S,2R,3S,4R)-3-((4-fluoro- 3- (trifluoromethyl)phenyl)carbamoyl) bicyclo[2.2.1]heptan-2-yl)-6- methoxybenzo[d]thiazole-7- carboxamide | 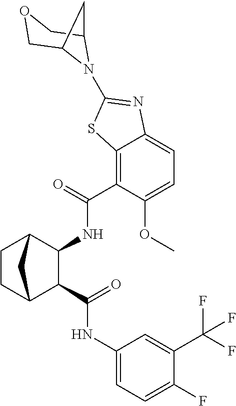 [SMILES: COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1] | 605 (M + H) |
| 4 | N-((1S,2R,3S,4R)-3-((4-Fluoro-3- (trifluoromethyl)phenyl)carbamoyl) bicyclo[2.2.1]heptan-2-yl)-6-methoxy- 2-(9-methyl-3-oxa-7,9- diazabicyclo[3.3.1]nonan-7- yl)benzo[d]thiazole-7- carboxamide | 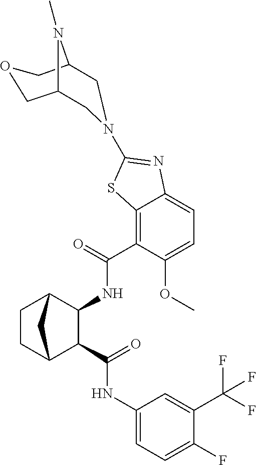 [SMILES: COc1ccc2nc(N3CC4COCC(C3)N4C)sc2c1C(=O)N[C@@H]1[C@H]2CC[C@H](C2)[C@@H]1C(=O)Nc1ccc(F)c(C(F)(F)F)c1] | 648 (M + H) |
| 5 | 2-(8-Oxa-3-azabicyclo[3.2.1]octan-3- yl)-N-((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 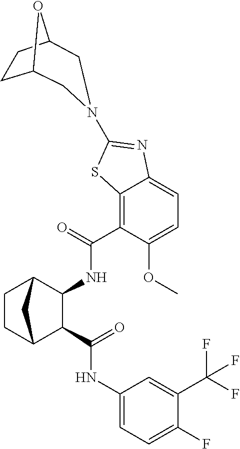 [SMILES: COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1] | 619 (M + H) |
| 6 | 2-((1S,4S)-2-Oxa-5- azabicyclo[2.2.1]heptan-5-yl)-N- ((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 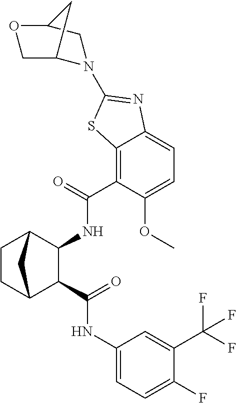 [SMILES: COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1] | 605 (M + H) |
| 7 | N-((1S,2R,3S,4R)-3-((4-Fluoro-3- (trifluoromethyl)phenyl)carbamoyl) bicyclo[2.2.1]heptan-2-yl)-6-methoxy- 2-(7-oxa-2-azaspiro[3.5]nonan-2- yl)benzo[d]thiazole-7- carboxamide | 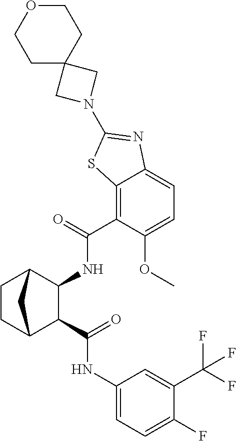 [SMILES: COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1] | 633 (M + H) |
| 8 | N-((1S,2R,3S,4R)-3-((4-Fluoro-3- (trifluoromethyl)phenyl)carbamoyl) bicyclo[2.2.1]heptan-2-yl)-6-methoxy-2- ((3aR,6aS)-tetrahydro-1H-furo[3,4- c]pyrrol-5(3H)-yl)benzo[d]thiazole-7- carboxamide | 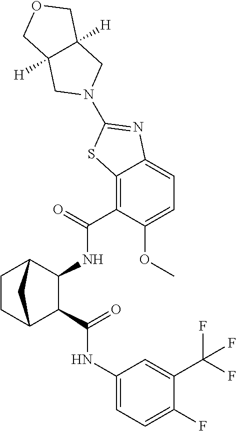 [SMILES: COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1] | 619 (M + H) |
| 9 | N-((1S,2R,3S,4R)-3-((4-Fluoro-3- (trifluoromethyl)phenyl)carbamoyl) bicyclo[2.2.1]heptan-2-yl)-2-((5aS,8aS)- hexahydro-2H- cyclopenta[b][1,4]oxazepin-5(5aH)- yl)-6-methoxybenzo[d]thiazole-7- carboxamide and N-((1S,2R,3S,4R)-3- ((4-fluoro-3- (trifluoromethyl)phenyl)carbamoyl) bicyclo[2.2.1]heptan-2-yl)-2- ((5aR,8aR)-hexahydro-2H- cyclopenta[b][1,4]oxazepin-5(5aH)- yl)-6-methoxybenzo[d]thiazole-7- carboxamide (mixture of isomers)^{a} | 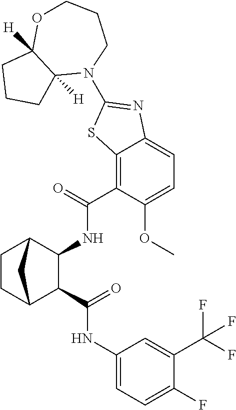 [SMILES: COc1ccc2nc(N3CCCO[C@H]4CCC[C@@H]43)sc2c1C(=O)N[C@@H]1[C@H]2CC[C@H](C2)[C@@H]1C(=O)Nc1ccc(F)c(C(F)(F)F)c1] | 647 (M + H) |
| | | 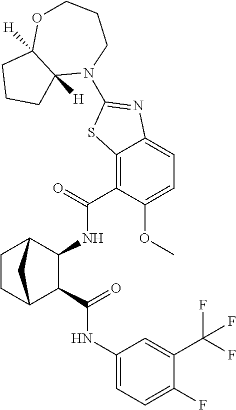 [SMILES: COc1ccc2nc(N3CCCO[C@@H]4CCC[C@H]43)sc2c1C(=O)N[C@@H]1[C@H]2CC[C@H](C2)[C@@H]1C(=O)Nc1ccc(F)c(C(F)(F)F)c1] | |
| 10 | 2-(1,1-Dioxido-1-thia-6- azaspiro[3.3]heptan-6-yl)-N- ((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 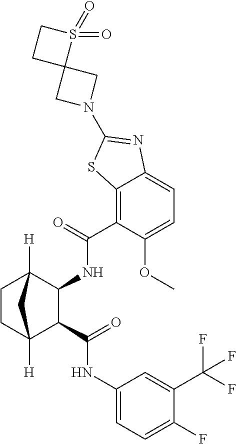 [SMILES: COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1] | 653 (M + H) |
| 11 | N-((1S,2R,3S,4R)-3-((4-Fluoro-3- (trifluoromethyl)phenyl)carbamoyl) bicyclo[2.2.1]heptan-2-yl)-6-methoxy- 2-(1-thia-6-azaspiro[3.3]heptan-6- yl)benzo[d]thiazole-7- carboxamide | 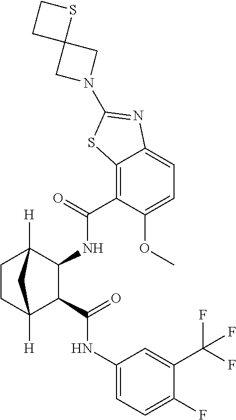 [SMILES: COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1] | 621 (M + H) |
| 12 | N-((1S,2R,3S,4R)-3-((4-Fluoro-3- (trifluoromethyl)phenyl)carbamoyl) bicyclo[2.2.1]heptan-2-yl)-6-methoxy- 2-(1-oxa-6-azaspiro[3.3]heptan-6- yl)benzo[d]thiazole-7- carboxamide | 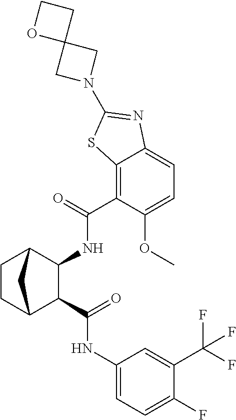 [SMILES: COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1] | 605 (M + H) |
| 13 | N-((1S,2R,3S,4R)-3-((4-Fluoro-3- (trifluoromethyl)phenyl)carbamoyl) bicyclo[2.2.1]heptan-2-yl)-2- (hexahydropyrano[3,4-c]pyrrol-2(3H)- yl)-6-methoxybenzo[d]thiazole-7- carboxamide (mixture of isomers) | 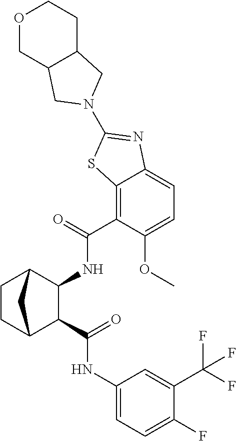 [SMILES: COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1] | 633 (M + H) |
| 14 | N-((1S,2R,3S,4R)-3-((4-Fluoro-3- (trifluoromethyl)phenyl)carbamoyl) bicyclo[2.2.1]heptan-2-yl)-2- ((3aS,7aR)-hexahydrofuro[3,2- c]pyridin-5(4H)-yl)-6- methoxybenzo[d]thiazole-7- carboxamide and N-((1S,2R,3S,4R)-3- ((4-fluoro-3- (trifluoromethyl)phenyl)carbamoyl) bicyclo[2.2.1]heptan-2-yl)-2- ((3aR,7aS)-hexahydrofuro[3,2- c]pyridin-5(4H)-yl)-6- methoxybenzo[d]thiazole-7- carboxamide (mixture of isomers)^{b} | 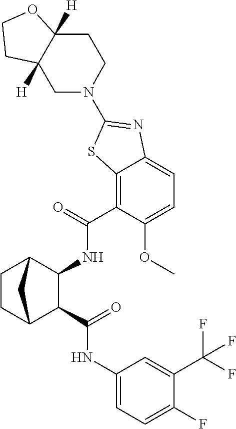 [SMILES: COc1ccc2nc(N3CC[C@H]4OCC[C@H]4C3)sc2c1C(=O)N[C@@H]1[C@H]2CC[C@H](C2)[C@@H]1C(=O)Nc1ccc(F)c(C(F)(F)F)c1] | 633 (M + H) |
| | | 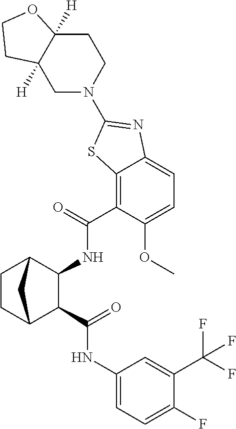 [SMILES: COc1ccc2nc(N3CC[C@@H]4OCC[C@@H]4C3)sc2c1C(=O)N[C@@H]1[C@H]2CC[C@H](C2)[C@@H]1C(=O)Nc1ccc(F)c(C(F)(F)F)c1] | |
| 15 | 2-(3-Oxa-8-azabicyclo[3.2.1]octan-8- yl)-N-((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 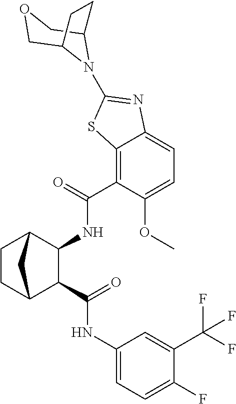 [SMILES: COc1ccc2nc(N3C4CCC3COC4)sc2c1C(=O)N[C@@H]1[C@H]2CC[C@H](C2)[C@@H]1C(=O)Nc1ccc(F)c(C(F)(F)F)c1] | 619 (M + H) |
| 16 | N-((1S,2R,3S,4R)-3-((4-Fluoro-3- (trifluoromethyl)phenyl)carbamoyl) bicyclo[2.2.1]heptan-2-yl)-6-methoxy- 2-(5-oxa-2-azaspiro[3.4]octan-2- yl)benzo[d]thiazole-7- carboxamide | 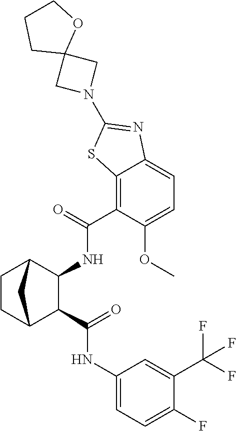 [SMILES: COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1] | 619 (M + H) |
| 17 | 2-(6-Oxa-3-azabicyclo[3.2.1]octan-3- yl)-N-((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 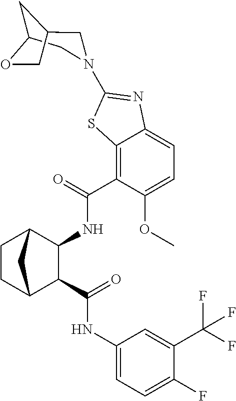 [SMILES: COc1ccc2nc(N3CC4COC(C4)C3)sc2c1C(=O)N[C@@H]1[C@H]2CC[C@H](C2)[C@@H]1C(=O)Nc1ccc(F)c(C(F)(F)F)c1] | 619 (M + H) |
| 18 | 2-(3-Oxa-7-azabicyclo[3.3.1]nonan-7- yl)-N-((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 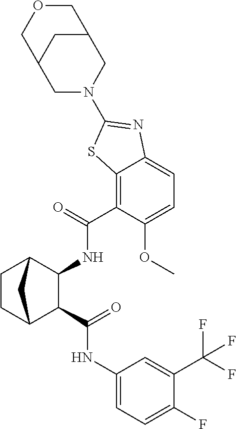 [SMILES: COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1] | 633 (M + H) |
| 19 | 2-(3-Oxa-9-azabicyclo[3.3.1]nonan-9- yl)-N-((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 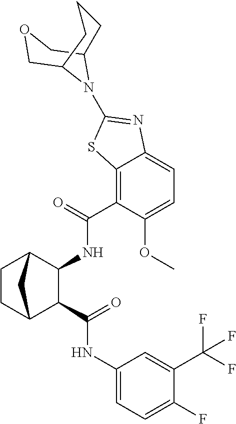 [SMILES: COc1ccc2nc(N3C4CCCC3COC4)sc2c1C(=O)N[C@@H]1[C@H]2CC[C@H](C2)[C@@H]1C(=O)Nc1ccc(F)c(C(F)(F)F)c1] | 633 (M + H) |
| 20 | 2-(6-Oxa-3-azabicyclo[3.1.1]heptan- 3-yl)-N-((1S,2R,3S,4R)-3-((4-fluoro- 3- (trifluoromethyl)phenyl)carbamoyl) bicyclo[2.2.1]heptan-2-yl)-6- methoxybenzo[d]thiazole-7- carboxamide | 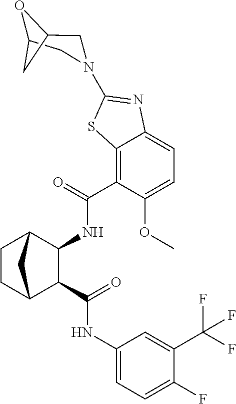 [SMILES: COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1] | 605 (M + H) |
| 21 | N-((1S,2R,3S,4R)-3-((4-Fluoro-3- (trifluoromethyl)phenyl)carbamoyl) bicyclo[2.2.1]heptan-2-yl)-6-methoxy- 2-(2-oxa-7-azaspiro[3.5]nonan-7- yl)benzo[d]thiazole-7- carboxamide | 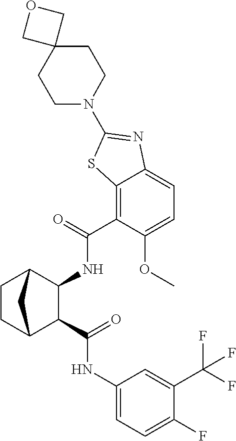 [SMILES: COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1] | 633 (M + H) |
| 22 | 2-(3-Oxa-7-azabicyclo[3.3.1]nonan-7- yl)-6-methoxy-N-((1S,2R,3S,4R)-3- ((3- (trifluoromethyl)bicyclo[1.1.1]pentan- 1-yl)carbamoyl)bicyclo[2.2.1]heptan- 2-yl)benzo[d]thiazole-7- carboxamide | 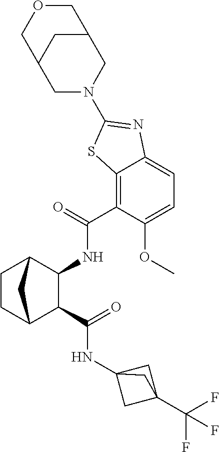 [SMILES: COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2] | 605 (M + H) |
| 23 | 2-(3-Oxa-7-azabicyclo[3.3.1]nonan-7- yl)-6-methoxy-N-((1S,2R,3S,4R)-3- ((1-(trifluoromethyl)-2- oxabicyclo[2.2.2]octan-4- yl)carbamoyl)bicyclo[2.2.1]heptan-2- yl)benzo[d]thiazole-7- carboxamide | 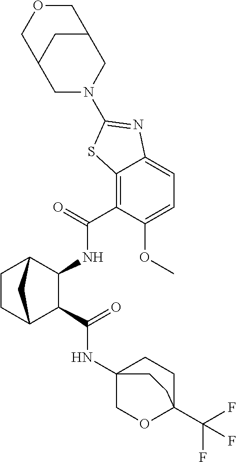 [SMILES: COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2] | 649 (M + H) |

^{a}Example 9 was isolated as a mixture of isomers using a racemic mixture of (5aR,8aR)-octahydro-2H-cyclopenta[b]
[1,4]oxazepane and (5aS,8aS)-octahydro-2H-cyclopenta[b][1,4]oxazepane as the starting amine.
^{b}Example 14 was isolated as a mixture of isomers using a racemic mixture of (3aR,7aS)-octahydrofuro[3,2-c]pyridine
hydrodochloride and (3aS,7aR)-octahydrofuro[3,2-c]pyridine hydrochloride as the starting amine.

### Example 24

### 7-(7-(((1S,2R,3S,4R)-3-((4-Fluoro-3-(trifluoromethyl)phenyl)carbamoyl)bicyclo[2.2.1]heptan-2-yl)carbamoyl)-6-methoxybenzo[d]thiazol-2-yl)-7-azaspiro[3.5]nonane-2-carboxylic Acid

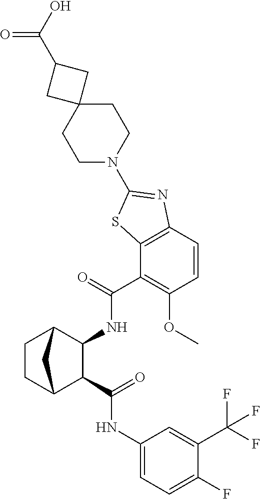 [SMILES: COc1ccc2nc(N3CCC4(CC3)CC(C(=O)O)C4)sc2c1C(=O)N[C@@H]1[C@H]2CC[C@H](C2)[C@@H]1C(=O)Nc1ccc(F)c(C(F)(F)F)c1]

To a stirring solution of methyl 7-(7-(((1S,2R,3S,4R)-3-((4-fluoro-3-(trifluoromethyl)phenyl)carbamoyl)bicyclo[2.2.1]heptan-2-yl)carbamoyl)-6-methoxybenzo[d]thiazol-2-yl)-7-azaspiro[3.5]nonane-2-carboxylate (64 mg, 93 μmol) in THF (0.7 mL) was added a solution of LiOH (11 mg, 0.46 mmol) in H₂O (0.3 mL). After 30 min, the reaction mixture was purified directly by reverse phase column chromatography (C18, gradient of 0-100% (5% MeOH/ACN) in 10 mM aqueous NH₄HCO₃ pH 10) to give the title compound (33.3 mg, 49.4 μmol, 53%). ES-MS m/z 675 (M+H).

The following was prepared essentially as described in Example 24 using the appropriate ester:

| Example | Chemical name | Structure | ES-MS m/z |
|---|---|---|---|
| 25 | 2-((7-(((1S,2R,3S,4R)-3-((4- Fluoro-3- (trifluoromethyl)phenyl) carbamoyl)bicyclo[2.2.1] heptan-2-yl)carbamoyl)-6- methoxybenzo[d]thiazol-2- yl)oxy)acetic acid | 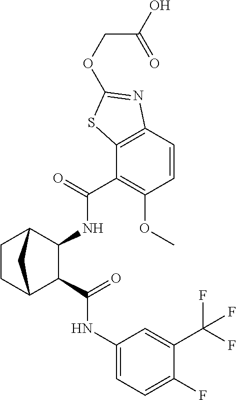 [SMILES: COc1ccc2nc(OCC(=O)O)sc2c1C(=O)N[C@@H]1[C@H]2CC[C@H](C2)[C@@H]1C(=O)Nc1ccc(F)c(C(F)(F)F)c1] | 582 (M + H) |

### Example 26

### 2-(3-Oxa-7-azabicyclo[3.3.1]nonan-7-yl)-N-(4-((4-fluoro-3-(trifluoromethyl)phenyl)carbamoyl)pyridin-3-yl)-6-methoxybenzo[d]thiazole-7-carboxamide

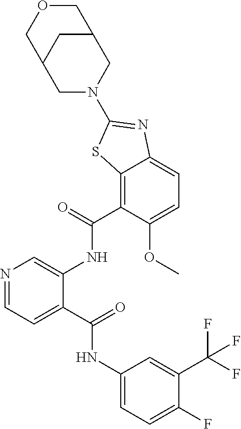 [SMILES: COc1ccc2nc(N3CC4COCC(C4)C3)sc2c1C(=O)Nc1cnccc1C(=O)Nc1ccc(F)c(C(F)(F)F)c1]

A solution of 4-fluoro-3-(trifluoromethyl)benzenamine (24 mg, 0.13 mmol, 17 μL), 3-(2-(3-oxa-7-azabicyclo[3.3.1]nonan-7-yl)-6-methoxybenzo[d]thiazole-7-carboxamido)isonicotinic acid (60 mg, 0.13 mmol), TCFH (89 mg, 0.32 mmol) and NMI (13 mg, 0.16 mmol, 12 μL) in ACN (1 mL) was stirred at room temperature for 48 h. The reaction mixture was then purified 3 times by silica gel chromatography (0-20% MeOH in DCM) to obtain the title compound (6.6 mg, 0.13 mmol, 8%). ES-MS m/z 616 (M+H).

### Example 27

### 2-(3-Oxa-7-azabicyclo[3.3.1]nonan-7-yl)-N-(5-fluoro-4-((4-fluoro-3-(trifluoromethyl)phenyl)carbamoyl)pyridin-3-yl)-6-methoxybenzo[d]thiazole-7-carboxamide

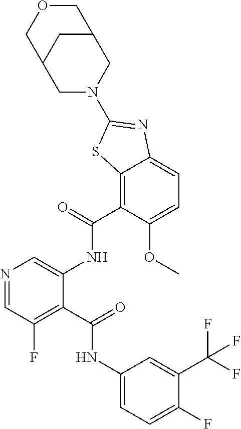 [SMILES: COc1ccc2nc(N3CC4COCC(C4)C3)sc2c1C(=O)Nc1cncc(F)c1C(=O)Nc1ccc(F)c(C(F)(F)F)c1]

A solution of 2-(3-oxa-7-azabicyclo[3.3.1]nonan-7-yl)-6-methoxybenzo[d]thiazole-7-carboxylic acid (65.0 mg, 0.2 mmol), 3-amino-5-fluoro-N-(4-fluoro-3-(trifluoromethyl)phenyl)isonicotinamide hydrochloride (74.7 mg, 0.2 mmol), and DiPEA (68.0 μL, 0.4 mmol) in ACN (1.0 mL) was stirred at 100° C. for 2 min. A solution of NMI (62.0 μL, 0.8 mmol) and TCFH (111 mg) in ACN (1 mL) was added to the reaction mixture and was stirred at 100° C. for 45 min. The reaction mixture was quenched with H₂O (20 mL) and EtOAc (20 mL) was added. The phases were separated and the aqueous phase was extracted with EtOAc (3×20 mL). The combined organic layers were washed with saturated aqueous NaCl, dried over Na₂SO₄, filtered and concentrated under vacuum. The residue was purified by silica gel chromatography (0-10% MeOH in EtOAc). The desired fractions were combined and concentrated. The residue was dissolved in 1 mL of DCM, the formed precipitate was filtered and rinsed with 1 mL of DCM. The precipitate was dried under vacuum overnight to afford the title compound (21.8 mg, 17%) as a white solid. ES-MS m/z 634 (M+H).

The compound in the following table was prepared essentially as described in Example 27 using the appropriate amine and carboxylic acid.

| Example | Chemical name | Structure | ES-MS m/z |
|---|---|---|---|
| 28 | 2-(3-Oxa-7- azabicyclo[3.3.1]nonan-7-yl)-N- ((1R,2S)-2-((4-fluoro-3- (trifluoromethyl)phenyl)carbamoyl) cyclopentyl)-6- methoxybenzo[d]thiazole-7- carboxamide | 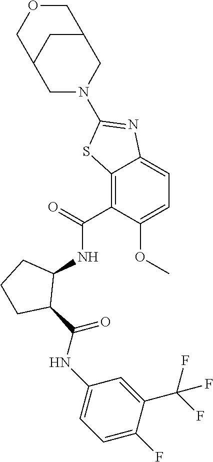 [SMILES: COc1ccc2nc(N3CC4COCC(C4)C3)sc2c1C(=O)N[C@@H]1CCC[C@@H]1C(=O)Nc1ccc(F)c(C(F)(F)F)c1] | 607 (M + H) |

### Example 29

### 2-(3-Oxa-6-azabicyclo[3.1.1]heptan-6-yl)-N-(5-fluoro-2-((3-(trifluoromethyl) bicyclo[1.1.1]pentan-1-yl)carbamoyl)phenyl)-6-methoxybenzo[d]thiazole-7-carboxamide

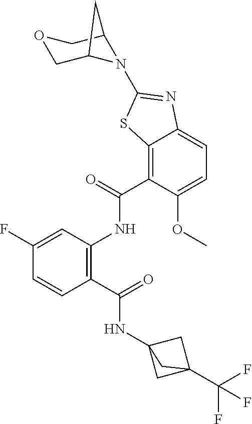 [SMILES: COc1ccc2nc(N3C4COCC3C4)sc2c1C(=O)Nc1cc(F)ccc1C(=O)NC12CC(C(F)(F)F)(C1)C2]

To 2-(2-(3-oxa-6-azabicyclo[3.1.1]heptan-6-yl)-6-methoxybenzo[d]thiazole-7-carboxamido)-4-fluorobenzoic acid (0.20 g, 0.45 mmol) was added NMP (5.0 mL), 3-(trifluoromethyl) bicyclo[1.1.1]pentan-1-amine hydrochloride (0.17 g, 0.90 mmol), T3P (0.57 g, 0.53 mL, 0.90 mmol), and DiPEA (0.17 g, 0.24 mL, 1.4 mmol). The reaction mixture was stirred at 50° C. overnight. The reaction mixture was diluted with EtOAc (100 mL) and washed with H₂O (3×50 mL) followed by saturated aqueous NaCl (50 mL). Organics were dried over Na₂SO₄, filtered and concentrated. The residue was purified by reverse phase flash chromatography using a high pH mobile phase to give the title compound (143 mg, 55%) as an off white solid. ES-MS m/z 577 (M+H).

The compounds in the following table were prepared essentially as described in Example 29 using the appropriate amine and carboxylic acid.

| Example | Chemical name | Structure | ES-MS m/z |
|---|---|---|---|
| 30 | 2-(3-Oxa-6- azabicyclo[3.1.1]heptan-6-yl)- N-(2-((2,2- difluorobenzo[d][1,3]dioxol-5- yl)carbamoyl)-5-fluorophenyl)- 6-methoxybenzo[d]thiazole-7- carboxamide | 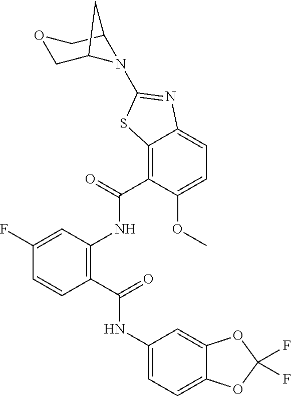 [SMILES: COc1ccc2nc(N3C4COCC3C4)sc2c1C(=O)Nc1cc(F)ccc1C(=O)Nc1ccc2c(c1)OC(F)(F)O2] | 599 (M + H) |
| 31 | 2-(3-Oxa-6- azabicyclo[3.1.1]heptan-6-yl)- N-(2-((2,2- difluorobenzo[d][1,3]dioxol-5- yl)carbamoyl)-3-fluorophenyl)- 6-methoxybenzo[d]thiazole-7- carboxamide | 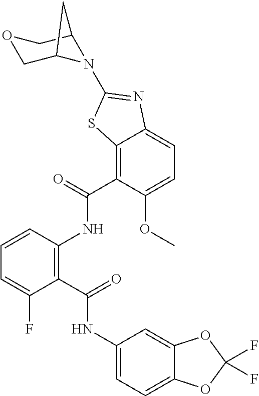 [SMILES: COc1ccc2nc(N3C4COCC3C4)sc2c1C(=O)Nc1cccc(F)c1C(=O)Nc1ccc2c(c1)OC(F)(F)O2] | 599 (M + H) |
| 32 | 2-(3-Oxa-6- azabicyclo[3.1.1]heptan-6-yl)- N-(2-((3- cyanobicyclo[1.1.1]pentan-1- yl)carbamoyl)-5-fluorophenyl)- 6-methoxybenzo[d]thiazole-7- carboxamide | 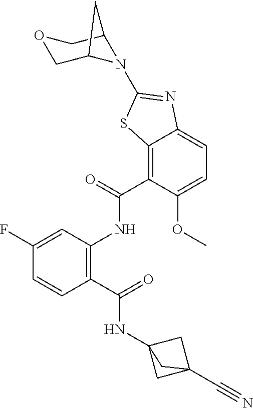 [SMILES: COc1ccc2nc(N3C4COCC3C4)sc2c1C(=O)Nc1cc(F)ccc1C(=O)NC12CC(C#N)(C1)C2] | 534 (M + H) |
| 33 | 2-(3-Oxa-6- azabicyclo[3.1.1]heptan-6-yl)-6- methoxy-N-(2-((3- (trifluoromethyl)bicyclo[1.1.1] pentan-1- yl)carbamoyl)phenyl)benzo [d]thiazole-7-carboxamide | 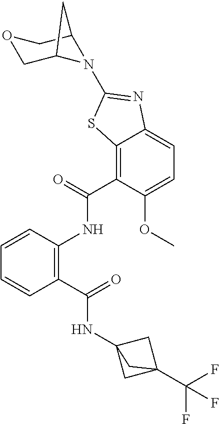 [SMILES: COc1ccc2nc(N3C4COCC3C4)sc2c1C(=O)Nc1ccccc1C(=O)NC12CC(C(F)(F)F)(C1)C2] | 559 (M + H) |
| 34 | 2-(3-Oxa-6- azabicyclo[3.1.1]heptan-6-yl)- N-(3,5-difluoro-2-((3- (trifluoromethyl)bicyclo[1.1.1] pentan-1-yl)carbamoyl)phenyl)- 6-methoxybenzo[d]thiazole-7- carboxamide | 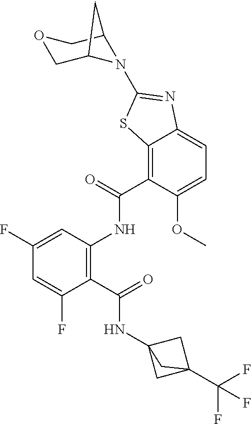 [SMILES: COc1ccc2nc(N3C4COCC3C4)sc2c1C(=O)Nc1cc(F)cc(F)c1C(=O)NC12CC(C(F)(F)F)(C1)C2] | 595 (M + H) |
| 35 | 2-(3-Oxa-6- azabicyclo[3.1.1]heptan-6-yl)- N-(5-fluoro-2-((1- (trifluoromethyl)-2- oxabicyclo[2.1.1]hexan-4- yl)carbamoyl)phenyl)-6- methoxybenzo[d]thiazole-7- carboxamide | 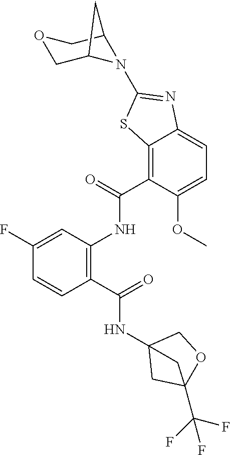 [SMILES: COc1ccc2nc(N3C4COCC3C4)sc2c1C(=O)Nc1cc(F)ccc1C(=O)NC12COC(C(F)(F)F)(C1)C2] | 593 (M + H) |
| 36 | 2-(3-Oxa-6- azabicyclo[3.1.1]heptan-6-yl)- N-(5-fluoro-2-((1- (trifluoromethyl)-2- oxabicyclo[2.2.2]octan-4- yl)carbamoyl)phenyl)-6- methoxybenzo[d]thiazole-7- carboxamide | 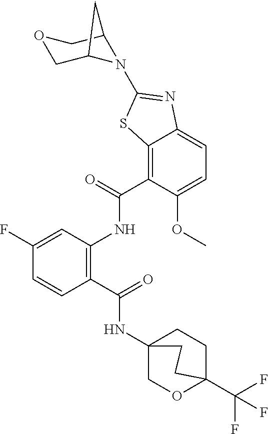 [SMILES: COc1ccc2nc(N3C4COCC3C4)sc2c1C(=O)Nc1cc(F)ccc1C(=O)NC12CCC(C(F)(F)F)(CC1)OC2] | 621 (M + H) |
| 37 | 2-(3-Oxa-6- azabicyclo[3.1.1]heptan-6-yl)-6- methoxy-N-(2-((1- (trifluoromethyl)-2- oxabicyclo[2.2.2]octan-4- yl)carbamoyl)phenyl)benzo [d]thiazole-7-carboxamide | 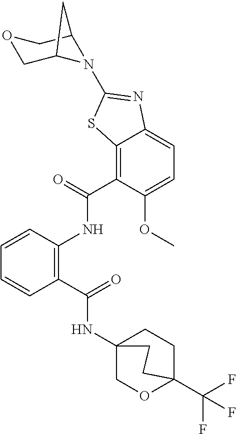 [SMILES: COc1ccc2nc(N3C4COCC3C4)sc2c1C(=O)Nc1ccccc1C(=O)NC12CCC(C(F)(F)F)(CC1)OC2] | 603 (M + H) |
| 38 | 2-(3-Oxa-6- azabicyclo[3.1.1]heptan-6-yl)- N-(5-chloro-2-((3- (trifluoromethyl)bicyclo[1.1.1] pentan-1-yl)carbamoyl)phenyl)- 6-methoxybenzo[d]thiazole-7- carboxamide | 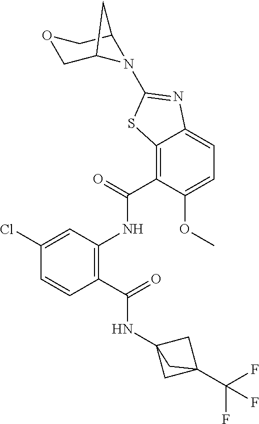 [SMILES: COc1ccc2nc(N3C4COCC3C4)sc2c1C(=O)Nc1cc(Cl)ccc1C(=O)NC12CC(C(F)(F)F)(C1)C2] | 593 (M + H) |
| 39 | 2-(3-Oxa-6- azabicyclo[3.1.1]heptan-6-yl)- N-(5-cyano-2-((3- (trifluoromethyl)bicyclo[1.1.1] pentan-1-yl)carbamoyl)phenyl)- 6-methoxybenzo[d]thiazole-7- carboxamide | 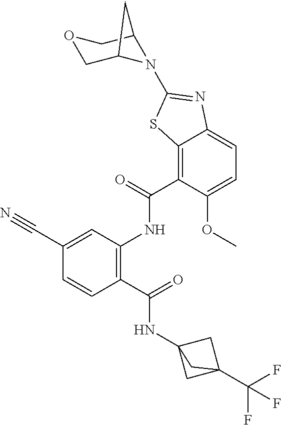 [SMILES: COc1ccc2nc(N3C4COCC3C4)sc2c1C(=O)Nc1cc(C#N)ccc1C(=O)NC12CC(C(F)(F)F)(C1)C2] | 584 (M + H) |
| 40 | 2-(3-Oxa-6- azabicyclo[3.1.1]heptan-6-yl)-6- methoxy-N-(4-(trifluoromethyl)- 2-((3- (trifluoromethyl)bicyclo[1.1.1] pentan-1- yl)carbamoyl)phenyl)benzo [d]thiazole-7-carboxamide | 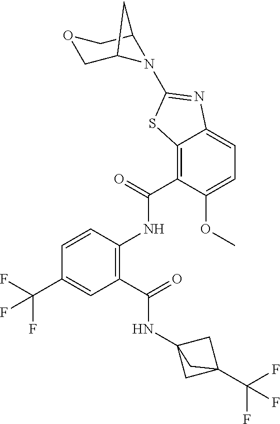 [SMILES: COc1ccc2nc(N3C4COCC3C4)sc2c1C(=O)Nc1ccc(C(F)(F)F)cc1C(=O)NC12CC(C(F)(F)F)(C1)C2] | 627 (M + H) |
| 41 | 2-(3-Oxa-6- azabicyclo[3.1.1]heptan-6-yl)-6- methoxy-N-(6-methyl-4-((3- (trifluoromethyl)bicyclo[1.1.1] pentan-1-yl)carbamoyl)pyridin-3- yl)benzo[d]thiazole-7- carboxamide | 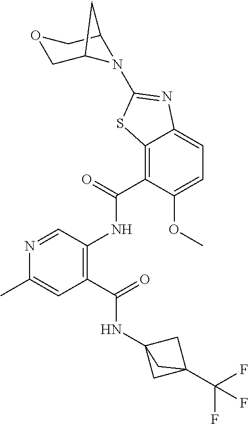 [SMILES: COc1ccc2nc(N3C4COCC3C4)sc2c1C(=O)Nc1cnc(C)cc1C(=O)NC12CC(C(F)(F)F)(C1)C2] | 574 (M + H) |
| 42 | 2-(3-Oxa-6- azabicyclo[3.1.1]heptan-6-yl)-6- methoxy-N-(5-methyl-2-((3- (trifluoromethyl)bicyclo[1.1.1] pentan-1- yl)carbamoyl)phenyl)benzo [d]thiazole-7-carboxamide | 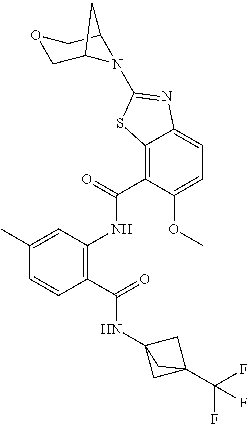 [SMILES: COc1ccc2nc(N3C4COCC3C4)sc2c1C(=O)Nc1cc(C)ccc1C(=O)NC12CC(C(F)(F)F)(C1)C2] | 573 (M + H) |
| 43 | 2-(3-Oxa-6- azabicyclo[3.1.1]heptan-6-yl)- N-(4,5-difluoro-2-((3- (trifluoromethyl)bicyclo[1.1.1] pentan-1-yl)carbamoyl)phenyl)- 6-methoxybenzo[d]thiazole-7- carboxamide | 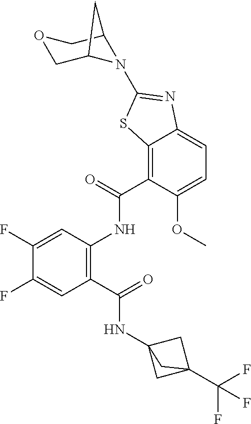 [SMILES: COc1ccc2nc(N3C4COCC3C4)sc2c1C(=O)Nc1cc(F)c(F)cc1C(=O)NC12CC(C(F)(F)F)(C1)C2] | 595 (M + H) |
| 44 | 2-(3-Oxa-6- azabicyclo[3.1.1]heptan-6-yl)- N-(4-fluoro-2-((3- (trifluoromethyl)bicyclo[1.1.1] pentan-1-yl)carbamoyl)phenyl)- 6-methoxybenzo[d]thiazole-7- carboxamide | 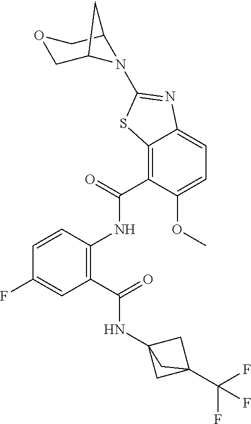 [SMILES: COc1ccc2nc(N3C4COCC3C4)sc2c1C(=O)Nc1ccc(F)cc1C(=O)NC12CC(C(F)(F)F)(C1)C2] | 577 (M + H) |
| 45 | 2-(3-Oxa-6- azabicyclo[3.1.1]heptan-6-yl)-6- methoxy-N-(5-methoxy-2-((3- (trifluoromethyl)bicyclo[1.1.1] pentan-1- yl)carbamoyl)phenyl)benzo [d]thiazole-7-carboxamide | 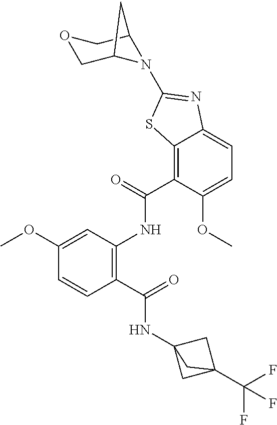 [SMILES: COc1ccc(C(=O)NC23CC(C(F)(F)F)(C2)C3)c(NC(=O)c2c(OC)ccc3nc(N4C5COCC4C5)sc23)c1] | 589 (M + H) |
| 46 | 2-(3-Oxa-6- azabicyclo[3.1.1]heptan-6-yl)-6- methoxy-N-(4-methyl-2-((3- (trifluoromethyl)bicyclo[1.1.1] pentan-1- yl)carbamoyl)phenyl)benzo [d]thiazole-7-carboxamide | 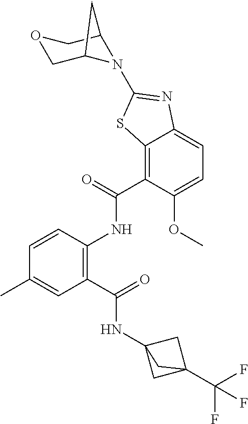 [SMILES: COc1ccc2nc(N3C4COCC3C4)sc2c1C(=O)Nc1ccc(C)cc1C(=O)NC12CC(C(F)(F)F)(C1)C2] | 573 (M + H) |
| 47 | 2-(3-Oxa-6- azabicyclo[3.1.1]heptan-6-yl)-6- methoxy-N-(5-(trifluoromethyl)- 2-((3- (trifluoromethyl)bicyclo[1.1.1] pentan-1- yl)carbamoyl)phenyl)benzo [d]thiazole-7-carboxamide | 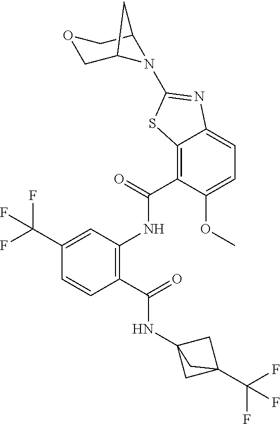 [SMILES: COc1ccc2nc(N3C4COCC3C4)sc2c1C(=O)Nc1cc(C(F)(F)F)ccc1C(=O)NC12CC(C(F)(F)F)(C1)C2] | 627 (M + H) |
| 48 | 2-(3-Oxa-6- azabicyclo[3.1.1]heptan-6-yl)- N-(5-(difluoromethoxy)-2-((3- (trifluoromethyl)bicyclo[1.1.1] pentan-1-yl)carbamoyl)phenyl)- 6-methoxybenzo[d]thiazole-7- carboxamide | 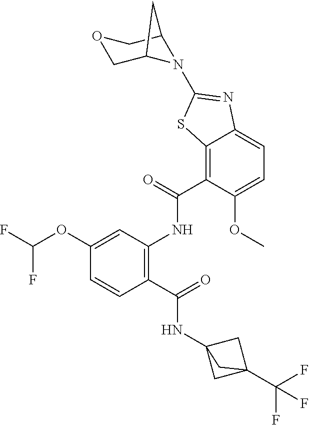 [SMILES: COc1ccc2nc(N3C4COCC3C4)sc2c1C(=O)Nc1cc(OC(F)F)ccc1C(=O)NC12CC(C(F)(F)F)(C1)C2] | 625 (M + H) |
| 49 | 2-(3-Oxa-6- azabicyclo[3.1.1]heptan-6-yl)-6- methoxy-N-(5-methoxy-2-((1- (trifluoromethyl)-2- oxabicyclo[2.2.2]octan-4- yl)carbamoyl)phenyl)benzo [d]thiazole-7-carboxamide | 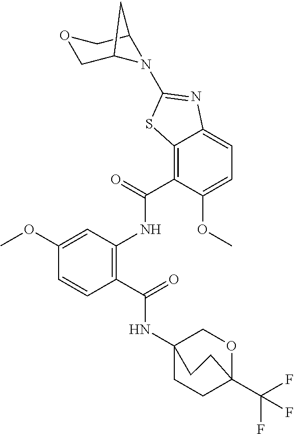 [SMILES: COc1ccc(C(=O)NC23CCC(C(F)(F)F)(CC2)OC3)c(NC(=O)c2c(OC)ccc3nc(N4C5COCC4C5)sc23)c1] | 633 (M + H) |
| 50 | 2-(3-Oxa-6- azabicyclo[3.1.1]heptan-6-yl)-6- methoxy-N-(4-methyl-2-((1- (trifluoromethyl)-2- oxabicyclo[2.2.2]octan-4- yl)carbamoyl)phenyl)benzo [d]thiazole-7-carboxamide | 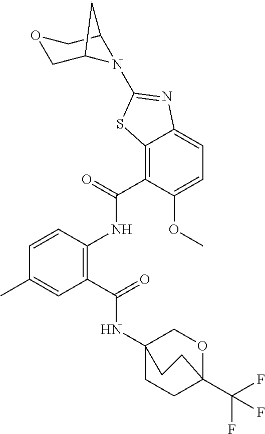 [SMILES: COc1ccc2nc(N3C4COCC3C4)sc2c1C(=O)Nc1ccc(C)cc1C(=O)NC12CCC(C(F)(F)F)(CC1)OC2] | 617 (M + H) |
| 51 | 2-(3-Oxa-6- azabicyclo[3.1.1]heptan-6-yl)- N-(4-chloro-2-((3- (trifluoromethyl)bicyclo[1.1.1] pentan-1-yl)carbamoyl)phenyl)- 6-methoxybenzo[d]thiazole-7- carboxamide | 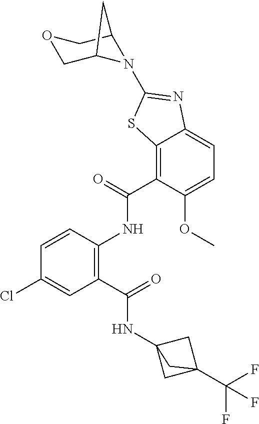 [SMILES: COc1ccc2nc(N3C4COCC3C4)sc2c1C(=O)Nc1ccc(Cl)cc1C(=O)NC12CC(C(F)(F)F)(C1)C2] | 593 (M + H) |
| 52 | 2-(3-Oxa-6- azabicyclo[3.1.1]heptan-6-yl)-6- methoxy-N-(4-methoxy-2-((3- (trifluoromethyl)bicyclo[1.1.1] pentan-1- yl)carbamoyl)phenyl)benzo [d]thiazole-7-carboxamide | 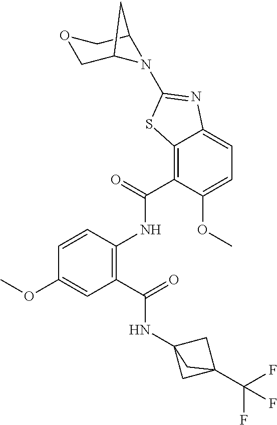 [SMILES: COc1ccc(NC(=O)c2c(OC)ccc3nc(N4C5COCC4C5)sc23)c(C(=O)NC23CC(C(F)(F)F)(C2)C3)c1] | 589 (M + H) |
| 53 | 2-(3-Oxa-6- azabicyclo[3.1.1]heptan-6-yl)- N-(5-fluoro-4-methyl-2-((3- (trifluoromethyl)bicyclo[1.1.1] pentan-1-yl)carbamoyl)phenyl)- 6-methoxybenzo[d]thiazole-7- carboxamide | 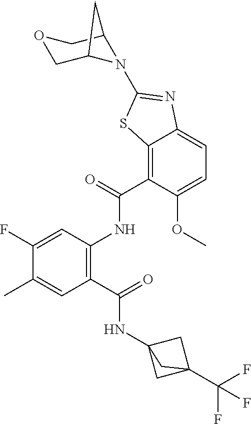 [SMILES: COc1ccc2nc(N3C4COCC3C4)sc2c1C(=O)Nc1cc(F)c(C)cc1C(=O)NC12CC(C(F)(F)F)(C1)C2] | 591 (M + H) |
| 54 | 2-(3-Oxa-6- azabicyclo[3.1.1]heptan-6-yl)-6- methoxy-N-(6-methyl-4-((1- (trifluoromethyl)-2- oxabicyclo[2.2.2]octan-4- yl)carbamoyl)pyridin-3- yl)benzo[d]thiazole-7- carboxamide | 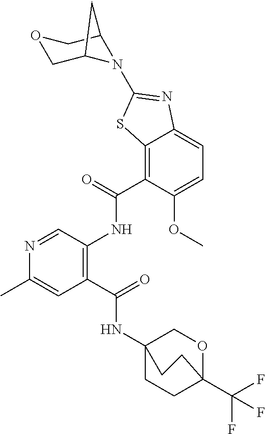 [SMILES: COc1ccc2nc(N3C4COCC3C4)sc2c1C(=O)Nc1cnc(C)cc1C(=O)NC12CCC(C(F)(F)F)(CC1)OC2] | 618 (M + H) |
| 55 | 2-(3-Oxa-6- azabicyclo[3.1.1]heptan-6-yl)- N-(2-((3- cyanobicyclo[1.1.1]pentan-1- yl)carbamoyl)-5-fluoro-4- methylphenyl)-6- methoxybenzo[d]thiazole-7- carboxamide | 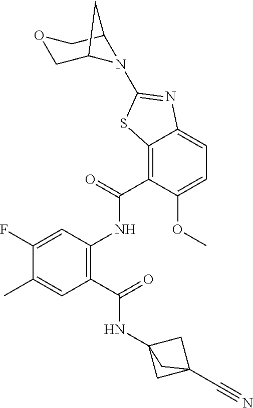 [SMILES: COc1ccc2nc(N3C4COCC3C4)sc2c1C(=O)Nc1cc(F)c(C)cc1C(=O)NC12CC(C#N)(C1)C2] | 548 (M + H) |
| 56 | 2-(3-Oxa-6- azabicyclo[3.1.1]heptan-6-yl)-6- methoxy-N-(7-((3- (trifluoromethyl)bicyclo[1.1.1] pentan-1-yl)carbamoyl)-2,3- dihydrobenzo[b][1,4]dioxin-6- yl)benzo[d]thiazole-7- carboxamide | 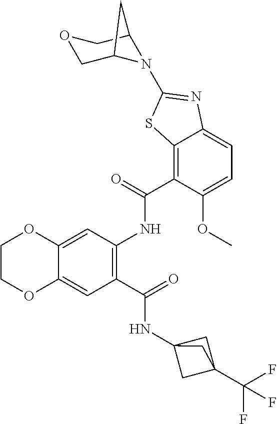 [SMILES: COc1ccc2nc(N3C4COCC3C4)sc2c1C(=O)Nc1cc2c(cc1C(=O)NC13CC(C(F)(F)F)(C1)C3)OCCO2] | 617 (M + H) |
| 57 | 2-(3-Oxa-6- azabicyclo[3.1.1]heptan-6-yl)-6- methoxy-N-(6-((3- (trifluoromethyl)bicyclo[1.1.1] pentan-1- yl)carbamoyl)benzo[d][1,3] dioxol-5-yl)benzo[d]thiazole-7- carboxamide | 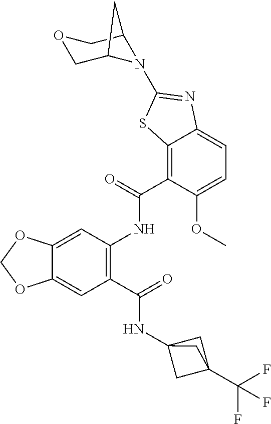 [SMILES: COc1ccc2nc(N3C4COCC3C4)sc2c1C(=O)Nc1cc2c(cc1C(=O)NC13CC(C(F)(F)F)(C1)C3)OCO2] | 603 (M + H) |
| 58 | 2-(3-Oxa-6- azabicyclo[3.1.1]heptan-6-yl)-6- methoxy-N-(7-((1- (trifluoromethyl)-2- oxabicyclo[2.2.2]octan-4- yl)carbamoyl)-2,3- dihydrobenzo[b][1,4]dioxin-6- yl)benzo[d]thiazole-7- carboxamide | 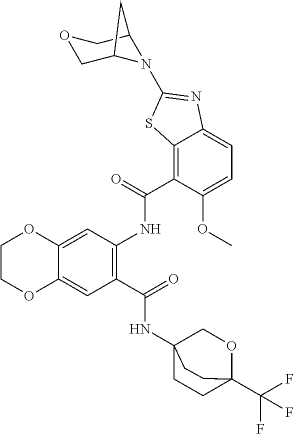 [SMILES: COc1ccc2nc(N3C4COCC3C4)sc2c1C(=O)Nc1cc2c(cc1C(=O)NC13CCC(C(F)(F)F)(CC1)OC3)OCCO2] | 661 (M + H) |
| 59 | 2-(3-Oxa-6- azabicyclo[3.1.1]heptan-6-yl)-6- methoxy-N-(6-((1- (trifluoromethyl)-2- oxabicyclo[2.2.2]octan-4- yl)carbamoyl)benzo[d][1,3] dioxol-5-yl)benzo[d]thiazole-7- carboxamide | 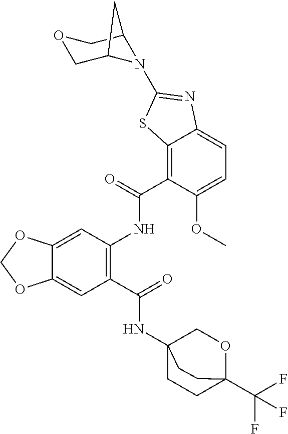 [SMILES: COc1ccc2nc(N3C4COCC3C4)sc2c1C(=O)Nc1cc2c(cc1C(=O)NC13CCC(C(F)(F)F)(CC1)OC3)OCO2] | 647 (M + H) |
| 60 | 2-(3-Oxa-6- azabicyclo[3.1.1]heptan-6-yl)-6- methoxy-N-(6-((1- (trifluoromethyl)-2- oxabicyclo[2.1.1]hexan-4- yl)carbamoyl)benzo[d][1,3] dioxol-5-yl)benzo[d]thiazole-7- carboxamide | 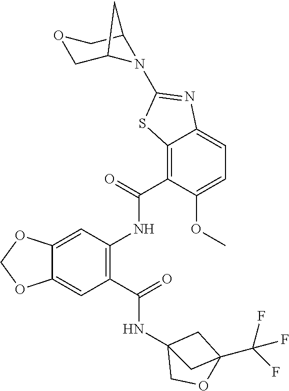 [SMILES: COc1ccc2nc(N3C4COCC3C4)sc2c1C(=O)Nc1cc2c(cc1C(=O)NC13COC(C(F)(F)F)(C1)C3)OCO2] | 619 (M + H) |
| 61 | 2-(3-Oxa-6- azabicyclo[3.1.1]heptan-6-yl)-N- (6-((3- cyanobicyclo[1.1.1]pentan-1- yl)carbamoyl)benzo[d][1,3] dioxol-5-yl)-6- methoxybenzo[d]thiazole-7- carboxamide | 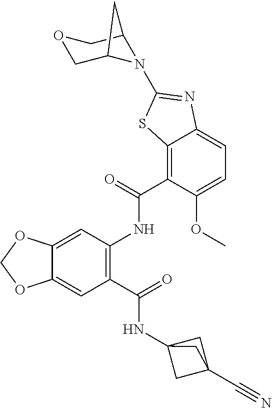 [SMILES: COc1ccc2nc(N3C4COCC3C4)sc2c1C(=O)Nc1cc2c(cc1C(=O)NC13CC(C#N)(C1)C3)OCO2] | 560 (M + H) |
| 62 | N-(6-(Benzo[d][1,3]dioxol-5- ylcarbamoyl)benzo[d][1,3] dioxol-5-yl)-2-(3-oxa-6- azabicyclo[3.1.1]heptan-6-yl)-6- methoxybenzo[d]thiazole-7- carboxamide | 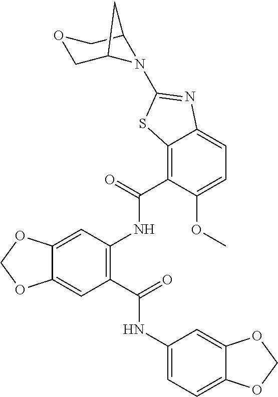 [SMILES: COc1ccc2nc(N3C4COCC3C4)sc2c1C(=O)Nc1cc2c(cc1C(=O)Nc1ccc3c(c1)OCO3)OCO2] | 589 (M + H) |

### Example 63

### N-((1S,2R,3S,4R)-3-((4-Fluoro-3-(trifluoromethyl)phenyl)carbamoyl)bicyclo[2.2.1]heptan-2-yl)-2-((1R,3S,5S)-3-hydroxy-6-azabicyclo[3.1.1]heptan-6-yl)-6-methoxybenzo[d]thiazole-7-carboxamide

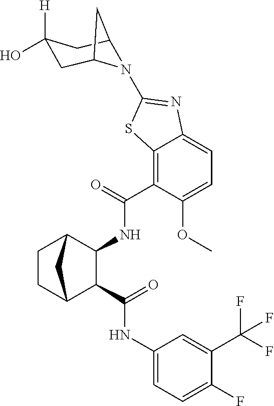 [SMILES: COc1ccc2nc(N3C4CC(O)CC3C4)sc2c1C(=O)N[C@@H]1[C@H]2CC[C@H](C2)[C@@H]1C(=O)Nc1ccc(F)c(C(F)(F)F)c1]

A solution of (1R,3S,5S)-6-azabicyclo[3.1.1]heptan-3-ol (41 mg, 0.36 mmol), 2-chloro-N-((1S,2R,3S,4R)-3-((4-fluoro-3-(trifluoromethyl)phenyl)carbamoyl)bicyclo[2.2.1]heptan-2-yl)-6-methoxybenzo[d]thiazole-7-carboxamide (65 mg, 0.12 mmol), and K₂CO₃ (50 mg, 0.36 mmol) in NMP (1 mL) was stirred at 80° C. for 18 h. After 18 h, the reaction mixture was purified directly by reverse phase column chromatography using a gradient of 0 to 100% (ACN+5% MeOH) in 10 mM aq. NH₄HCO₃ to provide the title compound (38 mg, 51%). ES-MS m/z 619 (M+H).

The compounds in the following table were prepared essentially as described in Example 63 using the appropriate amine.

| Example | Chemical name | Structure | ES-MS m/z |
|---|---|---|---|
| 64 | N-((1S,2R,3S,4R)-3-((4-Fluoro- 3- (trifluoromethyl)phenyl)carbamoyl) bicyclo[2.2.1]heptan-2-yl)-6- methoxy-2-(3-methyl-3,6- diazabicyclo[3.1.1]heptan-6- yl)benzo[d]thiazole-7- carboxamide | 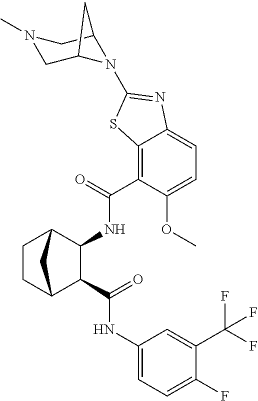 [SMILES: COc1ccc2nc(N3C4CC3CN(C)C4)sc2c1C(=O)N[C@@H]1[C@H]2CC[C@H](C2)[C@@H]1C(=O)Nc1ccc(F)c(C(F)(F)F)c1] | 618 (M + H) |
| 65 | 2-((1S,5S)-3,7-Dioxa-9- azabicyclo[3.1.1]nonan-9-yl)-N- ((1S,2R,3S,4R)-3-((4-fluoro-3- (trifluoromethyl)phenyl) carbamoyl)bicyclo[2.2.1]heptan- 2-yl)-6-methoxybenzo[d]thiazole-7- carboxamide | 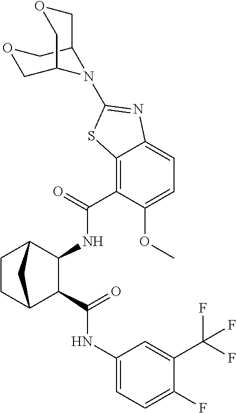 [SMILES: COc1ccc2nc(N3C4COCC3COC4)sc2c1C(=O)N[C@@H]1[C@H]2CC[C@H](C2)[C@@H]1C(=O)Nc1ccc(F)c(C(F)(F)F)c1] | 635 (M + H) |
| 66 | 2-(3-Thia-6- azabicyclo[3.1.1]heptan-6-yl)-N- ((1S,2R,3S,4R)-3-((4-fluoro-3- (trifluoromethyl)phenyl)carbamoyl) bicyclo[2.2.1]heptan-2-yl)-6- methoxybenzo[d]thiazole-7- carboxamide | 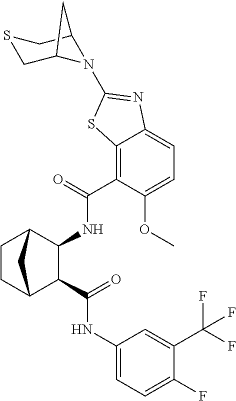 [SMILES: COc1ccc2nc(N3C4CSCC3C4)sc2c1C(=O)N[C@@H]1[C@H]2CC[C@H](C2)[C@@H]1C(=O)Nc1ccc(F)c(C(F)(F)F)c1] | 621 (M + H) |
| 67 | 2-(3-Oxa-6- azabicyclo[3.1.1]heptan-6-yl)-N- ((1R,2S)-2-((2,2- difluorobenzo[d][1,3]dioxol-5- yl)carbamoyl)cyclobutyl)-6- methoxybenzo[d]thiazole-7- carboxamide | 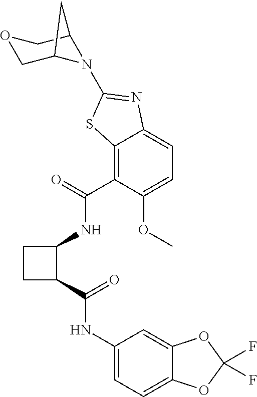 [SMILES: COc1ccc2nc(N3C4COCC3C4)sc2c1C(=O)N[C@@H]1CC[C@@H]1C(=O)Nc1ccc2c(c1)OC(F)(F)O2] | 559 (M + H) |

### Example 68

### 2-(3-Oxa-6-azabicyclo[3.1.1]heptan-6-yl)-N-(2-((3-cyanobicyclo[1.1.1]pentan-1-yl)carbamoyl)-5-(difluoromethoxy)phenyl)-6-methoxybenzo[d]thiazole-7-carboxamide

To a solution of 2-(2-(3-oxa-6-azabicyclo[3.1.1]heptan-6-yl)-6-methoxybenzo[d]thiazole-7-carboxamido)-4-(difluoromethoxy)benzoic acid (50 mg, 0.10 mmol) and 3-aminobicyclo[1.1.1]pentane-1-carbonitrile (22 mg, 0.20 mmol) in NMP (1 mL) was added PPACA (50% in EtOAc) (0.19 g, 0.31 mmol, 0.18 mL), followed by DiPEA (53 mg, 0.41 mmol, 70 μL). The reaction mixture was stirred at 45° C. for 3 days. The reaction mixture was diluted with EtOAc and washed with H₂O (3 times). The organic layer was dried over Na₂SO₄, filtered and concentrated. The residue was purified by reverse phase chromatography to afford the title product (14.4 mg, 24.8 μmol, 24%). ES-MS m/z 580 (M−H)

The compounds in the following table were prepared essentially as described in Example 68 using the appropriate amine and carboxylic acid.

| Example | Chemical name | Structure | ES-MS m/z |
|---|---|---|---|
| 69 | 2-(3-Oxa-6- azabicyclo[3.1.1]heptan-6-yl)-N- (4-((3- cyanobicyclo[1.1.1]pentan-1- yl)carbamoyl)-6- methoxypyridin-3-yl)-6- methoxybenzo[d]thiazole-7- carboxamide | 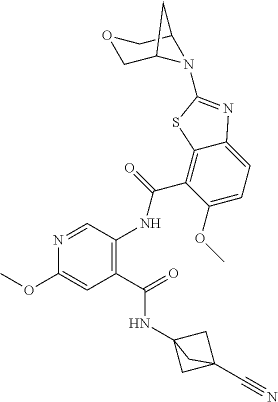 [SMILES: COc1cc(C(=O)NC23CC(C#N)(C2)C3)c(NC(=O)c2c(OC)ccc3nc(N4C5COCC4C5)sc23)cn1] | 547 (M + H) |
| 70 | 2-(3-Oxa-6- azabicyclo[3.1.1]heptan-6-yl)-6- methoxy-N-(6-methoxy-4-((1- (trifluoromethyl)-2- oxabicyclo[2.2.2]octan-4- yl)carbamoyl)pyridin-3- yl)benzo[d]thiazole-7- carboxamide | 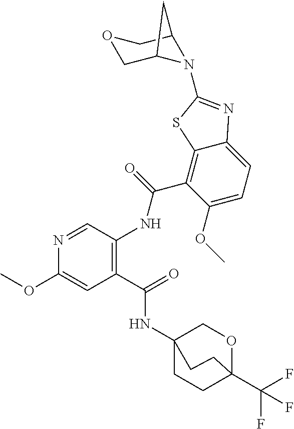 [SMILES: COc1cc(C(=O)NC23CCC(C(F)(F)F)(CC2)OC3)c(NC(=O)c2c(OC)ccc3nc(N4C5COCC4C5)sc23)cn1] | 634 (M + H) |
| 71 | 2-(3-Oxa-6- azabicyclo[3.1.1]heptan-6-yl)-N- (2-((3- cyanobicyclo[1.1.1]pentan-1- yl)carbamoyl)-5- methoxyphenyl)-6- methoxybenzo[d]thiazole-7- carboxamide | 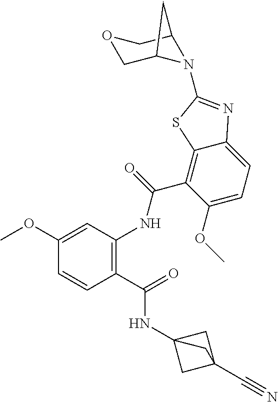 [SMILES: COc1ccc(C(=O)NC23CC(C#N)(C2)C3)c(NC(=O)c2c(OC)ccc3nc(N4C5COCC4C5)sc23)c1] | 546 (M − H) |
| 72 | 2-(3-Oxa-6- azabicyclo[3.1.1]heptan-6-yl)-N- (5-(difluoromethoxy)-2-((1- (trifluoromethyl)-2- oxabicyclo[2.2.2]octan-4- yl)carbamoyl)phenyl)-6- methoxybenzo[d]thiazole-7- carboxamide | 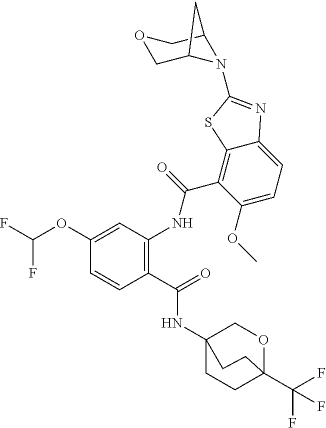 [SMILES: COc1ccc2nc(N3C4COCC3C4)sc2c1C(=O)Nc1cc(OC(F)F)ccc1C(=O)NC12CCC(C(F)(F)F)(CC1)OC2] | 669 (M + H) |
| 73 | 2-(3-Oxa-6- azabicyclo[3.1.1]heptan-6-yl)-N- (2-((3- cyanobicyclo[1.1.1]pentan-1- yl)carbamoyl)-3-fluoro-5- methoxyphenyl)-6- methoxybenzo[d]thiazole-7- carboxamide | 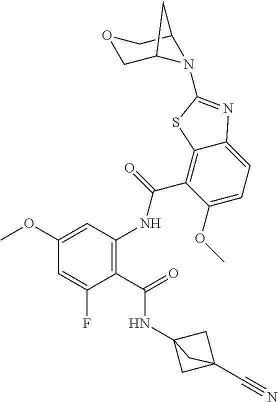 [SMILES: COc1cc(F)c(C(=O)NC23CC(C#N)(C2)C3)c(NC(=O)c2c(OC)ccc3nc(N4C5COCC4C5)sc23)c1] | 564 (M + H) |
| 74 | 2-(3-Oxa-6- azabicyclo[3.1.1]heptan-6-yl)-N- (3-fluoro-5-methoxy-2-((1- (trifluoromethyl)-2- oxabicyclo[2.2.2]octan-4- yl)carbamoyl)phenyl)-6- methoxybenzo[d]thiazole-7- carboxamide | 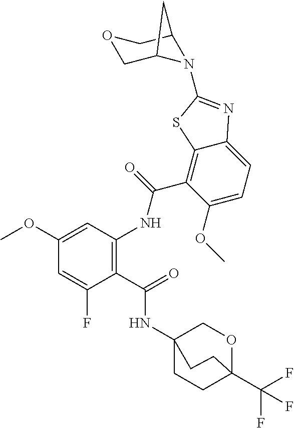 [SMILES: COc1cc(F)c(C(=O)NC23CCC(C(F)(F)F)(CC2)OC3)c(NC(=O)c2c(OC)ccc3nc(N4C5COCC4C5)sc23)c1] | 651 (M + H) |
| 75 | 2-(3-Oxa-6- azabicyclo[3.1.1]heptan-6-yl)-N- (2-((3- cyanobicyclo[1.1.1]pentan-1- yl)carbamoyl)-3,5- difluorophenyl)-6- methoxybenzo[d]thiazole-7- carboxamide | 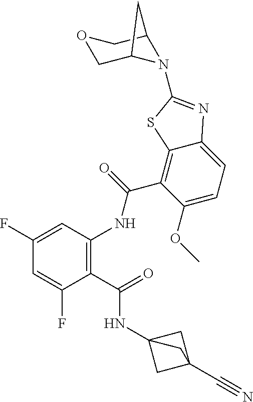 [SMILES: COc1ccc2nc(N3C4COCC3C4)sc2c1C(=O)Nc1cc(F)cc(F)c1C(=O)NC12CC(C#N)(C1)C2] | 552 (M − H) |
| 76 | 2-(3-Oxa-6- azabicyclo[3.1.1]heptan-6-yl)-N- (2-((2,2- difluorobenzo[d][1,3]dioxol-5- yl)carbamoyl)-4- (difluoromethoxy)phenyl)-6- methoxybenzo[d]thiazole-7- carboxamide | 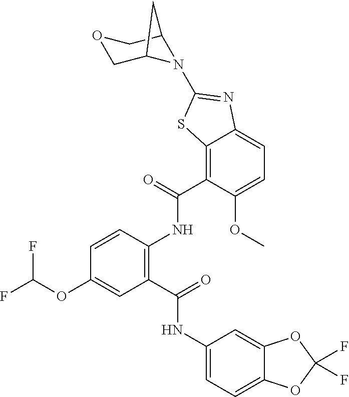 [SMILES: COc1ccc2nc(N3C4COCC3C4)sc2c1C(=O)Nc1ccc(OC(F)F)cc1C(=O)Nc1ccc2c(c1)OC(F)(F)O2] | 647 (M + H) |
| 77 | 2-(3-Oxa-6- azabicyclo[3.1.1]heptan-6-yl)-N- (4-((2,2- difluorobenzo[d][1,3]dioxol-5- yl)carbamoyl)-6- methoxypyridin-3-yl)-6- methoxybenzo[d]thiazole-7- carboxamide | 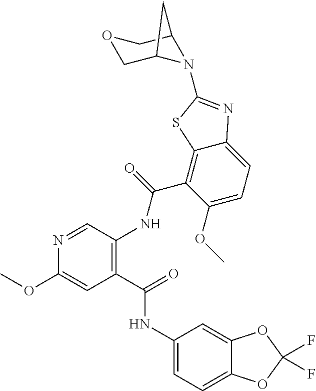 [SMILES: COc1cc(C(=O)Nc2ccc3c(c2)OC(F)(F)O3)c(NC(=O)c2c(OC)ccc3nc(N4C5COCC4C5)sc23)cn1] | 612 (M + H) |
| 78 | 2-(3-Oxa-6- azabicyclo[3.1.1]heptan-6-yl)-6- methoxy-N-(6-methoxy-4-((3- (trifluoromethyl)bicyclo[1.1.1] pentan-1-yl)carbamoyl)pyridin-3- yl)benzo[d]thiazole-7- carboxamide | 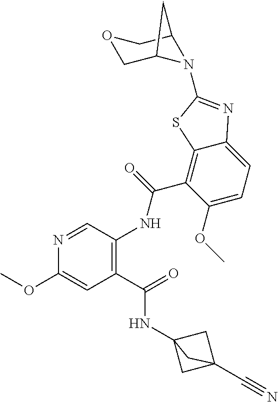 | 590 (M + H) |
| 79 | 2-(3-Oxa-6- azabicyclo[3.1.1]heptan-6-yl)-N- (3-fluoro-5-methoxy-2-((1- (trifluoromethyl)-2- oxabicyclo[2.1.1]hexan-4- yl)carbamoyl)phenyl)-6- methoxybenzo[d]thiazole-7- carboxamide | 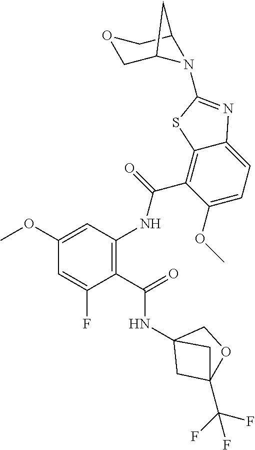 [SMILES: COc1cc(F)c(C(=O)NC23COC(C(F)(F)F)(C2)C3)c(NC(=O)c2c(OC)ccc3nc(N4C5COCC4C5)sc23)c1] | 623 (M + H) |
| 80 | 2-(3-Oxa-6- azabicyclo[3.1.1]heptan-6-yl)-6- methoxy-N-(6-methoxy-4-((1- (trifluoromethyl)-2- oxabicyclo[2.1.1]hexan-4- yl)carbamoyl)pyridin-3- yl)benzo[d]thiazole-7- carboxamide | 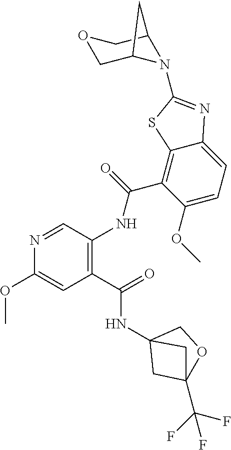 [SMILES: COc1cc(C(=O)NC23COC(C(F)(F)F)(C2)C3)c(NC(=O)c2c(OC)ccc3nc(N4C5COCC4C5)sc23)cn1] | 606 (M + H) |
| 81 | 2-(3-Oxa-6- azabicyclo[3.1.1]heptan-6-yl)-N- (4-((3- cyanobicyclo[1.1.1]pentan-1- yl)carbamoyl)-5-fluoro-6- methoxypyridin-3-yl)-6- methoxybenzo[d]thiazole-7- carboxamide | 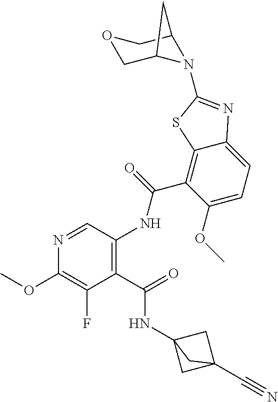 [SMILES: COc1ccc2nc(N3C4COCC3C4)sc2c1C(=O)Nc1cnc(OC)c(F)c1C(=O)NC12CC(C#N)(C1)C2] | 565 (M + H) |
| 82 | 2-(3-Oxa-6- azabicyclo[3.1.1]heptan-6-yl)-N- (5-fluoro-6-methoxy-4-((1- (trifluoromethyl)-2- oxabicyclo[2.2.2]octan-4- yl)carbamoyl)pyridin-3-yl)-6- methoxybenzo[d]thiazole-7- carboxamide | 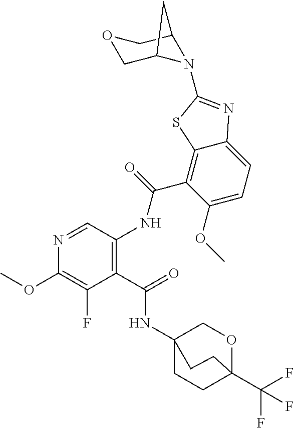 [SMILES: COc1ccc2nc(N3C4COCC3C4)sc2c1C(=O)Nc1cnc(OC)c(F)c1C(=O)NC12CCC(C(F)(F)F)(CC1)OC2] | 652 (M + H) |
| 83 | 2-(3-Oxa-6- azabicyclo[3.1.1]heptan-6-yl)-N- (4-((2,2- difluorobenzo[d][1,3]dioxol-5- yl)carbamoyl)-5-fluoro-6- methoxypyridin-3-yl)-6- methoxybenzo[d]thiazole-7- carboxamide | 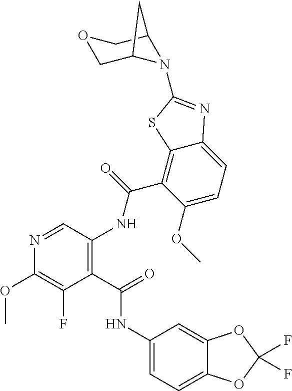 [SMILES: COc1ccc2nc(N3C4COCC3C4)sc2c1C(=O)Nc1cnc(OC)c(F)c1C(=O)Nc1ccc2c(c1)OC(F)(F)O2] | 630 (M + H) |
| 84 | 2-(3-Oxa-6- azabicyclo[3.1.1]heptan-6-yl)-N- (5-fluoro-4-methyl-2-((1- (trifluoromethyl)-2- oxabicyclo[2.1.1]hexan-4- yl)carbamoyl)phenyl)-6- methoxybenzo[d]thiazole-7- carboxamide | 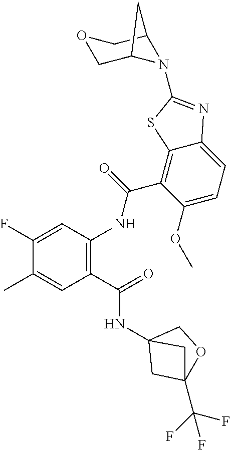 [SMILES: COc1ccc2nc(N3C4COCC3C4)sc2c1C(=O)Nc1cc(F)c(C)cc1C(=O)NC12COC(C(F)(F)F)(C1)C2] | 607 (M + H) |
| 85 | 2-(3-Oxa-6- azabicyclo[3.1.1]heptan-6-yl)-N- (2-((2,2- difluorobenzo[d][1,3]dioxol-5- yl)carbamoyl)-5- methoxyphenyl)-6- methoxybenzo[d]thiazole-7- carboxamide | 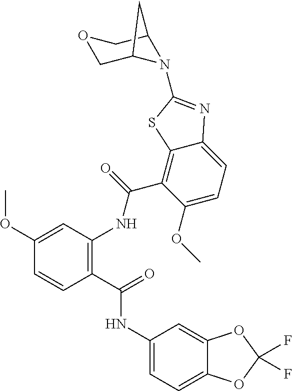 [SMILES: COc1ccc(C(=O)Nc2ccc3c(c2)OC(F)(F)O3)c(NC(=O)c2c(OC)ccc3nc(N4C5COCC4C5)sc23)c1] | 611 (M + H) |
| 86 | 2-(3-Oxa-6- azabicyclo[3.1.1]heptan-6-yl)-N- (3,5-difluoro-2-((1- (trifluoromethyl)-2- oxabicyclo[2.1.1]hexan-4- yl)carbamoyl)phenyl)-6- methoxybenzo[d]thiazole-7- carboxamide | 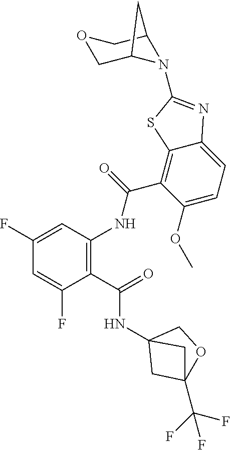 [SMILES: COc1ccc2nc(N3C4COCC3C4)sc2c1C(=O)Nc1cc(F)cc(F)c1C(=O)NC12COC(C(F)(F)F)(C1)C2] | 611 (M + H) |
| 87 | 2-(3-Oxa-6- azabicyclo[3.1.1]heptan-6-yl)-N- (4-((4-cyanobicyclo[2.2.2]octan- 1-yl)carbamoyl)-6- methoxypyridin-3-yl)-6- methoxybenzo[d]thiazole-7- carboxamide | 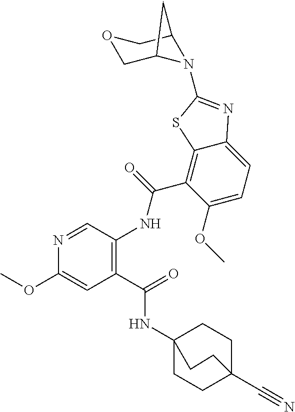 [SMILES: COc1cc(C(=O)NC23CCC(C#N)(CC2)CC3)c(NC(=O)c2c(OC)ccc3nc(N4C5COCC4C5)sc23)cn1] | 589 (M + H) |
| 88 | 2-(3-Oxa-6- azabicyclo[3.1.1]heptan-6-yl)-N- (4-((3- fluorobicyclo[1.1.1]pentan-1- yl)carbamoyl)-6- methoxypyridin-3-yl)-6- methoxybenzo[d]thiazole-7- carboxamide | 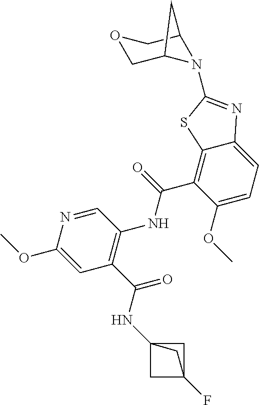 [SMILES: COc1cc(C(=O)NC23CC(F)(C2)C3)c(NC(=O)c2c(OC)ccc3nc(N4C5COCC4C5)sc23)cn1] | 540 (M + H) |
| 89 | 2-(3-Oxa-6- azabicyclo[3.1.1]heptan-6-yl)-N- (2-((3- cyanobicyclo[1.1.1]pentan-1- yl)carbamoyl)-4,5- difluorophenyl)-6- methoxybenzo[d]thiazole-7- carboxamide | 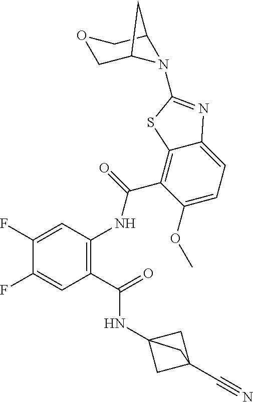 [SMILES: COc1ccc2nc(N3C4COCC3C4)sc2c1C(=O)Nc1cc(F)c(F)cc1C(=O)NC12CC(C#N)(C1)C2] | 552 (M + H) |

### Example 90

### 2-(3-Oxa-6-azabicyclo[3.1.1]heptan-6-yl)-N-(4-((4-cyanophenyl)carbamoyl)-6-methoxypyridin-3-yl)-6-methoxybenzo[d]thiazole-7-carboxamide

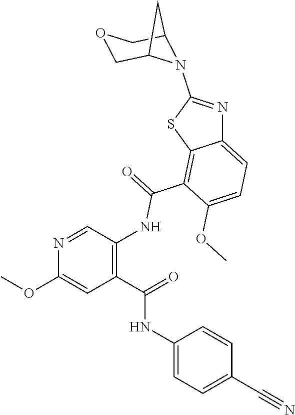 [SMILES: COc1cc(C(=O)Nc2ccc(C#N)cc2)c(NC(=O)c2c(OC)ccc3nc(N4C5COCC4C5)sc23)cn1]

To a solution of 5-(2-(3-oxa-6-azabicyclo[3.1.1]heptan-6-yl)-6-methoxybenzo[d]thiazole-7-carboxamido)-2-methoxyisonicotinic acid (100 mg, 219 μmol) in NMP (5 mL) were added 4-aminobenzonitrile (51.8 mg, 438 μmol), DMAP (40.1 mg, 329 μmol), PPACA (418 mg, 657 μmol, 387 μL) and DiPEA (113 mg, 876 μmol, 153 μL). The reaction mixture was stirred at 85° C. for two days. The residue was purified by high pH flash chromatography (C18 column). The desired fractions were combined, concentrated, and dried under reduced pressure to afford the title product (54 mg, 97 μmol, 44%) as a light tan solid. ES-MS m/z 557 (M+H).

The compounds in the following table were prepared essentially as described in Example 90 using the appropriate amine and carboxylic acid.

| Example | Chemical name | Structure | ES-MS m/z |
|---|---|---|---|
| 91 | 2-(3-Oxa-6- azabicyclo[3.1.1]heptan-6-yl)-N- (4-((4-fluoro-3- (trifluoromethyl)phenyl) carbamoyl)-6-methoxypyridin-3- yl)-6-methoxybenzo[d]thiazole-7- carboxamide | 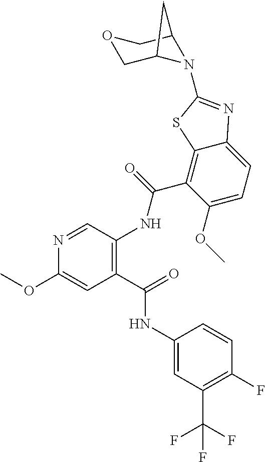 [SMILES: COc1cc(C(=O)Nc2ccc(F)c(C(F)(F)F)c2)c(NC(=O)c2c(OC)ccc3nc(N4C5COCC4C5)sc23)cn1] | 618 (M + H) |
| 92 | 2-(3-Oxa-6- azabicyclo[3.1.1]heptan-6-yl)-N- (4-((4-fluorophenyl)carbamoyl)- 6-methoxypyridin-3-yl)-6- methoxybenzo[d]thiazole-7- carboxamide | 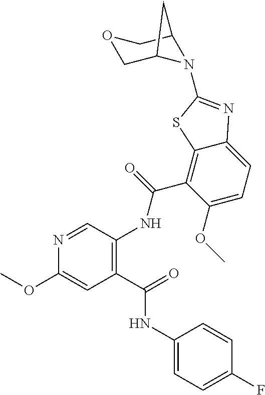 [SMILES: COc1cc(C(=O)Nc2ccc(F)cc2)c(NC(=O)c2c(OC)ccc3nc(N4C5COCC4C5)sc23)cn1] | 550 (M + H) |
| 93 | 2-(3-Oxa-6- azabicyclo[3.1.1]heptan-6-yl)-N- (4-((3-cyanophenyl)carbamoyl)- 6-methoxypyridin-3-yl)-6- methoxybenzo[d]thiazole-7- carboxamide | 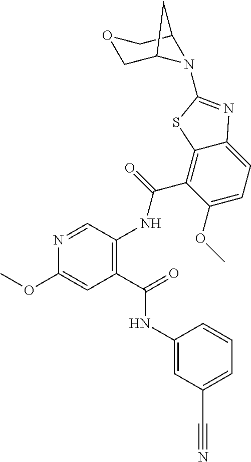 [SMILES: COc1cc(C(=O)Nc2cccc(C#N)c2)c(NC(=O)c2c(OC)ccc3nc(N4C5COCC4C5)sc23)cn1] | 557 (M + H) |
| 94 | 2-(3-Oxa-6- azabicyclo[3.1.1]heptan-6-yl)-N- (4-((2,2- difluorobenzo[d][1,3]dioxol-5- yl)carbamoyl)-6- methoxypyridin-3- yl)thiazolo[5,4-c]pyridine-4- carboxamide | 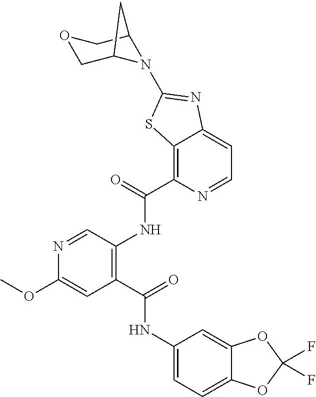 [SMILES: COc1cc(C(=O)Nc2ccc3c(c2)OC(F)(F)O3)c(NC(=O)c2nccc3nc(N4C5COCC4C5)sc23)cn1] | 583 (M + H) |
| 95 | 2-(3-Oxa-6- azabicyclo[3.1.1]heptan-6-yl)-N- (4-((2,2- difluorobenzo[d][1,3]dioxol-5- yl)carbamoyl)-6- methoxypyridin-3-yl)-6- ethoxybenzo[d]thiazole-7- carboxamide | 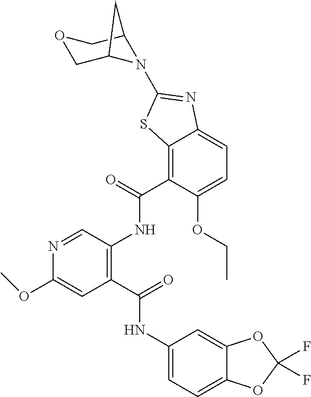 [SMILES: CCOc1ccc2nc(N3C4COCC3C4)sc2c1C(=O)Nc1cnc(OC)cc1C(=O)Nc1ccc2c(c1)OC(F)(F)O2] | 626 (M + H) |
| 96 | 2-(3-Oxa-6- azabicyclo[3.1.1]heptan-6-yl)-N- (4-((3,4- difluorophenyl)carbamoyl)-6- methoxypyridin-3-yl)-6- methoxybenzo[d]thiazole-7- carboxamide | 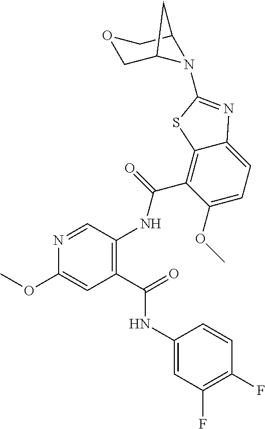 [SMILES: COc1cc(C(=O)Nc2ccc(F)c(F)c2)c(NC(=O)c2c(OC)ccc3nc(N4C5COCC4C5)sc23)cn1] | 568 (M + H) |
| 97 | 2-(3-Oxo-6- azabicyclo[3.1.1]heptan-6-yl)-6- methoxy-N-(6-methoxy-4-((3- ((trifluoromethyl)sulfonyl)phenyl) carbamoyl)pyridin-3- yl)benzo[d]thiazole-7- carboxamide | 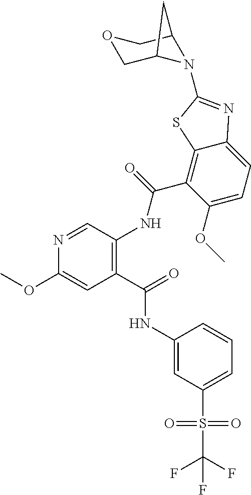 [SMILES: COc1cc(C(=O)Nc2cccc(S(=O)(=O)C(F)(F)F)c2)c(NC(=O)c2c(OC)ccc3nc(N4C5COCC4C5)sc23)cn1] | 664 (M + H) |
| 98 | 2-(3-Oxa-6- azabicyclo[3.1.1]heptan-6-yl)-N- (4-((3-cyano-4- fluorophenyl)carbamoyl)-6- methoxypyridin-3-yl)-6- methoxybenzo[d]thiazole-7- carboxamide | 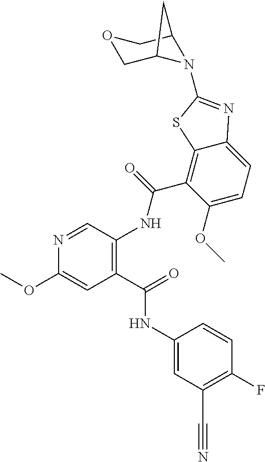 [SMILES: COc1cc(C(=O)Nc2ccc(F)c(C#N)c2)c(NC(=O)c2c(OC)ccc3nc(N4C5COCC4C5)sc23)cn1] | 575 (M + H) |
| 99 | 2-(3-Oxa-6- azabicyclo[3.1.1]heptan-6-yl)-N- (4-((4-cyano-3- fluorophenyl)carbamoyl)-6- methoxypyridin-3-yl)-6- methoxybenzo[d]thiazole-7- carboxamide | 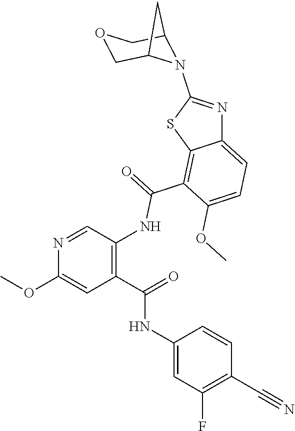 [SMILES: COc1cc(C(=O)Nc2ccc(C#N)c(F)c2)c(NC(=O)c2c(OC)ccc3nc(N4C5COCC4C5)sc23)cn1] | 575 (M + H) |
| 100 | 2-(3-Oxa-6- azabicyclo[3.1.1]heptan-6-yl)-6- methoxy-N-(6-methoxy-4- ((2,2,6- trifluorobenzo[d][1,3]dioxol-5- yl)carbamoyl)pyridin-3- yl)benzo[d]thiazole-7- carboxamide | 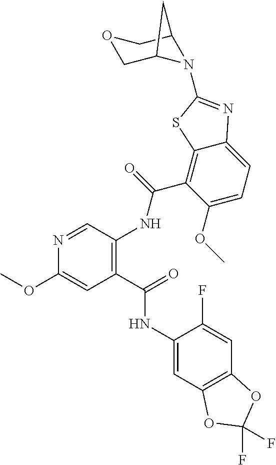 [SMILES: COc1cc(C(=O)Nc2cc3c(cc2F)OC(F)(F)O3)c(NC(=O)c2c(OC)ccc3nc(N4C5COCC4C5)sc23)cn1] | 630 (M + H) |
| 101 | 2-(3-Oxa-6- azabicyclo[3.1.1]heptan-6-yl)-6- ethoxy-N-(6-methoxy-4-((1- (trifluoromethyl)-2- oxabicyclo[2.2.2]octan-4- yl)carbamoyl)pyridin-3- yl)benzo[d]thiazole-7- carboxamide | 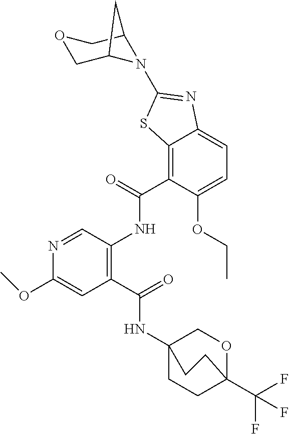 [SMILES: CCOc1ccc2nc(N3C4COCC3C4)sc2c1C(=O)Nc1cnc(OC)cc1C(=O)NC12CCC(C(F)(F)F)(CC1)OC2] | 648 (M + H) |
| 102 | 2-(3-Oxa-6- azabicyclo[3.1.1]heptan-6-yl)-N- (4-((2,2- difluorobenzo[d][1,3]dioxol-5- yl)carbamoyl)-6- methoxypyridin-3-yl)-6- isopropoxybenzo[d]thiazole-7- carboxamide | 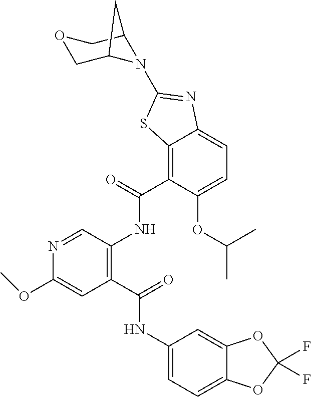 [SMILES: COc1cc(C(=O)Nc2ccc3c(c2)OC(F)(F)O3)c(NC(=O)c2c(OC(C)C)ccc3nc(N4C5COCC4C5)sc23)cn1] | 640 (M + H) |
| 103 | 2-(3-Oxa-6- azabicyclo[3.1.1]heptan-6-yl)-N- (4-((4-fluorobicyclo[2.2.2]octan- 1-yl)carbamoyl)-6- methoxypyridin-3-yl)-6- methoxybenzo[d]thiazole-7- carboxamide | 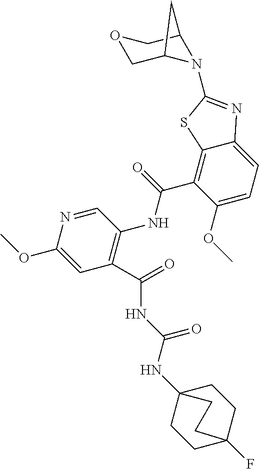 | 582 (M + H) |
| 104 | 2-(3-Oxa-6- azabicyclo[3.1.1]heptan-6-yl)-N- (4-((3-cyclopropyl-4- fluorophenyl)carbamoyl)-6- methoxypyridin-3-yl)-6- methoxybenzo[d]thiazole-7- carboxamide | 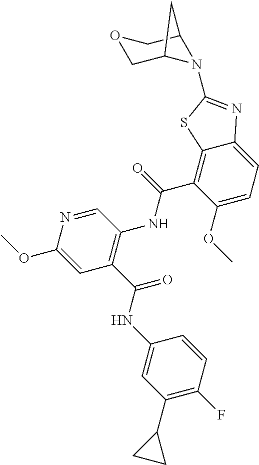 [SMILES: COc1cc(C(=O)Nc2ccc(F)c(C3CC3)c2)c(NC(=O)c2c(OC)ccc3nc(N4C5COCC4C5)sc23)cn1] | 590 (M + H) |
| 105 | 2-(3-Oxa-6- azabicyclo[3.1.1]heptan-6-yl)-N- (4-((3,3-difluoro-1- methylcyclobutyl)carbamoyl)-6- methoxypyridin-3-yl)-6- methoxybenzo[d]thiazole-7- carboxamide | 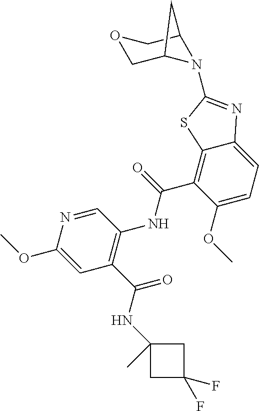 [SMILES: COc1cc(C(=O)NC2(C)CC(F)(F)C2)c(NC(=O)c2c(OC)ccc3nc(N4C5COCC4C5)sc23)cn1] | 560 (M + H) |
| 106 | N-(4-(Bicyclo[1.1.1]pentan-1- ylcarbamoyl)-6-methoxypyridin- 3-yl)-2-(3-oxa-6- azabicyclo[3.1.1]heptan-6-yl)-6- methoxybenzo[d]thiazole-7- carboxamide | 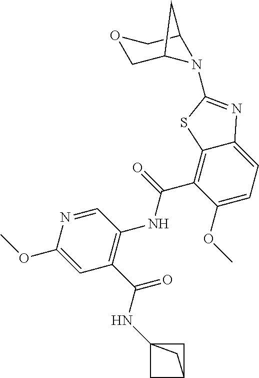 [SMILES: COc1cc(C(=O)NC23CC(C2)C3)c(NC(=O)c2c(OC)ccc3nc(N4C5COCC4C5)sc23)cn1] | 522 (M + H) |
| 107 | 2-(3-Oxa-6- azabicyclo[3.1.1]heptan-6-yl)-N- (4-((4-fluorocuban-1- yl)carbamoyl)-6- methoxypyridin-3-yl)-6- methoxybenzo[d]thiazole-7- carboxamide | 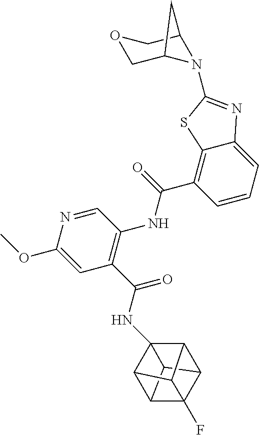 | 576 (M + H) |
| 108 | 2-(3-Oxa-6- azabicyclo[3.1.1]heptan-6-yl)-6- methoxy-N-(6-methoxy-4-((3- methoxybicyclo[1.1.1]pentan-1- yl)carbamoyl)pyridin-3- yl)benzo[d]thiazole-7- carboxamide | 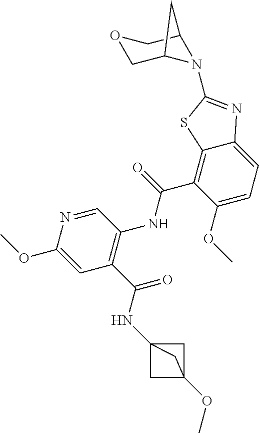 [SMILES: COc1cc(C(=O)NC23CC(OC)(C2)C3)c(NC(=O)c2c(OC)ccc3nc(N4C5COCC4C5)sc23)cn1] | 552 (M + H) |
| 109^{a} | 2-(3-Oxa-6- azabicyclo[3.1.1]heptan-6-yl)-6- methoxy-N-(6-methoxy-4-((1- methyl-3- (trifluoromethyl)cyclobutyl) carbamoyl)pyridin-3- yl)benzo[d]thiazole-7- carboxamide (Isomer 1) | 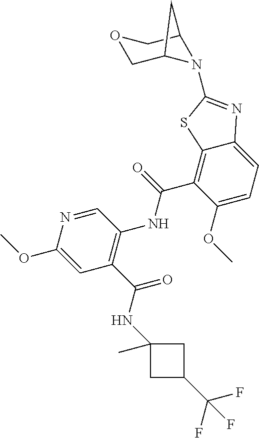 [SMILES: COc1cc(C(=O)NC2(C)CC(C(F)(F)F)C2)c(NC(=O)c2c(OC)ccc3nc(N4C5COCC4C5)sc23)cn1] | 592 (M + H) |
| 110^{a} | 2-(3-Oxa-6- azabicyclo[3.1.1]heptan-6-yl)-6- methoxy-N-(6-methoxy-4-((1- methyl-3- (trifluoromethyl)cyclobutyl) carbamoyl)pyridin-3- yl)benzo[d]thiazole-7- carboxamide (Isomer 2) | 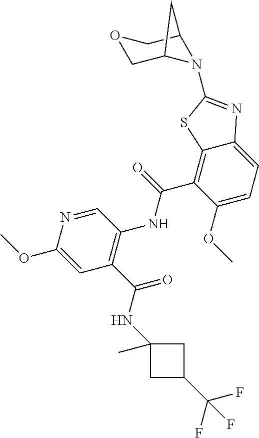 [SMILES: COc1cc(C(=O)NC2(C)CC(C(F)(F)F)C2)c(NC(=O)c2c(OC)ccc3nc(N4C5COCC4C5)sc23)cn1] | 592 (M + H) |
| 111 | N-(4-(Bicyclo[2.2.2]octan-1- ylcarbamoyl)-6-methoxypyridin- 3-yl)-2-(3-oxa-6- azabicyclo[3.1.1]heptan-6-yl)-6- methoxybenzo[d]thiazole-7- carboxamide | 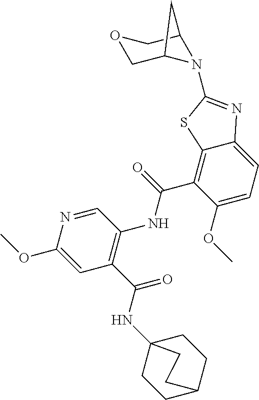 [SMILES: COc1cc(C(=O)NC23CCC(CC2)CC3)c(NC(=O)c2c(OC)ccc3nc(N4C5COCC4C5)sc23)cn1] | 564 (M + H) |
| 112 | 2-(3-Oxa-6- azabicyclo[3.1.1]heptan-6-yl)- N-(4-((3- cyclopropylbicyclo[1.1.1]pentan- 1yl)carbamoyl)-6- methoxypyridin-3-yl)-6- methoxybenzo[d]thiazole-7- carboxamide | 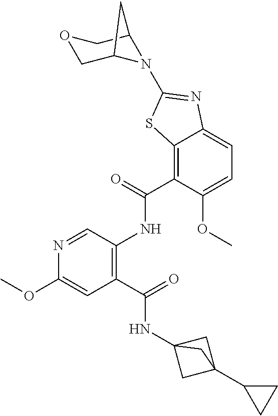 [SMILES: COc1cc(C(=O)NC23CC(C4CC4)(C2)C3)c(NC(=O)c2c(OC)ccc3nc(N4C5COCC4C5)sc23)cn1] | 562 (M + H) |
| 113 | 2-(3-Oxa-6- azabicyclo[3.1.1]heptan-6-yl)- N-(4-((3- ethylbicyclo[1.1.1]pentan- 1yl)carbamoyl)-6- methoxypyridin-3-yl)-6- methoxybenzo[d]thiazole-7- carboxamide | 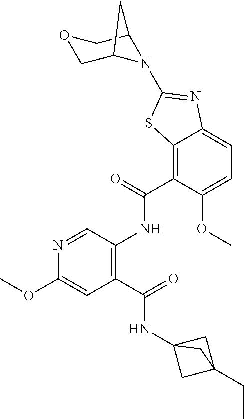 [SMILES: CCC12CC(NC(=O)c3cc(OC)ncc3NC(=O)c3c(OC)ccc4nc(N5C6COCC5C6)sc34)(C1)C2] | 550 (M + H) |
| 114 | 2-(3-Oxa-6- azabicyclo[3.1.1]heptan-6-yl)- N-(4- ((3(difluoromethyl)bicyclo[1.1.1] pentan-1-yl)carbamoyl)-6- methoxypyridin-3-yl)- 6methoxybenzo[d]thiazole-7- carboxamide | 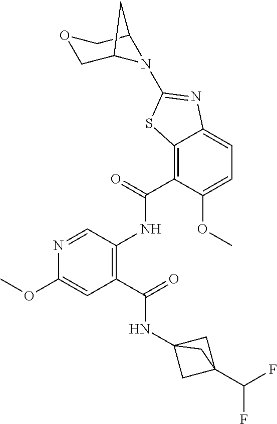 [SMILES: COc1cc(C(=O)NC23CC(C(F)F)(C2)C3)c(NC(=O)c2c(OC)ccc3nc(N4C5COCC4C5)sc23)cn1] | 572 (M + H) |
| 115 | 2-(3-Oxa-6- azabicyclo[3.1.1]heptan-6-yl)- N-(4- ((3(fluorophenyl)bicyclo[1.1.1] pentan-1-yl)carbamoyl)-6- methoxypyridin-3-yl)- 6methoxybenzo[d]thiazole-7- carboxamide | 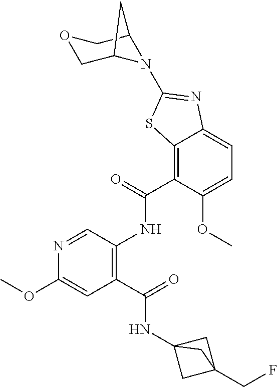 [SMILES: COc1cc(C(=O)NC23CC(CF)(C2)C3)c(NC(=O)c2c(OC)ccc3nc(N4C5COCC4C5)sc23)cn1] | 554 (M + H) |
| 116 | 2-(3-Oxa-6- azabicyclo[3.1.1]heptan-6-yl)-6- methoxy-N-(6-methoxy-4-((3- methylbicyclo[1.1.1]pentan-1- yl)carbamoyl)pyridin-3- yl)benzo[d]thiazole-7- carboxamide | 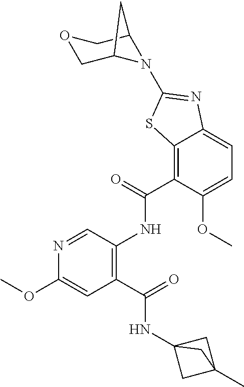 | 536 (M + H) |
| 117 | 2-(3-Oxa-6- azabicyclo[3.1.1]heptan-6-yl)- N-(4-((4-fluoro- 3methoxyphenyl)carbamoyl)-6- methoxypyridin-3-yl)-6- methoxybenzo[d]thiazole-7- carboxamide | 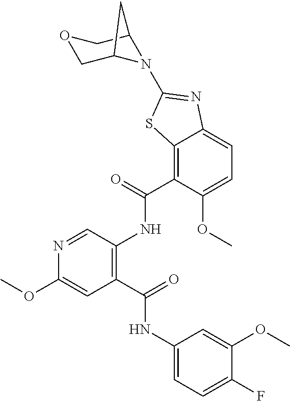 | 580 (M + H) |

^{a}Examples 109 and 110 were prepared from 5-(2-(3-oxa-6-azabicyclo[3.1.1]heptan-6-yl)-6-methoxybenzo[d]thiazole-7-
carboxamido)-2-methoxyisonicotinic acid 1-methyl-3-(trifluoromethyl)cyclobutan-1-amine hydrochloride (mixture of
isomers), and the product was purified by reverse phase flash chromatography using a gradient of 10 to 90% ACN in 10 mM
aq. NH₄HCO₃ to give Isomer 1 (first-eluting isomer, Example 109) and Isomer 2 (second-eluting isomer, Example 110).

### Example 118

### 2-(3-Oxa-6-azabicyclo[3.1.1]heptan-6-yl)-6-isopropoxy-N-(6-methoxy-4-((3-((trifluoromethyl) sulfonyl)phenyl)carbamoyl)pyridin-3-yl)benzo[d]thiazole-7-carboxamide

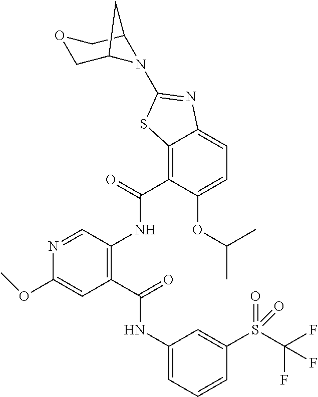 [SMILES: COc1cc(C(=O)Nc2cccc(S(=O)(=O)C(F)(F)F)c2)c(NC(=O)c2c(OC(C)C)ccc3nc(N4C5COCC4C5)sc23)cn1]

To a solution of 2-(3-oxa-6-azabicyclo[3.1.1]heptan-6-yl)-6-isopropoxybenzo[d]thiazole-7-carboxylic acid (0.060 g, 0.18 mmol) in ACN (1 mL) were added 5-amino-2-methoxy-N-(3-((trifluoromethyl) sulfonyl)phenyl)isonicotinamide (0.096 g, 0.26 mmol), NMI (52 mg, 0.63 mmol, 0.050 mL), and TCFH (0.070 g, 0.25 mmol). The reaction mixture was stirred for 16 h at RT. The reaction mixture was diluted with EtOAc. The organic phase was washed with sat. aq. NaHCO₃ solution and sat. aq. NaCl solution. The combined organic layers were dried over MgSO₄, filtered, and concentrated. The residue was taken up in ACN and a solid precipitated. The solid was collected and rinsed with ACN. The residue was dried under reduced pressure over the weekend to afford the title product (0.072 g, 0.10 mmol, 58%) as white solid. LC/MS m/z: 692 (M+H).

The following examples were prepared as described in Example 118 using the appropriate amine and carboxylic acid.

| Example | Chemical name | Structure | ES-MS m/z |
|---|---|---|---|
| 119 | 2-(3-Oxa-6-azabicyclo[3.1.1]heptan- 6-yl)-N-(4-((3- cyanobicyclo[1.1.1]pentan-1- yl)carbamoyl)-6-methoxypyridin-3- yl)-6-isopropoxybenzo[d]thiazole-7- carboxamide | 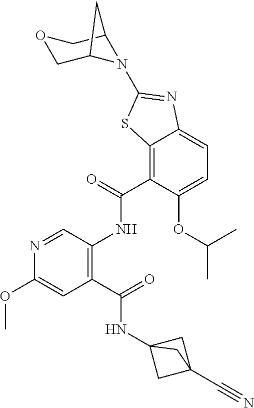 [SMILES: COc1cc(C(=O)NC23CC(C#N)(C2)C3)c(NC(=O)c2c(OC(C)C)ccc3nc(N4C5COCC4C5)sc23)cn1] | 575 (M + H) |
| 120 | 2-(3-Oxa-6-azabicyclo[3.1.1]heptan- 6-yl)-6-isopropoxy-N-(6-methoxy-4- ((3-methoxybicyclo[1.1.1]pentan-1- yl)carbamoyl)pyridin-3- yl)benzo[d]thiazole-7- carboxamide | 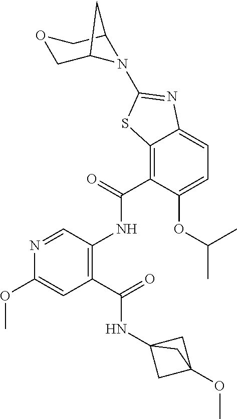 [SMILES: COc1cc(C(=O)NC23CC(OC)(C2)C3)c(NC(=O)c2c(OC(C)C)ccc3nc(N4C5COCC4C5)sc23)cn1] | 580 (M + H) |
| 121 | N-(4-((2,2- Difluorobenzo[d][1,3]dioxol-5- yl)carbamoyl)-6-methoxypyridin-3- yl)-2((1R,3r,5S)-3-hydroxy-8- azabicyclo[3.2.1]octan-8-yl)-6- methoxybenzo[d]thiazole- 7carboxamide | 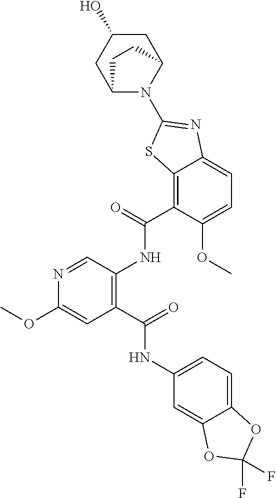 [SMILES: COc1cc(C(=O)Nc2ccc3c(c2)OC(F)(F)O3)c(NC(=O)c2c(OC)ccc3nc(N4[C@@H]5CC[C@H]4C[C@@H](O)C5)sc23)cn1] | 640 (M + H) |
| 122 | N-(4-((2,2- Difluorobenzo[d][1,3]dioxol-5- yl)carbamoyl)-6-methoxypyridin-3- yl)-2((1R,3s,5S)-3-hydroxy-8- azabicyclo[3.2.1]octan-8-yl)-6- methoxybenzo[d]thiazole- 7carboxamide | 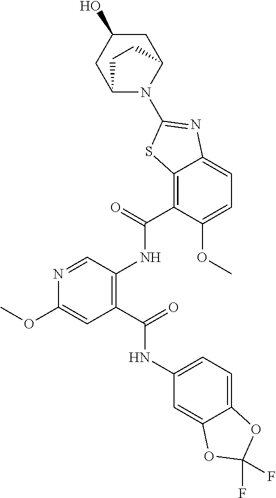 [SMILES: COc1cc(C(=O)Nc2ccc3c(c2)OC(F)(F)O3)c(NC(=O)c2c(OC)ccc3nc(N4[C@@H]5CC[C@H]4C[C@H](O)C5)sc23)cn1] | 640 (M + H) |
| 123 | 2-(3-Oxa-6-azabicyclo[3.1.1]heptan- 6-yl)-N-(6-chloro-4-((1- (trifluoromethyl)- 2oxabicyclo[2.2.2]octan-4- yl)carbamoyl)pyridazin-3-yl)-6- methoxybenzo[d]thiazole7- carboxamide | 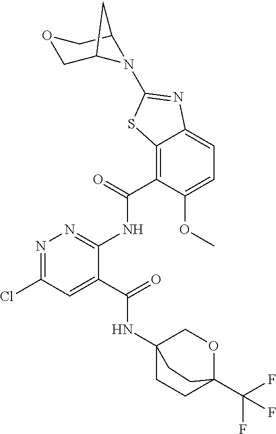 [SMILES: COc1ccc2nc(N3C4COCC3C4)sc2c1C(=O)Nc1nnc(Cl)cc1C(=O)NC12CCC(C(F)(F)F)(CC1)OC2] | 639 (M + H) |
| 124 | 2-(3-Oxa-6-azabicyclo[3.1.1]heptan- 6-yl)-N-(6-chloro-4-((2,2- difluorobenzo[d][1,3]dioxol-5- yl)carbamoyl)pyridazin-3-yl)-6- methoxybenzo[d]thiazole- 7carboxamide | 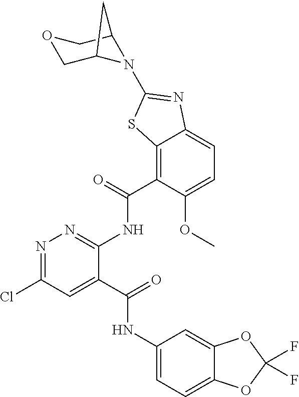 [SMILES: COc1ccc2nc(N3C4COCC3C4)sc2c1C(=O)Nc1nnc(Cl)cc1C(=O)Nc1ccc2c(c1)OC(F)(F)O2] | 617 (M + H) |
| 125^{a} | 2-(3-Oxa-6-azabicyclo[3.1.1]heptan- 6-yl)-6-isopropoxy-N-(6-methoxy-4- ((1(trifluoromethyl)-2- oxabicyclo[2.2.2]octan-4- yl)carbamoyl)pyridin- 3yl)benzo[d]thiazole-7- carboxamide | 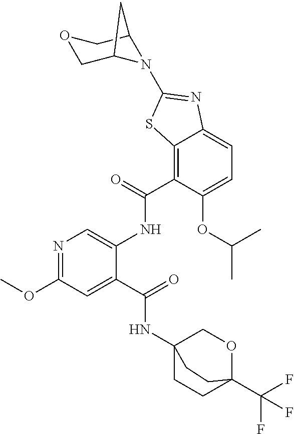 [SMILES: COc1cc(C(=O)NC23CCC(C(F)(F)F)(CC2)OC3)c(NC(=O)c2c(OC(C)C)ccc3nc(N4C5COCC4C5)sc23)cn1] | 662 (M + H) |

^{a}The product was purified by reverse phase flash chromatography using a gradient of 10 to 100% ACN in 10 mM aq. NH₄HCO₃.

### Example 126

### 2-(3-Oxa-6-azabicyclo[3.1.1]heptan-6-yl)-6-methoxy-N-(2-methoxy-5-((3 (trifluoromethyl) bicyclo[1.1.1]pentan-1-yl)carbamoyl)pyridin-4-yl)benzo[d]thiazole-7-carboxamide

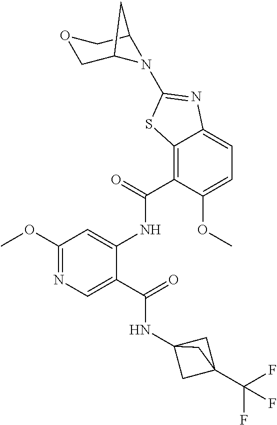 [SMILES: COc1cc(NC(=O)c2c(OC)ccc3nc(N4C5COCC4C5)sc23)c(C(=O)NC23CC(C(F)(F)F)(C2)C3)cn1]

4-(2-(3-Oxa-6-azabicyclo[3.1.1]heptan-6-yl)-6-methoxybenzo[d]thiazole-7carboxamido)-6-methoxynicotinic acid (55.4 mg, 121 μmol) and 3-(trifluoromethyl) bicyclo[1.1.1]pentan-1-amine hydrochloride (29 mg, 0.15 mmol) were dissolved in NMP (5 mL). PPACA (154 mg, 150 μL, 50 wt %, 243 μmol) and Et₃N (73.7 mg, 0.10 mL, 728 μmol) were added and the reaction was stirred at 20° C. for 30 min. The reaction mixture was partitioned between EtOAc and water. The organic layer was washed with sat. aq. NaCl, dried over Na₂SO₄, filtered, and concentrated. The residue was purified by reverse phase HPLC using a gradient of 56 to 81% ACN in 0.1% aq. formic acid. ES-MS m/z 590 (M+H).

### Example 127

### 2-(3-Oxa-6-azabicyclo[3.1.1]heptan-6-yl)-N-(5-((4-fluoro-3 (trifluoromethyl)phenyl)carbamoyl)-2-methoxypyridin-4-yl)-6methoxybenzo[d]thiazole-7-carboxamide

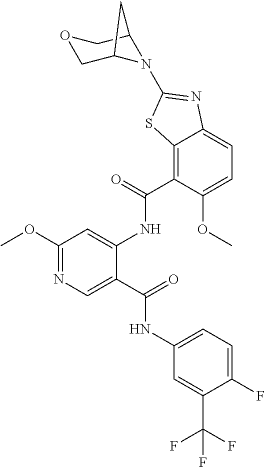 [SMILES: COc1cc(NC(=O)c2c(OC)ccc3nc(N4C5COCC4C5)sc23)c(C(=O)Nc2ccc(F)c(C(F)(F)F)c2)cn1]

A mixture of 4-(2-(3-oxa-6-azabicyclo[3.1.1]heptan-6-yl)-6-methoxybenzo[d]thiazole-7-carboxamido)-6-methoxynicotinic acid (28.4 mg, 62.2 μmol) and 4-fluoro-3-(trifluoromethyl) aniline (100 mg, 558 μmol) were dissolved in NMP (2 mL). PPACA (79.2 mg, 100 μL, 50 wt %, 124 μmol) and Et₃N (73 mg, 100 μL, 0.72 mmol) were added and the reaction was stirred at 60° C. for 30 min. The reaction mixture was partitioned between EtOAc and water. The organic layer was washed with sat. aq. NaCl, dried over Na₂SO₄, filtered, and concentrated. The residue was purified by reverse phase HPLC using a gradient of 53 to 78% ACN in 10 mM aq. NH₄HCO₃+5% MeOH. ES-MS m/z 618 (M+H).

### Example 128

### 2-(3-Oxa-6-azabicyclo[3.1.1]heptan-6-yl)-6-methoxy-N-(5-methoxy-2-((1 (trifluoromethyl)-2-oxabicyclo[2.1.1]hexan-4yl)carbamoyl)phenyl)benzo[d]thiazole-7-carboxamide

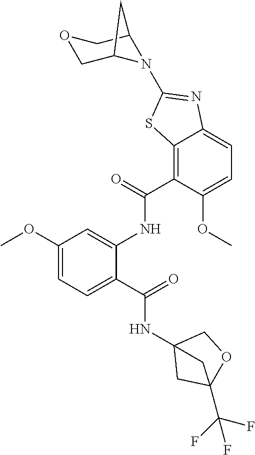 [SMILES: COc1ccc(C(=O)NC23COC(C(F)(F)F)(C2)C3)c(NC(=O)c2c(OC)ccc3nc(N4C5COCC4C5)sc23)c1]

A mixture of 2-(3-oxa-6-azabicyclo[3.1.1]heptan-6-yl)-6-methoxybenzo[d]thiazole-7-carboxylic acid (184 mg, 601 μmol) and 2-amino-4-methoxy-N-(1-(trifluoromethyl)-2-oxabicyclo[2.1.1]hexan-4-yl)benzamide (190 mg, 601 μmol) were dissolved in NMP (3 mL). PPACA (1.15 g, 1 mL, 50 wt %, 1.80 mmol) and Et₃N (243 mg, 0.33 mL, 2.40 mmol) were added and the reaction was stirred at 60° C. for 16 h. The reaction mixture was purified by reverse phase HPLC using a gradient of 50 to 75% ACN in 0.1% aq. formic acid. ES-MS m/z 605 (M+H).

### Example 129

### 2-(3-Oxa-6-azabicyclo[3.1.1]heptan-6-yl)-6-methoxy-N-(5-methoxy-2-((3-methoxybicyclo[1.1.1]pentan-1-yl)carbamoyl)phenyl)benzo[d]thiazole-7-carboxamide

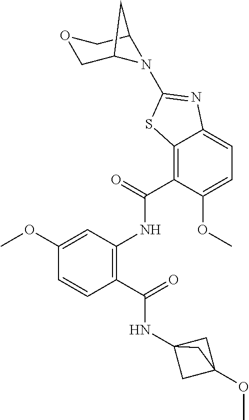

2-(3-Oxa-6-azabicyclo[3.1.1]heptan-6-yl)-6-methoxybenzo[d]thiazole-7-carboxylic acid (142 mg, 465 μmol) and 2-amino-4-methoxy-N-(3-methoxybicyclo[1.1.1]pentan-1-yl)benzamide (122 mg, 465 μmol) were dissolved in NMP (3 mL). PPACA (888 mg, 1 mL, 50 wt % 1.40 mmol) and Et₃N (188 mg, 0.26 mL, 1.86 mmol) were added and the reaction was stirred at 60° C. for 16 h. The reaction mixture was partitioned between EtOAc and water. The organic layer was dried over MgSO₄, filtered, and concentrated. The residue was purified by reverse phase HPLC using a gradient of 39 to 64% ACN in 10 mM aq. NH₄HCO₃+5% MeOH. ES-MS m/z 551 (M+H).

### Example 130

### 2-(3-Oxa-6-azabicyclo[3.1.1]heptan-6-yl)-6-methoxy-N-(4-((3methoxybicyclo[1.1.1]pentan-1-yl)carbamoyl)-6-(methylthio)pyridin-3yl)benzo[d]thiazole-7-carboxamide

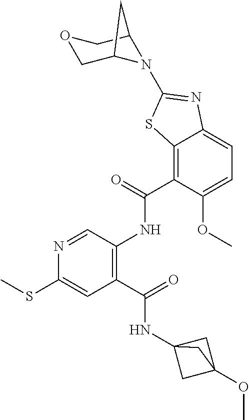

A mixture of 5-amino-N-(3-methoxybicyclo[1.1.1]pentan-1-yl)-2-(methylthio) isonicotinamide (20 mg, 72 μmol), 2-(3-oxa-6-azabicyclo[3.1.1]heptan-6-yl)-6-methoxybenzo[d]thiazole-7-carboxylic acid (26 mg, 86 μmol), EtOAc (0.72 mL), DiPEA (28 mg, 37 μL, 0.21 mmol), 4-(pyrrolidin-1-yl)pyridine (6.4 mg, 43 μmol) and PPACA (50 wt %, 68 mg, 62 μL, 0.11 mmol) was heated to 50° C. for 90 min, then additional PPACA (50 wt %, 23 mg, 21 μL, 0.037 mmol) was added. The reaction was heated to 50° C. overnight, then cooled to RT and diluted with EtOAc and 1:1 sat. aq. K₂CO₃: water. The mixture was filtered, the layers were separated and the organic layer was concentrated under reduced pressure. The residue was purified by reverse phase flash chromatography using a gradient of 20 to 100% ACN in 10 mM aq. NH₄HCO₃+5% MeOH to give the title compound (15 mg, 37%). ES-MS m/z 568 (M+H).

Biological Assay: The following assay demonstrate that the exemplified compounds are RXFP1 receptor agonists.

Human RXFP1 Cell cAMP HTRF Assay: Relaxin family peptide receptor 1 (RXFP1) expressing cells were generated by transfecting a tetracycline-inducible RXFP1 expression cassette (Accession NM_021634.4) into HEK293 cells and single cell clones that are responsive to Human Relaxin2 (cat #H-6784.0200, Bachem, Torrance, CA) were selected. RXFP1 expressing cells were treated with 1 μg/mL doxycycline for 24 h prior to compound dosing in order to induce RXFP1 expression. At the time of cell dosing, compounds were acoustically transferred into 384-well plates (ProxiPlate, 384-well, cat #6008289, Revvity, Waltham, MA) using the ECHO acoustic liquid handler (Beckman, Brea, CA). Cells were dosed in 10-point dose-response curves (3-fold dilution) starting at 10 μM for 1 h at 37° C. Final DMSO concentration was 0.6% in a 10 μL assay volume.

Assay buffer used for treatment was 1× Stimulation Buffer (provided with the Gs cAMP HighRange kit, cat #62AM6PEJ, Revvity), diluted in distilled water and containing 500 μM 3-Isobutyl-1-methylxanthine final (cat #15879250 MG, Sigma-Aldrich, St. Louis, MO). After 1 h incubation, cAMP levels were assessed using the CisBio Gs CAMP HighRange Kit (cat #62AM6PEJ) according to the manufacturer's instruction (Revvity). Briefly, 5 μL of D2-labeled CAMP and 5 μL of Eu³⁺-Cryptate-labeled anti-cAMP antibody diluted in lysis buffer (kit provided) were added to each well. Cell lysis solutions were incubated for 1 h at room temperature prior to detection on a PHERAstar FSX microplate reader (BMG, Ortenberg, Germany,). The HTRF ratio (Emission 665 nm/Emission 620 nm×10,000) was converted to CAMP units using a cAMP standard curve. Relative EC₅₀s were calculated relative to the top and bottom of the individual concentration response curves, determined via non-linear regression using a four-parameter logistic fit using Genedata Ver. 18.0.8 (Genedata, Basel, Switzerland).

In the above assay, compounds of Examples 1-130 exhibited an agonist activity in the RXFP1 receptor with a relative EC₅₀ of <50 nM. This data shows that compounds of Formula I as described herein are RXFP1 agonists in these human HEK293 cells.

We claim:

1. A compound of the formula: 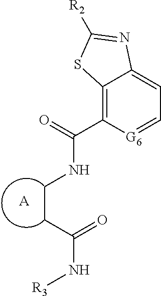
wherein:
Ring A is a group of the formula 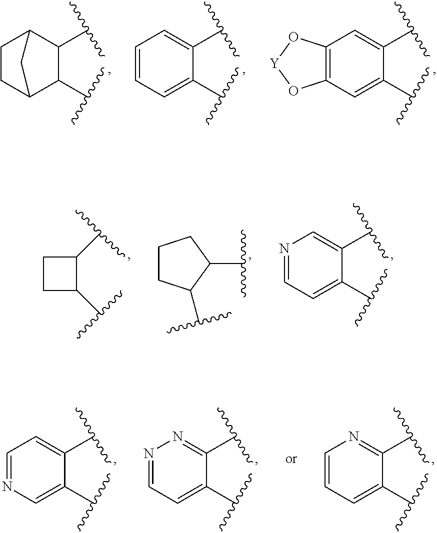
wherein each of 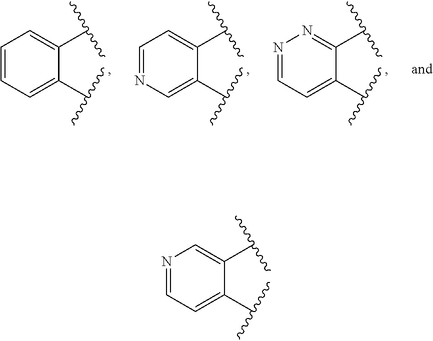 [SMILES: *c1ccccc1* ; *c1ccncc1* ; *c1ccnnc1* ; *c1ccncc1*]
is optionally substituted by one or more substituents selected from halogens, —CN, C₁₋₄ alkyl, —S—C₁₋₃ alkyl, and C₁₋₃ alkoxy, wherein each C₁₋₄ alkyl and C₁₋₃ alkoxy is independently optionally substituted by one or more substituents selected from halogens and —CN;
—Y— is a C₁₋₃ alkylene;
—G₆— is —C(R₁)— or —N—;
R₁ is C₁₋₃ alkoxy;
R₂ is —OCH₂COOH or an N-linked 4-7 membered heterocycle; wherein the N-linked 4-7 membered heterocycle is selected from an azetidine, pyrrolidine, morpholine, piperazine, piperidine, and oxazepane; wherein the heterocycle is optionally bridged by, fused with, or spiro-linked with a C₁₋₄ alkylenyl, or a 1-5 membered heteroalkyl; wherein the heterocycle, the C₁₋₄ alkylenyl, and the 1-5 membered heteroalkyl are each optionally substituted with 1-3 substituents each independently selected from halogen, oxo, C₁₋₄ alkyl, C₁₋₄ haloalkyl, —OH, and —COOH;
R₃ is selected from the group consisting of 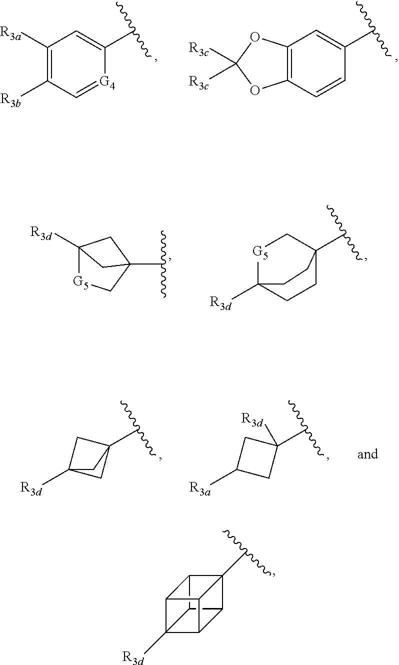
wherein 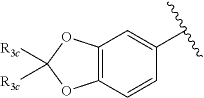
is optionally substituted with one to three halogens;
—G₄— is —CH— or —N—;
—G₅— is —CH₂— or —O—;
R₃ₐ, at each occurrence, is independently —H, halogen, —SF₅, —SO₂CF₃, —SCF₃, —CN, C₁₋₄ alkyl, C₃₋₆ cycloalkyl, or C₁₋₃ alkoxy, wherein the C₁₋₄ alkyl and C₁₋₃ alkoxy are optionally substituted with one, two or three halogens;
R_{3b} is independently —H, halogen, difluoromethyl, trifluoromethyl, methoxy, trifluoromethoxy, —CN, or C₁₋₄ alkyl;
R_{3c}, at each occurrence, is independently —H or halogen;
R_{3d} is —H, halogen, —CN, C₁₋₄ alkyl, C₃₋₆ cycloalkyl, or C₁₋₃ alkoxy, wherein the C₁₋₄ alkyl and C₁₋₃ alkoxy are optionally substituted with one, two, or three halogens; or a pharmaceutically acceptable salt thereof.

2. The compound according to claim 1 wherein:
Ring A is a group of the formula 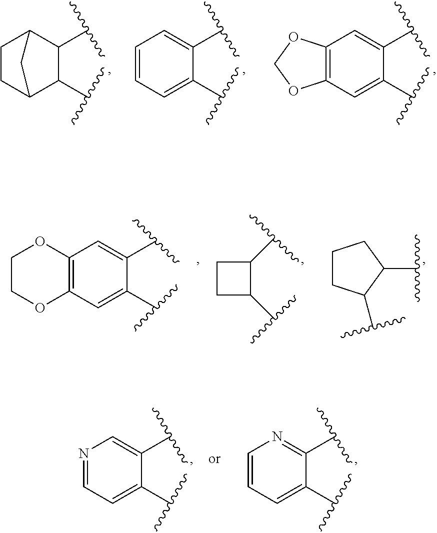
wherein each of 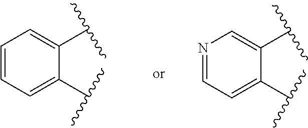 [SMILES: *c1ccccc1* ; *c1ccncc1*]
are optionally substituted by one or two halogens, C₁₋₄ alkyl, or C₁₋₃ alkoxy, wherein each C₁₋₄ alkyl or C₁₋₃ alkoxy is independently optionally substituted by one or more of halogens or —CN;
R₂ is a group of the formula 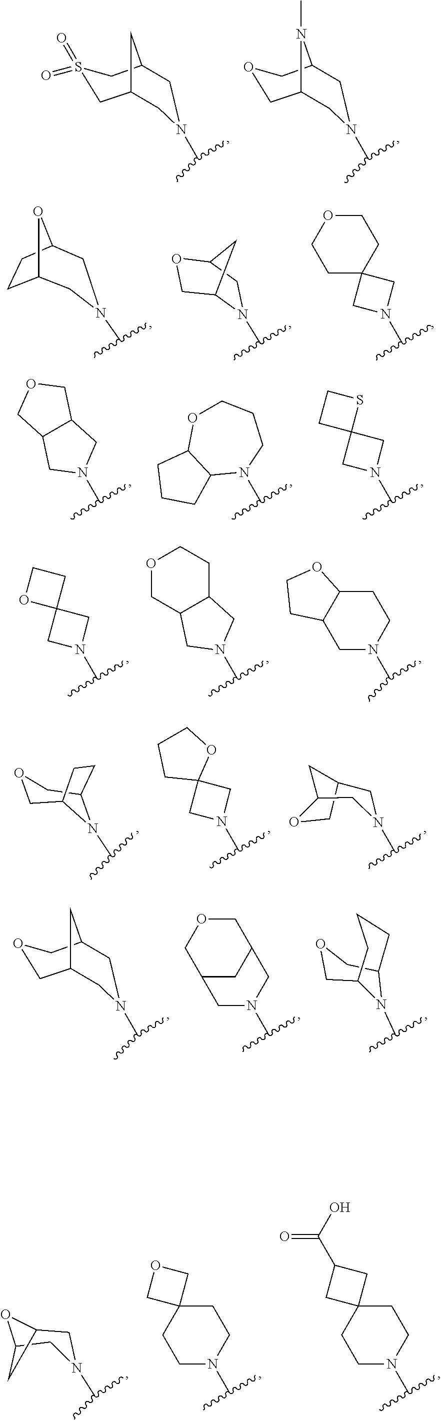 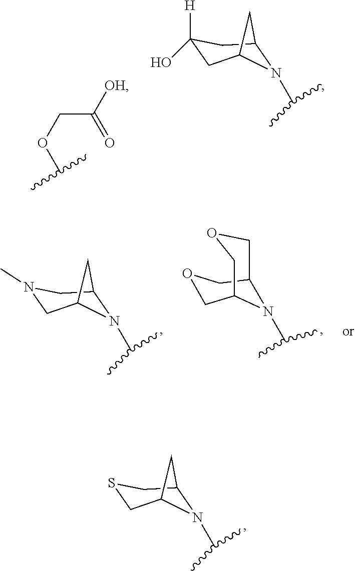
wherein 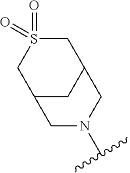 [SMILES: *N1CC2CC(C1)CS(=O)(=O)C2]
is optionally substituted with one or two halogens, wherein 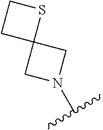 [SMILES: *N1CC2(CCS2)C1]
is optionally substituted with one or two oxo;
wherein R₃ is selected from the group consisting of 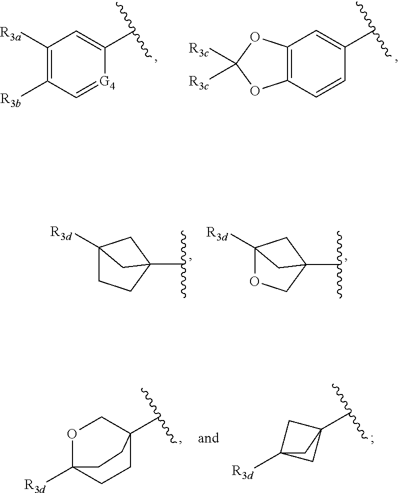
R₃ₐ, at each occurrence, is independently —H, halogen, —SF₅, —SO₂CF₃, —SCF₃, C₁₋₄ alkyl, or C₁₋₃ alkoxy, wherein the C₁₋₄ alkyl or C₁₋₃ alkoxy are optionally substituted with one or two halogens;
R_{3d} is —H, halogen, —CN, C₁₋₄ alkyl, or C₁₋₃ alkoxy, wherein the C₁₋₄ alkyl or C₁₋₃ alkoxy are optionally substituted with one or two halogens; and
—G₆— is —C(R₁)—; or a pharmaceutically acceptable salt thereof.

3. The compound according to claim 1 wherein:
Ring A is a group of the formula 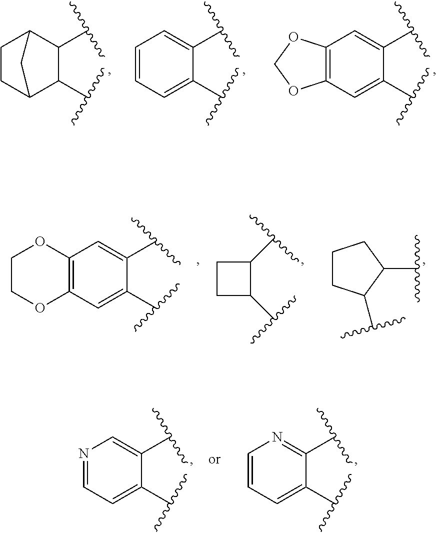
wherein each of 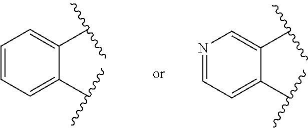 [SMILES: *c1ccccc1* ; *c1ccncc1*]
are optionally substituted by one or two halogens, C₁₋₄ alkyl, or C₁₋₃ alkoxy, wherein each C₁₋₄ alkyl or C₁₋₃ alkoxy is independently optionally substituted by one or more of halogens or —CN;
R₂ is a group of the formula 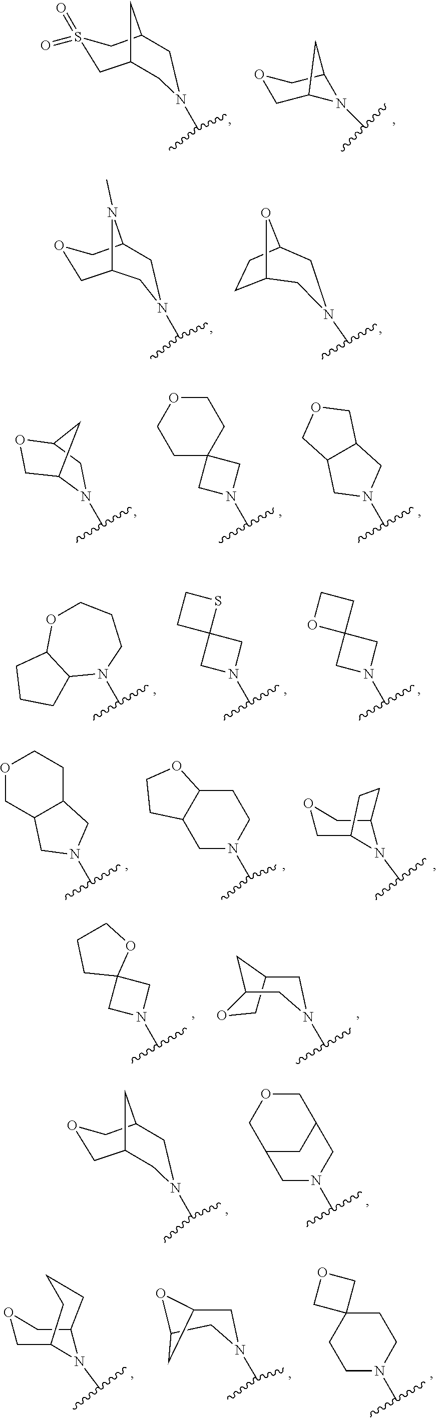 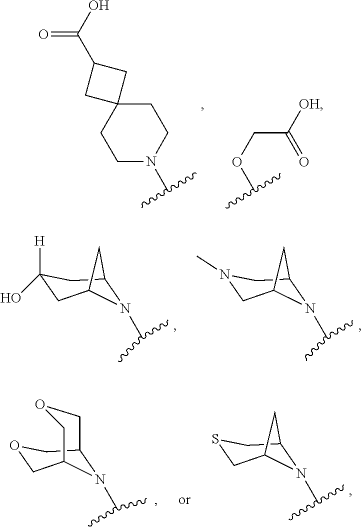
wherein 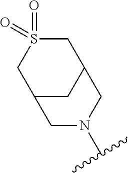 [SMILES: *N1CC2CC(C1)CS(=O)(=O)C2]
is optionally substituted with one or two halogens, wherein 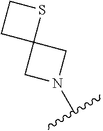 [SMILES: *N1CC2(CCS2)C1]
is optionally substituted with one or two oxo;
wherein R₃ is selected from the group consisting of 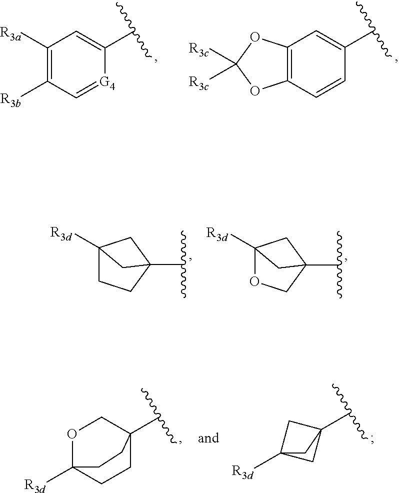
R₃ₐ, at each occurrence, is independently —H, halogen, —SF₅, —SO₂CF₃, —SCF₃, C₁₋₄ alkyl, or C₁₋₃ alkoxy, wherein the C₁₋₄ alkyl and C₁₋₃ alkoxy are optionally substituted with one or two halogens;
R_{3d} is —H, halogen, —CN, C₁₋₄ alkyl, or C₁₋₃ alkoxy, wherein the C₁₋₄ alkyl or C₁₋₃ alkoxy are optionally substituted with one or two halogens; and
—G₆— is —C(R₁)—; or a pharmaceutically acceptable salt thereof.

4. The compound according to claim 1 wherein Ring A is a group of the formula 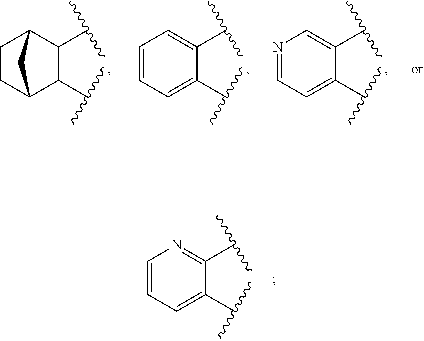 [SMILES: *C1C(*)[C@H]2CC[C@@H]1C2 ; *c1ccccc1* ; *c1ccncc1* ; *c1cccnc1*]
R₂ is —OCH₂COOH or an N-linked 4-7 membered heterocycle; wherein the N-linked 4-7 membered heterocycle is selected from an azetidine, pyrrolidine, morpholine, piperazine, piperidine, or oxazepane; wherein the heterocycle is optionally bridged by, fused with, or spiro-linked with a C₁₋₄ alkylenyl, or 1-5 membered heteroalkyl; wherein the heterocycle, the C₁₋₄ alkylenyl, and 1-5 membered heteroalkyl are each optionally substituted with 1-3 substituents each independently selected from halogen, C₁₋₄ alkyl, C₁₋₄ haloalkyl, and —COOH;
R₃ is a group of the formula: 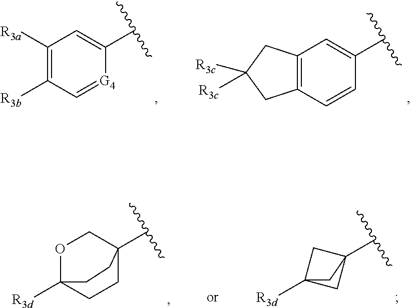
R₃ₐ is —H, halogen, —SF₅, —SO₂CF₃, —SCF₃, C₁₋₄ alkyl, or C₁₋₃ alkoxy, wherein the C₁₋₄ alkyl or C₁₋₃ alkoxy are optionally substituted with one or more halogens;
each R_{3c} is independently —H, or —F;
R_{3d} is —H, halogen, C₁₋₄ alkyl, or C₁₋₃ alkoxy, wherein the C₁₋₄ alkyl or C₁₋₃ alkoxy are optionally substituted with one or more halogens; and
—G₆— is —C(R₁)—; or a pharmaceutically acceptable salt thereof.

5. The compound according to claim 1 wherein Ring A is a group of the formula 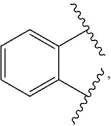 [SMILES: *c1ccccc1*]
wherein 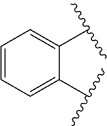 [SMILES: *c1ccccc1*]
is optionally substituted by one or two halogens, C₁₋₄ alkyl, or C₁₋₃ alkoxy, wherein each C₁₋₄ alkyl or C₁₋₃ alkoxy is independently optionally substituted by one or more selected from halogens and —CN;
or a pharmaceutically acceptable salt thereof.

6. The compound according to claim 1 wherein Ring A is a group of the formula 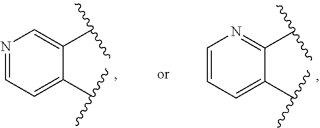 [SMILES: *c1ccncc1* ; *c1cccnc1*]
wherein 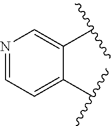 [SMILES: *c1ccncc1*]
is optionally substituted by one or two halogens, C₁₋₄ alkyl, or C₁₋₃ alkoxy, wherein each C₁₋₄ alkyl or C₁₋₃ alkoxy is independently optionally substituted by one or more substituents selected from halogens and —CN;
or a pharmaceutically acceptable salt thereof.

7. The compound according to claim 1 wherein Ring A is a group of the formula 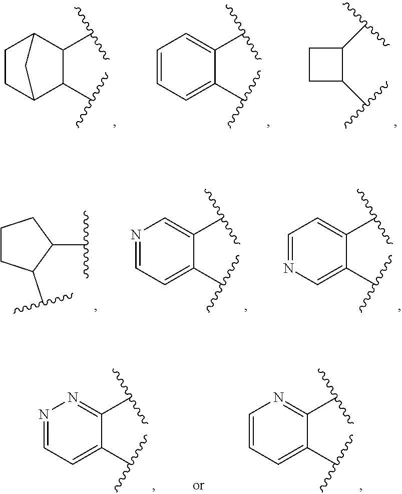
wherein each of 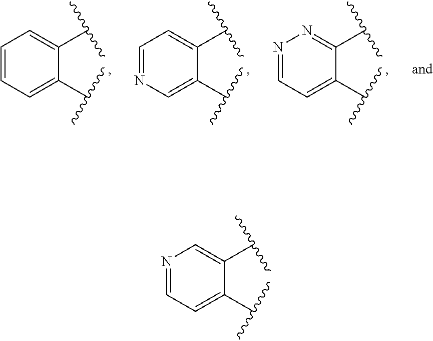 [SMILES: *c1ccccc1* ; *c1ccncc1* ; *c1ccnnc1* ; *c1ccncc1*]
is optionally substituted by one or more substituents selected from halogens, —CN, C₁₋₄ alkyl, —S—C₁₋₃ alkyl, and C₁₋₃ alkoxy, wherein each C₁₋₄ alkyl and C₁₋₃ alkoxy is independently optionally substituted by one or more substituents selected from halogens and —CN; or a pharmaceutically acceptable salt thereof.

8. The compound according to claim 1 wherein Ring A is a group of the formula 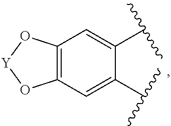
or a pharmaceutically acceptable salt thereof.

9. The compound according to claim 1, wherein —G₆— is —C(R₁)—, or a pharmaceutically acceptable salt thereof.

10. The compound according to claim 9, wherein R₁ is methoxy, ethoxy, or isopropoxy or a pharmaceutically acceptable salt thereof.

11. The compound according to claim 10, wherein R₁ is methoxy, or a pharmaceutically acceptable salt thereof.

12. The compound according to claim 1, wherein R₃ is selected from the group consisting of 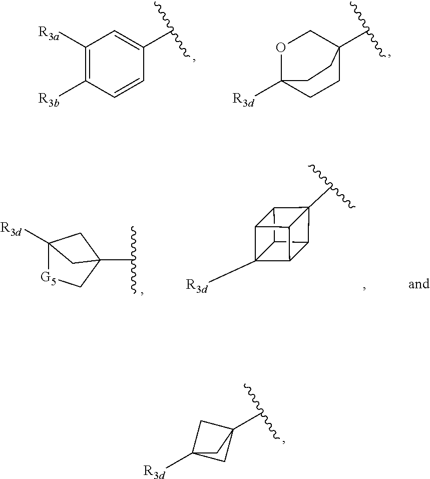
or a pharmaceutically acceptable salt thereof.

13. The compound according to claim 12, wherein R_{3d} is trifluoromethyl, or a pharmaceutically acceptable salt thereof.

14. The compound according to claim 1 of the formula 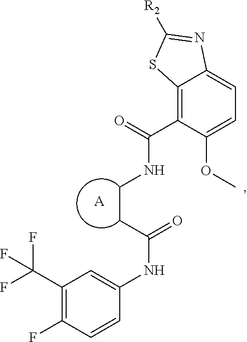
or a pharmaceutically acceptable salt thereof.

15. The compound according to claim 1, wherein R₂ is selected from the group consisting of 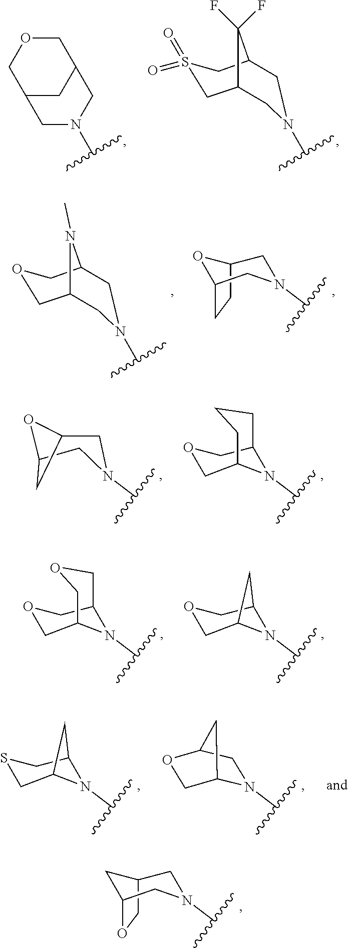
or a pharmaceutically acceptable salt thereof.

16. The compound according to claim 15 wherein R₂ is a group of the formula 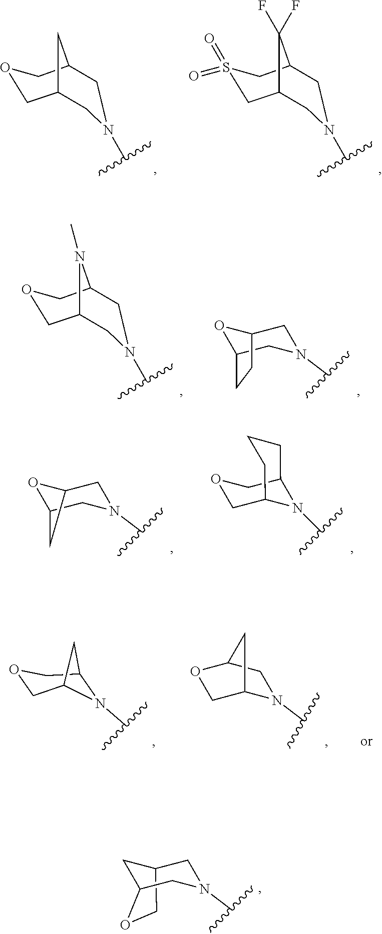
or a pharmaceutically acceptable salt thereof.

17. The compound according to claim 16 wherein R₂ is a group of the formula 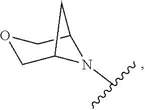 [SMILES: *N1C2COCC1C2]
or a pharmaceutically acceptable salt thereof.

18. The compound according to claim 1, wherein R₂ is selected from the group consisting of 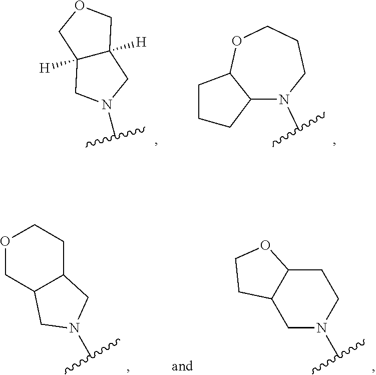 [SMILES: *N1C[C@H]2COC[C@H]2C1 ; *N1CCCOC2CCCC21 ; *N1CC2CCOCC2C1 ; *N1CCC2OCCC2C1]
or a pharmaceutically acceptable salt thereof.

19. The compound according to claim 1, wherein R₂ is selected from the group consisting of 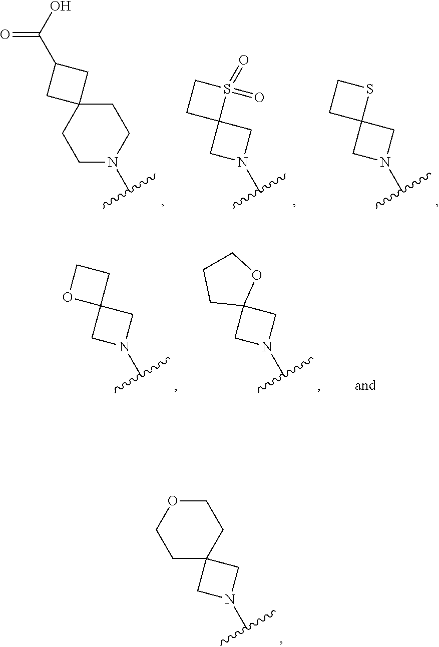 [SMILES: *N1CCC2(CC1)CC(C(=O)O)C2 ; *N1CC2(CCS2(=O)=O)C1 ; *N1CC2(CCS2)C1 ; *N1CC2(CCO2)C1 ; *N1CC2(CCCO2)C1 ; *N1CC2(CCOCC2)C1]
or a pharmaceutically acceptable salt thereof.

20. The compound according to claim 1, wherein R₂ is 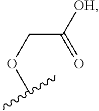 [SMILES: *OCC(=O)O]
or a pharmaceutically acceptable salt thereof.

21. The compound according to claim 1 selected from the group consisting of 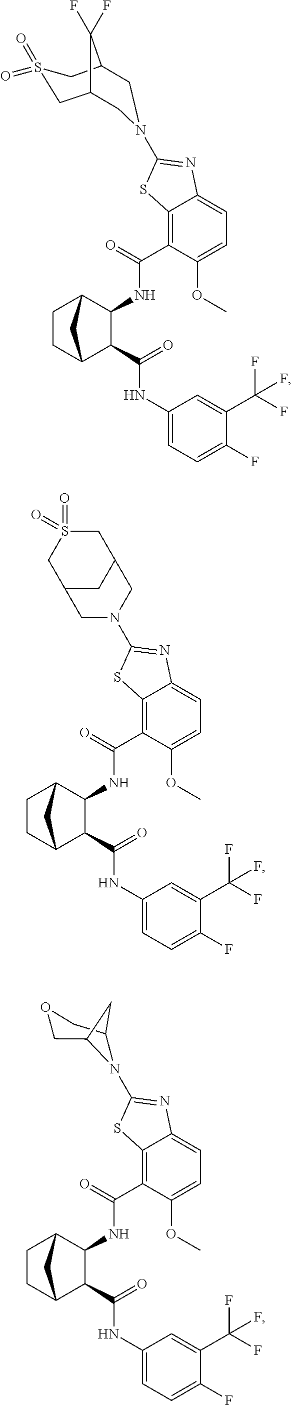 [SMILES: COc1ccc2nc(N3CC4CS(=O)(=O)CC(C3)C4(F)F)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)CS(=O)(=O)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3C4)sc2c1C(=O)N[C@@H]1[C@H]2CC[C@H](C2)[C@@H]1C(=O)Nc1ccc(F)c(C(F)(F)F)c1] 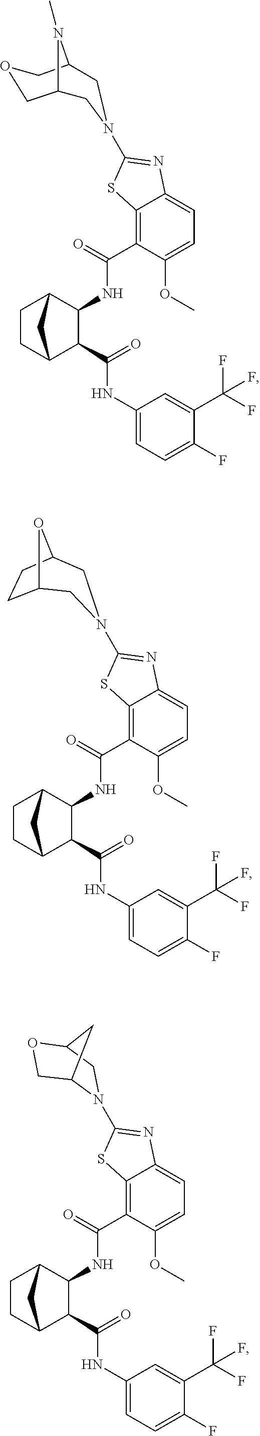 [SMILES: COc1ccc2nc(N3CC4COCC(C3)N4C)sc2c1C(=O)N[C@@H]1[C@H]2CC[C@H](C2)[C@@H]1C(=O)Nc1ccc(F)c(C(F)(F)F)c1 ; COc1ccc2nc(N3CC4CCC(C3)O4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC3CO4)sc2c1C(=O)N[C@@H]1[C@H]2CC[C@H](C2)[C@@H]1C(=O)Nc1ccc(F)c(C(F)(F)F)c1] 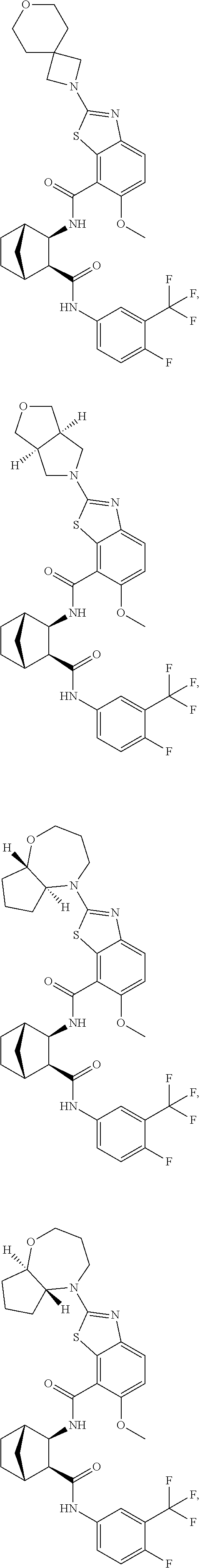 [SMILES: COc1ccc2nc(N3CC4(CCOCC4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C[C@H]4COC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CCCO[C@H]4CCC[C@@H]43)sc2c1C(=O)N[C@@H]1[C@H]2CC[C@H](C2)[C@@H]1C(=O)Nc1ccc(F)c(C(F)(F)F)c1 ; COc1ccc2nc(N3CCCO[C@@H]4CCC[C@H]43)sc2c1C(=O)N[C@@H]1[C@H]2CC[C@H](C2)[C@@H]1C(=O)Nc1ccc(F)c(C(F)(F)F)c1] 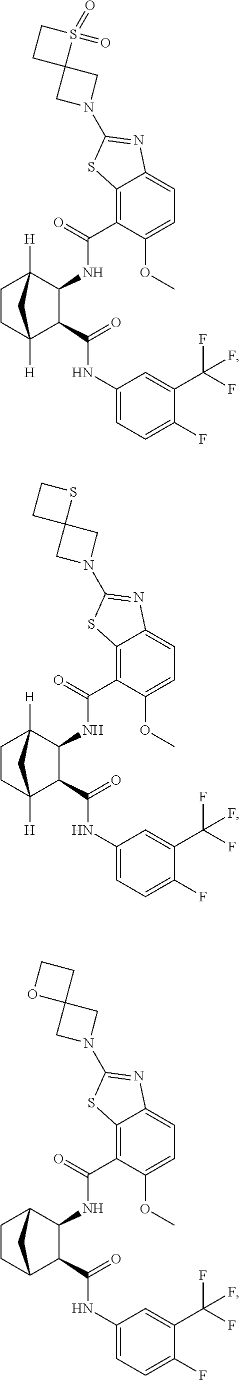 [SMILES: COc1ccc2nc(N3CC4(CCS4(=O)=O)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCS4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCO4)C3)sc2c1C(=O)N[C@@H]1[C@H]2CC[C@H](C2)[C@@H]1C(=O)Nc1ccc(F)c(C(F)(F)F)c1] 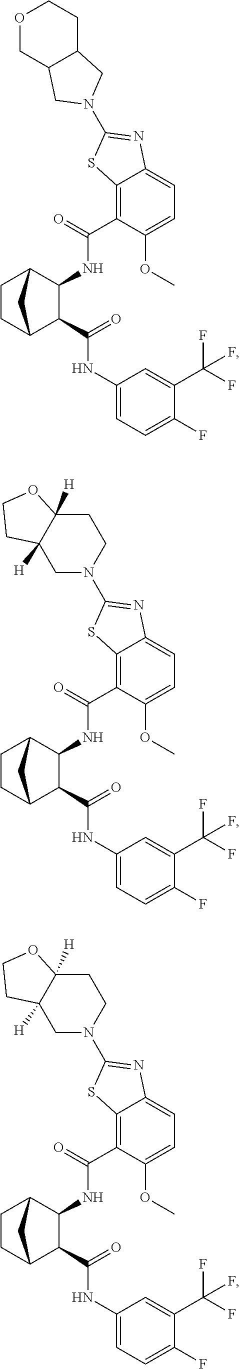 [SMILES: COc1ccc2nc(N3CC4CCOCC4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@H]4OCC[C@H]4C3)sc2c1C(=O)N[C@@H]1[C@H]2CC[C@H](C2)[C@@H]1C(=O)Nc1ccc(F)c(C(F)(F)F)c1 ; COc1ccc2nc(N3CC[C@@H]4OCC[C@@H]4C3)sc2c1C(=O)N[C@@H]1[C@H]2CC[C@H](C2)[C@@H]1C(=O)Nc1ccc(F)c(C(F)(F)F)c1] 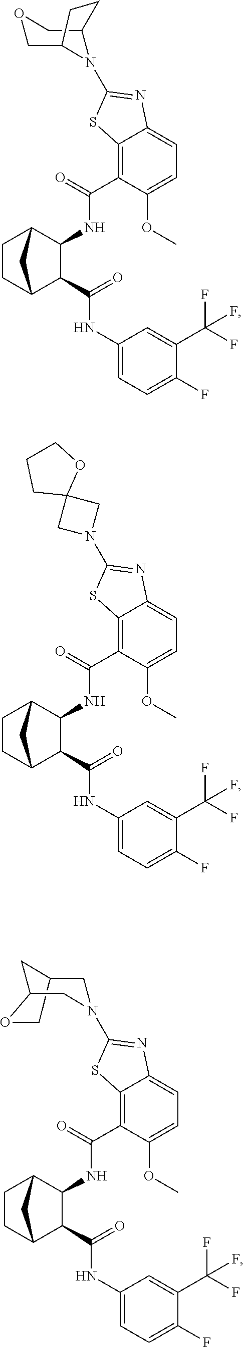 [SMILES: COc1ccc2nc(N3C4CCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4(CCCO4)C3)sc2c1C(=O)N[C@@H]1[C@H]2CC[C@H](C2)[C@@H]1C(=O)Nc1ccc(F)c(C(F)(F)F)c1 ; COc1ccc2nc(N3CC4COC(C4)C3)sc2c1C(=O)N[C@@H]1[C@H]2CC[C@H](C2)[C@@H]1C(=O)Nc1ccc(F)c(C(F)(F)F)c1] 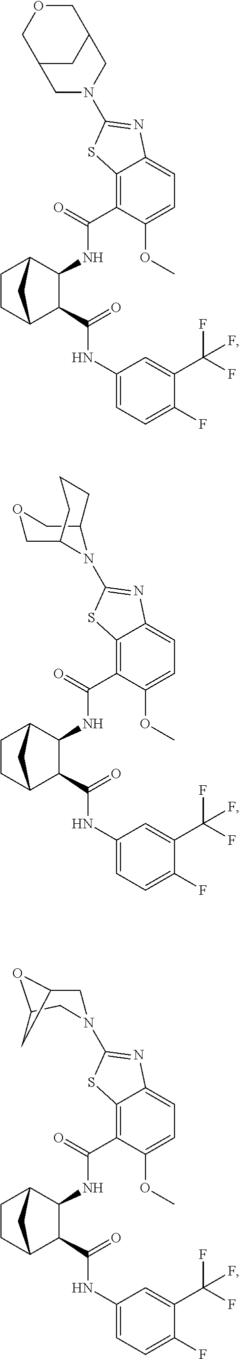 [SMILES: COc1ccc2nc(N3CC4COCC(C4)C3)sc2c1C(=O)N[C@@H]1[C@H]2CC[C@H](C2)[C@@H]1C(=O)Nc1ccc(F)c(C(F)(F)F)c1 ; COc1ccc2nc(N3C4CCCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4CC(C3)O4)sc2c1C(=O)N[C@@H]1[C@H]2CC[C@H](C2)[C@@H]1C(=O)Nc1ccc(F)c(C(F)(F)F)c1] 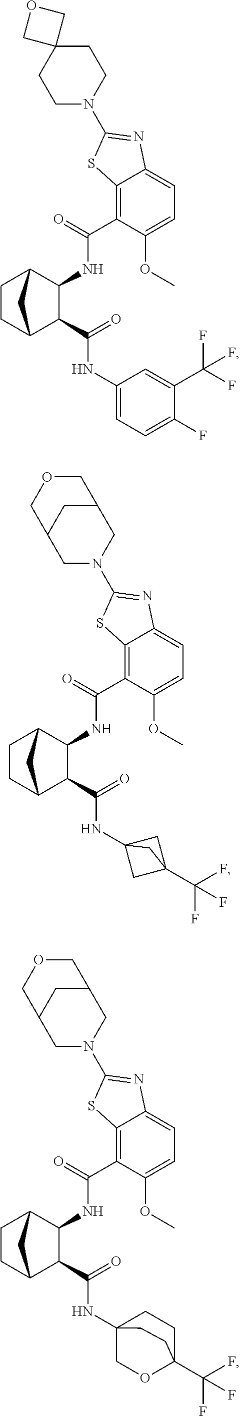 [SMILES: COc1ccc2nc(N3CCC4(CC3)COC4)sc2c1C(=O)N[C@@H]1[C@H]2CC[C@H](C2)[C@@H]1C(=O)Nc1ccc(F)c(C(F)(F)F)c1 ; COc1ccc2nc(N3CC4COCC(C4)C3)sc2c1C(=O)N[C@@H]1[C@H]2CC[C@H](C2)[C@@H]1C(=O)NC12CC(C(F)(F)F)(C1)C2 ; COc1ccc2nc(N3CC4COCC(C4)C3)sc2c1C(=O)N[C@@H]1[C@H]2CC[C@H](C2)[C@@H]1C(=O)NC12CCC(C(F)(F)F)(CC1)OC2] 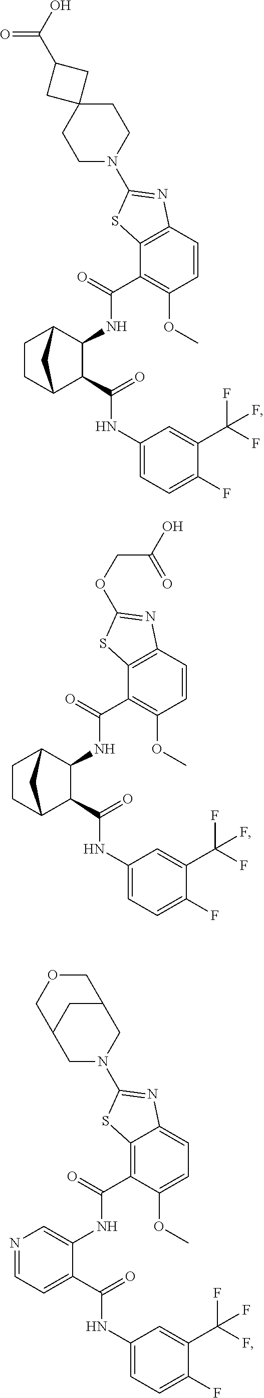 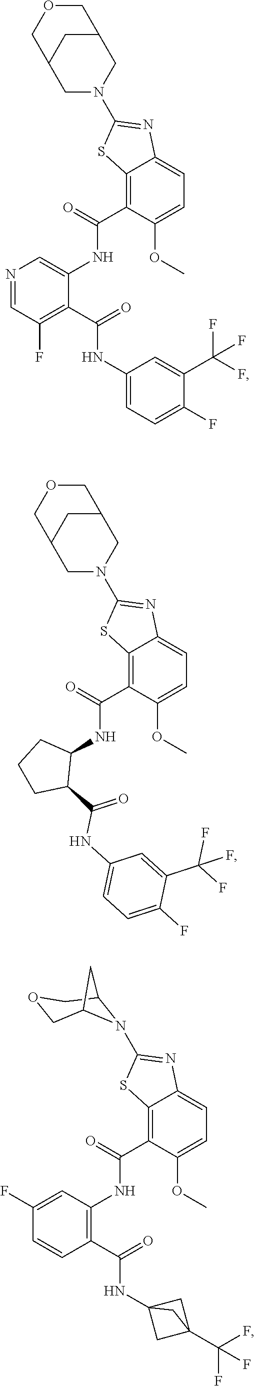 [SMILES: COc1ccc2nc(N3CC4COCC(C4)C3)sc2c1C(=O)Nc1cncc(F)c1C(=O)Nc1ccc(F)c(C(F)(F)F)c1 ; COc1ccc2nc(N3CC4COCC(C4)C3)sc2c1C(=O)N[C@@H]1CCC[C@@H]1C(=O)Nc1ccc(F)c(C(F)(F)F)c1 ; COc1ccc2nc(N3C4COCC3C4)sc2c1C(=O)Nc1cc(F)ccc1C(=O)NC12CC(C(F)(F)F)(C1)C2] 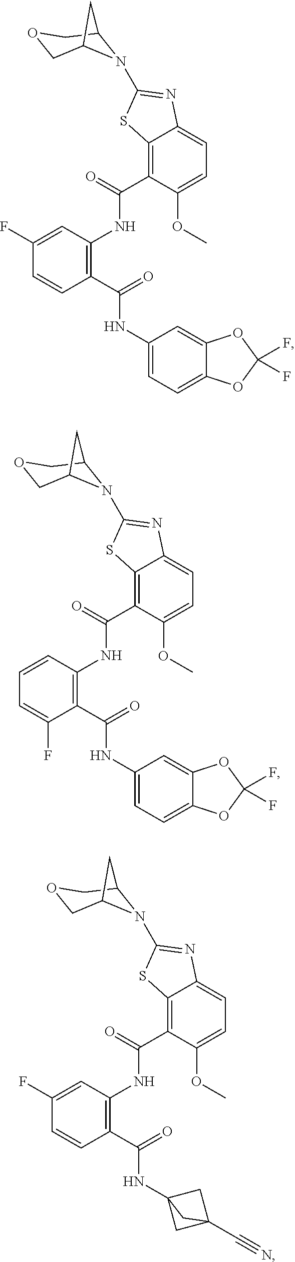 [SMILES: COc1ccc2nc(N3C4COCC3C4)sc2c1C(=O)Nc1cc(F)ccc1C(=O)Nc1ccc2c(c1)OC(F)(F)O2 ; COc1ccc2nc(N3C4COCC3C4)sc2c1C(=O)Nc1cccc(F)c1C(=O)Nc1ccc2c(c1)OC(F)(F)O2 ; COc1ccc2nc(N3C4COCC3C4)sc2c1C(=O)Nc1cc(F)ccc1C(=O)NC12CC(C#N)(C1)C2] 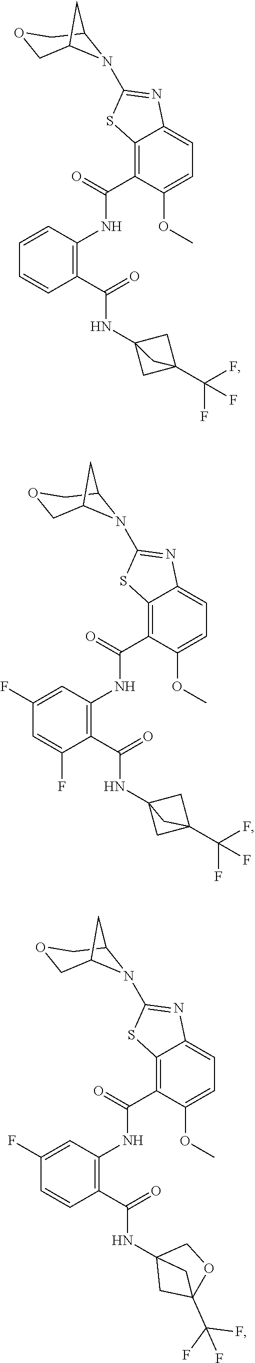 [SMILES: COc1ccc2nc(N3C4COCC3C4)sc2c1C(=O)Nc1ccccc1C(=O)NC12CC(C(F)(F)F)(C1)C2 ; COc1ccc2nc(N3C4COCC3C4)sc2c1C(=O)Nc1cc(F)cc(F)c1C(=O)NC12CC(C(F)(F)F)(C1)C2 ; COc1ccc2nc(N3C4COCC3C4)sc2c1C(=O)Nc1cc(F)ccc1C(=O)NC12COC(C(F)(F)F)(C1)C2] 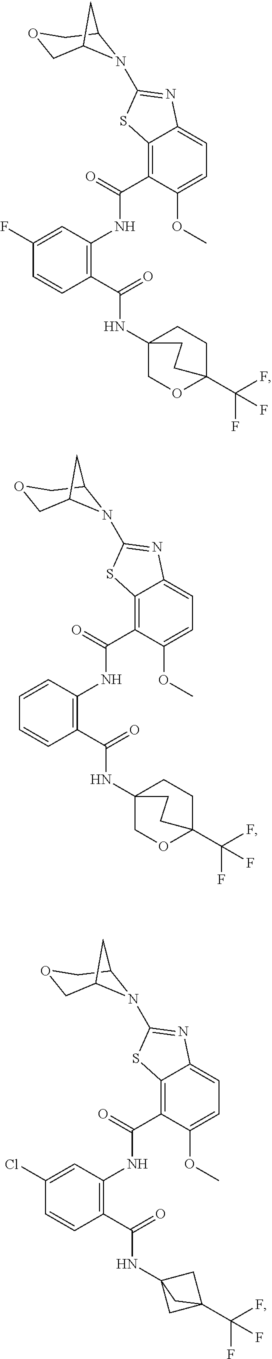 [SMILES: COc1ccc2nc(N3C4COCC3C4)sc2c1C(=O)Nc1cc(F)ccc1C(=O)NC12CCC(C(F)(F)F)(CC1)OC2 ; COc1ccc2nc(N3C4COCC3C4)sc2c1C(=O)Nc1ccccc1C(=O)NC12CCC(C(F)(F)F)(CC1)OC2 ; COc1ccc2nc(N3C4COCC3C4)sc2c1C(=O)Nc1cc(Cl)ccc1C(=O)NC12CC(C(F)(F)F)(C1)C2] 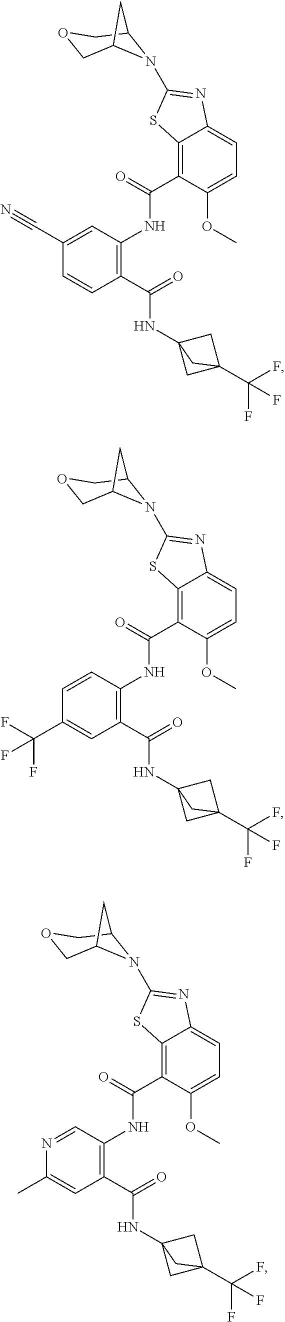 [SMILES: COc1ccc2nc(N3C4COCC3C4)sc2c1C(=O)Nc1cc(C#N)ccc1C(=O)NC12CC(C(F)(F)F)(C1)C2 ; COc1ccc2nc(N3C4COCC3C4)sc2c1C(=O)Nc1ccc(C(F)(F)F)cc1C(=O)NC12CC(C(F)(F)F)(C1)C2 ; COc1ccc2nc(N3C4COCC3C4)sc2c1C(=O)Nc1cnc(C)cc1C(=O)NC12CC(C(F)(F)F)(C1)C2] 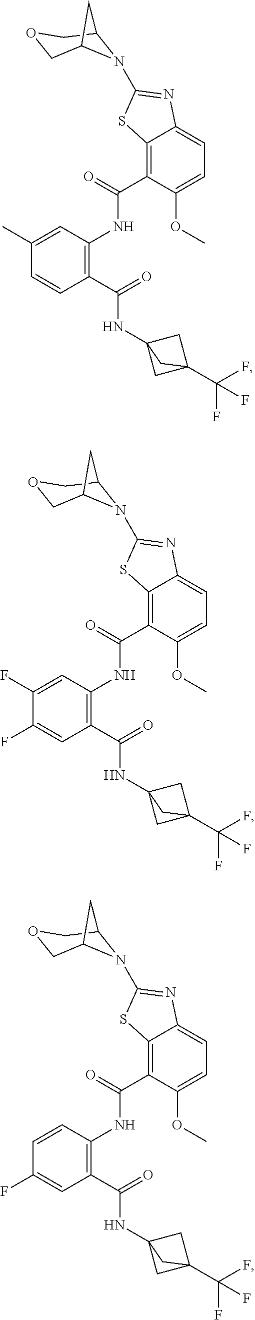 [SMILES: COc1ccc2nc(N3C4COCC3C4)sc2c1C(=O)Nc1cc(C)ccc1C(=O)NC12CC(C(F)(F)F)(C1)C2 ; COc1ccc2nc(N3C4COCC3C4)sc2c1C(=O)Nc1cc(F)c(F)cc1C(=O)NC12CC(C(F)(F)F)(C1)C2 ; COc1ccc2nc(N3C4COCC3C4)sc2c1C(=O)Nc1ccc(F)cc1C(=O)NC12CC(C(F)(F)F)(C1)C2] 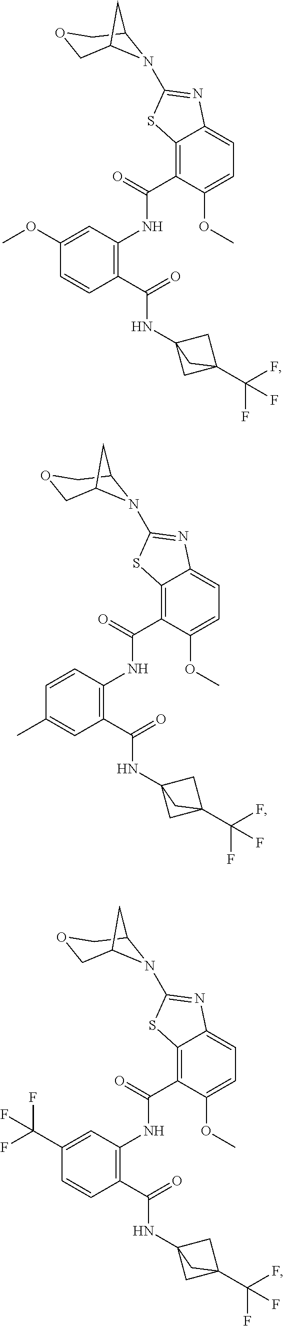 [SMILES: COc1ccc(C(=O)NC23CC(C(F)(F)F)(C2)C3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CC(C(F)(F)F)(C1)C2 ; COc1ccc2nc(N3C4COCC3C4)sc2c1C(=O)Nc1cc(C(F)(F)F)ccc1C(=O)NC12CC(C(F)(F)F)(C1)C2] 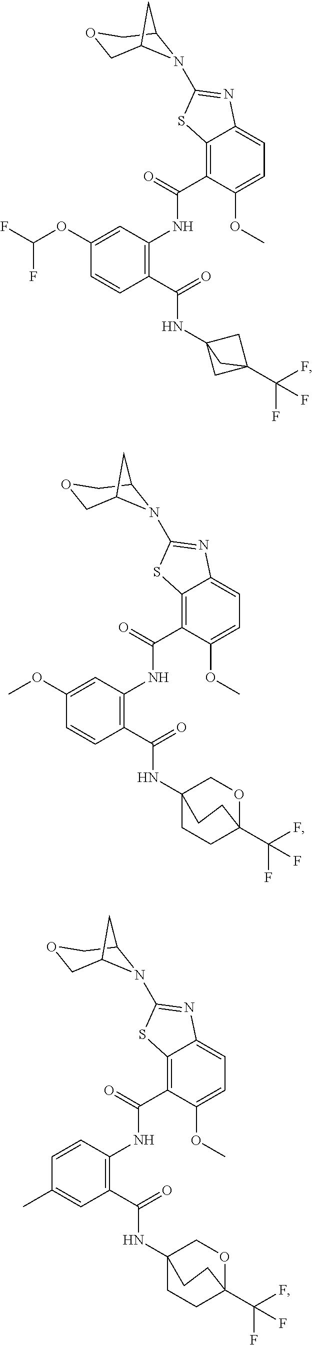 [SMILES: COc1ccc2nc(N3C4COCC3C4)sc2c1C(=O)Nc1cc(OC(F)F)ccc1C(=O)NC12CC(C(F)(F)F)(C1)C2 ; COc1ccc(C(=O)NC23CCC(C(F)(F)F)(CC2)OC3)c(NC(=O)c2c(OC)ccc3nc(N4C5COCC4C5)sc23)c1 ; COc1ccc2nc(N3C4COCC3C4)sc2c1C(=O)Nc1ccc(C)cc1C(=O)NC12CCC(C(F)(F)F)(CC1)OC2] 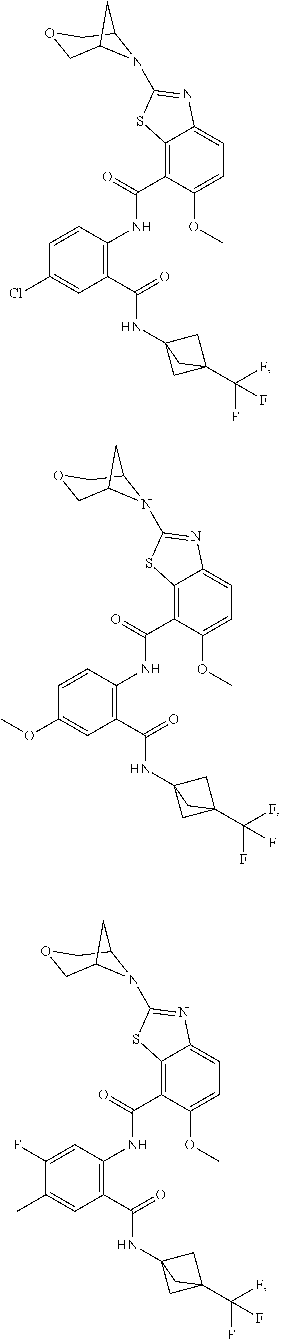 [SMILES: COc1ccc2nc(N3C4COCC3C4)sc2c1C(=O)Nc1ccc(Cl)cc1C(=O)NC12CC(C(F)(F)F)(C1)C2 ; COc1ccc(NC(=O)c2c(OC)ccc3nc(N4C5COCC4C5)sc23)c(C(=O)NC23CC(C(F)(F)F)(C2)C3)c1 ; COc1ccc2nc(N3C4COCC3C4)sc2c1C(=O)Nc1cc(F)c(C)cc1C(=O)NC12CC(C(F)(F)F)(C1)C2] 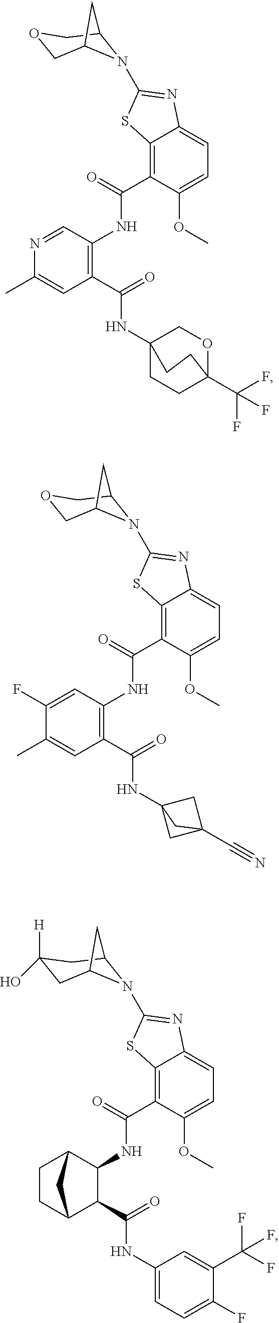 [SMILES: COc1ccc2nc(N3C4COCC3C4)sc2c1C(=O)Nc1cnc(C)cc1C(=O)NC12CCC(C(F)(F)F)(CC1)OC2 ; COc1ccc2nc(N3C4COCC3C4)sc2c1C(=O)Nc1cc(F)c(C)cc1C(=O)NC12CC(C#N)(C1)C2 ; COc1ccc2nc(N3C4CC(O)CC3C4)sc2c1C(=O)N[C@@H]1[C@H]2CC[C@H](C2)[C@@H]1C(=O)Nc1ccc(F)c(C(F)(F)F)c1] 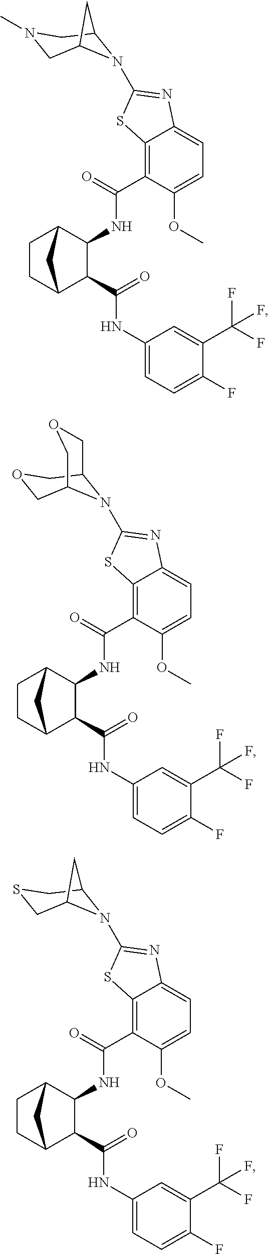 [SMILES: COc1ccc2nc(N3C4CC3CN(C)C4)sc2c1C(=O)N[C@@H]1[C@H]2CC[C@H](C2)[C@@H]1C(=O)Nc1ccc(F)c(C(F)(F)F)c1 ; COc1ccc2nc(N3C4COCC3COC4)sc2c1C(=O)N[C@@H]1[C@H]2CC[C@H](C2)[C@@H]1C(=O)Nc1ccc(F)c(C(F)(F)F)c1 ; COc1ccc2nc(N3C4CSCC3C4)sc2c1C(=O)N[C@@H]1[C@H]2CC[C@H](C2)[C@@H]1C(=O)Nc1ccc(F)c(C(F)(F)F)c1] 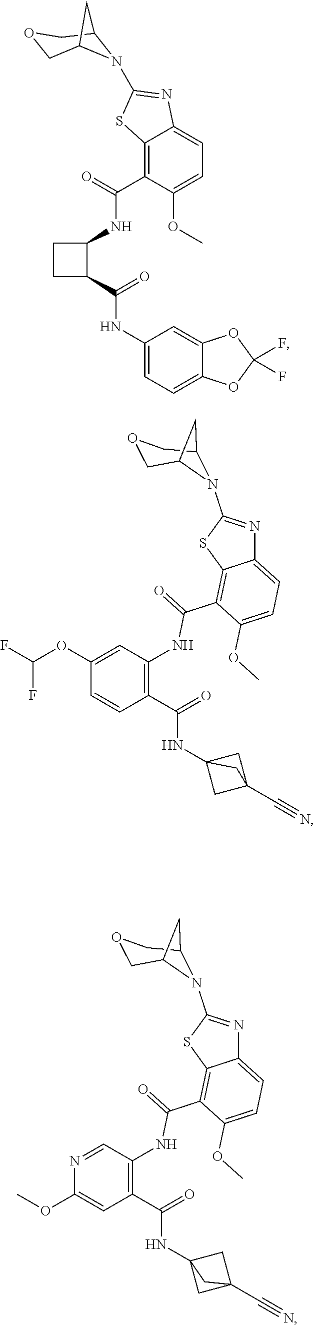 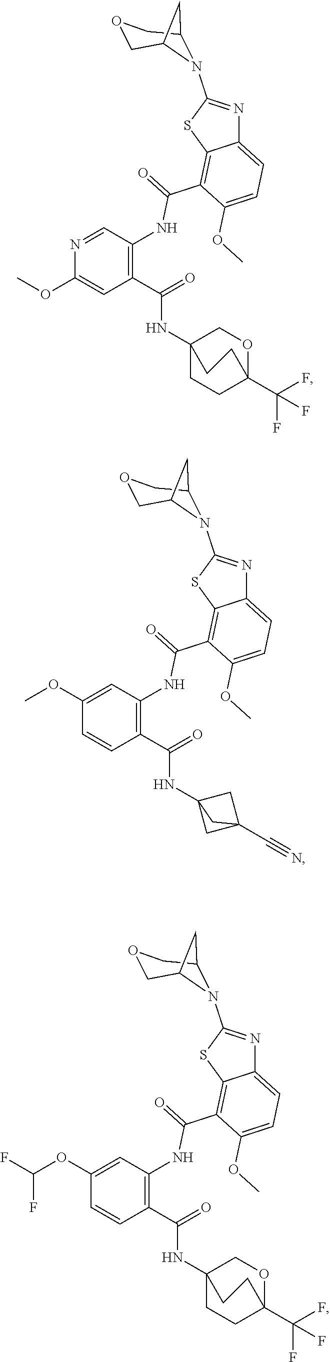 [SMILES: COc1cc(C(=O)NC23CCC(C(F)(F)F)(CC2)OC3)c(NC(=O)c2c(OC)ccc3nc(N4C5COCC4C5)sc23)cn1 ; COc1ccc(C(=O)NC23CC(C#N)(C2)C3)c(NC(=O)c2c(OC)ccc3nc(N4C5COCC4C5)sc23)c1 ; COc1ccc2nc(N3C4COCC3C4)sc2c1C(=O)Nc1cc(OC(F)F)ccc1C(=O)NC12CCC(C(F)(F)F)(CC1)OC2] 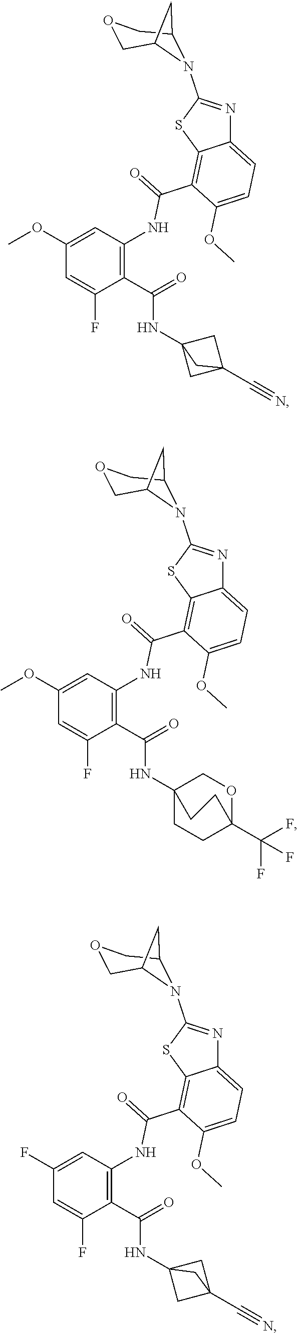 [SMILES: COc1cc(F)c(C(=O)NC23CC(C#N)(C2)C3)c(NC(=O)c2c(OC)ccc3nc(N4C5COCC4C5)sc23)c1 ; COc1cc(F)c(C(=O)NC23CCC(C(F)(F)F)(CC2)OC3)c(NC(=O)c2c(OC)ccc3nc(N4C5COCC4C5)sc23)c1 ; COc1ccc2nc(N3C4COCC3C4)sc2c1C(=O)Nc1cc(F)cc(F)c1C(=O)NC12CC(C#N)(C1)C2] 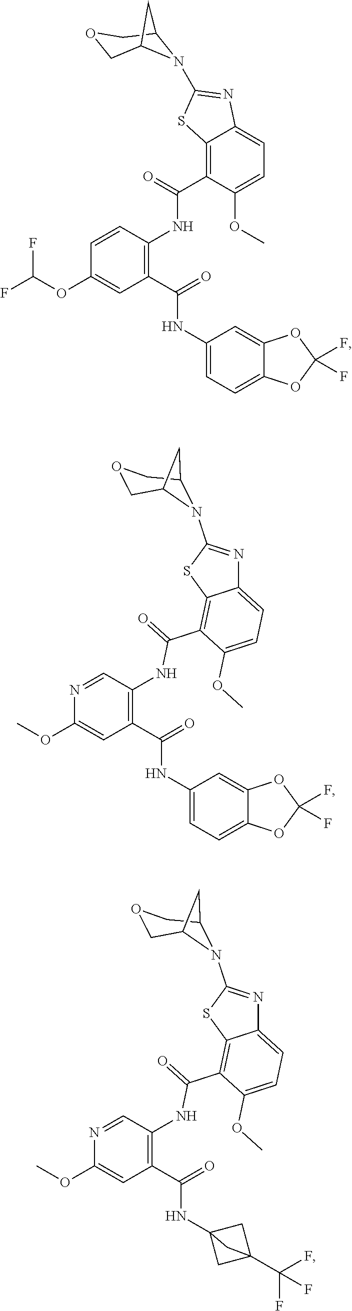 [SMILES: COc1ccc2nc(N3C4COCC3C4)sc2c1C(=O)Nc1ccc(OC(F)F)cc1C(=O)Nc1ccc2c(c1)OC(F)(F)O2 ; COc1cc(C(=O)Nc2ccc3c(c2)OC(F)(F)O3)c(NC(=O)c2c(OC)ccc3nc(N4C5COCC4C5)sc23)cn1 ; COc1cc(C(=O)NC23CC(C(F)(F)F)(C2)C3)c(NC(=O)c2c(OC)ccc3nc(N4C5COCC4C5)sc23)cn1] 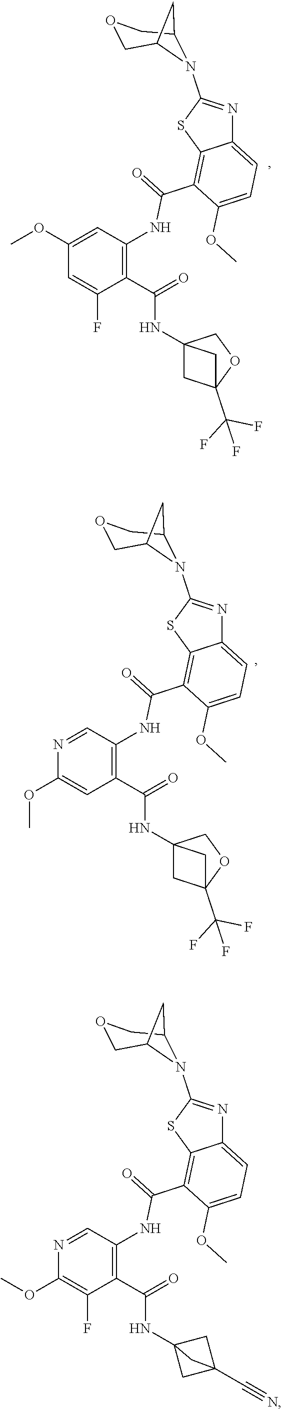 [SMILES: COc1cc(F)c(C(=O)NC23COC(C(F)(F)F)(C2)C3)c(NC(=O)c2c(OC)ccc3nc(N4C5COCC4C5)sc23)c1 ; COc1cc(C(=O)NC23COC(C(F)(F)F)(C2)C3)c(NC(=O)c2c(OC)ccc3nc(N4C5COCC4C5)sc23)cn1 ; COc1ccc2nc(N3C4COCC3C4)sc2c1C(=O)Nc1cnc(OC)c(F)c1C(=O)NC12CC(C#N)(C1)C2] 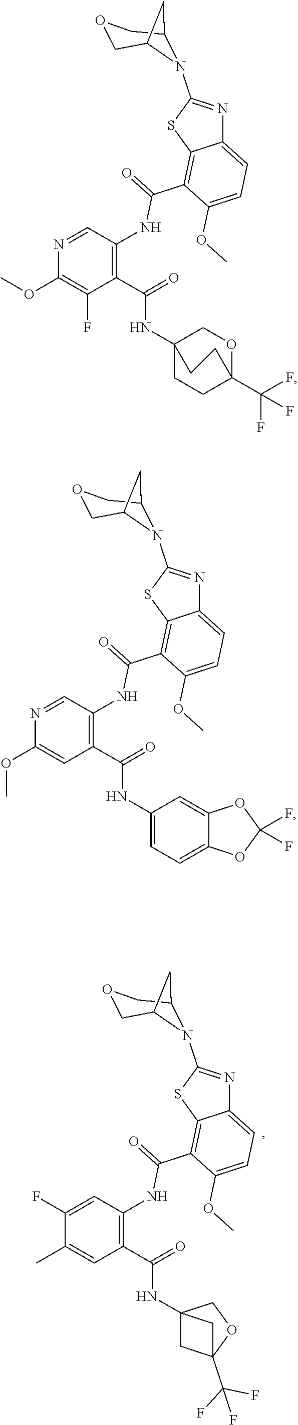 [SMILES: COc1ccc2nc(N3C4COCC3C4)sc2c1C(=O)Nc1cnc(OC)c(F)c1C(=O)NC12CCC(C(F)(F)F)(CC1)OC2 ; COc1cc(C(=O)Nc2ccc3c(c2)OC(F)(F)O3)c(NC(=O)c2c(OC)ccc3nc(N4C5COCC4C5)sc23)cn1 ; COc1ccc2nc(N3C4COCC3C4)sc2c1C(=O)Nc1cc(F)c(C)cc1C(=O)NC12COC(C(F)(F)F)(C1)C2] 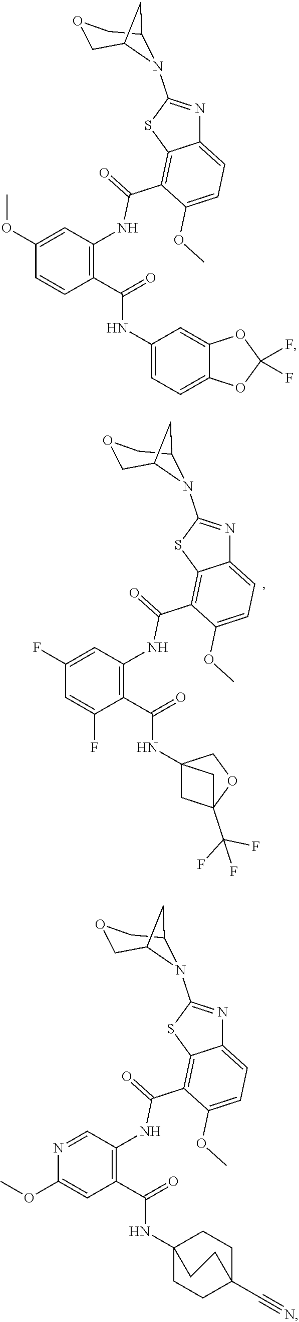 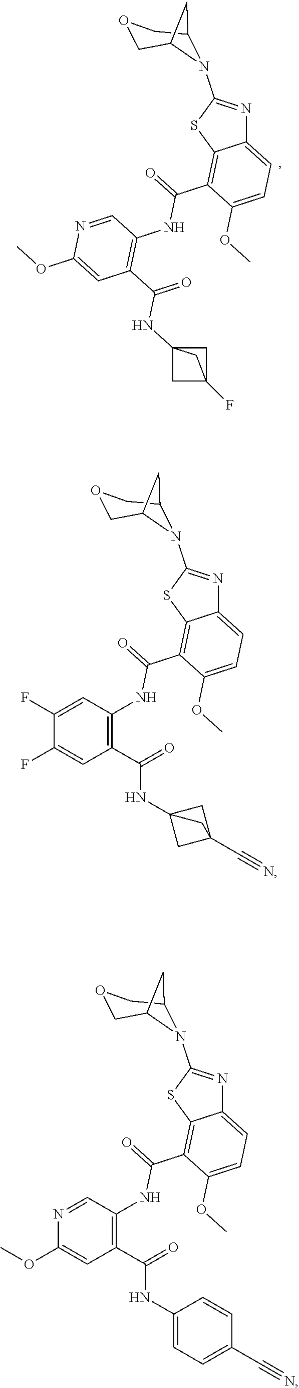 [SMILES: COc1cc(C(=O)NC23CC(F)(C2)C3)c(NC(=O)c2c(OC)ccc3nc(N4C5COCC4C5)sc23)cn1 ; COc1ccc2nc(N3C4COCC3C4)sc2c1C(=O)Nc1cc(F)c(F)cc1C(=O)NC12CC(C#N)(C1)C2 ; COc1cc(C(=O)Nc2ccc(C#N)cc2)c(NC(=O)c2c(OC)ccc3nc(N4C5COCC4C5)sc23)cn1] 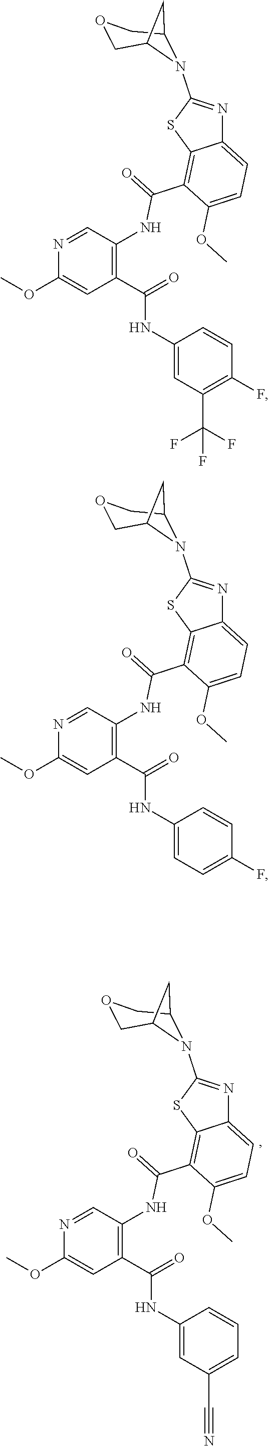 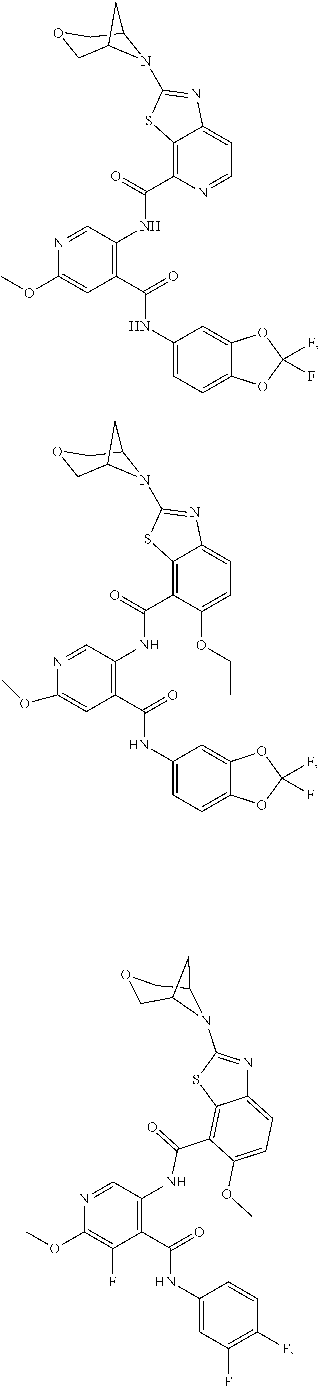 [SMILES: COc1cc(C(=O)Nc2ccc3c(c2)OC(F)(F)O3)c(NC(=O)c2nccc3nc(N4C5COCC4C5)sc23)cn1 ; CCOc1ccc2nc(N3C4COCC3C4)sc2c1C(=O)Nc1cnc(OC)cc1C(=O)Nc1ccc2c(c1)OC(F)(F)O2 ; COc1ccc2nc(N3C4COCC3C4)sc2c1C(=O)Nc1cnc(OC)c(F)c1C(=O)Nc1ccc(F)c(F)c1] 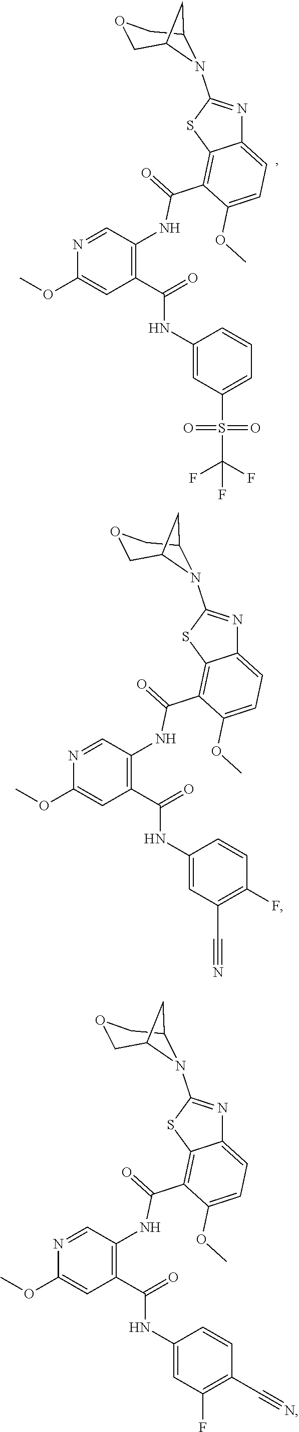 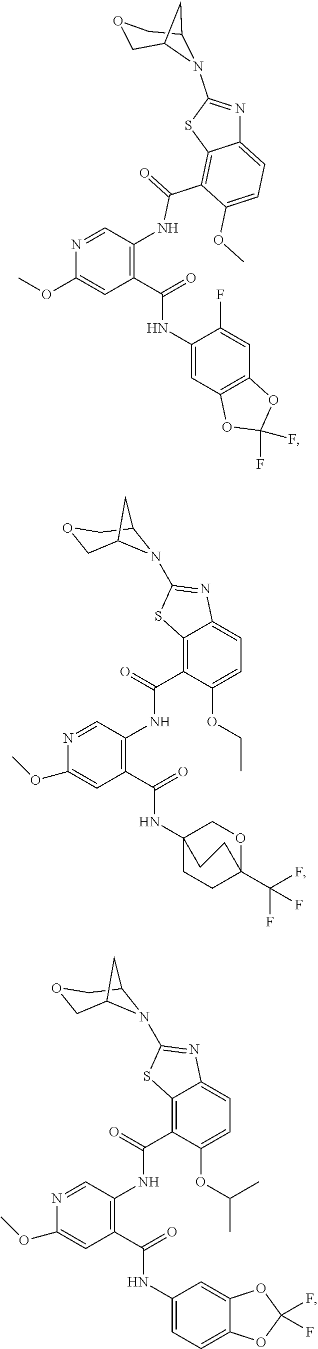 [SMILES: COc1cc(C(=O)Nc2cc3c(cc2F)OC(F)(F)O3)c(NC(=O)c2c(OC)ccc3nc(N4C5COCC4C5)sc23)cn1 ; CCOc1ccc2nc(N3C4COCC3C4)sc2c1C(=O)Nc1cnc(OC)cc1C(=O)NC12CCC(C(F)(F)F)(CC1)OC2 ; COc1cc(C(=O)Nc2ccc3c(c2)OC(F)(F)O3)c(NC(=O)c2c(OC(C)C)ccc3nc(N4C5COCC4C5)sc23)cn1] 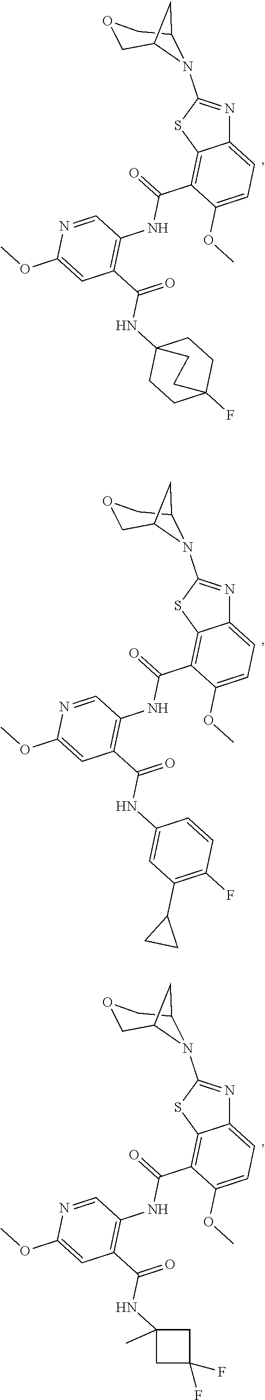 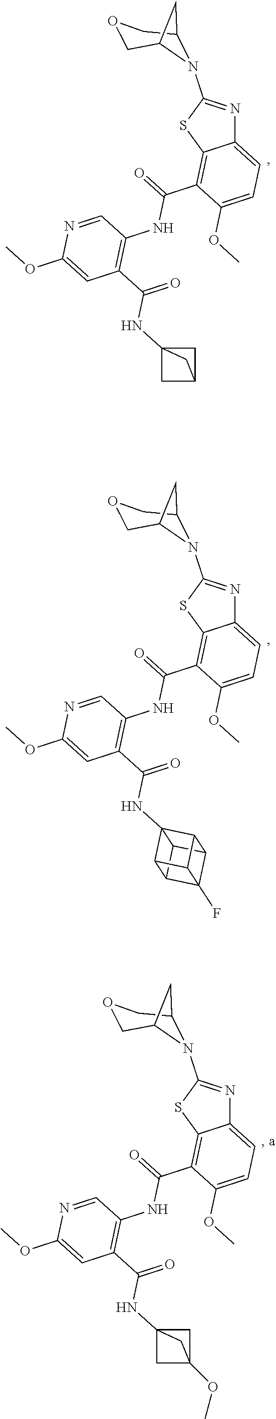 [SMILES: COc1cc(C(=O)NC23CC(C2)C3)c(NC(=O)c2c(OC)ccc3nc(N4C5COCC4C5)sc23)cn1 ; COc1cc(C(=O)NC23C4C5C2C2C3C4C52F)c(NC(=O)c2c(OC)ccc3nc(N4C5COCC4C5)sc23)cn1 ; COc1cc(C(=O)NC23CC(OC)(C2)C3)c(NC(=O)c2c(OC)ccc3nc(N4C5COCC4C5)sc23)cn1] 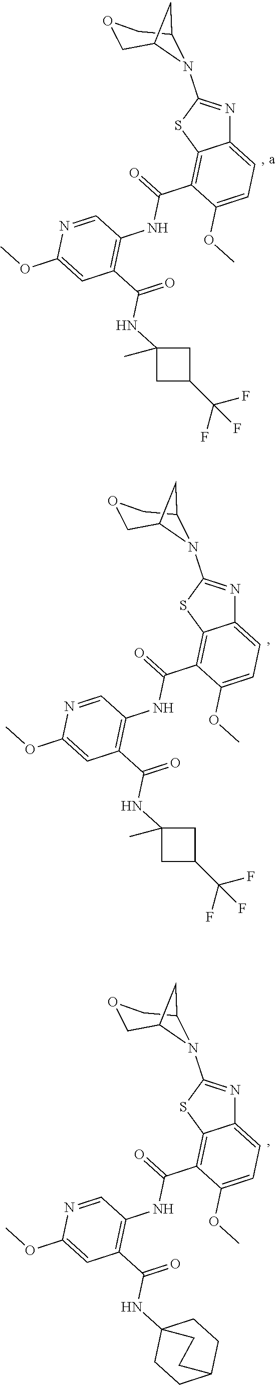 [SMILES: COc1cc(C(=O)NC2(C)CC(C(F)(F)F)C2)c(NC(=O)c2c(OC)ccc3nc(N4C5COCC4C5)sc23)cn1 ; COc1cc(C(=O)NC2(C)CC(C(F)(F)F)C2)c(NC(=O)c2c(OC)ccc3nc(N4C5COCC4C5)sc23)cn1 ; COc1cc(C(=O)NC23CCC(CC2)CC3)c(NC(=O)c2c(OC)ccc3nc(N4C5COCC4C5)sc23)cn1] 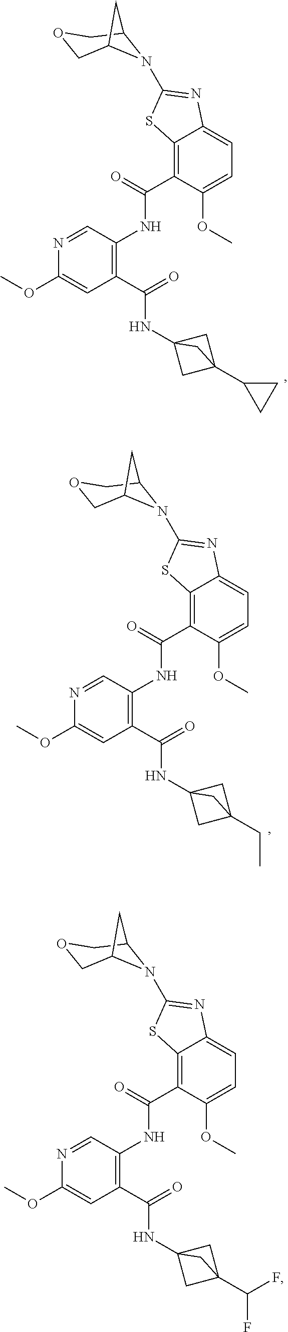 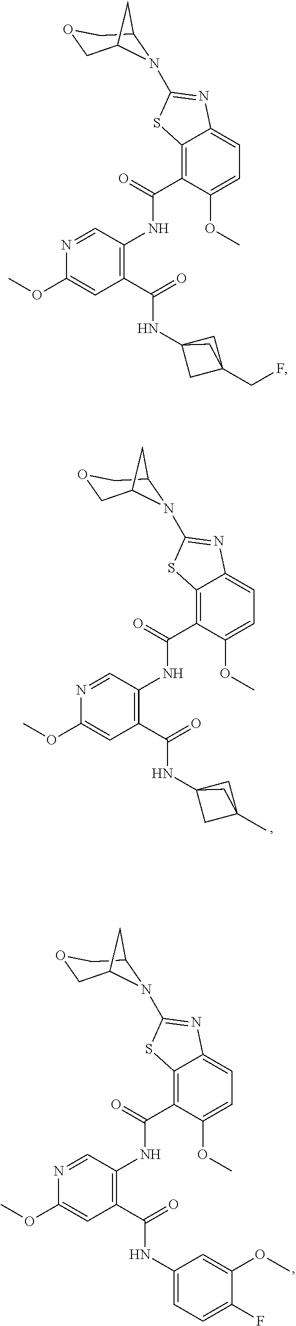 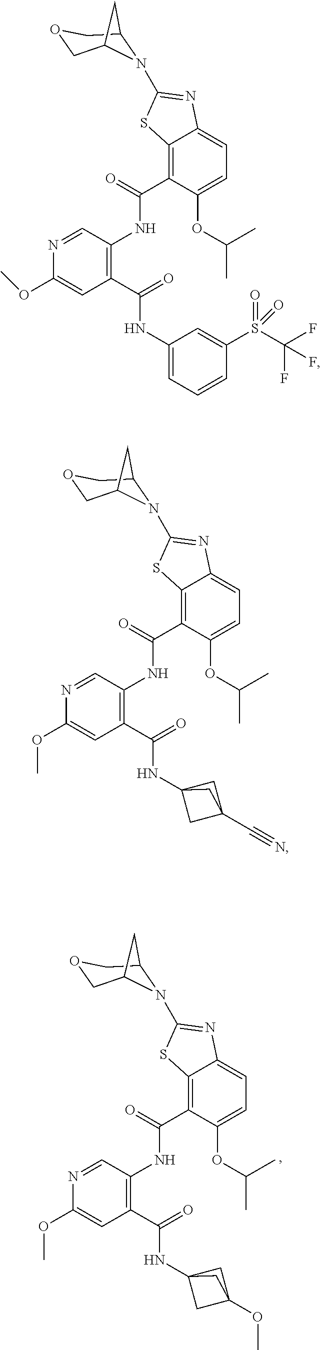 [SMILES: COc1cc(C(=O)Nc2cccc(S(=O)(=O)C(F)(F)F)c2)c(NC(=O)c2c(OC(C)C)ccc3nc(N4C5COCC4C5)sc23)cn1 ; COc1cc(C(=O)NC23CC(C#N)(C2)C3)c(NC(=O)c2c(OC(C)C)ccc3nc(N4C5COCC4C5)sc23)cn1 ; COc1cc(C(=O)NC23CC(OC)(C2)C3)c(NC(=O)c2c(OC(C)C)ccc3nc(N4C5COCC4C5)sc23)cn1] 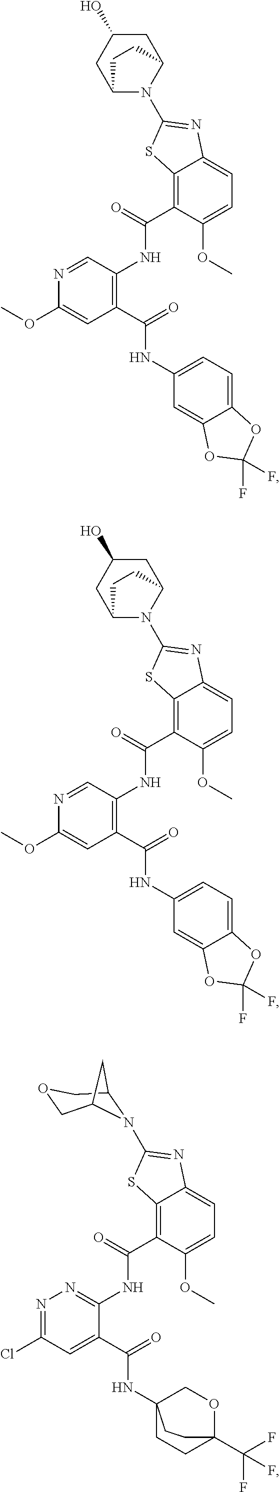 [SMILES: COc1cc(C(=O)Nc2ccc3c(c2)OC(F)(F)O3)c(NC(=O)c2c(OC)ccc3nc(N4[C@@H]5CC[C@H]4C[C@@H](O)C5)sc23)cn1 ; COc1cc(C(=O)Nc2ccc3c(c2)OC(F)(F)O3)c(NC(=O)c2c(OC)ccc3nc(N4[C@@H]5CC[C@H]4C[C@H](O)C5)sc23)cn1 ; COc1ccc2nc(N3C4COCC3C4)sc2c1C(=O)Nc1nnc(Cl)cc1C(=O)NC12CCC(C(F)(F)F)(CC1)OC2] 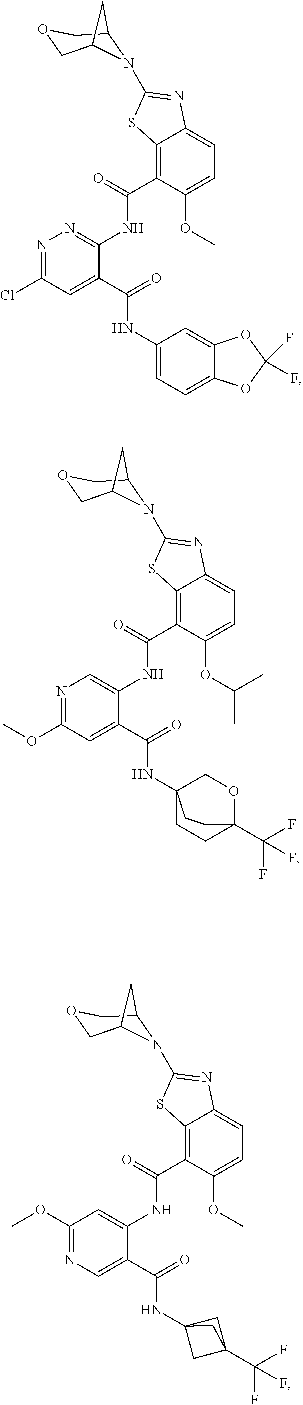 [SMILES: COc1ccc2nc(N3C4COCC3C4)sc2c1C(=O)Nc1nnc(Cl)cc1C(=O)Nc1ccc2c(c1)OC(F)(F)O2 ; COc1cc(C(=O)NC23CCC(C(F)(F)F)(CC2)OC3)c(NC(=O)c2c(OC(C)C)ccc3nc(N4C5COCC4C5)sc23)cn1 ; COc1cc(NC(=O)c2c(OC)ccc3nc(N4C5COCC4C5)sc23)c(C(=O)NC23CC(C(F)(F)F)(C2)C3)cn1] 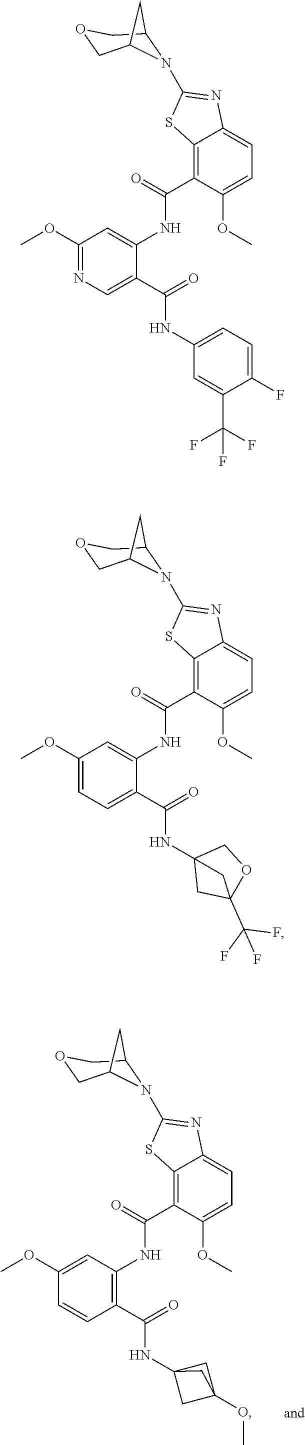 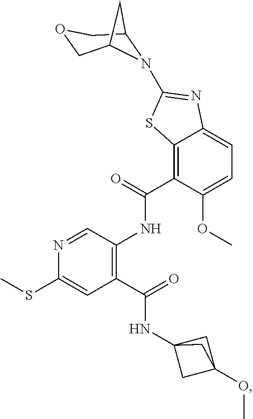
or a pharmaceutically acceptable salt thereof.

22. A pharmaceutical composition comprising a compound according to claim 1, or a pharmaceutically acceptable salt thereof, and a pharmaceutically acceptable carrier, diluent, or excipient.

23. A method of treating cardiovascular, pulmonary and/or renal conditions, diseases, and/or disorders in an individual, the method comprising administering to the individual an effective amount of a compound of claim 1.

24. A method according to claim 23, wherein the disease or disorder to be treated is a cardiovascular condition, disease, or disorder selected from acute heart failure, chronic heart failure, atherosclerosis, coronary artery disease, diabetes, stroke, hypercholesterolemia, hypertension, ischemia, vasoconstriction, or ventricular hypertrophy.

25. A method according to claim 23, wherein the disease or disorder to be treated is a pulmonary condition, disease, or disorder selected from pulmonary hypertension or chronic obstructive pulmonary disease (COPD).

26. A method according to claim 23, wherein the disease or disorder to be treated is a renal condition, disease, or disorder selected from acute kidney disease, chronic kidney disease, or diabetes nephropathy.
